(19) United States
(12) Patent Application Publication
(10) Pub. No.: US 2025/0341513 A1
(43) Pub. Date: Nov. 6, 2025

(54) Bordetella T Cells Epitopes, Megapools and Uses Thereof

(71) Applicant: LA JOLLA INSTITUTE FOR IMMUNOLOGY, La Jolla, CA (US)

(72) Inventors: Alessandro Sette, La Jolla, CA (US); Ricardo Da Silva Antunes, La Jolla, CA (US)

(21) Appl. No.: 18/873,483

(22) PCT Filed: Jun. 28, 2023
(86) PCT No.: PCT/US2023/069274

§ 371 Date: Dec. 10, 2024

Related U.S. Application Data
(60) Provisional application No. 63/356,446, filed on Jun. 28, 2022.
(60) Provisional application No. 63/451,520, filed on Mar. 10, 2023.

(51) Int. Cl. G01N 33/50; C07K 14/235; G01N 33/569

(52) U.S. Cl. CPC G01N 33/5091; C07K 14/235; G01N 33/56911; G01N 2333/235; G01N 2469/20

(57) ABSTRACT

The present invention includes compositions, including epitope megapools, and methods for detecting the presence of: a Bordetella or an immune response relevant to a Bordetella infection including T cells responsive to one or more Bordetella peptides or proteins comprising, consisting of, or consisting essentially of: one or more amino acid sequences, fusion proteins, a pool of 2 or more peptides, or polynucleotides that expression the amino acid sequences selected from those set forth in any one of Tables 1-20 (SEQ ID NOS: 1 to 2598). The invention further provides vaccines, diagnostics, therapies, and kits, comprising such proteins or peptides.

### CROSS-REFERENCE TO RELATED APPLICATIONS

This application claims priority to U.S. Provisional Application Ser. No. 63/356,446, filed Jun. 28, 2022, and U.S. Provisional Application Ser. No. 63/451,520, filed Mar. 10, 2023 the entire contents of which are incorporated herein by reference.

### STATEMENT OF GOVERNMENT SUPPORT

The inventions described in the present disclosure were made with government support under Contract No. 75N93019C00066, awarded by the National Institutes of Health. The government has certain rights in the invention.

### TECHNICAL FIELD OF THE INVENTION

The present invention relates in general to the field of proteins and peptides that are T cell epitopes and/or antigens for Bordetella, including epitopes and antigens from B. pertussis, and more particularly, to compositions and methods for the prevention, treatment, diagnosis, kits, and uses of such T cell epitopes and antigens, including megapools, for use in detecting and characterizing B. pertussis specific responses in infection and following vaccination.

### INCORPORATION-BY-REFERENCE OF MATERIALS FILED ON COMPACT DISC

The present application includes a Sequence Listing which has been submitted in ASCII format via EFS-Web and is hereby incorporated by reference in its entirety. Said ASCII copy, created on July ______, 2023, is named LJII2021.xml and is ______ bytes in size.

### BACKGROUND OF THE INVENTION

Without limiting the scope of the invention, its background is described in connection with the Gram-negative, aerobic, pathogenic, encapsulated coccobacillus of the genus Bordetella.

A need remains for identifying antigens and T cell epitopes for use in diagnostics, treatments, vaccines, kits, etc., for Bordetella related diseases and conditions, including whooping cough. There is additionally a specific need in the art for optimized megapools for use in detecting and characterizing B. pertussis specific responses in infection and following vaccination.

### SUMMARY OF THE INVENTION

In one embodiment, the present invention includes a composition comprising: one or more peptides or proteins, comprising, consisting of, or consisting essentially of an amino acid sequence selected from the sequences set forth in any one of Tables 1-20 (SEQ ID NOS: 1 to 2598), or a subsequence, portion, homologue, variant or derivative thereof, a fusion protein comprising one or more amino acid sequences selected from any one of those sequences set forth in Tables 1-20; a pool of 2 or more or more peptides comprising, consisting of, or consisting essentially of amino acid sequences selected from any one of those sequences set forth in Tables 1-20; or a polynucleotide that encodes one or more peptides or proteins, comprising, consisting of, or consisting essentially of an amino acid sequence selected from any one of those sequences set forth in Tables 1-20, or a subsequence, portion, homologue, variant or derivative thereof. In one aspect, the one or more peptides or proteins comprises, or wherein the fusion protein comprises 2 or more or more amino acid sequences selected from any one of those sequences set forth in Tables 1-20, or a subsequence, portion, homologue, variant or derivative thereof. In another aspect, the amino acid sequence is selected from a Bordetella T cell epitope selected from any one of those sequences set forth in Tables 1-20. In another aspect, the composition comprises one or more B. pertussis peptides amino acid sequences selected from any one of those sequences set forth in Tables 1-20, or a subsequence, portion, homologue, variant or derivative thereof; a fusion protein comprising one or more amino acid sequences selected from any one of those sequences set forth in Tables 1-20; or a pool of 2 or more peptides selected from any one of those sequences set forth in Tables 1-20. In another aspect, the peptide or protein comprises a Bordetella T cell epitope. In another aspect, the one or more peptides or proteins comprises a Bordetella CD8+ or CD4+ T cell epitope. In another aspect, the Bordetella is B. pertussis and the B. pertussis T cell epitope is not conserved in another Bordetella. In another aspect, the Bordetella is B. pertussis and the B. pertussis T cell epitope is conserved in another Bordetella. In another aspect, the one or more peptides or proteins has a length from about 9-15, 15-20, 20-25, 25-30, 30-40, 40-50, 50-75 or 75-100 amino acids. In another aspect, the one or more peptides or proteins elicits, stimulates, induces, promotes, increases or enhances a T cell response to a Bordetella. In another aspect, the one or more peptides or proteins that elicits, stimulates, induces, promotes, increases or enhances the T cell response to the Bordetella is a Bordetella protein or peptide, or a variant, homologue, derivative or subsequence thereof. In another aspect, the composition further comprises formulating the one or more peptides or proteins into an immunogenic formulation with an adjuvant. In another aspect, the adjuvant is selected from the group consisting of adjuvant is selected from the group consisting of alum, aluminum hydroxide, aluminum phosphate, calcium phosphate hydroxide, cytosine-guanosine oligonucleotide (CpG-ODN) sequence, granulocyte macrophage colony stimulating factor (GM-CSF), monophosphoryl lipid A (MPL), poly(I:C), MF59, Quil A, N-acetyl muramyl-L-alanyl-D-isoglutamine (MDP), FIA, montanide, poly (DL-lactide-coglycolide), squalene, virosome, AS03, ASO4, IL-1, IL-2, IL-3, IL-4, IL-5, IL-6, IL-7, IL-8, IL-10, IL-12, IL-15, IL-17, IL-18, STING, CD40L, pathogen-associated molecular patterns (PAMPs), damage-associated molecular pattern molecules (DAMPs), Freund's complete adjuvant, Freund's incomplete adjuvant, transforming growth factor (TGF)-beta antibody or antagonists, A2aR antagonists, lipopolysaccharides (LPS), Fas ligand, Trail, lymphotactin, Mannan (M-FP), APG-2, Hsp70 and Hsp90, pattern recognition receptor ligands, TLR3 ligands, TLR4 ligands, TLR5 ligands, TLR7/8 ligands, and TLR9 ligands. In another aspect, the composition further comprises a modulator of immune response. In another aspect, the modulator of immune response is a modulator of the innate immune response. In another aspect, the modulator is Interleukin-6 (IL-6), Interferon-gamma (IFN-γ), Transforming growth factor beta (TGF-β), or Interleukin-10 (IL-10), or an agonist or antagonist thereof.

In another embodiment, the present invention includes a composition comprising monomers or multimers of: peptides or proteins comprising, consisting of, or consisting essentially of: one or more amino acid sequences selected from any one of those sequences set forth in Tables 1-20, concatemers, subsequences, portions, homologues, variants or derivatives thereof, a fusion protein comprising one or more amino acid sequences selected from any one of those sequences set forth in Tables 1-20; or a polynucleotide that encodes one or more peptides or proteins, comprising, consisting of, or consisting essentially of an amino acid sequence selected from any one of those sequences set forth in Tables 1-20, or a subsequence, portion, homologue, variant or derivative thereof.

In another embodiment, the present invention includes a composition comprising one or more peptide-major histocompatibility complex (MHC) monomers or multimers, wherein the peptide-MHC monomer or multimer comprises a peptide comprising, consisting of, or consisting essentially of an amino acid sequence selected from any one of those sequences set forth in Tables 1-20, in a groove of the MHC monomer or multimer.

In another embodiment, the present invention includes a composition comprising: one or more peptides or proteins comprising, consisting of, or consisting essentially of an amino acid sequence selected from any one of those sequences set forth in Tables 1-20, or a subsequence, portion, homologue, variant or derivative thereof, a fusion protein comprising one or more amino acid sequences selected from any one of those sequences set forth in Tables 1-20; a pool of 2 or more peptides selected from any one of those sequences set forth in Tables 1-20; a polynucleotide that encodes one or more peptides or proteins, comprising, consisting of, or consisting essentially of an amino acid sequence selected from any one of those sequences set forth in Tables 1-20, or a subsequence, portion, homologue, variant or derivative thereof. In one aspect, the one or more peptides or proteins comprises, or wherein the fusion protein comprises, 2 or more amino acid sequences selected from any one of those sequences set forth in Tables 1-20, or a subsequence, portion, homologue, variant or derivative thereof. In another aspect, the protein or peptide comprises a B. pertussis T cell epitope. In another aspect, the one or more peptides or proteins comprises a B. pertussis CD8+ or CD4+ T cell epitope. In another aspect, the B. pertussis T cell epitope is not conserved in another Bordetella. In another aspect, the B. pertussis T cell epitope is conserved in another Bordetella. In another aspect, the one or more peptides or proteins has a length from about 9-15, 15-20, 20-25, 25-30, 30-40, 40-50, 50-75 or 75-100 amino acids. In another aspect, the one or more peptides or proteins elicits, stimulates, induces, promotes, increases or enhances a T cell response to B. pertussis. In another aspect, the one or more peptides or proteins that elicits, stimulates, induces, promotes, increases or enhances the T cell response to B. pertussis is a B. pertussis protein or peptide, or a variant, homologue, derivative or subsequence thereof. In another aspect, the composition further comprises formulating the one or more peptides or proteins into an immunogenic formulation with an adjuvant. In another aspect, the adjuvant is selected from the group consisting of adjuvant is selected from the group consisting of alum, aluminum hydroxide, aluminum phosphate, calcium phosphate hydroxide, cytosine-guanosine oligonucleotide (CpG-ODN) sequence, granulocyte macrophage colony stimulating factor (GM-CSF), monophosphoryl lipid A (MPL), poly(I:C), MF59, Quil A, N-acetyl muramyl-L-alanyl-D-isoglutamine (MDP), FIA, montanide, poly (DL-lactide-coglycolide), squalene, virosome, ASO3, ASO4, IL-1, IL-2, IL-3, IL-4, IL-5, IL-6, IL-7, IL-8, IL-10, IL-12, IL-15, IL-17, IL-18, STING, CD40L, pathogen-associated molecular patterns (PAMPs), damage-associated molecular pattern molecules (DAMPs), Freund's complete adjuvant, Freund's incomplete adjuvant, transforming growth factor (TGF)-beta antibody or antagonists, A2aR antagonists, lipopolysaccharides (LPS), Fas ligand, Trail, lymphotactin, Mannan (M-FP), APG-2, Hsp70 and Hsp90, pattern recognition receptor ligands, TLR3 ligands, TLR4 ligands, TLR5 ligands, TLR7/8 ligands, and TLR9 ligands. In another aspect, the composition further comprises a modulator of immune response. In another aspect, the modulator of immune response is a modulator of the innate immune response. In another aspect, the modulator is Interleukin-6 (IL-6), Interferon-gamma (IFN-γ), Transforming growth factor beta (TGF-β), or Interleukin-10 (IL-10), or an agonist or antagonist thereof.

In another embodiment, the present invention includes a composition comprising monomers or multimers of: one or more peptides or proteins comprising, consisting of, or consisting essentially of: one or more B. pertussis amino acid sequences selected from any one of those sequences set forth in Tables 1-20, concatemers, subsequences, portions, homologues, variants or derivatives thereof, a fusion protein comprising one or more amino acid sequences selected from any one of those sequences set forth in Tables 1-20; or a polynucleotide that encodes one or more peptides or proteins, comprising, consisting of, or consisting essentially of an amino acid sequence selected from any one of those sequences set forth in Tables 1-20, or a subsequence, portion, homologue, variant or derivative thereof.

In another embodiment, the present invention includes a composition comprising one or more peptide-major histocompatibility complex (MHC) monomers or multimers, wherein the peptide-MHC monomer or multimer comprises a peptide comprising, consisting of, or consisting essentially of an amino acid sequence selected from any one of those sequences set forth in Tables 1-20, in a groove of the (MHC) monomer or multimer.

In another embodiment, the present invention includes a method for detecting the presence of: (i) a Bordetella or (ii) an immune response relevant to Bordetella infections, vaccines or therapies, including T cells responsive to one or more Bordetella peptides, comprising: providing one or more proteins or peptides for detection of an amount or a relative amount of, and/or the activity of, and/or the state of antigen-specific T-cells; contacting a biological sample suspected of having Bordetella-specific T-cells to one or more proteins or peptides for detection; and detecting an amount or a relative amount of, and/or the activity of, and/or the state of antigen-specific T-cells in the biological sample, wherein the one or more proteins or peptides for detection comprise one or more amino acid sequences set forth in any one of Tables 1-20, or comprise a pool of 2 or more or more amino acid sequences set forth in any one of Tables 1-20. In one aspect, detecting the amount or a relative amount of, and/or activity of antigen-specific T-cells comprises one or more steps of identification or detection of the antigen-specific T-cells and measuring the amount of the antigen-specific T-cells. In another aspect, the one or more peptides or proteins comprises 2 or more amino acid sequences selected from any one of Tables 1-20. In another aspect, the detecting the amount or a relative amount of, and/or activity of antigen-specific T-cells comprises indirect detection and/or direct detection. In another aspect, the method of detecting an immune response relevant to the Bordetella comprises the following steps: providing an MHC monomer or an MHC multimer; contacting a population T-cells to the MHC monomer or MHC multimer; and measuring the number, activity or state of T-cells specific for the MHC monomer or MHC multimer. In one aspect, the MHC monomer or MHC multimer comprises a protein or peptide of the Bordetella. In another aspect, the protein or peptide comprises a CD8+ or CD4+ T cell epitope. In another aspect, the T cell epitope is not conserved in another Bordetella. In another aspect, the T cell epitope is conserved in another Bordetella. In another aspect, the protein or peptide has a length from about 9-15, 15-20, 20-25, 25-30, 30-40, 40-50, 50-75 or 75-100 amino acids. In another aspect, the proteins or peptides comprise 2 or more amino acid sequences selected from any one of those sequences set forth in Tables 1-20, or a subsequence, portion, homologue, variant or derivative thereof. In another aspect, the method further comprises detecting the presence or amount of the one or more peptides in a biological sample, or a response thereto, which is diagnostic of a Bordetella infection. In another aspect, the detecting an amount or a relative amount of, and/or the activity of, and/or the state of antigen-specific T-cells in the biological sample comprises measuring one or more of a cytokine or lymphokine secretion assay, T cell proliferation, immunoprecipitation, immunoassay, ELISA, radioimmunoassay, immunofluorescence assay, Western Blot, FACS analysis, a competitive immunoassay, a noncompetitive immunoassay, a homogeneous immunoassay a heterogeneous immunoassay, a bioassay, a reporter assay, a luciferase assay, a microarray, a surface plasmon resonance detector, a florescence resonance energy transfer, immunocytochemistry, or a cell mediated assay, or a cytokine proliferation assay. In another aspect, the method further comprises administering a treatment comprising the composition of one or more proteins, peptides or multimers to the subject from which the biological sample was drawn that increases the amount or relative amount of, and/or activity of the antigen-specific T-cells.

In another embodiment, the present invention includes a method for detecting the presence of: (i) B. pertussis or (ii) an immune response relevant to B. pertussis infections, vaccines or therapies, including T cells responsive to one or more B. pertussis peptides, comprising: providing one or more proteins or peptides for detection of an amount or a relative amount of, and/or the activity of, and/or the state of antigen-specific T-cells; contacting a biological sample suspected of having B. pertussis-specific T-cells to one or more proteins or peptides for detection; and detecting an amount or a relative amount of, and/or the activity of, and/or the state of antigen-specific T-cells in the biological sample, wherein the one or more proteins or peptides for detection comprise one or more amino acid sequences set forth in those sequences set forth in any one of Tables 1-20, or comprise a pool of 2 or more amino acid sequences set forth in those sequences set forth in any one of Tables 1-20. In one aspect, detecting the amount or a relative amount of, and/or activity of antigen-specific T-cells comprises one or more steps of identification or detection of the antigen-specific T-cells and measuring the amount of the antigen-specific T-cells. In another aspect, the one or more peptides or proteins comprises 2 or more amino acid sequences selected from any one of those sequences set forth in Tables 1-20. In another aspect, detecting the amount or a relative amount of, and/or activity of antigen-specific T-cells comprises indirect detection and/or direct detection. In another aspect, detecting an immune response relevant to B. pertussis comprises the following steps: providing an MHC monomer or an MHC multimer; contacting a population T-cells to the MHC monomer or MHC multimer; and measuring the number, activity or state of T-cells specific for the MHC monomer or MHC multimer. In another aspect, the MHC monomer or MHC multimer comprises a protein or peptide of B. pertussis. In another aspect, the protein or peptide comprises a B. pertussis CD8+ or CD4+ T cell epitope. In another aspect, the B. pertussis T cell epitope is not conserved in another Bordetella. In another aspect, the B. pertussis T cell epitope is conserved in another Bordetella. In another aspect, the protein or peptide has a length from about 9-15, 15-20, 20-25, 25-30, 30-40, 40-50, 50-75 or 75-100 amino acids. In another aspect, the proteins or peptides comprise 2 or more amino acid sequences selected from any one of those sequences set forth in Tables 1-20, or a subsequence, portion, homologue, variant or derivative thereof. In another aspect, the method further comprises detecting the presence or amount of the one or more peptides in a biological sample, or a response thereto, which is diagnostic of a B. pertussis infection. In another aspect, detecting an amount or a relative amount of, and/or the activity of, and/or the state of antigen-specific T-cells in the biological sample comprises measuring one or more of a cytokine or lymphokine secretion assay, T cell proliferation, immunoprecipitation, immunoassay, ELISA, radioimmunoassay, immunofluorescence assay, Western Blot, FACS analysis, a competitive immunoassay, a noncompetitive immunoassay, a homogeneous immunoassay a heterogeneous immunoassay, a bioassay, a reporter assay, a luciferase assay, a microarray, a surface plasmon resonance detector, a florescence resonance energy transfer, immunocytochemistry, or a cell mediated assay, or a cytokine proliferation assay. In another aspect, the method further comprises administering a treatment comprising the composition of one or more proteins, peptides or multimers to the subject from which the biological sample was drawn that increases the amount or relative amount of, and/or activity of the antigen-specific T-cells.

In another embodiment, the present invention includes a method detecting a Bordetella infection or exposure in a subject, the method comprising, consisting of, or consisting essentially of: contacting a biological sample from a subject with a composition of composition of one or more proteins, peptides or multimers; and determining if the composition elicits an immune response from the contacted cells, wherein the presence of an immune response indicates that the subject has been exposed to or infected with Bordetella. In one aspect, the sample comprises T cells. In another aspect, the response comprises inducing, increasing, promoting or stimulating anti-Bordetella activity of T cells. In another aspect, the T cells are CD8+ or CD4+ T cells. In another aspect, the method comprises determining whether the subject has been infected by or exposed to the Bordetella more than once by determining if the subject elicits a secondary T cell immune response profile that is different from a primary T cell immune response profile. In another aspect, the method further comprises diagnosing a Bordetella infection or exposure in a subject, the method comprising contacting a biological sample from a subject with a composition of composition of one or more proteins, peptides or multimers, and determining if the composition elicits a T cell immune response, wherein the T cell immune response identifies that the subject has been infected with or exposed to a Bordetella. In another aspect, the method is conducted three or more days following the date of suspected infection by or exposure to a Bordetella.

In another embodiment, the present invention includes a method detecting B. pertussis infection or exposure in a subject, the method comprising, consisting of, or consisting essentially of: contacting a biological sample from a subject with a composition of composition of one or more proteins, peptides or multimers; and determining if the composition elicits an immune response from the contacted cells, wherein the presence of an immune response indicates that the subject has been exposed to or infected with B. pertussis. In another aspect, the sample comprises T cells. In another aspect, the response comprises inducing, increasing, promoting or stimulating anti-B. pertussis activity of T cells. In another aspect, the T cells are CD8+ or CD4+ T cells. In another aspect, the method comprises determining whether the subject has been infected by or exposed to B. pertussis more than once by determining if the subject elicits a secondary T cell immune response profile that is different from a primary T cell immune response profile. In another aspect, the method further comprises diagnosing a B. pertussis infection or exposure in a subject, the method comprising contacting a biological sample from a subject with a composition of one or more proteins, peptides or multimers; and determining if the composition elicits a T cell immune response, wherein the T cell immune response identifies that the subject has been infected with or exposed to B. pertussis. In another aspect, the method is conducted three or more days following the date of suspected infection by or exposure to a Bordetella.

In another embodiment, the present invention includes a kit for the detection of Bordetella or an immune response to Bordetella in a subject comprising, consisting of or consisting essentially of: one or more T cells that specifically detect the presence of: one or more amino acid sequences selected from any one of those sequences set forth in Tables 1-20, or a subsequence, portion, homologue, variant or derivative thereof, or a fusion protein comprising one or more amino acid sequences selected from any one of those sequences set forth in Tables 1-20; or a pool of 2 or more or more peptides selected from the amino acid sequences set forth in any one of Tables 1-20. In one aspect, the one or more amino acid sequences are selected from a Bordetella T cell epitope set forth in any one of Tables 1-20. In another aspect, the composition comprises: one or more amino acid sequences selected from any one of those sequences set forth in Tables 1-20, or a subsequence, portion, homologue, variant or derivative thereof, a fusion protein comprising one or more amino acid sequences selected from any one of those sequences set forth in Tables 1-20; or a pool of 2 or more peptides selected from the amino acid sequences set forth in those sequences set forth in any one of Tables 1-20. In another aspect, the amino acid sequence comprises a Bordetella CD8+ or CD4+ T cell epitope. In another aspect, the T cell epitope is not conserved in another Bordetella. In another aspect, the T cell epitope is conserved in another Bordetella. In another aspect, the fusion protein has a length from about 9-15, 15-20, 20-25, 25-30, 30-40, 40-50, 50-75 or 75-100 amino acids. In another aspect, the kit includes instruction for a diagnostic method, a process, a composition, a product, a service or component part thereof for the detection of: (i) Bordetella or (ii) an immune response relevant to Bordetella infections, vaccines or therapies, including T cells responsive to Bordetella. In another aspect, the kit includes reagents for detecting an amount or a relative amount of, and/or the activity of, and/or the state of antigen-specific T-cells in the biological sample comprises measuring one or more of a cytokine or lymphokine secretion assay, T cell proliferation, immunoprecipitation, immunoassay, ELISA, radioimmunoassay, immunofluorescence assay, Western Blot, FACS analysis, a competitive immunoassay, a noncompetitive immunoassay, a homogeneous immunoassay a heterogeneous immunoassay, a bioassay, a reporter assay, a luciferase assay, a microarray, a surface plasmon resonance detector, a florescence resonance energy transfer, immunocytochemistry, or a cell mediated assay, or a cytokine proliferation assay. In another aspect, the kit includes reagents for determining a Human Leukocyte Antigen (HLA) profile of a subject, and selecting peptides that are presented by the HLA profile of the subject for detecting an immune response to Bordetella.

In another embodiment, the present invention includes a kit for the detection of B. pertussis or an immune response to B. pertussis in a subject comprising, consisting of or consisting essentially of: one or more T cells that specifically detect the presence of: one or more amino acid sequences selected from any one of those sequences set forth in Tables 1-20, or a subsequence, portion, homologue, variant or derivative thereof; a fusion protein comprising one or more amino acid sequences selected from any one of those sequences set forth in Tables 1-20; or a pool of 2 or more peptides selected from the amino acid sequences set forth in those sequences set forth in any one of Tables 1-20. In another aspect, the amino acid sequence comprises a B. pertussis CD8+ or CD4+ T cell epitope. In another aspect, the B. pertussis T cell epitope is not conserved in another Bordetella. In another aspect, the B. pertussis T cell epitope is conserved in another Bordetella. In another aspect, the fusion protein has a length from about 9-15, 15-20, 20-25, 25-30, 30-40, 40-50, 50-75 or 75-100 amino acids. In another aspect, the kit includes instruction for a diagnostic method, a process, a composition, a product, a service or component part thereof for the detection of: (i) B. pertussis or (ii) an immune response relevant to B. pertussis infections, vaccines or therapies, including T cells responsive to B. pertussis. In another aspect, the kit includes reagents for detecting an amount or a relative amount of, and/or the activity of, and/or the state of antigen-specific T-cells in the biological sample comprises measuring one or more of a cytokine or lymphokine secretion assay, T cell proliferation, immunoprecipitation, immunoassay, ELISA, radioimmunoassay, immunofluorescence assay, Western Blot, FACS analysis, a competitive immunoassay, a noncompetitive immunoassay, a homogeneous immunoassay a heterogeneous immunoassay, a bioassay, a reporter assay, a luciferase assay, a microarray, a surface plasmon resonance detector, a florescence resonance energy transfer, immunocytochemistry, or a cell mediated assay, or a cytokine proliferation assay. In another aspect, the kit includes reagents for determining a Human Leukocyte Antigen (HLA) profile of a subject, and selecting peptides that are presented by the HLA profile of the subject for detecting an immune response to B. pertussis.

In another embodiment, the present invention includes a method of stimulating, inducing, promoting, increasing, or enhancing an immune response against a Bordetella in a subject, comprising: administering a composition of one or more proteins, peptides, multimers or a polynucleotide that expresses the protein, peptide or multimers, in an amount sufficient to stimulate, induce, promote, increase, or enhance an immune response against the Bordetella in the subject. In another aspect, the immune response provides the subject with protection against a Bordetella infection or pathology, or one or more physiological conditions, disorders, illnesses, diseases or symptoms caused by or associated with Bordetella infection or pathology. In another aspect, the immune response is specific to: one or more B. pertussis peptides selected from the amino acid sequences set forth in any one of Tables 1-20, or a subsequence, portion, homologue, variant or derivative thereof.

In another embodiment, the present invention includes a method of stimulating, inducing, promoting, increasing, or enhancing an immune response against B. pertussis in a subject, comprising: administering a composition of proteins, peptides, multimers or a polynucleotide that expresses the protein, peptide or multimers, in an amount sufficient to stimulate, induce, promote, increase, or enhance an immune response against B. pertussis in the subject. In one aspect, the immune response provides the subject with protection against a B. pertussis infection or pathology, or one or more physiological conditions, disorders, illnesses, diseases or symptoms caused by or associated with B. pertussis infection or pathology. In another aspect, the immune response is specific to: one or more B. pertussis peptides selected from the amino acid sequences set forth in those sequences set forth in any one of Tables 1-20, or a subsequence, portion, homologue, variant or derivative thereof.

In another embodiment, the present invention includes a method of stimulating, inducing, promoting, increasing, or enhancing an immune response against B. pertussis in a subject, comprising: administering to a subject an amount of a protein or peptide comprising, consisting of or consisting essentially of an amino acid sequence of a B. pertussis protein or peptide, or a variant, homologue, derivative or subsequence thereof, wherein the protein or peptide comprises at least two peptides selected from the amino acid sequences set forth in any one of Tables 1-20 or a subsequence, portion, homologue, variant or derivative thereof, in an amount sufficient to prevent, stimulate, induce, promote, increase, immunize against, or enhance an immune response against B. pertussis in the subject. In one aspect, the immune response provides the subject with protection against B. pertussis infection or pathology, or one or more physiological conditions, disorders, illnesses, diseases or symptoms caused by or associated with B. pertussis infection or pathology.

In another embodiment, the present invention includes a method of treating, preventing, or immunizing a subject against B. pertussis infection, comprising administering to a subject an amount of a protein or peptide comprising, consisting of, or consisting essentially of an amino acid sequence of a Bordetella protein or peptide, or a variant, homologue, derivative or subsequence thereof, wherein the protein or peptide comprises at least two amino acid sequences selected from any one of Tables 1-20 or a subsequence, portion, homologue, variant or derivative thereof, in an amount sufficient to treat, prevent, or immunize the subject for B. pertussis infection, wherein the protein or peptide comprises or consists of a Bordetella T cell epitope that elicits, stimulates, induces, promotes, increases, or enhances an anti-B. pertussis T cell immune response. In one aspect, the one or more amino acid sequences are selected from any one of those sequences set forth in Tables 1-20, or a subsequence, portion, homologue, variant or derivative thereof; a fusion protein comprising one or more amino acid sequences selected from any one of those sequences set forth in Tables 1-20; or a pool of 2 or more peptides selected from the amino acid sequences set forth in those sequences set forth in any one of Tables 1-20. In one aspect, the anti-B. pertussis T cell response is a CD8+, a CD4+ T cell response, or both. In another aspect, the T cell epitope is conserved across two or more clinical isolates of B. pertussis or two or more circulating forms of B. pertussis. In another aspect, the B. pertussis infection is an acute infection. In another aspect, the subject is a mammal or a human. In another aspect, the method reduces B. pertussis bacterial titer, increases or stimulates B. pertussis bacterial clearance, reduces or inhibits B. pertussis bacterial proliferation, reduces or inhibits increases in B. pertussis bacterial titer or B. pertussis bacterial proliferation, reduces the amount of a B. pertussis bacterial protein or the amount of a B. pertussis bacterial nucleic acid, or reduces or inhibits synthesis of a B. pertussis bacterial protein or a B. pertussis bacterial nucleic acid. In another aspect, the method reduces one or more adverse physiological conditions, disorders, illness, diseases, symptoms or complications caused by or associated with B. pertussis infection or pathology. In another aspect, the method improves one or more adverse physiological conditions, disorders, illness, diseases, symptoms or complications caused by or associated with B. pertussis infection or pathology. In another aspect, the symptom is fever or chills, cough, shortness of breath or difficulty breathing, fatigue, muscle or body aches, headache, new loss of taste or smell, sore throat, congestion or runny nose, nausea or vomiting, or diarrhea. In another aspect, the method reduces or inhibits susceptibility to B. pertussis infection or pathology. In another aspect, the protein or peptide, or a subsequence, portion, homologue, variant or derivative thereof, is administered prior to, substantially contemporaneously with or following exposure to or infection of the subject with B. pertussis. In another aspect, a plurality of B. pertussis T cell epitopes are administered prior to, substantially contemporaneously with or following exposure to or infection of the subject with B. pertussis. In another aspect, the protein or peptide, or a subsequence, portion, homologue, variant or derivative thereof is administered within 2-72 hours, 2-48 hours, 4-24 hours, 4-18 hours, or 6-12 hours after a symptom of B. pertussis infection or exposure develops. In another aspect, the protein or peptide, or a subsequence, portion, homologue, variant or derivative thereof is administered prior to exposure to or infection of the subject with B. pertussis. In another aspect, the method further comprises administering a modulator of immune response prior to, substantially contemporaneously with or following the administration to the subject of an amount of a protein or peptide. In another aspect, the modulator of immune response is a modulator of the innate immune response. In another aspect, the modulator is IL-6, IFN-γ, TGF-3, or IL-10, or an agonist or antagonist thereof.

In another embodiment, the present invention includes a method of treating, preventing, or immunizing a subject against B. pertussis infection, comprising administering to a subject the composition of one or more proteins, peptides or multimers in an amount sufficient to treat, prevent, or immunize the subject for B. pertussis infection. In one aspect, the B. pertussis infection is an acute infection. In another aspect, the method reduces B. pertussis bacterial titer, increases or stimulates B. pertussis bacterial clearance, reduces or inhibits B. pertussis bacterial proliferation, reduces or inhibits increases in B. pertussis bacterial titer or B. pertussis bacterial proliferation, reduces the amount of a B. pertussis bacterial protein or the amount of a B. pertussis bacterial nucleic acid, or reduces or inhibits synthesis of a B. pertussis bacterial protein or a B. pertussis bacterial nucleic acid. In another aspect, the method reduces one or more adverse physiological conditions, disorders, illness, diseases, symptoms or complications caused by or associated with B. pertussis infection or pathology. In another aspect, the method improves one or more adverse physiological conditions, disorders, illness, diseases, symptoms or complications caused by or associated with B. pertussis infection or pathology. In another aspect, the symptom is fever or chills, cough, shortness of breath or difficulty breathing, fatigue, muscle or body aches, headache, new loss of taste or smell, sore throat, congestion or runny nose, nausea, vomiting, or diarrhea. In another aspect, the method reduces or inhibits susceptibility to B. pertussis infection or pathology. In another aspect, the composition is administered prior to, substantially contemporaneously with or following exposure to or infection of the subject with B. pertussis. In another aspect, the composition is administered prior to, substantially contemporaneously with or following exposure to or infection of the subject with B. pertussis. In another aspect, the composition is administered within 2-72 hours, 2-48 hours, 4-24 hours, 4-18 hours, or 6-12 hours after a symptom of B. pertussis infection or exposure develops. In another aspect, the composition is administered prior to exposure to or infection of the subject with B. pertussis.

In another embodiment, the present invention includes a peptide or peptides that are immunoprevalent or immunodominant in a bacteria obtained by a method consisting of, or consisting essentially of: obtaining an amino acid sequence of the bacteria; determining one or more sets of overlapping peptides spanning one or more bacteria antigen using unbiased selection; synthesizing one or more pools of bacteria peptides comprising the one or more sets of overlapping peptides; combining the one or more pools of bacteria peptides with Class I major histocompatibility proteins (MHC), Class II MHC, or both Class I and Class II MHC to form peptide-MHC complexes; contacting the peptide-MHC complexes with T cells from subjects exposed to the bacteria; determining which pools triggered cytokine release by the T cells; and deconvoluting from the pool of peptides that elicited cytokine release by the T cells, which peptide or peptides are immunoprevalent or immunodominant in the pool. In one aspect, the bacteria is a Bordetella. In another aspect, the Bordetella is B. pertussis. In another aspect, the immunodominant peptides are selected from 1, 2 or more peptides selected from the amino acid sequences set forth in any one of Tables 1-20. In another aspect, the immunodominant peptides are selected from 1, 2 or more peptides selected from the amino acid sequences set forth in those sequences set forth in any one of Tables 1-20.

In another embodiment, the present invention includes a method of selecting an immunoprevalent or immunodominant peptide or protein of a bacteria comprising, consisting of, or consisting essentially of: obtaining an amino acid sequence of the bacteria; determining one or more sets of overlapping peptides spanning one or more bacteria antigen using unbiased selection; synthesizing one or more pools of bacteria peptides comprising the one or more sets of overlapping peptides; combining the one or more pools of bacteria peptides with Class I major histocompatibility proteins (MHC), Class II MHC, or both Class I and Class II MHC to form peptide-MHC complexes; contacting the peptide-MHC complexes with T cells from subjects exposed to the bacteria; determining which pools triggered cytokine release by the T cells; and deconvoluting from the pool of peptides that elicited cytokine release by the T cells, which peptide or peptides are immunoprevalent or immunodominant in the pool. In one aspect, the bacteria is a Bordetella. In another aspect, the Bordetella is B. pertussis. In another aspect, the immunodominant peptides are selected from 1, 2 or more peptides selected from the amino acid sequences set forth in any one of Tables 1-20. In another aspect, the immunodominant peptides are selected from 1, 2 or more peptides selected from the amino acid sequences set forth in those sequences set forth in any one of Tables 1-20.

In another embodiment, the present invention includes a polynucleotide that expresses one or more peptides or proteins, comprising, consisting of, or consisting essentially of an amino acid sequence selected from any one of those sequences set forth in Tables 1-20, or a subsequence, portion, homologue, variant or derivative thereof; a fusion protein comprising one or more amino acid sequences selected from any one of those sequences set forth in Tables 1-20; or a pool of 2 or more or more peptides comprising, consisting of, or consisting essentially of amino acid sequences selected from any one of those sequences set forth in Tables 1-20. In one aspect, the vector comprises the polynucleotide of claim that expresses one or more peptides or proteins, comprising, consisting of, or consisting essentially of an amino acid sequence selected from any one of those sequences set forth in Tables 1-20, or a subsequence, portion, homologue, variant or derivative thereof, a fusion protein comprising one or more amino acid sequences selected from any one of those sequences set forth in Tables 1-20; or a pool of 2 or more or more peptides comprising, consisting of, or consisting essentially of amino acid sequences selected from any one of those sequences set forth in Tables 1-20, a bacterial vector, or a host cell the comprises the same.

In another embodiment, the present invention includes a polynucleotide that expresses one or more peptides or proteins comprising, consisting of, or consisting essentially of an amino acid sequence selected from any one of those sequences set forth in Tables 1-20, or a subsequence, portion, homologue, variant or derivative thereof, a fusion protein comprising one or more amino acid sequences selected from any one of those sequences set forth in Tables 1-20; or a pool of 2 or more peptides selected from any one of those sequences set forth in Tables 1-20. In one aspect, the vector comprises the polynucleotide of claim that expresses one or more peptides or proteins comprising, consisting of, or consisting essentially of an amino acid sequence selected from any one of those sequences set forth in Tables 1-20, or a subsequence, portion, homologue, variant or derivative thereof, a fusion protein comprising one or more amino acid sequences selected from any one of those sequences set forth in Tables 1-20; or a pool of 2 or more peptides selected from any one of those sequences set forth in Tables 1-20, a bacterial vector, or a host cell that comprises the same.

### BRIEF DESCRIPTION OF THE DRAWINGS

For a more complete understanding of the features and advantages of the present invention, reference is now made to the detailed description of the invention along with the accompanying figures and in which:

FIGS. 1A-ID. Schematics of BP whole genome-wide library screening. An example of the entire BP peptide library screening and epitope identification for a representative individual donor using AIM assay is shown. (FIG. 1A) Screening of entire library organized in 133 pools of 188 15-mer peptides (MegaPools; MP). (FIG. 1B) Deconvolution of one positive representative MP (MP #39) into 8 pools of 22-24 individual peptides (MesoPools; MS). (FIG. 1C) Deconvolution of one positive representative MS (MS #39.7) for assessment of individual peptide response (n=24). (FIG. 1D) Overall map of CD4+ T cell reactivity showing the position of each individual epitope identified across the aligned BP genome, using the Tohama I and D420 BP strains as reference. Associated percentage of response (magnitude) for each pool/peptide is indicated in y axis. Dotted lines represent the cut-off value associated with the threshold of positivity (TP).

FIGS. 2A-2D. Large breadth of BP-specific CD4+ T cell responses in humans. (FIG. 2A) Dominance of epitope response across the entire cohort (n=40) indicated by proportion of donors who responded to the specified number of epitopes. (FIG. 2B) Dominance of antigen response indicated by proportion of donors who responded to the specified number of ORFs. (FIG. 2C) Breadth of epitope response ranked on the basis of % of total response (Black dashed line). Grey dotted lines indicate the top 50, 75 and 90 percent of total response and associated number of epitopes. (FIG. 2D) Breadth of antigen response ranked on the basis of % of total response (Black dashed line). Grey dotted lines indicate the top 50, 75 and 90 percent of total response and associated number of ORFs.

FIG. 3. Immunodominance of BP specific-CD4+ T cell responses. Overall map of CD4+ T cell responses by antigen (ORF) reactivity across the entire cohort (n=40) showing the position of each individual ORF identified across the aligned BP genome, using the Tohama I and D420 BP strains as reference. Associated percentage of total response (all antigens recognized) for each ORF is indicated in y axis. Each bar represents an individual ORF and annotation of specific antigens is shown (red—aP vaccine antigens; green—dominant non-aP vaccine antigens).

FIGS. 4A-4F. aP and non-aP vaccine antigens are similarly recognized in aP- and wP-primed donors. Graphs show comparison of responses between aP- and wP-primed donors in terms of (FIGS. 4A,4D) magnitude, (FIGS. 4B,4E) number of epitopes, and (FIGS. 4C,4F) number of ORFs for aP vaccine antigens (Grey triangles, upper panel) or non-aP vaccine antigens (Grey circles, bottom panel), respectively. Each symbol denotes an individual donor (n=40; 20 in each group). Bars represent geometric mean±geometric SD. p values calculated by Mann-Whitney statistical analysis are indicated.

FIGS. 5A-5L. Sequence conservation is not a major driver of immunogenicity. Peptide homology amongst different BP strains or Bordetella genus was evaluated for the entire se of peptides tested in this study. (FIG. 5A) percent of homology across different BP strains for non-reactive, subdominant or dominant non-aP vaccine derived peptides (FIG. 5B) percent of dominant peptides across different BP strains for peptide conservation in non-Ap vaccine derived peptides. (FIG. 5C) percent of variable peptides across different BP strains for non-reactive, subdominant or dominant non-Ap vaccine derived peptides. (FIG. 5D) percent of homology across different BP strains for non-reactive, subdominant or dominant aP vaccine derived peptides. (FIG. 5E) percent of dominant peptides across different BP strains for peptide conservation in aP vaccine derived peptides. (FIG. 5F) percent of conserved peptides across different BP strains for non-reactive, subdominant or dominant aP vaccine derived peptides. (FIG. 5G) percent of homology across different Bordetella for non-reactive, subdominant or dominant non-aP vaccine derived peptides. (FIG. 5H) percent of dominant peptides across different Bordetella for peptide conservation in non-aP vaccine derived peptides. (FIG. 5I) percent of variable peptides across different Bordetella for non-reactive, subdominant or dominant non-Ap vaccine derived peptides. (FIG. 5J) percent of homology across different Bordetella for non-reactive, subdominant or dominant aP vaccine derived peptides. (FIG. 5K) percent of dominant peptides across different Bordetella for peptide conservation in aP vaccine derived peptides. (FIG. 5L) percent of variable peptides across different Bordetella for non-reactive, subdominant or dominant aP vaccine derived peptides. p values calculated by Kruskal-Wallis test adjusted with Dunn's test for multiple comparisons are indicated.

FIGS. 6A-6C. Th2 polarization is specific to the aP vaccine antigens, in individuals originally primed with aP vaccine. Antigen specific CD4+ T cell responses from aP and non-aP vaccine antigens were detected with PT(E)VAC and PT(E)R peptide pools respectively. (FIG. 6A) percentage of AIM+(OX40+CD25+) CD4+ T cells after stimulation of PBMCs with peptide pools (n=20). (FIG. 6B) percentage of ICS+cytokine+ (CD154+) CD4+ T cells after stimulation of PBMCs with peptide pools (n=40). Black dotted lines represent the cut-off value associated with the threshold of positivity (TP) and percentage of donor recognition is indicated for each stimuli. (FIG. 6C) Polarization of CD4+ T cell responses represented as ratio of IFNγ/IL-4 cytokine response for each individual pool (n=40; 20 for aP and 20 for wP groups). Red dotted line indicates a Ratio=1. In all graphs, each circle represents a donor. Tick black lines represent geometric mean±geometric SD, and p values were calculated by Mann-Whitney.

FIGS. 7A-7C. non-aP vaccine antigen responses are not polarized as function of priming childhood vaccination. Antigen specific CD4+ T cell responses from 15 individual non-aP vaccine antigens were detected with overlapping (O) peptide pools and represented as the sum of all MPs responses (PT(0)1-15) or as each individual MP response (ANT1-ANT15). PT(E)VAC pool was used as control respectively. (FIG. 7A) percentage of AIM+ (OX40+CD25+) CD4+ T cells after stimulation of PBMCs with peptide pools (n=20). (FIG. 7B) percentage of ICS+cytokine+ (CD154+) CD4+ T cells after stimulation of PBMCs with peptide pools (PT(E)VAC, n=40; PT(O)1-15, n=20; ANT1, 3, 8, and 9, n=34; all other ANT, n=20). Black dotted lines represent the cut-off value associated with the threshold of positivity (TP) and percentage of donor recognition is indicated for each stimuli. (FIG. 7C) Polarization of CD4+ T cell responses represented as ratio of IFNγ/IL-4 cytokine response for each individual pool (n=34; 17 for aP and 17 for wP groups). Red dotted line indicates a Ratio=1. In all graphs, each circle represents a donor. Tick black lines represent geometric mean±geometric SD, and p values were calculated by Mann-Whitney.

FIG. 8. Schematic of BP genome-wide screening and summary of experimental design and strategy. CD4+ T cell reactivity spanning the entire BP proteome was assayed with a library of 24,877 peptides in 3 sequential steps, directly ex vivo using a high throughput Activation Induced Marker (AIM) assay flow cytometry methodology. Dot plots in the bottom right, show representative AIM+ CD4+ T cell responses after stimulation with a single peptide (red box) or with negative (DMSO) and positive (PHA) controls.

FIGS. 9A-9B. Immunodominance is associated with both magnitude and donor recognition. (FIG. 9A) Overall map of CD4+ T cell responses at antigen (ORF) level by percent of total magnitude (black bars, left axis) or percent of donor recognition (grey bars, right axis), across the entire cohort (n=40). Each bar represents an individual ORF identified across the aligned BP genome, using the Tohama I and D420 BP strains as reference. Dotted line represents a frequency of recognition of 5%. (FIG. 9B) Graph shows correlation between percentages of total magnitude and donor recognition. Each circle represents an individual ORF. R and p value expresses Spearman's rank correlation coefficient test.

FIGS. 10A-10B. Immunodominant most reactive antigens have the longest sequences. Graphs shows correlation between protein size (kDa) and (FIG. 10A) percent of total antigen reactivity or (FIG. 10B) percent of donor recognition. Each circle represents responses of each of the 15 immunodominant overlapping peptide pools tested by AIM assay across all donors (n=20). R and p values express Spearman's rank correlation coefficient test and the best fit is represented by a linear regression line (red).

FIG. 11. Illustrative flow cytometry gating strategy for the assessment of antigen-specific CD4+ T cell responses by AIM and ICS assays. Representative gating of reactive OX40+CD25+ and cytokine+(IFNγ, IL-2, TNFα and IL-4) CD154+ CD4+ T cells from donor PBMCs is shown. Briefly, for both AIM and ICS, mononuclear cells were gated out of all events followed by subsequent singlet gating. Live CD3+ cells were gated as Live/Dead−CD14−CD8−CD19-CD3+. Cells were then gated as CD4+CD3+. For AIM assay, antigen-specific cells were defined as OX40+CD25+ CD4+ T cells (AIM+) after antigen stimulation, and frequencies calculated as percent of total CD4+ T cells after background subtraction. For ICS assay, antigen-specific cytokine producing cells were defined as Cytokine+ and CD154+ CD4+ T cells after antigen stimulation, and frequencies calculated as percent of total CD4+ T cells after background subtraction.

FIGS. 12A-12C. Experimentally defined epitope pools (PT(E)VAC and PT(E)R) detect BP-specific responses in vaccinated subjects primed with acellular (aP) or whole-cell (wP) vaccines in childhood.

FIG. 13. Novel identified antigens have equal or higher reactivity than aP vaccine antigens.

FIG. 14. Overlapping peptide pools derived from the 19 most immunodominant antigens not contained in the aP vaccine, can be used to detect BP-specific responses.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention. Numerous variations, changes, and substitutions will now occur to those skilled in the art without departing from the invention. It should be understood that various alternatives to the embodiments of the invention described herein may be employed in practicing the invention. To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims. Unless specifically stated or obvious from context, as used herein, the term “or” is understood to be inclusive.

### Example 1

Bordetella pertussis (BP), the causative agent of whooping cough, infects human hosts' lungs and upper airways. The recent increase in cases of whooping cough in the US suggests that the current administered BP acellular (aP) vaccine, which replaced a whole-cell (wP) vaccine in the early 90s, might have limitations in the quality and effectiveness of protection. Previously, the inventors have demonstrated that detection and quantification of BP-specific CD4+ T cells to antigens currently included in aP vaccines can be easily and rapidly achieved with high sensitivity and specificity in a variety of different T cell assays through the use of a peptide pool to stimulate PBMCs (Dan et al., J Immunol 2016, da Silva Antunes et al., JCI 2018, Silva Antunes et al., Cytokine 2021). In particular, stemming from experimentally characterized epitopes (Bancroft et al., Cell Immunol 2016), a set of 132 peptides derived from aP vaccine antigens were defined [PT(E)VAC]. The antigens included are the filamentous hemagglutinin (FHA), pertactin (PRN), pertussis toxin (PT), and fimbrial proteins 2 and 3 (Fim2/3). Recently, the inventors have performed the first full genome screening of human T cell reactivity to BP to identify novel targets and provide knowledge towards the direction of a new and improved BP vaccine design (da Silva Antunes et al., JIR, 2020, and da Silva Antunes et al. in preparation). The study readily and unbiasedly detected reactivity to epitopes and antigens currently included in the aP vaccine and, in addition, identified many other epitopes and antigens not included in aP vaccines. Importantly, the inventors have identified 19 antigens with reactivity as prominent or even more immunodominant than the aP vaccine antigens. These discoveries enabled the unique opportunity to generate epitope pools to characterize and discriminate responses specific to aP vaccine antigens from other immunodominant and immunogenic BP antigens not contained in aP vaccines. Specifically, and in addition to the [PT(E)VAC]epitope pool (Table 1), the inventors generated a new epitope pool of 170 experimentally defined peptides covering the most immunogenic peptides across the entire BP genome, and not containing aP vaccine antigens [PT(E)R](Table 2). In parallel, to establish the patterns of immunodominance of different BP antigens, the inventors further generated 19 sets of BP-specific CD4+ T cells epitopes pools using overlapping peptides covering the entire sequence of the novel antigens. The inventors tested the sensitivity and performance of the BP-specific peptide pools after short-culture stimulation of PBMCs using activation induced marker (AIM) and intracellular cellular staining (ICS) assays. PBMCs were obtained from 2 distinct cohorts including aP- or wP-primed individuals in childhood. The previous and new epitope pools will allow studying BP-specific responses in aP vaccination or boost settings and in the context of whole-cell vaccination schemes, exposition/colonization, infection, and human challenge studies.

TABLE 1. PT(E)VAC—Acelullar vaccine B. pertussis Megapool—Peptides defined based on threshold of magnitude from the 5 known vaccine antigens responses.

TABLE 1
PT(E)VAC-Acelullar vaccine B. pertussis Megapool-
Peptides defined based on threshold of magnitude
from the 5 known vaccine antigens responses

| SEQ ID NO: | Sequence | Pool no. |
|---|---|---|
| 1 | GADLIIANPNGISVNG | 2 |
| 2 | VVARLVKLQGAVSSKQ | 2 |
| 3 | GKPLADIAVVAGANRY | 2 |
| 4 | VVAGANRYDHATRRAT | 2 |
| 5 | DHATRRATPIAAGARG | 2 |
| 6 | SSDSGLGVRQLGSLSS | 2 |
| 7 | RQLGSLSSPSAITVSS | 2 |
| 8 | GQVRATSAGAMTVRDV | 5 |
| 9 | GFLKSAGAMTVNGRDA | 5 |
| 10 | VRLDGAHAGGQLRVSS | 5 |
| 11 | VAELKSLDNISVTGGE | 5 |
| 12 | NISVTGGERVSVQSVN | 5 |
| 13 | RVSVQSVNSASRVAIS | 5 |
| 14 | IDVRGGSTVAANSLHA | 6 |
| 15 | RSMTLGIVDTTGDLQA | 7 |
| 16 | SASRARIDSTGSVGIG | 7 |
| 17 | KVAKKLFLNGTLRAVN | 8 |
| 18 | SVVSDAALVADGGPIV | 8 |
| 19 | QRIEAQRIENRGTFQS | 9 |
| 20 | AAQVTQRGGAANLTSR | 9 |
| 21 | HDTRFSNKIRLMGPLQ | 9 |
| 22 | IRLMGPLQVNAGGAVS | 10 |
| 23 | TSRGGFDNEGKMESNK | 10 |
| 24 | FTVQAQRIDNSGTMAA | 10 |
| 25 | PHLRNTGQVVAGHDIH | 11 |
| 26 | VVAGHDIHIINSAKLE | 11 |
| 27 | IINSAKLENTGRVDAR | 11 |
| 28 | NDIALDVADFTNTGSL | 11 |
| 29 | DFTNTGSLYAEHDATL | 11 |
| 30 | ILPVAEGTLRVKAKSL | 11 |
| 31 | LRVKAKSLTTEIETGN | 11 |
| 32 | PGSLIAEVQENIDNKQ | 11 |
| 33 | VANEANALLWAAGELT | 11 |
| 34 | LWAAGELTVKAQNITN | 11 |
| 35 | VKAQNITNKRAALIEA | 11 |
| 36 | AVALLNKLGRIRAGED | 12 |
| 37 | MHLDAPRIENTAKLSG | 12 |
| 38 | GKKAGTIAAPWYGGDL | 12 |
| 39 | VGKDLYLNAGARKDEH | 12 |
| 40 | ELLDYLLDQNRYEYIW | 13 |
| 41 | QNRYEYIWGLYPTYTE | 13 |
| 42 | RGHTLESAEGRKIFGE | 13 |
| 43 | EGRKIFGEYKKLQGEY | 13 |
| 44 | GGMDAETKEVDGIIQE | 13 |
| 45 | EVDGIIQEFAADLRTV | 13 |
| 46 | FAADLRTVYAKQADQA | 13 |
| 47 | VAQRYKSQIDAVRLQA | 14 |
| 48 | IDAVRLQAIQPGRVTL | 14 |
| 49 | IQPGRVTLAKALSAAL | 14 |
| 50 | GAEIAFYPKEQTVLAA | 14 |
| 51 | GAIHNGENAAQNRGRP | 14 |
| 52 | DALAAVLVNPHIFTRI | 14 |
| 53 | NPHIFTRIGAAQTSLA | 14 |
| 54 | LASLASLDAAQGLEVS | 15 |
| 55 | AARVAGDNYFDTTLVR | 15 |
| 56 | GKPLADIAVIAGANRY | 24 |
| 57 | HADDGTIVITGTITDT | 25 |
| 58 | VQVRISNLNDSKITMG | 26 |
| 59 | TMRYLASYVKKNGDVE | 26 |
| 60 | EASAITTYVGFSVVYP | 26 |
| 61 | ANDGTIVITGSISDQT | 27 |
| 62 | FRLANLNGQHIRMGTD | 28 |
| 63 | SKSYTLRYLASYVKKP | 28 |
| 64 | DAAQITSYVGFSVVYP | 28 |
| 65 | TTLAMALGALGAAPAA | 29 |
| 66 | ALGAAPAAHADWNNQS | 29 |
| 67 | HADWNNQSIVKTGERQ | 29 |
| 68 | IVKTGERQHGIHIQGS | 29 |
| 69 | HGIHIQGSDPGGVRTA | 29 |
| 70 | GRQAQGILLENPAAEL | 29 |
| 71 | GIRRFLGTVTVKAGKL | 29 |
| 72 | VTVKAGKLVADHATLA | 29 |
| 73 | NVGDTWDDDGIALYVA | 29 |
| 74 | DGIALYVAGEQAQASI | 29 |
| 75 | GEQAQASIADSTLQGA | 29 |
| 76 | ADSTLQGAGGVQIERG | 29 |
| 77 | GGVQIERGANVTVQRS | 30 |
| 78 | ANVTVQRSAIVDGGLH | 30 |
| 79 | PEDLPPSRVVLRDTNV | 30 |
| 80 | VVLRDTNVTAVPASGA | 30 |
| 81 | TAVPASGAPAAVSVLG | 30 |
| 82 | GHITGGRAAGVAAMQG | 30 |
| 83 | AGVAAMQGAVVHLQRA | 30 |
| 84 | AVVHLQRATIRRGDAP | 30 |
| 85 | TIRRGDAPAGGAVPGG | 30 |
| 86 | AGGAVPGGAVPGGAVP | 30 |
| 87 | AVPGGAVPGGFGPGGF | 30 |
| 88 | GPVLDGWYGVDVSGSS | 30 |
| 89 | VELAQSIVEAPELGAA | 30 |
| 90 | EAPELGAAIRVGRGAR | 30 |
| 91 | SAPHGNVIETGGARRF | 31 |
| 92 | ETGGARRFAPQAAPLS | 31 |
| 93 | APQAAPLSITLQAGAH | 31 |
| 94 | AQGKALLYRVLPEPVK | 31 |
| 95 | SIGPLDVALASQARWT | 31 |
| 96 | LASQARWTGATRAVDS | 31 |
| 97 | GATRAVDSLSIDNATW | 31 |
| 98 | LSIDNATWVMTDNSNV | 31 |
| 99 | AEAGRFKVLTVNTLAG | 31 |
| 100 | LTVNTLAGSGLFRMNV | 31 |
| 101 | RNSGSEPASANTLLLV | 32 |
| 102 | PAGRELSAAANAAVNT | 32 |
| 103 | LFDDGIRRFLGTVTVK | 35 |
| 104 | AGGGVPGGAVPGGAVP | 35 |
| 105 | VYRYDSRPPEDVFQNG | 36 |
| 106 | NVLDHLTGRSCQVGSS | 36 |
| 107 | NSAFVSTSSSRRYTEV | 36 |
| 108 | SSRRYTEVYLEHRMQE | 36 |
| 109 | YLEHRMQEAVEAERAG | 36 |
| 110 | SYFEYVDTYGDNAGRI | 36 |
| 111 | YGDNAGRILAGALATY | 36 |
| 112 | LAGALATYQSEYLAHR | 36 |
| 113 | QSEYLAHRRIPPENIR | 37 |
| 114 | RIPPENIRRVTRVYHN | 37 |
| 115 | RVTRVYHNGITGETTT | 37 |
| 116 | GITGETTTTEYSNARY | 37 |
| 117 | PNPYTSRRSVASIVGT | 37 |
| 118 | WSERAGEAMVLVYYES | 37 |
| 119 | GEAMVLVYYESIAYSF | 37 |
| 120 | SVASIVGTLVRMAPVM | 37 |
| 121 | YYSNVTATRLLSSTNS | 39 |
| 122 | RLCAVFVRSGQPVIGA | 39 |
| 123 | SGQPVIGACTSPYDGK | 39 |
| 124 | CTSPYDGKYWSMYSRL | 39 |
| 125 | YWSMYSRLRKMLYLIY | 39 |
| 126 | RKMLYLIYVAGISVRV | 39 |
| 127 | HVSKEEQYYDYEDATF | 39 |
| 128 | TQHGSPYGRCANKTRA | 39 |
| 129 | HYYSKVTATRLLASTN | 41 |
| 130 | SRLCAVFVRDGQSVIG | 41 |
| 131 | VHVSKEEQYYDYEDAT | 41 |
| 132 | VYKYDSRPPEDVFONG | 44 |

TABLE 2. PT(E)R—Novel B. pertussis Megapool—Peptides defined based on the threshold of magnitude from novel and previously uncharacterized antigen responses.

TABLE 2
PT(E)R-Novel B. pertussis Megapool-Peptides defined based on the threshold of magnitude
from novel and previously uncharacterized antigen responses

| SEQ | | |
|---|---|---|
| ID | | |
| NO: | Peptide Sequence | ORF Description |
| 133 | MGVDGLRLDAVPYLV | NP_880087.1 malto-oligosyltrehalose trehalohydrolase [B. |
| | | pertussis Tohama I] |
| 134 | LMPEFISSLAIAGVD | NP_879836.1 D-alanyl-D-alanine carboxypeptidase [B. pertussis |
| | | Tohama I] |
| 135 | LSLVVQEINGPRLAT | NP_879836.1 D-alanyl-D-alanine carboxypeptidase [B. pertussis |
| | | Tohama I] |
| 136 | RLLFTPDAAARKWVP | NP_879836.1 D-alanyl-D-alanine carboxypeptidase [B. pertussis |
| | | Tohama I] |
| 137 | AMKRAMQNAMRLGA | NP_882129.1 30S ribosomal protein S3 [B. pertussis Tohama I] |
| | Q | |
| 138 | FTHIELLPVMAHPFG | NP_880086.1 1,4-alpha-glucan (glycogen) branching enzyme |
| 139 | IQRFVQAWPVVSSRL | NP_881595.1 cell division protein FtsQ [B. pertussis Tohama I] |
| 140 | QTPFHLVSSERSLTG | NP_879348.1 membrane protein insertase YidC [B. pertussis |
| | | Tohama I] |
| 141 | NGFITAYAHNRALLV | NP_880436.1 peptidase [B. pertussis Tohama I] |
| 142 | MEVFYGTVAVRVPLS | NP_882154.1 thiol:disulfide interchange protein [B. pertussis |
| | | Tohama I] |
| 143 | ALLRELRLRGVKQIG | NP_879836.1 D-alanyl-D-alanine carboxypeptidase [B. pertussis |
| | | Tohama I] |
| 144 | PWILWVHDLSVRDPF | NP_879348.1 membrane protein insertase YidC [B. pertussis |
| | | Tohama I] |
| 145 | ASVMKLVTTWAALSE | NP_879836.1 D-alanyl-D-alanine carboxypeptidase [B. pertussis |
| | | Tohama I] |
| 146 | FTGVVDLVKMKAIIW | NP_882120.1 elongation factor G [B. pertussis Tohama I] |
| 147 | IRSRIKVKSLNFMRG | NP_881388.1 hypothetical protein BP2788 [B. pertussis Tohama I] |
| 148 | VKRFGRFVGRRRNER | NP_882125.1 50S ribosomal protein L23 [B. pertussis Tohama I] |
| 149 | MMVRFFVSIGVLAWA | NP_879841.1 membrane protein [B. pertussis Tohama I] |
| 150 | SRAILLSTVLSVPTI | NP_882038.1 membrane protein [B. pertussis Tohama I] |
| 151 | QEYVFLTYALDSDVI | NP_881891.1 hypothetical protein BP3358 [B. pertussis Tohama I] |
| 152 | VVLALVRNALGVQQV | NP_880884.1 type III secretion system protein [B. pertussis |
| | | Tohama I] |
| 153 | GWLTIIAKPLFTLMT | NP_879348.1 membrane protein insertase YidC [B. pertussis |
| | | Tohama I] |
| 154 | AIAWLVVAARAWRR | NP_882241.1 membrane protein [B. pertussis Tohama I] |
| | R | |
| 155 | ENLKADLQRLMGVPV | NP_882129.1 30S ribosomal protein S3 [B. pertussis Tohama I] |
| 156 | IAYRLLTHNRALTLF | NP_880300.1 DNA mismatch repair protein MutS [B. pertussis |
| | | Tohama I] |
| 157 | QVLREVLGAMRYWL | NP_880087.1 malto-oligosyltrehalose trehalohydrolase [B. |
| | D | pertussis Tohama I] |
| 158 | VPSLSILAEPPVAVV | NP_879766.1 sulfate-binding protein [B. pertussis Tohama I] |
| 159 | SGGLKVTKTYTLHRG | NP_879348.1 membrane protein insertase YidC [B. pertussis |
| | | Tohama I] |
| 160 | TAVCFLLSLETAMRS | NP_879354.1 integrase [B. pertussis Tohama I] |
| 161 | VEVRPVRRLALAMRW | NP_882119.1 30S ribosomal protein S7 [B. pertussis Tohama I] |
| 162 | LPKAFSIYRDTRVWI | NP_881188.1 recombination-associated protein [B. pertussis |
| | | Tohama I] |
| 163 | HPTGFRLAVTRNWTS | NP_882129.1 30S ribosomal protein S3 [B. pertussis Tohama I] |
| 164 | RLMGLPHYHRLLYAL | NP_881082.1 membrane efflux protein [B. pertussis Tohama I] |
| 165 | TFTPIQVASIAALDG | NP_881385.1 aminotransferase [B. pertussis Tohama I] |
| 166 | MSRHAIRSTLAGLTL | NP_880438.1 exported protein [B. pertussis Tohama I] |
| 167 | HAGPAFKGNVTLAIE | NP_881862.1 inner membrane protein [B. pertussis Tohama I] |
| 168 | KAVFFPLAAASYRSM | NP_879348.1 membrane protein insertase YidC [B. pertussis |
| | | Tohama I] |
| 169 | GLPLTLILATFGIAL | NP_882327.1 amino acid ABC transporter permease [B. |
| | | pertussis Tohama I] |
| 170 | TPRLILEGATAHNLN | NP_881788.1 excinuclease ABC subunit [B. pertussis Tohama I] |
| 171 | MPYLNLIPNPAPPFV | NP_879614.1 hypothetical protein BP0799 [B. pertussis Tohama I] |
| 172 | ERRYIIAPRGLEVGA | NP_882126.1 50S ribosomal protein L2 [B. pertussis Tohama I] |
| 173 | DLKSILIIGAGPIII | NP_880195.1 carbamoyl phosphate synthase large subunit [B. |
| | | pertussis Tohama I] |
| 174 | IFGRDFNEALVHQIV | NP_882124.1 50S ribosomal protein L4 [B. pertussis Tohama I] |
| 175 | RIPWLDVIPGAIVTA | NP_882012.1 serum resistance protein [B. pertussis Tohama I] |
| 176 | CQMALMENAISRYLN | NP_881186.1 hypothetical protein BP2559 [B. pertussis Tohama I] |
| 177 | MKRTYQPSVTRRKRT | NP_879345.1 50S ribosomal protein L34 [B. pertussis Tohama I] |
| 178 | VRAVAGYVLGASGKR | NP_879836.1 D-alanyl-D-alanine carboxypeptidase [B. pertussis |
| | | Tohama I] |
| 179 | QAYLEYLYTPAAQEI | NP_879766.1 sulfate-binding protein [B. pertussis Tohama I] |
| 180 | GDSWGVLFSHPADFT | NP_879765.1 antioxidant protein [B. pertussis Tohama I] |
| 181 | LSSIALAESNALDKR | NP_882013.1 BrkA autotransporter [B. pertussis Tohama I] |
| 182 | QIRLPHLRDIGALLT | NP_882012.1 serum resistance protein [B. pertussis Tohama I] |
| 183 | KKVRLTITYPASTGR | NP_879765.1 antioxidant protein [B. pertussis Tohama I] |
| 184 | LRVVKVVYFDDPVDQ | NP_879664.1 NADH-quinone oxidoreductase subunit N [B. |
| | | pertussis Tohama I] |
| 185 | QRSILSLNGALILVL | NP_879664.1 NADH-quinone oxidoreductase subunit N [B. |
| | | pertussis Tohama I] |
| 186 | AGRGLFVSGTLARLM | NP_882010.1 NADH dehydrogenase [B. pertussis Tohama I] |
| 187 | QAKVMMIMPLVFGG | NP_879348.1 membrane protein insertase YidC [B. pertussis |
| | M | Tohama I] |
| 188 | DFTRLMHANGRIIAA | NP_881952.1 amidase [B. pertussis Tohama I] |
| 189 | EGVVVAPSRLKSLVI | NP_881684.1 hypothetical protein BP3118 [B. pertussis Tohama I] |
| 190 | SLNFMRGRTFLNKYL | NP_881388.1 hypothetical protein BP2788 [B. pertussis Tohama I] |
| 191 | MNAERLMQVILAPIV | NP_882125.1 50S ribosomal protein L23 [B. pertussis Tohama I] |
| 192 | LDGNGVFVLNTNVAA | NP_882013.1 BrkA autotransporter [B. pertussis Tohama I] |
| 193 | GWFTMKFVWPPLTKA | NP_881826.1 ATP synthase subunit B [B. pertussis Tohama I] |
| 194 | FSLYRLALSQPEYAS | NP_879836.1 D-alanyl-D-alanine carboxypeptidase [B. pertussis |
| | | Tohama I] |
| 195 | TLGLYHYRHRRVPDY | NP_879666.1 TonB-dependent receptor BfrD [B. pertussis |
| | | Tohama I] |
| 196 | GMPITRFLNAVRVAL | NP_881737.1 GntR family transcriptional regulator [B. pertussis |
| | | Tohama I] |
| 197 | NNIRGILKTTAVKAP | NP_882014.1 molecular chaperone GroEL [B. pertussis Tohama I] |
| 198 | KAMLEDIAILTGGTV | NP_882014.1 molecular chaperone GroEL [B. pertussis Tohama I] |
| 199 | DYVVRLHSKLTPASL | NP_879346.1 ribonuclease P protein component [B. pertussis |
| | | Tohama I] |
| 200 | SKIAIAMTKAFLAVN | NP_881559.1 ribonucleotide-diphosphate reductase subunit |
| | | alpha [B. pertussis Tohama I] |
| 201 | KSKYAIDWSPFLGAK | NP_879903.1 2-oxoglutarate dehydrogenase complex subunit E1 |
| | | [B. pertussis Tohama I] |
| 202 | DLLWVRLNWARLRR | NP_879541.1 phospholipase [B. pertussis Tohama I] |
| | H | |
| 203 | LVCSLLNLLPPAWII | NP_881921.1 inner membrane protein [B. pertussis Tohama I] |
| 204 | GLTWFAVLGSHAALQ | NP_880533.1 hypothetical protein BP1830 [B. pertussis Tohama I] |
| 205 | VGRIYRADAKQKKML | NP_879878.1 amino acids binding protein [B. pertussis |
| | | Tohama I] |
| 206 | PFVRDGNLLYISGQV | NP_880545.1 hypothetical protein BP1843 [B. pertussis Tohama I] |
| 207 | GYTLLFVAVTSAINQ | NP_880586.1 exported protein [B. pertussis Tohama I] |
| 208 | LDMLLFDRSGHRAAL | NP_880332.1 LysR family transcriptional regulator [B. pertussis |
| | | Tohama I] |
| 209 | MKRYHVDANQARRV | NP_879857.1 exopolyphosphatase [B. pertussis Tohama I] |
| | R | |
| 210 | EKIYVVQTSISVVQR | NP_881682.1 modification methylase [B. pertussis Tohama I] |
| 211 | VRRLGVIAINTALEF | NP_880701.1 acyl-CoA transferase [B. pertussis Tohama I] |
| 212 | FLIGILLILVFGVQL | NP_882302.1 transport system permease [B. pertussis Tohama I] |
| 213 | EVWLAADSSKFQRQA | NP_881259.1 glycerol-3-phosphate regulon repressor protein [B. |
| | | pertussis Tohama I] |
| 214 | MSWHWIFLINIPIGI | NP_882322.1 hypothetical protein BP3824 [B. pertussis Tohama I] |
| 215 | SLLAMLARAHDSPLT | NP_882289.1 type IV secretion system protein PtIC [B. pertussis |
| | | Tohama I] |
| 216 | MCDFYAVRAENSTLW | NP_880653.1 4-aminobutyrate aminotransferase [B. pertussis |
| | | Tohama I] |
| 217 | PDKFDIVVPSLSILA | NP_879766.1 sulfate-binding protein [B. pertussis Tohama I] |
| 218 | VWGPASLIVSKPIWN | NP_880562.1 exported protein [B. pertussis Tohama I] |
| 219 | LGRLGLRFGRRIALA | NP_882013.1 BrkA autotransporter [B. pertussis Tohama I] |
| 220 | LFPIFADLTGRRVLV | NP_879840.1 siroheme synthase [B. pertussis Tohama I] |
| 221 | AHWDFLAAMASADL | NP_880700.1 LysR family transcriptional regulator [B. pertussis |
| | G | Tohama I] |
| 222 | AERYRFFSYGDAMFI | NP_879835.1 S-adenosylmethionine -tRNA ribosyltransferase- |
| | | isomerase [B. pertussis Tohama I] |
| 223 | AGIQIFQLAETLQSL | NP_881678.1 MerR family transcriptional regulator [B. pertussis |
| | | Tohama I] |
| 224 | AAMLIEIKSRMLLPV | NP_882320.1 hypothetical protein BP3822 [B. pertussis Tohama I] |
| 225 | RPRLLAMIRSVRDHM | NP_879389.1 transcriptional regulator [B. pertussis Tohama I] |
| 226 | QAQLRSIEAAIATYR | NP_879580.1 cyclolysin secretion protein CyaD [B. pertussis |
| | | Tohama I] |
| 227 | GEGYVFYENRAYGVA | NP_879578.1 bifunctional hemolysin-adenylate cyclase [B. |
| | | pertussis Tohama I] |
| 228 | RVAFDGAEPRLVDTS | NP_880233.1 threonine--tRNA ligase [B. pertussis Tohama I] |
| 229 | ANRFLRRLWALGYAQ | NP_880711.1 leucine--tRNA ligase [B. pertussis Tohama I] |
| 230 | IGRDYASQIAAVRVV | NP_882331.1 cell division protein FtsX [B. pertussis Tohama I] |
| 231 | LPVMLLADSGLAADR | NP_880325.1 hypothetical protein BP1592 [B. pertussis Tohama I] |
| 232 | IRIDLRNIRSPIIVF | NP_879799.1 hypothetical protein BP1005 [B. pertussis Tohama I] |
| 233 | TGLFIVACAAFTPVT | NP_882322.1 hypothetical protein BP3824 [B. pertussis Tohama I] |
| 234 | WLFLKMLEKGIAYRK | NP_880711.1 leucine--tRNA ligase [B. pertussis Tohama I] |
| 235 | GQIDMMFAQLPAVLP | NP_882177.1 exported protein [B. pertussis Tohama I] |
| 236 | VFFLKYLLSSQSAIL | NP_882160.1 cytochrome c asssembly protein [B. pertussis |
| | | Tohama I] |
| 237 | SGMRRFWKPSLALVW | NP_882327.1 amino acid ABC transporter permease [B. |
| | | pertussis Tohama I] |
| 238 | RAILLKRHNSGFWLI | NP_881921.1 inner membrane protein [B. pertussis Tohama I] |
| 239 | GVLLLQDMAAIPMLV | NP_881082.1 membrane efflux protein [B. pertussis Tohama I] |
| 240 | LELVVDYGWLTIIAK | NP_879348.1 membrane protein insertase YidC [B. pertussis |
| | | Tohama I] |
| 241 | AKYQEIVKISGASLN | NP_882325.1 exported protein [B. pertussis Tohama I] |
| 242 | TALVAERTLILSLVT | NP_881551.1 integral membrane protein [B. pertussis Tohama I] |
| 243 | YQRIQYNTVVSACMK | NP_880711.1 leucine--tRNA ligase [B. pertussis Tohama I] |
| 244 | IKPVFALSSYRTKES | NP_881685.1 integrase [B. pertussis Tohama I] |
| 245 | YSSLNVAQALQLAAW | NP_880587.1 methyltransferase [B. pertussis Tohama I] |
| 246 | LGEFALTRTFKGHAA | NP_882127.1 30S ribosomal protein S19 [B. pertussis Tohama I] |
| 247 | GYRYRAVDALLTNFH | NP_879835.1 S-adenosylmethionine--tRNA ribosyltransferase- |
| | | isomerase [B. pertussis Tohama I] |
| 248 | QRLALIVDPMLATGG | NP_879837.1 uracil phosphoribosyltransferase [B. pertussis |
| | | Tohama I] |
| 249 | PLPGMYRDIARRYDV | NP_878949.1 D-glycero-beta-D-manno-heptose-1,7- |
| | | bisphosphate 7-phosphatase |
| 250 | LYQGLELGASTRIAR | NP_882171.1 ferric siderophore receptor [B. pertussis Tohama I] |
| 251 | AVQLMTVHAAKGLEF | NP_880471.1 DNA helicase II [B. pertussis Tohama I] |
| 252 | HGMKILDGALAKVAK | NP_880538.1 alanine--tRNA ligase [B. pertussis Tohama I] |
| 253 | ALFVTQSTVSKMIRQ | NP_880700.1 LysR family transcriptional regulator [B. pertussis |
| | | Tohama I] |
| 254 | ADVRPLRSATVQRLV | NP_879848.1 hypothetical protein BP1063 [B. pertussis Tohama I] |
| 255 | GAVPFLLAMLLQVGF | NP_882322.1 hypothetical protein BP3824 [B. pertussis Tohama I] |
| 256 | ILGFFIIAALDALRV | NP_880430.1 permease [B. pertussis Tohama I] |
| 257 | RRGELVRVLPDWRSP | NP_879350.1 LysR family transcriptional regulator [B. pertussis |
| | | Tohama I] |
| 258 | SYRSMARMKQVAPRL | NP_879348.1 membrane protein insertase YidC [B. pertussis |
| | | Tohama I] |
| 259 | GALNGILRGVQQPII | NP_879578.1 bifunctional hemolysin-adenylate cyclase [B. |
| | | pertussis Tohama I] |
| 260 | TAARIAKAPRLKLAI | NP_880248.1 formate dehydrogenase [B. pertussis Tohama I] |
| 261 | MRLGLLSVMSWLVTL | NP_880589.1 membrane protein [B. pertussis Tohama I] |
| 262 | DDLIDQIRQRGIAAL | NP_880783.1 hypothetical protein BP2128 [B. pertussis Tohama I] |
| 263 | DSDVILTVSGANTYI | NP_881891.1 hypothetical protein BP3358 [B. pertussis Tohama I] |
| 264 | LRVFESVLVAARAHG | NP_881872.1 ABC transporter ATP-binding protein [B. pertussis |
| | | Tohama I] |
| 265 | FRLIWLYLRMRWLSR | NP_882288.1 type IV secretion system protein PtlB [B. pertussis |
| | | Tohama I] |
| 266 | RGRKRFVQGLRNRRL | NP_881889.1 phage lysozyme [B. pertussis Tohama I] |
| 267 | PVTLAFALTTRVRDL | NP_880587.1 methyltransferase [B. pertussis Tohama I] |
| 268 | ELLQVQONLYAARTI | NP_879348.1 membrane protein insertase YidC [B. pertussis |
| | | Tohama I] |
| 269 | VQAAINAARSLLPTS | NP_882311.1 AcrB/AcrD/AcrF family protein [B. pertussis |
| | | Tohama I] |
| 270 | LGGLFLLFKGTMELH | NP_880589.1 membrane protein [B. pertussis Tohama I] |
| 271 | PLIRHKLGIMRRADL | NP_879837.1 uracil phosphoribosyltransferase [B. pertussis |
| | | Tohama I] |
| 272 | FDVIAQAISGMMSIT | NP_881186.1 hypothetical protein BP2559 [B. pertussis Tohama I] |
| 273 | INIPWSFHAGYRYSF | NP_882013.1 BrkA autotransporter [B. pertussis Tohama I] |
| 274 | VSDMGPLLLSRMLEL | NP_880945.1 ATP-binding protein [B. pertussis Tohama I] |
| 275 | PAGFYYKTFMWPAKF | NP_881143.1 sarcosine oxidase subunit alpha [B. pertussis |
| | | Tohama I] |
| 276 | EDGFLRSFGTGRHFP | NP_880359.1 capsular polysaccharide export protein [B. |
| | | pertussis Tohama I] |
| 277 | LSWIVYLRRQIRQRK | NP_880569.1 virulence sensor protein BvgS [B. pertussis |
| | | Tohama I] |
| 278 | RGRGIALLPSMASEA | NP_879350.1 LysR family transcriptional regulator [B. pertussis |
| | | Tohama I] |
| 279 | TANRRLAALRRFYAW | NP_880275.1 tyrosine recombinase XerD [B. pertussis Tohama I] |
| 280 | ARSIHTVKRLVFVLL | NP_880697.1 integral membrane protein [B. pertussis Tohama I] |
| 281 | TAVMLLLTFVPELVL | NP_880560.1 membrane protein [B. pertussis Tohama I] |
| 282 | TTVVNQVAKARAQEI | NP_881188.1 recombination-associated protein [B. pertussis |
| | | Tohama I] |
| 283 | DKVMKHLARLWGFR | NP_881742.1 SpoVR family protein [B. pertussis Tohama I] |
| | V | |
| 284 | APWIWLRYIGATRLG | NP_880872.1 alanine racemase [B. pertussis Tohama I] |
| 285 | PETHAILRRIRRVID | NP_880087.1 malto-oligosyltrehalose trehalohydrolase [B. |
| | | pertussis Tohama I] |
| 286 | GRVDSIIMRIADVQL | NP_882303.1 transport system permease [B. pertussis Tohama I] |
| 287 | DAMRQLQSAPAGPVV | NP_879350.1 LysR family transcriptional regulator [B. pertussis |
| | | Tohama I] |
| 288 | PMINLLFTPVVKRFR | NP_880700.1 LysR family transcriptional regulator [B. pertussis |
| | | Tohama I] |
| 289 | GKLLDAMLQSVGMSR | NP_882069.1 bacteriophage-related DNA polymerase [B. |
| | | pertussis Tohama I] |
| 290 | VYASFWVIVTQIVVL | NP_880589.1 membrane protein [B. pertussis Tohama I] |
| 291 | EEMLRFGVMPKIALL | NP_879899.1 NADP-dependent malic enzyme [B. pertussis |
| | | Tohama I] |
| 292 | FHRLNVIRLRLPPLR | NP_880331.1 nitrogen regulation protein NR(I) [B. pertussis |
| | | Tohama I] |
| 293 | FFLCAILYVRPAKRA | NP_879871.1 membrane protein [B. pertussis Tohama I] |
| 294 | LPVLDVMQTMPSFVY | NP_880722.1 binding-protein-dependent transport protein [B. |
| | | pertussis Tohama I] |
| 295 | MKILNAIRRGLALAG | NP_880217.1 exported protein [B. pertussis Tohama I] |
| 296 | LILRFLVGVALAGIY | NP_881741.1 membrane protein [B. pertussis Tohama I] |
| 297 | KGKFQMFQIGRIGGY | NP_880698.1 exported protein [B. pertussis Tohama I] |
| 298 | AGAWLVVAATDDRA | NP_879840.1 siroheme synthase [B. pertussis Tohama I] |
| | V | |
| 299 | KRIMFRRAMKRAMQ | NP_882129.1 30S ribosomal protein S3 [B. pertussis Tohama I] |
| | N | |
| 300 | PSVYSIMGAIALVSW | NP_880933.1 integral membrane transport protein [B. pertussis |
| | | Tohama I] |
| 301 | IALLCYADGERRYII | NP_882126.1 50S ribosomal protein L2 [B. pertussis Tohama I] |
| 302 | LFKQMLMVSGFDRYY | NP_879539.1 aspartate--tRNA ligase [B. pertussis Tohama I] |

TABLE 3. PT(O) ANT 1—Overlappinhg peptides covering the entire sequence of NP_879836.1 D-alanyl-D-alanine carboxypeptidase [B. pertussis Tohama I].

TABLE 3
PT(O) ANT 1-Overlapping peptides covering the entire sequence of NP_879836.1
D-alanyl-D-alanine carboxypeptidase [B. pertussis Tohama I]

| SEQ ID | | | |
|---|---|---|---|
| NO: | Peptide | Start | End |
| 303 | MRRAGKQGKWQQWLA | 1 | 15 |
| 304 | KQGKWQQWLAGVMLA | 6 | 20 |
| 305 | QQWLAGVMLALGAAG | 11 | 25 |
| 306 | GVMLALGAAGAAAQG | 16 | 30 |
| 307 | LGAAGAAAQGLPSSL | 21 | 35 |
| 308 | AAAQGLPSSLVAAWK | 26 | 40 |
| 309 | LPSSLVAAWKATKLP | 31 | 45 |
| 310 | VAAWKATKLPDQSLS | 36 | 50 |
| 311 | ATKLPDQSLSLVVQE | 41 | 55 |
| 312 | DQSLSLVVQEINGPR | 46 | 60 |
| 313 | LVVQEINGPRLATLN | 51 | 65 |
| 314 | INGPRLATLNAKEPR | 56 | 70 |
| 315 | LATLNAKEPRNPASV | 61 | 75 |
| 316 | AKEPRNPASVMKLVT | 66 | 80 |
| 317 | NPASVMKLVTTWAAL | 71 | 85 |
| 318 | MKLVTTWAALSELGP | 76 | 90 |
| 319 | TWAALSELGPSYAWR | 81 | 95 |
| 320 | SELGPSYAWRTEFLT | 86 | 100 |
| 321 | SYAWRTEFLTEPGNR | 91 | 105 |
| 322 | TEFLTEPGNRPDAHG | 96 | 110 |
| 323 | EPGNRPDAHGVLRGP | 101 | 115 |
| 324 | PDAHGVLRGPLYLRA | 106 | 120 |
| 325 | VLRGPLYLRAGGDPQ | 111 | 125 |
| 326 | LYLRAGGDPQLLLQD | 116 | 130 |
| 327 | GGDPQLLLQDLWALL | 121 | 135 |
| 328 | LLLQDLWALLRELRL | 126 | 140 |
| 329 | LWALLRELRLRGVKQ | 131 | 145 |
| 330 | RELRLRGVKQIGDLV | 136 | 150 |
| 331 | RGVKQIGDLVVDRSI | 141 | 155 |
| 332 | IGDLVVDRSIFGQVA | 146 | 160 |
| 333 | VDRSIFGQVAIDPGA | 151 | 165 |
| 334 | FGQVAIDPGAFDGAS | 156 | 170 |
| 335 | IDPGAFDGASDRAYN | 161 | 175 |
| 336 | FDGASDRAYNASPDA | 166 | 180 |
| 337 | DRAYNASPDALMVGF | 171 | 185 |
| 338 | ASPDALMVGFGAQRL | 176 | 190 |
| 339 | LMVGFGAQRLLFTPD | 181 | 195 |
| 340 | GAQRLLFTPDAAARK | 186 | 200 |
| 341 | LFTPDAAARKWVPMI | 191 | 205 |
| 342 | AAARKWVPMIDPPLP | 196 | 210 |
| 343 | WVPMIDPPLPGLRLE | 201 | 215 |
| 344 | DPPLPGLRLEGAVEW | 206 | 220 |
| 345 | GLRLEGAVEWSDVRC | 211 | 225 |
| 346 | GAVEWSDVRCPGPPV | 216 | 230 |
| 347 | SDVRCPGPPVVGTEP | 221 | 235 |
| 348 | PGPPVVGTEPVVTQQ | 226 | 240 |
| 349 | VGTEPVVTQQGVSIR | 231 | 245 |
| 350 | VVTQQGVSIRLSGKV | 236 | 250 |
| 351 | GVSIRLSGKVAGSCG | 241 | 255 |
| 352 | LSGKVAGSCGEFSLY | 246 | 260 |
| 353 | AGSCGEFSLYRLALS | 251 | 265 |
| 354 | EFSLYRLALSQPEYA | 256 | 270 |
| 355 | RLALSQPEYASAVFR | 261 | 275 |
| 356 | QPEYASAVFRLLWRE | 266 | 280 |
| 357 | SAVFRLLWRELGGTL | 271 | 285 |
| 358 | LLWRELGGTLKGQIR | 276 | 290 |
| 359 | LGGTLKGQIRSGVVP | 281 | 295 |
| 360 | KGQIRSGVVPPDAVV | 286 | 300 |
| 361 | SGVVPPDAVVLASHD | 291 | 305 |
| 362 | PDAVVLASHDSPTLG | 296 | 310 |
| 363 | LASHDSPTLGEAIRT | 301 | 315 |
| 364 | SPTLGEAIRTINKRS | 306 | 320 |
| 365 | EAIRTINKRSNNVMA | 311 | 325 |
| 366 | INKRSNNVMARTLLL | 316 | 330 |
| 367 | NNVMARTLLLTLGAE | 321 | 335 |
| 368 | RTLLLTLGAERGRRP | 326 | 340 |
| 369 | TLGAERGRRPATVES | 331 | 345 |
| 370 | RGRRPATVESSGVVA | 336 | 350 |
| 371 | ATVESSGVVARTVLG | 341 | 355 |
| 372 | SGVVARTVLGAQGLE | 346 | 360 |
| 373 | RTVLGAQGLEMPELV | 351 | 365 |
| 374 | AQGLEMPELVIDNGS | 356 | 370 |
| 375 | MPELVIDNGSGLSRE | 361 | 375 |
| 376 | IDNGSGLSREGRVSA | 366 | 380 |
| 377 | GLSREGRVSADSLAS | 371 | 385 |
| 378 | GRVSADSLASMLTVA | 376 | 390 |
| 379 | DSLASMLTVAWNSPL | 381 | 395 |
| 380 | MLTVAWNSPLMPEFI | 386 | 400 |
| 381 | WNSPLMPEFISSLAI | 391 | 405 |
| 382 | MPEFISSLAIAGVDG | 396 | 410 |
| 383 | SSLAIAGVDGTVRRR | 401 | 415 |
| 384 | AGVDGTVRRRLKGNG | 406 | 420 |
| 385 | TVRRRLKGNGAQGMA | 411 | 425 |
| 386 | LKGNGAQGMAHLKTG | 416 | 430 |
| 387 | AQGMAHLKTGSLRDV | 421 | 435 |
| 388 | HLKTGSLRDVRAVAG | 426 | 440 |
| 389 | SLRDVRAVAGYVLGA | 431 | 445 |
| 390 | RAVAGYVLGASGKRY | 436 | 450 |
| 391 | YVLGASGKRYVVVSM | 441 | 455 |
| 392 | SGKRYVVVSMVNHEN | 446 | 460 |
| 393 | VVVSMVNHENAAAVR | 451 | 465 |
| 394 | VNHENAAAVRSFDDA | 456 | 470 |
| 395 | AAAVRSFDDALVAWL | 461 | 475 |
| 396 | VRSFDDALVAWLAEQ | 464 | 478 |
| | Full Sequence | | |

| | |
|---|---|
| 2599 | MRRAGKQGKWQQWLAGVMLALGAAGAAAQGLPSSLVAAWKATKLPDQSLSLV |
| | VQEINGPRLATLNAKEPRNPASVMKLVTTWAALSELGPSYAWRTEFLTEPGNRPD |
| | AHGVLRGPLYLRAGGDPQLLLQDLWALLRELRLRGVKQIGDLVVDRSIFGQVAID |
| | PGAFDGASDRAYNASPDALMVGFGAQRLLFTPDAAARKWVPMIDPPLPGLRLEGA |
| | VEWSDVRCPGPPVVGTEPVVTQQGVSIRLSGKVAGSCGEFSLYRLALSQPEYASAV |
| | FRLLWRELGGTLKGQIRSGVVPPDAVVLASHDSPTLGEAIRTINKRSNNVMARTLL |
| | LTLGAERGRRPATVESSGVVARTVLGAQGLEMPELVIDNGSGLSREGRVSADSLAS |
| | MLTVAWNSPLMPEFISSLAIAGVDGTVRRRLKGNGAQGMAHLKTGSLRDVRAVA |
| | GYVLGASGKRYVVVSMVNHENAAAVRSFDDALVAWLAEQ |

TABLE 4. PT(O) ANT 2—Overlapping peptides covering the entire sequence of NP_879348.1 membrane protein insertase YidC [B. pertussis Tohama I].

TABLE 4
PT(O) ANT 2-Overlapping peptides covering
the entire sequence of NP_879348.1
membrane protein insertase
YidC [B. pertussis Tohama I]

| SEQ | | | |
|---|---|---|---|
| ID | | | |
| NO: | Peptide | Start | End |
| 397 | MDIRRTVLWMIFSFS | 1 | 15 |
| 398 | TVLWMIFSFSLLLLW | 6 | 20 |
| 399 | IFSFSLLLLWNNWQI | 11 | 25 |
| 400 | LLLLWNNWQIHNGKP | 16 | 30 |
| 401 | NNWQIHNGKPSLFGG | 21 | 35 |
| 402 | HNGKPSLFGGPAPEA | 26 | 40 |
| 403 | SLFGGPAPEAAATQQ | 31 | 45 |
| 404 | PAPEAAATQQPKADA | 36 | 50 |
| 405 | AATQQPKADANGTAA | 41 | 55 |
| 406 | PKADANGTAASSTAS | 46 | 60 |
| 407 | NGTAASSTASIPSSP | 51 | 65 |
| 408 | SSTASIPSSPAAAPA | 56 | 70 |
| 409 | IPSSPAAAPAAASVP | 61 | 75 |
| 410 | AAAPAAASVPGAAAP | 66 | 80 |
| 411 | AASVPGAAAPAAAKS | 71 | 85 |
| 412 | GAAAPAAAKSEQVVI | 76 | 90 |
| 413 | AAAKSEQVVITTDVL | 81 | 95 |
| 414 | EQVVITTDVLRLTFD | 86 | 100 |
| 415 | TTDVLRLTFDSNGAQ | 91 | 105 |
| 416 | RLTFDSNGAQLIRAE | 96 | 110 |
| 417 | SNGAQLIRAELLKYP | 101 | 115 |
| 418 | LIRAELLKYPSSSQS | 106 | 120 |
| 419 | LLKYPSSSQSDKPTV | 111 | 125 |
| 420 | SSSQSDKPTVLMDRS | 116 | 130 |
| 421 | DKPTVLMDRSADLVY | 121 | 135 |
| 422 | LMDRSADLVYVAQTG | 126 | 140 |
| 423 | ADLVYVAQTGVVGAP | 131 | 145 |
| 424 | VAQTGVVGAPQGESF | 136 | 150 |
| 425 | VVGAPQGESFPTHQT | 141 | 155 |
| 426 | QGESFPTHQTPFHLV | 146 | 160 |
| 427 | PTHQTPFHLVSSERS | 151 | 165 |
| 428 | PFHLVSSERSLTGDT | 156 | 170 |
| 429 | SSERSLTGDTLDVVF | 161 | 175 |
| 430 | LTGDTLDVVFEAESG | 166 | 180 |
| 431 | LDVVFEAESGGLKVT | 171 | 185 |
| 432 | EAESGGLKVTKTYTL | 176 | 190 |
| 433 | GLKVTKTYTLHRGRY | 181 | 195 |
| 434 | KTYTLHRGRYDVDVR | 186 | 200 |
| 435 | HRGRYDVDVRHAMAN | 191 | 205 |
| 436 | DVDVRHAMANTGGAP | 196 | 210 |
| 437 | HAMANTGGAPLNPAL | 201 | 215 |
| 438 | TGGAPLNPALYLQLE | 206 | 220 |
| 439 | LNPALYLQLERDGTD | 211 | 225 |
| 440 | YLQLERDGTDPAGTS | 216 | 230 |
| 441 | RDGTDPAGTSSFYHT | 221 | 235 |
| 442 | PAGTSSFYHTFTGVA | 226 | 240 |
| 443 | SFYHTFTGVAVYSEQ | 231 | 245 |
| 444 | FTGVAVYSEQDKFQK | 236 | 250 |
| 445 | VYSEQDKFQKVTFSD | 241 | 255 |
| 446 | DKFQKVTFSDIEKKK | 246 | 260 |
| 447 | VTFSDIEKKKGTYIK | 251 | 265 |
| 448 | IEKKKGTYIKQADNG | 256 | 270 |
| 449 | GTYIKQADNGWIGIV | 261 | 275 |
| 450 | QADNGWIGIVQHYFA | 266 | 280 |
| 451 | WIGIVQHYFATAWIP | 271 | 285 |
| 452 | QHYFATAWIPAQGKQ | 276 | 290 |
| 453 | TAWIPAQGKQRTNEL | 281 | 295 |
| 454 | AQGKQRTNELLQVQQ | 286 | 300 |
| 455 | RTNELLQVQONLYAA | 291 | 305 |
| 456 | LQVQONLYAARTIEA | 296 | 310 |
| 457 | NLYAARTIEAVGTIA | 301 | 315 |
| 458 | RTIEAVGTIAPGSSA | 306 | 320 |
| 459 | VGTIAPGSSANVDAH | 311 | 325 |
| 460 | PGSSANVDAHLWVGP | 316 | 330 |
| 461 | NVDAHLWVGPQDQKA | 321 | 335 |
| 462 | LWVGPQDQKAMAAVA | 326 | 340 |
| 463 | QDQKAMAAVAPGLEL | 331 | 345 |
| 464 | MAAVAPGLELVVDYG | 336 | 350 |
| 465 | PGLELVVDYGWLTII | 341 | 355 |
| 466 | VVDYGWLTIIAKPLF | 346 | 360 |
| 467 | WLTIIAKPLFTLMTW | 351 | 365 |
| 468 | AKPLFTLMTWLHGLL | 356 | 370 |
| 469 | TLMTWLHGLLGNWGW | 361 | 375 |
| 470 | LHGLLGNWGWTIVAL | 366 | 380 |
| 471 | GNWGWTIVALTVIIK | 371 | 385 |
| 472 | TIVALTVIIKAVFFP | 376 | 390 |
| 473 | TVIIKAVFFPLAAAS | 381 | 395 |
| 474 | AVFFPLAAASYRSMA | 386 | 400 |
| 475 | LAAASYRSMARMKQV | 391 | 405 |
| 476 | YRSMARMKQVAPRLQ | 396 | 410 |
| 477 | RMKQVAPRLQALKEK | 401 | 415 |
| 478 | APRLQALKEKYGDDR | 406 | 420 |
| 479 | ALKEKYGDDRQKLNQ | 411 | 425 |
| 480 | YGDDRQKLNQAMMEM | 416 | 430 |
| 481 | QKLNQAMMEMYRTEK | 421 | 435 |
| 482 | AMMEMYRTEKINPLG | 426 | 440 |
| 483 | YRTEKINPLGGCLPM | 431 | 445 |
| 484 | INPLGGCLPMVVQIP | 436 | 450 |
| 485 | GCLPMVVQIPVFIAL | 441 | 455 |
| 486 | VVQIPVFIALYWVLL | 446 | 460 |
| 487 | VFIALYWVLLASVEM | 451 | 465 |
| 488 | YWVLLASVEMRGAPW | 456 | 470 |
| 489 | ASVEMRGAPWILWVH | 461 | 475 |
| 490 | RGAPWILWVHDLSVR | 466 | 480 |
| 491 | ILWVHDLSVRDPFFI | 471 | 485 |
| 492 | DLSVRDPFFILPAIM | 476 | 490 |
| 493 | DPFFILPAIMMATMF | 481 | 495 |
| 494 | LPAIMMATMFLQIKL | 486 | 500 |
| 495 | MATMFLQIKLNPTPP | 491 | 505 |
| 496 | LQIKLNPTPPDPVQA | 496 | 510 |
| 497 | NPTPPDPVQAKVMMI | 501 | 515 |
| 498 | DPVQAKVMMIMPLVF | 506 | 520 |
| 499 | KVMMIMPLVFGGMMF | 511 | 525 |
| 500 | MPLVFGGMMFFFPAG | 516 | 530 |
| 501 | GGMMFFFPAGLVLYW | 521 | 535 |
| 502 | FFPAGLVLYWCVNNT | 526 | 540 |
| 503 | LVLYWCVNNTLSIAQ | 531 | 545 |
| 504 | CVNNTLSIAQQWTIT | 536 | 550 |
| 505 | LSIAQQWTITRNLER | 541 | 555 |
| 506 | QWTITRNLERQAAAA | 546 | 560 |
| 507 | ITRNLERQAAAAANR | 549 | 563 |

| | |
|---|---|
| | Full Sequence |
| 2600 | MDIRRTVLWMIFSFSLLLLWNNWQIHNGKPSLFGGPAPEA |
| | AATQQPKADANGTAASSTASIPSSPAAAPAAASVPGAAAP |
| | AAAKSEQVVITTDVLRLTFDSNGAQLIRAELLKYPSSSQS |
| | DKPTVLMDRSADLVYVAQTGVVGAPQGESFPTHQTPFHLV |
| | SSERSLTGDTLDVVFEAESGGLKVTKTYTLHRGRYDVDVR |
| | HAMANTGGAPLNPALYLQLERDGTDPAGTSSFYHTFTGVA |
| | VYSEQDKFQKVTFSDIEKKKGTYIKQADNGWIGIVQHYFA |
| | TAWIPAQGKQRTNELLQVQONLYAARTIEAVGTIAPGSSA |
| | NVDAHLWVGPQDQKAMAAVAPGLELVVDYGWLTIIAKPLF |
| | TLMTWLHGLLGNWGWTIVALTVIIKAVFFPLAAASYRSMA |
| | RMKQVAPRLQALKEKYGDDRQKLNQAMMEMYRTEKINPLG |
| | GCLPMVVQIPVFIALYWVLLASVEMRGAPWILWVHDLSVR |
| | DPFFILPAIMMATMFLQIKLNPTPPDPVQAKVMMIMPLVF |
| | GGMMFFFPAGLVLYWCVNNTLSIAQQWTITRNLERQAAAA |
| | ANR |

TABLE 5. PT(O) ANT 3—Overlapping peptides covering the entire sequence of NP_880087.1 malto-oligosyltrehalose trehalohydrolase [B. pertussis Tohama I].

TABLE 5
PT(O) ANT 3-Overlapping peptides covering
the entire sequence of NP_880087.1 malto-
trehalohydrolase [B. pertussis Tohama I]

| SEQ | | | |
|---|---|---|---|
| ID | | | |
| NO: | Peptide | Start | End |
| 508 | MPATHPAPDPLWYKD | 1 | 15 |
| 509 | PAPDPLWYKDAVIYQ | 6 | 20 |
| 510 | LWYKDAVIYQLHVKS | 11 | 25 |
| 511 | AVIYQLHVKSFFDAN | 16 | 30 |
| 512 | LHVKSFFDANDDGVG | 21 | 35 |
| 513 | FFDANDDGVGDFAGL | 26 | 40 |
| 514 | DDGVGDFAGLLAKLD | 31 | 45 |
| 515 | DFAGLLAKLDYIVEL | 36 | 50 |
| 516 | LAKLDYIVELGVNTI | 41 | 55 |
| 517 | YIVELGVNTIWLLPF | 46 | 60 |
| 518 | GVNTIWLLPFYPSPR | 51 | 65 |
| 519 | WLLPFYPSPRRDDGY | 56 | 70 |
| 520 | YPSPRRDDGYDIADY | 61 | 75 |
| 521 | RDDGYDIADYRGVHP | 66 | 80 |
| 522 | DIADYRGVHPDYGSL | 71 | 85 |
| 523 | RGVHPDYGSLADARL | 76 | 90 |
| 524 | DYGSLADARLLVRAA | 81 | 95 |
| 525 | ADARLLVRAAHARGL | 86 | 100 |
| 526 | LVRAAHARGLRVITE | 91 | 105 |
| 527 | HARGLRVITELVINH | 96 | 110 |
| 528 | RVITELVINHTSDQH | 101 | 115 |
| 529 | LVINHTSDQHPWFQR | 106 | 120 |
| 530 | TSDQHPWFQRARAAR | 111 | 125 |
| 531 | PWFQRARAARPGSAH | 116 | 130 |
| 532 | ARAARPGSAHRAYYV | 121 | 135 |
| 533 | PGSAHRAYYVWSDDD | 126 | 140 |
| 534 | RAYYVWSDDDKAYAG | 131 | 145 |
| 535 | WSDDDKAYAGTRIIF | 136 | 150 |
| 536 | KAYAGTRIIFCDTEK | 141 | 155 |
| 537 | TRIIFCDTEKSNWTW | 146 | 160 |
| 538 | CDTEKSNWTWDPVAG | 151 | 165 |
| 539 | SNWTWDPVAGAYFWH | 156 | 170 |
| 540 | DPVAGAYFWHRFYSH | 161 | 175 |
| 541 | AYFWHRFYSHQPDLN | 166 | 180 |
| 542 | RFYSHQPDLNYDNPQ | 171 | 185 |
| 543 | QPDLNYDNPQVLREV | 176 | 190 |
| 544 | YDNPQVLREVLGAMR | 181 | 195 |
| 545 | VLREVLGAMRYWLDM | 186 | 200 |
| 546 | LGAMRYWLDMGVDGL | 191 | 205 |
| 547 | YWLDMGVDGLRLDAV | 196 | 210 |
| 548 | GVDGLRLDAVPYLVE | 201 | 215 |
| 549 | RLDAVPYLVEREGTN | 206 | 220 |
| 550 | PYLVEREGTNNENLP | 211 | 225 |
| 551 | REGTNNENLPETHAI | 216 | 230 |
| 552 | NENLPETHAILRRIR | 221 | 235 |
| 553 | ETHAILRRIRRVIDS | 226 | 240 |
| 554 | LRRIRRVIDSEYPGR | 231 | 245 |
| 555 | RVIDSEYPGRMLLAE | 236 | 250 |
| 556 | EYPGRMLLAEANQWP | 241 | 255 |
| 557 | MLLAEANQWPEDAQE | 246 | 260 |
| 558 | ANQWPEDAQEYFGAG | 251 | 265 |
| 559 | EDAQEYFGAGDECHM | 256 | 270 |
| 560 | YFGAGDECHMAFHFP | 261 | 275 |
| 561 | DECHMAFHFPLMPRM | 266 | 280 |
| 562 | AFHFPLMPRMYMAIA | 271 | 285 |
| 563 | LMPRMYMAIAQEDRL | 276 | 290 |
| 564 | YMAIAQEDRLPVTDI | 281 | 295 |
| 565 | QEDRLPVTDIIRQTP | 286 | 300 |
| 566 | PVTDIIRQTPSIAPQ | 291 | 305 |
| 567 | IRQTPSIAPQCQWAI | 296 | 310 |
| 568 | SIAPQCQWAIFLRNH | 301 | 315 |
| 569 | CQWAIFLRNHDELTL | 306 | 320 |
| 570 | FLRNHDELTLEMVTS | 311 | 325 |
| 571 | DELTLEMVTSRERDY | 316 | 330 |
| 572 | EMVTSRERDYLWNVY | 321 | 335 |
| 573 | RERDYLWNVYAAEPR | 326 | 340 |
| 574 | LWNVYAAEPRARINL | 331 | 345 |
| 575 | AAEPRARINLGIRRR | 336 | 350 |
| 576 | ARINLGIRRRLAPLL | 341 | 355 |
| 577 | GIRRRLAPLLERDRR | 346 | 360 |
| 578 | LAPLLERDRRRIELM | 351 | 365 |
| 579 | ERDRRRIELMNSLLL | 356 | 370 |
| 580 | RIELMNSLLLSMPGT | 361 | 375 |
| 581 | NSLLLSMPGTPVLYY | 366 | 380 |
| 582 | SMPGTPVLYYGDELG | 371 | 385 |
| 583 | PVLYYGDELGMGDNI | 376 | 390 |
| 584 | GDELGMGDNIHLGDR | 381 | 395 |
| 585 | MGDNIHLGDRDGVRT | 386 | 400 |
| 586 | HLGDRDGVRTPMQWS | 391 | 405 |
| 587 | DGVRTPMQWSPDRNG | 396 | 410 |
| 588 | PMQWSPDRNGGFSRA | 401 | 415 |
| 589 | PDRNGGFSRADPERL | 406 | 420 |
| 590 | GFSRADPERLPLPLL | 411 | 425 |
| 591 | DPERLPLPLLMGPLY | 416 | 430 |
| 592 | PLPLLMGPLYGYEAV | 421 | 435 |
| 593 | MGPLYGYEAVNVEAQ | 426 | 440 |
| 594 | GYEAVNVEAQQRDPH | 431 | 445 |
| 595 | NVEAQQRDPHSLLNW | 436 | 450 |
| 596 | QRDPHSLLNWTRRML | 441 | 455 |
| 597 | SLLNWTRRMLAKRRQ | 446 | 460 |
| 598 | TRRMLAKRRQSHVFG | 451 | 465 |
| 599 | AKRRQSHVFGRGELS | 456 | 470 |
| 600 | SHVFGRGELSFLYPG | 461 | 475 |
| 601 | RGELSFLYPGNRKIL | 466 | 480 |
| 602 | FLYPGNRKILAYLRT | 471 | 485 |
| 603 | NRKILAYLRTWEDTV | 476 | 490 |
| 604 | AYLRTWEDTVVLCVA | 481 | 495 |
| 605 | WEDTVVLCVANLSQA | 486 | 500 |
| 606 | VLCVANLSQAAQPVE | 491 | 505 |
| 607 | NLSQAAQPVELHLSE | 496 | 510 |
| 608 | AQPVELHLSEYAGRV | 501 | 515 |
| 609 | LHLSEYAGRVPVEML | 506 | 520 |
| 610 | YAGRVPVEMLGGTAF | 511 | 525 |
| 611 | PVEMLGGTAFPQIGE | 516 | 530 |
| 612 | GGTAFPQIGELPYLL | 521 | 535 |
| 613 | PQIGELPYLLTLPPF | 526 | 540 |
| 614 | LPYLLTLPPFGFYWL | 531 | 545 |
| 615 | TLPPFGFYWLDLSAG | 536 | 550 |
| 616 | GFYWLDLSAGAAPPA | 541 | 555 |
| 617 | DLSAGAAPPAWHSEL | 546 | 560 |
| 618 | AAPPAWHSELPPQMP | 551 | 565 |
| 619 | WHSELPPQMPESITL | 556 | 570 |
| 620 | PPQMPESITLVSRGA | 561 | 575 |
| 621 | ESITLVSRGAGAALR | 566 | 580 |
| 622 | VSRGAGAALRLTEAS | 571 | 585 |
| 623 | GAALRLTEASRRQLE | 576 | 590 |
| 624 | LTEASRRQLEADVLP | 581 | 595 |
| 625 | RRQLEADVLPAYLQR | 586 | 600 |
| 626 | ADVLPAYLQRQRWYA | 591 | 605 |
| 627 | AYLQRQRWYAARRKP | 596 | 610 |
| 628 | QRWYAARRKPGVMRL | 601 | 615 |
| 629 | ARRKPGVMRLAYSVP | 606 | 620 |
| 630 | GVMRLAYSVPLNDDV | 611 | 625 |
| 631 | AYSVPLNDDVESYYE | 616 | 630 |
| 632 | LNDDVESYYEAEIEV | 621 | 635 |
| 633 | ESYYEAEIEVSDDGP | 626 | 640 |
| 634 | AEIEVSDDGPPRRFH | 631 | 645 |
| 635 | SDDGPPRRFHTPVAL | 636 | 650 |
| 636 | PRRFHTPVALAWQDD | 641 | 655 |
| 637 | TPVALAWQDDTAAQY | 646 | 660 |
| 638 | AWQDDTAAQYPLARV | 651 | 665 |
| 639 | TAAQYPLARVRRGAQ | 656 | 670 |
| 640 | PLARVRRGAQLGTLT | 661 | 675 |
| 641 | RRGAQLGTLTDASLQ | 666 | 680 |
| 642 | LGTLTDASLQPGYAR | 671 | 685 |
| 643 | DASLQPGYARVLLAA | 676 | 690 |
| 644 | PGYARVLLAALTAGR | 681 | 695 |
| 645 | VLLAALTAGRDIQAG | 686 | 700 |
| 646 | LTAGRDIQAGGEPAV | 691 | 705 |
| 647 | DIQAGGEPAVRLRFL | 696 | 710 |
| 648 | GEPAVRLRFLPEPGL | 701 | 715 |
| 649 | RLRFLPEPGLADLAL | 706 | 720 |
| 650 | PEPGLADLALRDDSE | 711 | 725 |
| 651 | ADLALRDDSEVRALS | 716 | 730 |
| 652 | RDDSEVRALSADQSN | 721 | 735 |
| 653 | VRALSADQSNSSLLV | 726 | 740 |
| 654 | ADQSNSSLLVGERVV | 731 | 745 |
| 655 | SSLLVGERVVFKLLR | 736 | 750 |
| 656 | GERVVFKLLRELHAG | 741 | 755 |
| 657 | FKLLRELHAGPHPEA | 746 | 760 |
| 658 | ELHAGPHPEAEMTRY | 751 | 765 |
| 659 | PHPEAEMTRYLTQAG | 756 | 770 |
| 660 | EMTRYLTQAGYAHTP | 761 | 775 |
| 661 | LTQAGYAHTPALLGE | 766 | 780 |
| 662 | YAHTPALLGEVVRVQ | 771 | 785 |
| 663 | ALLGEVVRVQGDQAP | 776 | 790 |
| 664 | VVRVQGDQAPHTLAL | 781 | 795 |
| 665 | GDQAPHTLALAHAYV | 786 | 800 |
| 666 | HTLALAHAYVVNEGD | 791 | 805 |
| 667 | AHAYVVNEGDAWNWT | 796 | 810 |
| 668 | VNEGDAWNWTVAYLK | 801 | 815 |
| 669 | AWNWTVAYLKRTLDA | 806 | 820 |
| 670 | VAYLKRTLDAAILTG | 811 | 825 |
| 671 | RTLDAAILTGASADD | 816 | 830 |
| 672 | AILTGASADDYQQEL | 821 | 835 |
| 673 | ASADDYQQELAGYEV | 826 | 840 |
| 674 | YQQELAGYEVLAGTI | 831 | 845 |
| 675 | AGYEVLAGTIGQRLA | 836 | 850 |
| 676 | LAGTIGQRLAQMHSV | 841 | 855 |
| 677 | GQRLAQMHSVLARAG | 846 | 860 |
| 678 | QMHSVLARAGELPGF | 851 | 865 |
| 679 | LARAGELPGFAPRPA | 856 | 870 |
| 680 | ELPGFAPRPASERDA | 861 | 875 |
| 681 | APRPASERDAALAGE | 866 | 880 |
| 682 | SERDAALAGERAVAQ | 871 | 885 |
| 683 | ALAGERAVAQLDRAL | 876 | 890 |
| 684 | RAVAQLDRALQALRA | 881 | 895 |
| 685 | LDRALQALRACESGL | 886 | 900 |
| 686 | QALRACESGLAPASH | 891 | 905 |
| 687 | CESGLAPASHACAQW | 896 | 910 |
| 688 | APASHACAQWLFEHR | 901 | 915 |
| 689 | ACAQWLFEHRDRLAA | 906 | 920 |
| 690 | LFEHRDRLAAHIMTL | 911 | 925 |
| 691 | DRLAAHIMTLAQAET | 916 | 930 |
| 692 | HIMTLAQAETGALRI | 921 | 935 |
| 693 | AQAETGALRIRVHGD | 926 | 940 |
| 694 | GALRIRVHGDFHLGQ | 931 | 945 |
| 695 | RVHGDFHLGQILVAQ | 936 | 950 |
| 696 | FHLGQILVAQTDAYL | 941 | 955 |
| 697 | ILVAQTDAYLIDFEG | 946 | 960 |
| 698 | TDAYLIDFEGEPARP | 951 | 965 |
| 699 | IDFEGEPARPMAERR | 956 | 970 |
| 700 | EPARPMAERRQLSSP | 961 | 975 |
| 701 | MAERRQLSSPFKDVA | 966 | 980 |
| 702 | QLSSPFKDVAGILRS | 971 | 985 |
| 703 | FKDVAGILRSFDYAV | 976 | 990 |
| 704 | GILRSFDYAVAELSR | 981 | 995 |
| 705 | FDYAVAELSRDDPLG | 986 | 1000 |
| 706 | AELSRDDPLGGAPRD | 991 | 1005 |
| 707 | DDPLGGAPRDFNTGV | 996 | 1010 |
| 708 | GAPRDFNTGVAEPAD | 1001 | 1015 |
| 709 | FNTGVAEPADPASAP | 1006 | 1020 |
| 710 | AEPADPASAPRETRE | 1011 | 1025 |
| 711 | PASAPRETREALLAR | 1016 | 1030 |
| 712 | RETREALLARFRQRA | 1021 | 1035 |
| 713 | ALLARFRQRAGAALL | 1026 | 1040 |
| 714 | FRQRAGAALLASYGN | 1031 | 1045 |
| 715 | GAALLASYGNAIDPV | 1036 | 1050 |
| 716 | ASYGNAIDPVLALPP | 1041 | 1055 |
| 717 | AIDPVLALPPERAQA | 1046 | 1060 |
| 718 | LALPPERAQALTCLY | 1051 | 1065 |
| 719 | ERAQALTCLYLLEKA | 1056 | 1070 |
| 720 | LTCLYLLEKAAYEIC | 1061 | 1075 |
| 721 | LLEKAAYEICYESAY | 1066 | 1080 |
| 722 | AYEICYESAYRPERL | 1071 | 1085 |
| 723 | YESAYRPERLPVPIH | 1076 | 1090 |
| 724 | RPERLPVPIHGLAET | 1081 | 1095 |
| 725 | PVPIHGLAETARAAL | 1086 | 1100 |
| 726 | GLAETARAALLAAAV | 1091 | 1105 |
| 727 | ARAALLAAAVDHDEG | 1096 | 1110 |
| 728 | ALLAAAVDHDEGPAP | 1099 | 1113 |

| | |
|---|---|
| | Full Sequence |
| 2601 | MPATHPAPDPLWYKDAVIYQLHVKSFFDANDDGVGDFAGL |
| | LAKLDYIVELGVNTIWLLPFYPSPRRDDGYDIADYRGVHP |
| | DYGSLADARLLVRAAHARGLRVITELVINHTSDQHPWFQR |
| | ARAARPGSAHRAYYVWSDDDKAYAGTRIIFCDTEKSNWTW |
| | DPVAGAYFWHRFYSHQPDLNYDNPQVLREVLGAMRYWLDM |
| | GVDGLRLDAVPYLVEREGTNNENLPETHAILRRIRRVIDS |
| | EYPGRMLLAEANQWPEDAQEYFGAGDECHMAFHFPLMPRM |
| | YMAIAQEDRLPVTDIIRQTPSIAPQCQWAIFLRNHDELTL |
| | EMVTSRERDYLWNVYAAEPRARINLGIRRRLAPLLERDRR |
| | RIELMNSLLLSMPGTPVLYYGDELGMGDNIHLGDRDGVRT |
| | PMQWSPDRNGGFSRADPERLPLPLLMGPLYGYEAVNVEAQ |
| | QRDPHSLLNWTRRMLAKRRQSHVFGRGELSFLYPGNRKIL |
| | AYLRTWEDTVVLCVANLSQAAQPVELHLSEYAGRVPVEML |
| | GGTAFPQIGELPYLLTLPPFGFYWLDLSAGAAPPAWHSEL |
| | PPQMPESITLVSRGAGAALRLTEASRRQLEADVLPAYLQR |
| | QRWYAARRKPGVMRLAYSVPLNDDVESYYEAEIEVSDDGP |
| | PRRFHTPVALAWQDDTAAQYPLARVRRGAQLGTLTDASLQ |
| | PGYARVLLAALTAGRDIQAGGEPAVRLRFLPEPGLADLAL |
| | RDDSEVRALSADQSNSSLLVGERVVFKLLRELHAGPHPEA |
| | EMTRYLTQAGYAHTPALLGEVVRVQGDQAPHTLALAHAYV |
| | VNEGDAWNWTVAYLKRTLDAAILTGASADDYQQELAGYEV |
| | LAGTIGQRLAQMHSVLARAGELPGFAPRPASERDAALAGE |
| | RAVAQLDRALQALRACESGLAPASHACAQWLFEHRDRLAA |
| | HIMTLAQAETGALRIRVHGDFHLGQILVAQTDAYLIDFEG |
| | EPARPMAERRQLSSPFKDVAGILRSFDYAVAELSRDDPLG |
| | GAPRDFNTGVAEPADPASAPRETREALLARFRQRAGAALL |
| | ASYGNAIDPVLALPPERAQALTCLYLLEKAAYEICYESAY |
| | RPERLPVPIHGLAETARAALLAAAVDHDEGPAP |

TABLE 6 PT(O) ANT 4—Overlapping peptides covering the entire sequence of NP880569.1 virulence sensor protein BvgS [B. pertussis Tohama I].

TABLE 6
PT(O) ANT 4-Overlapping peptides covering
the entire sequence of NP_880569.1 virulence
sensor protein BvgS [B. pertussis Tohama I]

| SEQ ID | | | |
|---|---|---|---|
| NO: | Peptide | Start | End |
| 729 | MPAPHRLYPRSLICL | 1 | 15 |
| 730 | RLYPRSLICLAQALL | 6 | 20 |
| 731 | SLICLAQALLAWALL | 11 | 25 |
| 732 | AQALLAWALLAWAPA | 16 | 30 |
| 733 | AWALLAWAPAQASQE | 21 | 35 |
| 734 | AWAPAQASQELTLVG | 26 | 40 |
| 735 | QASQELTLVGKAAVP | 31 | 45 |
| 736 | LTLVGKAAVPDVEVA | 36 | 50 |
| 737 | KAAVPDVEVALDGDD | 41 | 55 |
| 738 | DVEVALDGDDWRWLA | 46 | 60 |
| 739 | LDGDDWRWLARKRVL | 51 | 65 |
| 740 | WRWLARKRVLTLGVY | 56 | 70 |
| 741 | RKRVLTLGVYAPDIP | 61 | 75 |
| 742 | TLGVYAPDIPPFDVT | 66 | 80 |
| 743 | APDIPPFDVTYGERY | 71 | 85 |
| 744 | PFDVTYGERYEGLTA | 76 | 90 |
| 745 | YGERYEGLTADYMAI | 81 | 95 |
| 746 | EGLTADYMAIIAHNL | 86 | 100 |
| 747 | DYMAIIAHNLGMQAK | 91 | 105 |
| 748 | JAHNLGMQAKVLRYP | 96 | 110 |
| 749 | GMQAKVLRYPTREQA | 101 | 115 |
| 750 | VLRYPTREQALSALE | 106 | 120 |
| 751 | TREQALSALESGQID | 111 | 125 |
| 752 | LSALESGQIDLIGTV | 116 | 130 |
| 753 | SGQIDLIGTVNGTDG | 121 | 135 |
| 754 | LIGTVNGTDGRQQSL | 126 | 140 |
| 755 | NGTDGRQQSLRLSVP | 131 | 145 |
| 756 | RQQSLRLSVPYAADH | 136 | 150 |
| 757 | RLSVPYAADHPVIVM | 141 | 155 |
| 758 | YAADHPVIVMPIGAR | 146 | 160 |
| 759 | PVIVMPIGARHVPAS | 151 | 165 |
| 760 | PIGARHVPASNLAGQ | 156 | 170 |
| 761 | HVPASNLAGQRLAVD | 161 | 175 |
| 762 | NLAGQRLAVDINYLP | 166 | 180 |
| 763 | RLAVDINYLPKETLA | 171 | 185 |
| 764 | INYLPKETLARAYPQ | 176 | 190 |
| 765 | KETLARAYPQATLHY | 181 | 195 |
| 766 | RAYPQATLHYFPSSE | 186 | 200 |
| 767 | ATLHYFPSSEQALAA | 191 | 205 |
| 768 | FPSSEQALAAVAYGQ | 196 | 210 |
| 769 | QALAAVAYGQADVFI | 201 | 215 |
| 770 | VAYGQADVFIGDALT | 206 | 220 |
| 771 | ADVFIGDALTTSHLV | 211 | 225 |
| 772 | GDALTTSHLVSQSYF | 216 | 230 |
| 773 | TSHLVSQSYFNDVRV | 221 | 235 |
| 774 | SQSYFNDVRVVAPAH | 226 | 240 |
| 775 | NDVRVVAPAHIATGG | 231 | 245 |
| 776 | VAPAHIATGGESFGV | 236 | 250 |
| 777 | IATGGESFGVRADNT | 241 | 255 |
| 778 | ESFGVRADNTRLLRV | 246 | 260 |
| 779 | RADNTRLLRVVNAVL | 251 | 265 |
| 780 | RLLRVVNAVLEAIPP | 256 | 270 |
| 781 | VNAVLEAIPPSEHRS | 261 | 275 |
| 782 | EAIPPSEHRSLIYRW | 266 | 280 |
| 783 | SEHRSLIYRWGLGSS | 271 | 285 |
| 784 | LIYRWGLGSSISLDF | 276 | 290 |
| 785 | GLGSSISLDFAHPAY | 281 | 295 |
| 786 | ISLDFAHPAYSAREQ | 286 | 300 |
| 787 | AHPAYSAREQQWMAD | 291 | 305 |
| 788 | SAREQQWMADHPVVK | 296 | 310 |
| 789 | QWMADHPVVKVAVLN | 301 | 315 |
| 790 | HPVVKVAVLNLFAPF | 306 | 320 |
| 791 | VAVLNLFAPFTLFRT | 311 | 325 |
| 792 | LFAPFTLFRTDEQFG | 316 | 330 |
| 793 | TLFRTDEQFGGISAA | 321 | 335 |
| 794 | DEQFGGISAAVLQLL | 326 | 340 |
| 795 | GISAAVLQLLQLRTG | 331 | 345 |
| 796 | VLQLLQLRTGLDFEI | 336 | 350 |
| 797 | QLRTGLDFEIIGVDT | 341 | 355 |
| 798 | LDFEIIGVDTVEELI | 346 | 360 |
| 799 | IGVDTVEELIAKLRS | 351 | 365 |
| 800 | VEELIAKLRSGEADM | 356 | 370 |
| 801 | AKLRSGEADMAGALF | 361 | 375 |
| 802 | GEADMAGALFVNSAR | 366 | 380 |
| 803 | AGALFVNSARESFLS | 371 | 385 |
| 804 | VNSARESFLSFSRPY | 376 | 390 |
| 805 | ESFLSFSRPYVRNGM | 381 | 395 |
| 806 | FSRPYVRNGMVIVTR | 386 | 400 |
| 807 | VRNGMVIVTRQDPDA | 391 | 405 |
| 808 | VIVTRQDPDAPVDAD | 396 | 410 |
| 809 | QDPDAPVDADHLDGR | 401 | 415 |
| 810 | PVDADHLDGRTVALV | 406 | 420 |
| 811 | HLDGRTVALVRNSAA | 411 | 425 |
| 812 | TVALVRNSAAIPLLQ | 416 | 430 |
| 813 | RNSAAIPLLORRYPQ | 421 | 435 |
| 814 | IPLLORRYPQAKVVT | 426 | 440 |
| 815 | RRYPQAKVVTADNPS | 431 | 445 |
| 816 | AKVVTADNPSEAMLM | 436 | 450 |
| 817 | ADNPSEAMLMVANGQ | 441 | 455 |
| 818 | EAMLMVANGQADAVV | 446 | 460 |
| 819 | VANGQADAVVQTQIS | 451 | 465 |
| 820 | ADAVVQTQISASYYV | 456 | 470 |
| 821 | QTQISASYYVNRYFA | 461 | 475 |
| 822 | ASYYVNRYFAGKLRI | 466 | 480 |
| 823 | NRYFAGKLRIASALD | 471 | 485 |
| 824 | GKLRIASALDLPPAE | 476 | 490 |
| 825 | ASALDLPPAEIALAT | 481 | 495 |
| 826 | LPPAEIALATTRGQT | 486 | 500 |
| 827 | IALATTRGQTELMSI | 491 | 505 |
| 828 | TRGQTELMSILNKAL | 496 | 510 |
| 829 | ELMSILNKALYSISN | 501 | 515 |
| 830 | LNKALYSISNDELAS | 506 | 520 |
| 831 | YSISNDELASIISRW | 511 | 525 |
| 832 | DELASIISRWRGSDG | 516 | 530 |
| 833 | IISRWRGSDGDPRTW | 521 | 535 |
| 834 | RGSDGDPRTWYAYRN | 526 | 540 |
| 835 | DPRTWYAYRNEIYLL | 531 | 545 |
| 836 | YAYRNEIYLLIGLGL | 536 | 550 |
| 837 | EIYLLIGLGLLSALL | 541 | 555 |
| 838 | IGLGLLSALLFLSWI | 546 | 560 |
| 839 | LSALLFLSWIVYLRR | 551 | 565 |
| 840 | FLSWIVYLRRQIRQR | 556 | 570 |
| 841 | VYLRRQIRQRKRAER | 561 | 575 |
| 842 | QIRQRKRAERALNDQ | 566 | 580 |
| 843 | KRAERALNDQLEFMR | 571 | 585 |
| 844 | ALNDQLEFMRVLIDG | 576 | 590 |
| 845 | LEFMRVLIDGTPNPI | 581 | 595 |
| 846 | VLIDGTPNPIYVRDK | 586 | 600 |
| 847 | TPNPIYVRDKEGRML | 591 | 605 |
| 848 | YVRDKEGRMLLCNDA | 596 | 610 |
| 849 | EGRMLLCNDAYLDTF | 601 | 615 |
| 850 | LCNDAYLDTFGVTAD | 606 | 620 |
| 851 | YLDTFGVTADAVLGK | 611 | 625 |
| 852 | GVTADAVLGKTIPEA | 616 | 630 |
| 853 | AVLGKTIPEANVVGD | 621 | 635 |
| 854 | TIPEANVVGDPALAR | 626 | 640 |
| 855 | NVVGDPALAREMHEF | 631 | 645 |
| 856 | PALAREMHEFLLTRV | 636 | 650 |
| 857 | EMHEFLLTRVAAERE | 641 | 655 |
| 858 | LLTRVAAEREPRFED | 646 | 660 |
| 859 | AAEREPRFEDRDVTL | 651 | 665 |
| 860 | PRFEDRDVTLHGRTR | 656 | 670 |
| 861 | RDVTLHGRTRHVYQW | 661 | 675 |
| 862 | HGRTRHVYQWTIPYG | 666 | 680 |
| 863 | HVYQWTIPYGDSLGE | 671 | 685 |
| 864 | TIPYGDSLGELKGII | 676 | 690 |
| 865 | DSLGELKGIIGGWID | 681 | 695 |
| 866 | LKGIIGGWIDITERA | 686 | 700 |
| 867 | GGWIDITERAELLRK | 691 | 705 |
| 868 | ITERAELLRKLHDAK | 696 | 710 |
| 869 | ELLRKLHDAKESADA | 701 | 715 |
| 870 | LHDAKESADAANRAK | 706 | 720 |
| 871 | ESADAANRAKTTFLA | 711 | 725 |
| 872 | ANRAKTTFLATMSHE | 716 | 730 |
| 873 | TTFLATMSHEIRTPM | 721 | 735 |
| 874 | TMSHEIRTPMNAIIG | 726 | 740 |
| 875 | IRTPMNAIIGMLELA | 731 | 745 |
| 876 | NAIIGMLELALLRPT | 736 | 750 |
| 877 | MLELALLRPTDQEPD | 741 | 755 |
| 878 | LLRPTDQEPDRQSIQ | 746 | 760 |
| 879 | DQEPDRQSIQVAYDS | 751 | 765 |
| 880 | RQSIQVAYDSARSLL | 756 | 770 |
| 881 | VAYDSARSLLELIGD | 761 | 775 |
| 882 | ARSLLELIGDILDIA | 766 | 780 |
| 883 | ELIGDILDIAKIEAG | 771 | 785 |
| 884 | ILDIAKIEAGKFDLA | 776 | 790 |
| 885 | KIEAGKFDLAPVRTA | 781 | 795 |
| 886 | KFDLAPVRTALRVLP | 786 | 800 |
| 887 | PVRTALRVLPEGAIR | 791 | 805 |
| 888 | LRVLPEGAIRVFDGL | 796 | 810 |
| 889 | EGAIRVFDGLARQKG | 801 | 815 |
| 890 | VFDGLARQKGIELVL | 806 | 820 |
| 891 | ARQKGIELVLKTDIV | 811 | 825 |
| 892 | IELVLKTDIVGVDDV | 816 | 830 |
| 893 | KTDIVGVDDVLIDPL | 821 | 835 |
| 894 | GVDDVLIDPLRMKQV | 826 | 840 |
| 895 | LIDPLRMKQVLSNLV | 831 | 845 |
| 896 | RMKQVLSNLVGNAIK | 836 | 850 |
| 897 | LSNLVGNAIKFTTEG | 841 | 855 |
| 898 | GNAIKFTTEGQVVLA | 846 | 860 |
| 899 | FTTEGQVVLAVTARP | 851 | 865 |
| 900 | QVVLAVTARPDGDAA | 856 | 870 |
| 901 | VTARPDGDAAHVQFS | 861 | 875 |
| 902 | DGDAAHVQFSVSDTG | 866 | 880 |
| 903 | HVQFSVSDTGCGISE | 871 | 885 |
| 904 | VSDTGCGISEADQRQ | 876 | 890 |
| 905 | CGISEADQRQLFKPF | 881 | 895 |
| 906 | ADQRQLFKPFSQVGG | 886 | 900 |
| 907 | LFKPFSQVGGSAEAG | 891 | 905 |
| 908 | SQVGGSAEAGPAPGT | 896 | 910 |
| 909 | SAEAGPAPGTGLGLS | 901 | 915 |
| 910 | PAPGTGLGLSISRRL | 906 | 920 |
| 911 | GLGLSISRRLVELMG | 911 | 925 |
| 912 | ISRRLVELMGGTLVM | 916 | 930 |
| 913 | VELMGGTLVMRSAPG | 921 | 935 |
| 914 | GTLVMRSAPGVGTTV | 926 | 940 |
| 915 | RSAPGVGTTVSVDLR | 931 | 945 |
| 916 | VGTTVSVDLRLTMVE | 936 | 950 |
| 917 | SVDLRLTMVEKSVQA | 941 | 955 |
| 918 | LTMVEKSVQAAPPAA | 946 | 960 |
| 919 | KSVQAAPPAAATAAT | 951 | 965 |
| 920 | APPAAATAATPSKPQ | 956 | 970 |
| 921 | ATAATPSKPQVSLRV | 961 | 975 |
| 922 | PSKPQVSLRVLVVDD | 966 | 980 |
| 923 | VSLRVLVVDDHKPNL | 971 | 985 |
| 924 | LVVDDHKPNLMLLRQ | 976 | 990 |
| 925 | HKPNLMLLRQQLDYL | 981 | 995 |
| 926 | MLLRQQLDYLGQRVI | 986 | 1000 |
| 927 | QLDYLGQRVIAADSG | 991 | 1005 |
| 928 | GQRVIAADSGEAALA | 996 | 1010 |
| 929 | AADSGEAALALWREH | 1001 | 1015 |
| 930 | EAALALWREHAFDVV | 1006 | 1020 |
| 931 | LWREHAFDVVITDCN | 1011 | 1025 |
| 932 | AFDVVITDCNMPGIS | 1016 | 1030 |
| 933 | ITDCNMPGISGYELA | 1021 | 1035 |
| 934 | MPGISGYELARRIRA | 1026 | 1040 |
| 935 | GYELARRIRAAEAAP | 1031 | 1045 |
| 936 | RRIRAAEAAPGYGRT | 1036 | 1050 |
| 937 | AEAAPGYGRTRCILF | 1041 | 1055 |
| 938 | GYGRTRCILFGFTAS | 1046 | 1060 |
| 939 | RCILFGFTASAQMDE | 1051 | 1065 |
| 940 | GFTASAQMDEAQRCR | 1056 | 1070 |
| 941 | AQMDEAQRCRAAGMD | 1061 | 1075 |
| 942 | AQRCRAAGMDDCLFK | 1066 | 1080 |
| 943 | AAGMDDCLFKPIGVD | 1071 | 1085 |
| 944 | DCLFKPIGVDALRQR | 1076 | 1090 |
| 945 | PIGVDALRQRLNEAV | 1081 | 1095 |
| 946 | ALRQRLNEAVARAAL | 1086 | 1100 |
| 947 | LNEAVARAALPTPPS | 1091 | 1105 |
| 948 | ARAALPTPPSPQAAA | 1096 | 1110 |
| 949 | PTPPSPQAAAPATDD | 1101 | 1115 |
| 950 | PQAAAPATDDATPTA | 1106 | 1120 |
| 951 | PATDDATPTAFSAES | 1111 | 1125 |
| 952 | ATPTAFSAESILALT | 1116 | 1130 |
| 953 | FSAESILALTQNDEA | 1121 | 1135 |
| 954 | ILALTONDEALIRQL | 1126 | 1140 |
| 955 | QNDEALIRQLLEEVI | 1131 | 1145 |
| 956 | LIRQLLEEVIRTNRA | 1136 | 1150 |
| 957 | LEEVIRTNRADVDQL | 1141 | 1155 |
| 958 | RTNRADVDQLQKLHQ | 1146 | 1160 |
| 959 | DVDQLQKLHQQADWP | 1151 | 1165 |
| 960 | QKLHQQADWPKVSDM | 1156 | 1170 |
| 961 | QADWPKVSDMAHRLA | 1161 | 1175 |
| 962 | KVSDMAHRLAGGARV | 1166 | 1180 |
| 963 | AHRLAGGARVVDAKA | 1171 | 1185 |
| 964 | GGARVVDAKAMIDTV | 1176 | 1190 |
| 965 | VDAKAMIDTVLALEK | 1181 | 1195 |
| 966 | MIDTVLALEKKAQGQ | 1186 | 1200 |
| 967 | LALEKKAQGQAGPSP | 1191 | 1205 |
| 968 | KAQGQAGPSPEIDGL | 1196 | 1210 |
| 969 | AGPSPEIDGLVRTLA | 1201 | 1215 |
| 970 | EIDGLVRTLAAQSAA | 1206 | 1220 |
| 971 | VRTLAAQSAALETQL | 1211 | 1225 |
| 972 | AQSAALETQLRAWLE | 1216 | 1230 |
| 973 | LETQLRAWLEQRPHQ | 1221 | 1235 |
| 974 | QLRAWLEQRPHQDQP | 1224 | 1238 |

| | |
|---|---|
| | Full Sequence |
| 2602 | MPAPHRLYPRSLICLAQALLAWALLAWAPAQASQELTLVG |
| | KAAVPDVEVALDGDDWRWLARKRVLTLGVYAPDIPPFDVT |
| | YGERYEGLTADYMAIIAHNLGMQAKVLRYPTREQALSALE |
| | SGQIDLIGTVNGTDGRQQSLRLSVPYAADHPVIVMPIGAR |
| | HVPASNLAGQRLAVDINYLPKETLARAYPQATLHYFPSSE |
| | QALAAVAYGQADVFIGDALTTSHLVSQSYFNDVRVVAPAH |
| | IATGGESFGVRADNTRLLRVVNAVLEAIPPSEHRSLIYRW |
| | GLGSSISLDFAHPAYSAREQQWMADHPVVKVAVLNLFAPF |
| | TLFRTDEQFGGISAAVLQLLQLRTGLDFEIIGVDTVEELI |
| | AKLRSGEADMAGALFVNSARESFLSFSRPYVRNGMVIVTR |
| | QDPDAPVDADHLDGRTVALVRNSAAIPLLQRRYPQAKVVT |
| | ADNPSEAMLMVANGQADAVVQTQISASYYVNRYFAGKLRI |
| | ASALDLPPAEIALATTRGQTELMSILNKALYSISNDELAS |
| | IISRWRGSDGDPRTWYAYRNEIYLLIGLGLLSALLFLSWI |
| | VYLRRQIRQRKRAERALNDQLEFMRVLIDGTPNPIYVRDK |
| | EGRMLLCNDAYLDTFGVTADAVLGKTIPEANVVGDPALAR |
| | EMHEFLLTRVAAEREPRFEDRDVTLHGRTRHVYQWTIPYG |
| | DSLGELKGIIGGWIDITERAELLRKLHDAKESADAANRAK |
| | TTFLATMSHEIRTPMNAIIGMLELALLRPTDQEPDRQSIQ |
| | VAYDSARSLLELIGDILDIAKIEAGKFDLAPVRTALRVLP |
| | EGAIRVFDGLARQKGIELVLKTDIVGVDDVLIDPLRMKQV |
| | LSNLVGNAIKFTTEGQVVLAVTARPDGDAAHVQFSVSDTG |
| | CGISEADQRQLFKPFSQVGGSAEAGPAPGTGLGLSISRRL |
| | VELMGGTLVMRSAPGVGTTVSVDLRLTMVEKSVQAAPPAA |
| | ATAATPSKPQVSLRVLVVDDHKPNLMLLRQQLDYLGQRVI |
| | AADSGEAALALWREHAFDVVITDCNMPGISGYELARRIRA |
| | AEAAPGYGRTRCILFGFTASAQMDEAQRCRAAGMDDCLFK |
| | PIGVDALRQRLNEAVARAALPTPPSPQAAAPATDDATPTA |
| | FSAESILALTQNDEALIRQLLEEVIRTNRADVDQLQKLHQ |
| | QADWPKVSDMAHRLAGGARVVDAKAMIDTVLALEKKAQGQ |
| | AGPSPEIDGLVRTLAAQSAALETQLRAWLEQRPHQDQP |

Table 7. PT(O) ANTS5—Overlapping peptides covering the entire sequence of NP_880086.1 1,4-alpha-glucan branching enzyme GlgB.

TABLE 7
PT(O) ANT 5-Overlapping peptides covering
the entire sequence of NP_880086.1 1,4-
alpha-glucan branching enzyme GlgB

| SEQ | | | |
|---|---|---|---|
| ID | | | |
| NO: | Peptide | Start | End |
| 975 | MMRDSPSIQGTLDAA | 1 | 15 |
| 976 | PSIQGTLDAATQHAL | 6 | 20 |
| 977 | TLDAATQHALLAGRH | 11 | 25 |
| 978 | TQHALLAGRHADPFS | 16 | 30 |
| 979 | LAGRHADPFSVLGPH | 21 | 35 |
| 980 | ADPFSVLGPHQAGAH | 26 | 40 |
| 981 | VLGPHQAGAHTVVRV | 31 | 45 |
| 982 | QAGAHTVVRVLAPGA | 36 | 50 |
| 983 | TVVRVLAPGARTVMA | 41 | 55 |
| 984 | LAPGARTVMAVLPGG | 46 | 60 |
| 985 | RTVMAVLPGGQRTPL | 51 | 65 |
| 986 | VLPGGQRTPLLPMQP | 56 | 70 |
| 987 | QRTPLLPMQPGLFEN | 61 | 75 |
| 988 | LPMQPGLFENTVPGL | 66 | 80 |
| 989 | GLFENTVPGLQPGAP | 71 | 85 |
| 990 | TVPGLQPGAPAAYRL | 76 | 90 |
| 991 | QPGAPAAYRLCIEWE | 81 | 95 |
| 992 | AAYRLCIEWEGGIQH | 86 | 100 |
| 993 | CIEWEGGIQHTADPY | 91 | 105 |
| 994 | GGIQHTADPYAFGPV | 96 | 110 |
| 995 | TADPYAFGPVLDAAQ | 101 | 115 |
| 996 | AFGPVLDAAQLDHCA | 106 | 120 |
| 997 | LDAAQLDHCAAGGWR | 111 | 125 |
| 998 | LDHCAAGGWRYLAGL | 116 | 130 |
| 999 | AGGWRYLAGLLGAHA | 121 | 135 |
| 1000 | YLAGLIGAHAASVDG | 126 | 140 |
| 1001 | LGAHAASVDGCAGTR | 131 | 145 |
| 1002 | ASVDGCAGTRFALWA | 136 | 150 |
| 1003 | CAGTRFALWAPNARR | 141 | 155 |
| 1004 | FALWAPNARRVAVVG | 146 | 160 |
| 1005 | PNARRVAVVGDFNGW | 151 | 165 |
| 1006 | VAVVGDFNGWDGRRH | 156 | 170 |
| 1007 | DFNGWDGRRHAMRLR | 161 | 175 |
| 1008 | DGRRHAMRLRYPAGV | 166 | 180 |
| 1009 | AMRLRYPAGVWELFL | 171 | 185 |
| 1010 | YPAGVWELFLPDVGP | 176 | 190 |
| 1011 | WELFLPDVGPGARYK | 181 | 195 |
| 1012 | PDVGPGARYKFQVLG | 186 | 200 |
| 1013 | GARYKFQVLGADGHT | 191 | 205 |
| 1014 | FQVLGADGHTVLKAD | 196 | 210 |
| 1015 | ADGHTVLKADPLARQ | 201 | 215 |
| 1016 | VLKADPLARQAEAPP | 206 | 220 |
| 1017 | PLARQAEAPPATASI | 211 | 225 |
| 1018 | AEAPPATASIVPDER | 216 | 230 |
| 1019 | ATASIVPDERPFAWT | 221 | 235 |
| 1020 | VPDERPFAWTDKAWM | 226 | 240 |
| 1021 | PFAWTDKAWMEQRAA | 231 | 245 |
| 1022 | DKAWMEQRAARQRCD | 236 | 250 |
| 1023 | EQRAARQRCDAPISI | 241 | 255 |
| 1024 | RQRCDAPISIYEVHA | 246 | 260 |
| 1025 | APISIYEVHAGSWFD | 251 | 265 |
| 1026 | YEVHAGSWFDDAGAP | 256 | 270 |
| 1027 | GSWFDDAGAPRWQNL | 261 | 275 |
| 1028 | DAGAPRWQNLAARLP | 266 | 280 |
| 1029 | RWQNLAARLPEYARS | 271 | 285 |
| 1030 | AARLPEYARSLGFTH | 276 | 290 |
| 1031 | EYARSLGFTHIELLP | 281 | 295 |
| 1032 | LGFTHIELLPVMAHP | 286 | 300 |
| 1033 | IELLPVMAHPFGGSW | 291 | 305 |
| 1034 | VMAHPFGGSWGYQPL | 296 | 310 |
| 1035 | FGGSWGYQPLGLFAP | 301 | 315 |
| 1036 | GYQPLGLFAPAAAHG | 306 | 320 |
| 1037 | GLFAPAAAHGAPADF | 311 | 325 |
| 1038 | AAAHGAPADFAHFVD | 316 | 330 |
| 1039 | APADFAHFVDRCHEA | 321 | 335 |
| 1040 | AHFVDRCHEAGLGVI | 326 | 340 |
| 1041 | RCHEAGLGVILDWVP | 331 | 345 |
| 1042 | GLGVILDWVPAHFPD | 336 | 350 |
| 1043 | LDWVPAHFPDDAHGL | 341 | 355 |
| 1044 | AHFPDDAHGLARLDG | 346 | 360 |
| 1045 | DAHGLARLDGTPLYE | 351 | 365 |
| 1046 | ARLDGTPLYEHADPR | 356 | 370 |
| 1047 | TPLYEHADPREGRHP | 361 | 375 |
| 1048 | HADPREGRHPDWNTL | 366 | 380 |
| 1049 | EGRHPDWNTLIYNYG | 371 | 385 |
| 1050 | DWNTLIYNYGRREVR | 376 | 390 |
| 1051 | IYNYGRREVRTFLIA | 381 | 395 |
| 1052 | RREVRTFLIASAIHW | 386 | 400 |
| 1053 | TFLIASAIHWLRHYH | 391 | 405 |
| 1054 | SAIHWLRHYHVDGLR | 396 | 410 |
| 1055 | LRHYHVDGLRVDAVA | 401 | 415 |
| 1056 | VDGLRVDAVASMLYR | 406 | 420 |
| 1057 | VDAVASMLYRDYSRP | 411 | 425 |
| 1058 | SMLYRDYSRPAGQWI | 416 | 430 |
| 1059 | DYSRPAGQWIPNRHG | 421 | 435 |
| 1060 | AGQWIPNRHGRRENL | 426 | 440 |
| 1061 | PNRHGRRENLEAIDF | 431 | 445 |
| 1062 | RRENLEAIDFLRELN | 436 | 450 |
| 1063 | EAIDFLRELNAAVGV | 441 | 455 |
| 1064 | LRELNAAVGVQCPGA | 446 | 460 |
| 1065 | AAVGVQCPGAITVAE | 451 | 465 |
| 1066 | QCPGAITVAEESTAW | 456 | 470 |
| 1067 | ITVAEESTAWPGVTA | 461 | 475 |
| 1068 | ESTAWPGVTAPVANG | 466 | 480 |
| 1069 | PGVTAPVANGGLGFD | 471 | 485 |
| 1070 | PVANGGLGFDYKWNM | 476 | 490 |
| 1071 | GLGFDYKWNMGWMHD | 481 | 495 |
| 1072 | YKWNMGWMHDTLRYM | 486 | 500 |
| 1073 | GWMHDTLRYMRRDPI | 491 | 505 |
| 1074 | TLRYMRRDPIHRRHH | 496 | 510 |
| 1075 | RRDPIHRRHHHHDLS | 501 | 515 |
| 1076 | HRRHHHHDLSFGMVY | 506 | 520 |
| 1077 | HHDLSFGMVYAYAER | 511 | 525 |
| 1078 | FGMVYAYAERFVLPL | 516 | 530 |
| 1079 | AYAERFVLPLSHDEV | 521 | 535 |
| 1080 | FVLPLSHDEVVHGKG | 526 | 540 |
| 1081 | SHDEVVHGKGSLLGK | 531 | 545 |
| 1082 | VHGKGSLLGKMPGER | 536 | 550 |
| 1083 | SLLGKMPGERAAQLA | 541 | 555 |
| 1084 | MPGERAAQLAQLRLY | 546 | 560 |
| 1085 | AAQLAQLRLYYAFMW | 551 | 565 |
| 1086 | QLRLYYAFMWAHPGK | 556 | 570 |
| 1087 | YAFMWAHPGKKLLFM | 561 | 575 |
| 1088 | AHPGKKLLFMGGEFG | 566 | 580 |
| 1089 | KLLFMGGEFGQQGEW | 571 | 585 |
| 1090 | GGEFGQQGEWNHDAM | 576 | 590 |
| 1091 | QQGEWNHDAMLQWSL | 581 | 595 |
| 1092 | NHDAMLQWSLLDDPA | 586 | 600 |
| 1093 | LOWSLLDDPAHRGLQ | 591 | 605 |
| 1094 | LDDPAHRGLQRLVAD | 596 | 610 |
| 1095 | HRGLQRLVADLNHVY | 601 | 615 |
| 1096 | RLVADLNHVYATLPE | 606 | 620 |
| 1097 | LNHVYATLPELHCRD | 611 | 625 |
| 1098 | ATLPELHCRDADPSG | 616 | 630 |
| 1099 | LHCRDADPSGFAWIV | 621 | 635 |
| 1100 | ADPSGFAWIVGDDAD | 626 | 640 |
| 1101 | FAWIVGDDADNSVLA | 631 | 645 |
| 1102 | GDDADNSVLAFARVD | 636 | 650 |
| 1103 | NSVLAFARVDASHCL | 641 | 655 |
| 1104 | FARVDASHCLVAVCN | 646 | 660 |
| 1105 | ASHCLVAVCNFTPVP | 651 | 665 |
| 1106 | VAVCNFTPVPRPGYR | 656 | 670 |
| 1107 | FTPVPRPGYRFGVPH | 661 | 675 |
| 1108 | RPGYRFGVPHAGDWR | 666 | 680 |
| 1109 | FGVPHAGDWRVRVDT | 671 | 685 |
| 1110 | AGDWRVRVDTGATRY | 676 | 690 |
| 1111 | VRVDTGATRYGGAGG | 681 | 695 |
| 1112 | GATRYGGAGGGPPIC | 686 | 700 |
| 1113 | GGAGGGPPICLRSEP | 691 | 705 |
| 1114 | GPPICLRSEPIPAHG | 696 | 710 |
| 1115 | LRSEPIPAHGHPQSL | 701 | 715 |
| 1116 | IPAHGHPQSLVLDLP | 706 | 720 |
| 1117 | HPQSLVLDLPGFTAL | 711 | 725 |
| 1118 | VLDLPGFTALYLRHS | 716 | 730 |
| 1119 | LDLPGFTALYLRHSE | 717 | 731 |

| | |
|---|---|
| | Full Sequence |
| 2603 | MMRDSPSIQGTLDAATQHALLAGRHADPFSVLGPHQAGAHT |
| | VVRVLAPGARTVMAVLPGGQRTPLLPMQPGLFENTVPGLQ |
| | PGAPAAYRLCIEWEGGIQHTADPYAFGPVLDAAQLDHCAA |
| | GGWRYLAGLLGAHAASVDGCAGTRFALWAPNARRVAVVGD |
| | FNGWDGRRHAMRLRYPAGVWELFLPDVGPGARYKFQVLGA |
| | DGHTVLKADPLARQAEAPPATASIVPDERPFAWTDKAWME |
| | QRAARQRCDAPISIYEVHAGSWFDDAGAPRWQNLAARLPE |
| | YARSLGFTHIELLPVMAHPFGGSWGYQPLGLFAPAAAHGA |
| | PADFAHFVDRCHEAGLGVILDWVPAHFPDDAHGLARLDGT |
| | PLYEHADPREGRHPDWNTLIYNYGRREVRTFLIASAIHWL |
| | RHYHVDGLRVDAVASMLYRDYSRPAGQWIPNRHGRRENLE |
| | AIDFLRELNAAVGVQCPGAITVAEESTAWPGVTAPVANGG |
| | LGFDYKWNMGWMHDTLRYMRRDPIHRRHHHHDLSFGMVYA |
| | YAERFVLPLSHDEVVHGKGSLLGKMPGERAAQLAQLRLYY |
| | AFMWAHPGKKLLFMGGEFGQQGEWNHDAMLQWSLLDDPAH |
| | RGLQRLVADLNHVYATLPELHCRDADPSGFAWIVGDDADN |
| | SVLAFARVDASHCLVAVCNFTPVPRPGYRFGVPHAGDWRV |
| | RVDTGATRYGGAGGGPPICLRSEPIPAHGHPQSLVLDLPG |
| | FTALYLRHSE |

TABLE 8 PT(O) ANT 6—Overlapping peptides covering the entire sequence of NP_881921.1 membrane protein [B. pertussis Tohama I

TABLE 8
PT(O) ANT 6-Overlapping peptides covering
the entire sequence of NP_881921.1 inner
protein [B. pertussis Tohama I

| SEQ | | | |
|---|---|---|---|
| ID | | | |
| NO: | Peptide | Start | End |
| 1120 | MTVPRPESIIPPAGN | 1 | 15 |
| 1121 | PESIIPPAGNAATGA | 6 | 20 |
| 1122 | PPAGNAATGAAGIAR | 11 | 25 |
| 1123 | AATGAAGIARAFKRA | 16 | 30 |
| 1124 | AGIARAFKRALVSQC | 21 | 35 |
| 1125 | AFKRALVSQCHPNML | 26 | 40 |
| 1126 | LVSQCHPNMLFAVLL | 31 | 45 |
| 1127 | HPNMLFAVLLPFLIA | 36 | 50 |
| 1128 | FAVLLPFLIALLGAI | 41 | 55 |
| 1129 | PFLIALLGAILLLWL | 46 | 60 |
| 1130 | LLGAILLLWLFWTPL | 51 | 65 |
| 1131 | LLLWLFWTPLNEWLR | 56 | 70 |
| 1132 | FWTPLNEWLRFEASQ | 61 | 75 |
| 1133 | NEWLRFEASQWQAIN | 66 | 80 |
| 1134 | FEASQWQAINQVDDW | 71 | 85 |
| 1135 | WQAINQVDDWMVAAG | 76 | 90 |
| 1136 | QVDDWMVAAGLFSLK | 81 | 95 |
| 1137 | MVAAGLFSLKIYLVP | 86 | 100 |
| 1138 | LFSLKIYLVPVIAAA | 91 | 105 |
| 1139 | IYLVPVIAAAILLPI | 96 | 110 |
| 1140 | VIAAAILLPISGILG | 101 | 115 |
| 1141 | ILLPISGILGLAIAA | 106 | 120 |
| 1142 | SGILGLAIAAVFVMP | 111 | 125 |
| 1143 | LAIAAVFVMPLVLRH | 116 | 130 |
| 1144 | VFVMPLVLRHVGGRE | 121 | 135 |
| 1145 | LVLRHVGGREYAGLA | 126 | 140 |
| 1146 | VGGREYAGLARQGRN | 131 | 145 |
| 1147 | YAGLARQGRNATAVS | 136 | 150 |
| 1148 | RQGRNATAVSVWNAL | 141 | 155 |
| 1149 | ATAVSVWNALWVSLA | 146 | 160 |
| 1150 | VWNALWVSLAFGAGW | 151 | 165 |
| 1151 | WVSLAFGAGWLLTLP | 156 | 170 |
| 1152 | FGAGWLLTLPFWLIP | 161 | 175 |
| 1153 | LLTLPFWLIPPMVVI | 166 | 180 |
| 1154 | FWLIPPMVVILSVFW | 171 | 185 |
| 1155 | PMVVILSVFWWAFAF | 176 | 190 |
| 1156 | LSVFWWAFAFTRMLR | 181 | 195 |
| 1157 | WAFAFTRMLRLDAIV | 186 | 200 |
| 1158 | TRMLRLDAIVEHASP | 191 | 205 |
| 1159 | LDAIVEHASPAERAI | 196 | 210 |
| 1160 | EHASPAERAILLKRH | 201 | 215 |
| 1161 | AERAILLKRHNSGFW | 206 | 220 |
| 1162 | LLKRHNSGFWLIGLV | 211 | 225 |
| 1163 | NSGFWLIGLVCSLLN | 216 | 230 |
| 1164 | LIGLVCSLLNLLPPA | 221 | 235 |
| 1165 | CSLLNLLPPAWIILP | 226 | 240 |
| 1166 | LLPPAWIILPVFSGL | 23 | 245 |
| 1167 | WIILPVFSGLVYAHY | 236 | 250 |
| 1168 | VFSGLVYAHYGLDAL | 24 | 255 |
| 1169 | VYAHYGLDALQRLRQ | 246 | 260 |
| 1170 | GLDALQRLRQERAID | 251 | 265 |
| 1171 | LDALQRLRQERAIDV | 252 | 266 |

| | |
|---|---|
| | Full Sequence |
| 2604 | MTVPRPESIIPPAGNAATGAAGIARAFKRALVSQCHPNMLF |
| | AVLLPFLIALLGAILLLWLFWTPLNEWLRFEASQWQAINQ |
| | VDDWMVAAGLFSLKIYLVPVIAAAILLPISGILGLAIAAV |
| | FVMPLVLRHVGGREYAGLARQGRNATAVSVWNALWVSLAF |
| | GAGWLLTLPFWLIPPMVVILSVFWWAFAFTRMLRLDAIVE |
| | HASPAERAILLKRHNSGFWLIGLVCSLLNLLPPAWIILPV |
| | FSGLVYAHYGLDALQRLRQERAIDV |

TABLE 9 PT(O) ANT 7—Overlapping peptides covering the entire sequence of NP_882013.1 BrkA autotransporter [B. pertussis Tohama I].

TABLE 9
PT(O) ANT 7-Overlapping peptides covering
the entire sequence of NP_882013.1 BrkA
autotransporter [B. pertussis Tohama I]

| SEQ ID | | | |
|---|---|---|---|
| NO: | Peptide | Start | End |
| 1172 | MYLDRFRQCPSSLQI | 1 | 15 |
| 1173 | FRQCPSSLQIPRSAW | 6 | 20 |
| 1174 | SSLQIPRSAWRLHAL | 11 | 25 |
| 1175 | PRSAWRLHALAAALA | 16 | 30 |
| 1176 | RLHALAAALALAGMA | 21 | 35 |
| 1177 | AAALALAGMARLAPA | 26 | 40 |
| 1178 | LAGMARLAPAAAQAP | 31 | 45 |
| 1179 | RLAPAAAQAPQPPVA | 36 | 50 |
| 1180 | AAQAPQPPVAGAPHA | 41 | 55 |
| 1181 | QPPVAGAPHAQDAGQ | 46 | 60 |
| 1182 | GAPHAQDAGQEGEFD | 51 | 65 |
| 1183 | QDAGQEGEFDHRDNT | 56 | 70 |
| 1184 | EGEFDHRDNTLIAVF | 61 | 75 |
| 1185 | HRDNTLIAVFDDGVG | 66 | 80 |
| 1186 | LIAVFDDGVGINLDD | 71 | 85 |
| 1187 | DDGVGINLDDDPDEL | 76 | 90 |
| 1188 | INLDDDPDELGETAP | 81 | 95 |
| 1189 | DPDELGETAPPTLKD | 86 | 100 |
| 1190 | GETAPPTLKDIHISV | 91 | 105 |
| 1191 | PTLKDIHISVEHKNP | 96 | 110 |
| 1192 | IHISVEHKNPMSKPA | 101 | 115 |
| 1193 | EHKNPMSKPAIGVRV | 106 | 120 |
| 1194 | MSKPAIGVRVSGAGR | 111 | 125 |
| 1195 | IGVRVSGAGRALTLA | 116 | 130 |
| 1196 | SGAGRALTLAGSTID | 121 | 135 |
| 1197 | ALTLAGSTIDATEGG | 126 | 140 |
| 1198 | GSTIDATEGGIPAVV | 131 | 145 |
| 1199 | ATEGGIPAVVRRGGT | 136 | 150 |
| 1200 | IPAVVRRGGTLELDG | 141 | 155 |
| 1201 | RRGGTLELDGVTVAG | 146 | 160 |
| 1202 | LELDGVTVAGGEGME | 151 | 165 |
| 1203 | VTVAGGEGMEPMTVS | 156 | 170 |
| 1204 | GEGMEPMTVSDAGSR | 161 | 175 |
| 1205 | PMTVSDAGSRLSVRG | 166 | 180 |
| 1206 | DAGSRLSVRGGVLGG | 171 | 185 |
| 1207 | LSVRGGVLGGEAPGV | 176 | 190 |
| 1208 | GVLGGEAPGVGLVRA | 181 | 195 |
| 1209 | EAPGVGLVRAAQGGQ | 186 | 200 |
| 1210 | GLVRAAQGGQASIID | 191 | 205 |
| 1211 | AQGGQASIIDATLQS | 196 | 210 |
| 1212 | ASIIDATLQSILGPA | 201 | 215 |
| 1213 | ATLQSILGPALIADG | 206 | 220 |
| 1214 | ILGPALIADGGSISV | 211 | 225 |
| 1215 | LIADGGSISVAGGSI | 216 | 230 |
| 1216 | GSISVAGGSIDMDMG | 221 | 235 |
| 1217 | AGGSIDMDMGPGFPP | 226 | 240 |
| 1218 | DMDMGPGFPPPPPPL | 231 | 245 |
| 1219 | PGFPPPPPPLPGAPL | 236 | 250 |
| 1220 | PPPPLPGAPLAAHPP | 241 | 255 |
| 1221 | PGAPLAAHPPLDRVA | 246 | 260 |
| 1222 | AAHPPLDRVAAVHAG | 251 | 265 |
| 1223 | LDRVAAVHAGQDGKV | 256 | 270 |
| 1224 | AVHAGQDGKVTLREV | 261 | 275 |
| 1225 | QDGKVTLREVALRAH | 266 | 280 |
| 1226 | TLREVALRAHGPQAT | 271 | 285 |
| 1227 | ALRAHGPQATGVYAY | 276 | 290 |
| 1228 | GPQATGVYAYMPGSE | 281 | 295 |
| 1229 | GVYAYMPGSEITLQG | 286 | 300 |
| 1230 | MPGSEITLQGGTVSV | 291 | 305 |
| 1231 | ITLQGGTVSVQGDDG | 296 | 310 |
| 1232 | GTVSVQGDDGAGVVA | 301 | 315 |
| 1233 | QGDDGAGVVAGAGLL | 306 | 320 |
| 1234 | AGVVAGAGLLDALPP | 311 | 325 |
| 1235 | GAGLLDALPPGGTVR | 316 | 330 |
| 1236 | DALPPGGTVRLDGTT | 321 | 335 |
| 1237 | GGTVRLDGTTVSTDG | 326 | 340 |
| 1238 | LDGTTVSTDGANTDA | 331 | 345 |
| 1239 | VSTDGANTDAVLVRG | 336 | 350 |
| 1240 | ANTDAVLVRGDAARA | 341 | 355 |
| 1241 | VLVRGDAARAEVVNT | 346 | 360 |
| 1242 | DAARAEVVNTVLRTA | 351 | 365 |
| 1243 | EVVNTVLRTAKSLAA | 356 | 370 |
| 1244 | VLRTAKSLAAGVSAQ | 361 | 375 |
| 1245 | KSLAAGVSAQHGGRV | 366 | 380 |
| 1246 | GVSAQHGGRVTLRQT | 371 | 385 |
| 1247 | HGGRVTLRQTRIETA | 376 | 390 |
| 1248 | TLRQTRIETAGAGAE | 381 | 395 |
| 1249 | RIETAGAGAEGISVL | 386 | 400 |
| 1250 | GAGAEGISVLGFEPQ | 391 | 405 |
| 1251 | GISVLGFEPQSGSGP | 396 | 410 |
| 1252 | GFEPQSGSGPASVDM | 401 | 415 |
| 1253 | SGSGPASVDMQGGSI | 406 | 420 |
| 1254 | ASVDMQGGSITTTGN | 411 | 425 |
| 1255 | QGGSITTTGNRAAGI | 416 | 430 |
| 1256 | TTTGNRAAGIALTHG | 421 | 435 |
| 1257 | RAAGIALTHGSARLE | 426 | 440 |
| 1258 | ALTHGSARLEGVAVR | 431 | 445 |
| 1259 | SARLEGVAVRAEGSG | 436 | 450 |
| 1260 | GVAVRAEGSGSSAAQ | 441 | 455 |
| 1261 | AEGSGSSAAQLANGT | 446 | 460 |
| 1262 | SSAAQLANGTLVVSA | 451 | 465 |
| 1263 | LANGTLVVSAGSLAS | 456 | 470 |
| 1264 | LVVSAGSLASAQSGA | 461 | 475 |
| 1265 | GSLASAQSGAISVTD | 466 | 480 |
| 1266 | AQSGAISVTDTPLKL | 471 | 485 |
| 1267 | ISVTDTPLKLMPGAL | 476 | 490 |
| 1268 | TPLKLMPGALASSTV | 481 | 495 |
| 1269 | MPGALASSTVSVRLT | 486 | 500 |
| 1270 | ASSTVSVRLTDGATA | 491 | 505 |
| 1271 | SVRLTDGATAQGGNG | 496 | 510 |
| 1272 | DGATAQGGNGVFLQQ | 501 | 515 |
| 1273 | QGGNGVFLQQHSTIP | 506 | 520 |
| 1274 | VFLQQHSTIPVAVAL | 511 | 525 |
| 1275 | HSTIPVAVALESGAL | 516 | 530 |
| 1276 | VAVALESGALARGDI | 521 | 535 |
| 1277 | ESGALARGDIVADGN | 526 | 540 |
| 1278 | ARGDIVADGNKPLDA | 531 | 545 |
| 1279 | VADGNKPLDAGISLS | 536 | 550 |
| 1280 | KPLDAGISLSVASGA | 541 | 555 |
| 1281 | GISLSVASGAAWHGA | 546 | 560 |
| 1282 | VASGAAWHGATQVLQ | 551 | 565 |
| 1283 | AWHGATQVLQSATLG | 556 | 570 |
| 1284 | TQVLQSATLGKGGTW | 561 | 575 |
| 1285 | SATLGKGGTWVVNAD | 566 | 580 |
| 1286 | KGGTWVVNADSRVQD | 571 | 585 |
| 1287 | VVNADSRVQDMSMRG | 576 | 590 |
| 1288 | SRVQDMSMRGGRVEF | 581 | 595 |
| 1289 | MSMRGGRVEFQAPAP | 586 | 600 |
| 1290 | GRVEFQAPAPEASYK | 591 | 605 |
| 1291 | QAPAPEASYKTLTLQ | 596 | 610 |
| 1292 | EASYKTLTLQTLDGN | 601 | 615 |
| 1293 | TLTLQTLDGNGVFVL | 606 | 620 |
| 1294 | TLDGNGVFVLNTNVA | 611 | 625 |
| 1295 | GVFVLNTNVAAGQND | 616 | 630 |
| 1296 | NTNVAAGQNDQLRVT | 621 | 635 |
| 1297 | AGQNDQLRVTGRADG | 626 | 640 |
| 1298 | QLRVTGRADGQHRVL | 631 | 645 |
| 1299 | GRADGQHRVLVRNAG | 636 | 650 |
| 1300 | QHRVLVRNAGGEADS | 641 | 655 |
| 1301 | VRNAGGEADSRGARL | 646 | 660 |
| 1302 | GEADSRGARLGLVHT | 651 | 665 |
| 1303 | RGARLGLVHTQGQGN | 656 | 670 |
| 1304 | GLVHTQGQGNATFRL | 661 | 675 |
| 1305 | QGQGNATFRLANVGK | 666 | 680 |
| 1306 | ATFRLANVGKAVDLG | 671 | 685 |
| 1307 | ANVGKAVDLGTWRYS | 676 | 690 |
| 1308 | AVDLGTWRYSLAEDP | 681 | 695 |
| 1309 | TWRYSLAEDPKTHVW | 686 | 700 |
| 1310 | LAEDPKTHVWSLQRA | 691 | 705 |
| 1311 | KTHVWSLQRAGQALS | 696 | 710 |
| 1312 | SLQRAGQALSGAANA | 701 | 715 |
| 1313 | GQALSGAANAAVNAA | 706 | 720 |
| 1314 | GAANAAVNAADLSSI | 711 | 725 |
| 1315 | AVNAADLSSIALAES | 716 | 730 |
| 1316 | DLSSIALAESNALDK | 721 | 735 |
| 1317 | ALAESNALDKRLGEL | 726 | 740 |
| 1318 | NALDKRLGELRLRAD | 731 | 745 |
| 1319 | RLGELRLRADAGGPW | 736 | 750 |
| 1320 | RLRADAGGPWARTFS | 741 | 755 |
| 1321 | AGGPWARTFSERQQI | 746 | 760 |
| 1322 | ARTFSERQQISNRHA | 751 | 765 |
| 1323 | ERQQISNRHARAYDQ | 756 | 770 |
| 1324 | SNRHARAYDQTVSGL | 761 | 775 |
| 1325 | RAYDQTVSGLEIGLD | 766 | 780 |
| 1326 | TVSGLEIGLDRGWSA | 771 | 785 |
| 1327 | EIGLDRGWSASGGRW | 776 | 790 |
| 1328 | RGWSASGGRWYAGGL | 781 | 795 |
| 1329 | SGGRWYAGGLLGYTY | 786 | 800 |
| 1330 | YAGGLLGYTYADRTY | 791 | 805 |
| 1331 | LGYTYADRTYPGDGG | 796 | 810 |
| 1332 | ADRTYPGDGGGKVKG | 801 | 815 |
| 1333 | PGDGGGKVKGLHVGG | 806 | 820 |
| 1334 | GKVKGLHVGGYAAYV | 811 | 825 |
| 1335 | LHVGGYAAYVGDGGY | 816 | 830 |
| 1336 | YAAYVGDGGYYLDTV | 821 | 835 |
| 1337 | GDGGYYLDTVLRLGR | 826 | 840 |
| 1338 | YLDTVLRLGRYDQQY | 831 | 845 |
| 1339 | LRLGRYDQQYNIAGT | 836 | 850 |
| 1340 | YDQQYNIAGTDGGRV | 841 | 855 |
| 1341 | NIAGTDGGRVTADYR | 846 | 860 |
| 1342 | DGGRVTADYRTSGAA | 851 | 865 |
| 1343 | TADYRTSGAAWSLEG | 856 | 870 |
| 1344 | TSGAAWSLEGGRRFE | 861 | 875 |
| 1345 | WSLEGGRRFELPNDW | 866 | 880 |
| 1346 | GRRFELPNDWFAEPQ | 871 | 885 |
| 1347 | LPNDWFAEPQAEVML | 876 | 890 |
| 1348 | FAEPQAEVMLWRTSG | 881 | 895 |
| 1349 | AEVMLWRTSGKRYRA | 886 | 900 |
| 1350 | WRTSGKRYRASNGLR | 891 | 905 |
| 1351 | KRYRASNGLRVKVDA | 896 | 910 |
| 1352 | SNGLRVKVDANTATL | 901 | 915 |
| 1353 | VKVDANTATLGRLGL | 906 | 920 |
| 1354 | NTATLGRLGLRFGRR | 911 | 925 |
| 1355 | GRLGLRFGRRIALAG | 916 | 930 |
| 1356 | RFGRRIALAGGNIVQ | 921 | 935 |
| 1357 | IALAGGNIVQPYARL | 926 | 940 |
| 1358 | GNIVQPYARLGWTQE | 931 | 945 |
| 1359 | PYARLGWTQEFKSTG | 936 | 950 |
| 1360 | GWTQEFKSTGDVRTN | 941 | 955 |
| 1361 | FKSTGDVRTNGIGHA | 946 | 960 |
| 1362 | DVRTNGIGHAGAGRH | 951 | 965 |
| 1363 | GIGHAGAGRHGRVEL | 956 | 970 |
| 1364 | GAGRHGRVELGAGVD | 961 | 975 |
| 1365 | GRVELGAGVDAALGK | 966 | 980 |
| 1366 | GAGVDAALGKGHNLY | 971 | 985 |
| 1367 | AALGKGHNLYASYEY | 976 | 990 |
| 1368 | GHNLYASYEYAAGDR | 981 | 995 |
| 1369 | ASYEYAAGDRINIPW | 986 | 1000 |
| 1370 | AAGDRINIPWSFHAG | 991 | 1005 |
| 1371 | INIPWSFHAGYRYSF | 996 | 1010 |

| | |
|---|---|
| | Full Sequence |
| 2605 | MYLDRFRQCPSSLQIPRSAWRLHALAAALALAGMARLAPAA |
| | AQAPQPPVAGAPHAQDAGQEGEFDHRDNTLIAVFDDGVGI |
| | NLDDDPDELGETAPPTLKDIHISVEHKNPMSKPAIGVRVS |
| | GAGRALTLAGSTIDATEGGIPAVVRRGGTLELDGVTVAGG |
| | EGMEPMTVSDAGSRLSVRGGVLGGEAPGVGLVRAAQGGQA |
| | SIIDATLQSILGPALIADGGSISVAGGSIDMDMGPGFPPP |
| | PPPLPGAPLAAHPPLDRVAAVHAGQDGKVTLREVALRAHG |
| | PQATGVYAYMPGSEITLQGGTVSVQGDDGAGVVAGAGLLD |
| | ALPPGGTVRLDGTTVSTDGANTDAVLVRGDAARAEVVNTV |
| | LRTAKSLAAGVSAQHGGRVTLRQTRIETAGAGAEGISVLG |
| | FEPQSGSGPASVDMQGGSITTTGNRAAGIALTHGSARLEG |
| | VAVRAEGSGSSAAQLANGTLVVSAGSLASAQSGAISVTDT |
| | PLKLMPGALASSTVSVRLTDGATAQGGNGVFLQQHSTIPV |
| | AVALESGALARGDIVADGNKPLDAGISLSVASGAAWHGAT |
| | QVLQSATLGKGGTWVVNADSRVQDMSMRGGRVEFQAPAPE |
| | ASYKTLTLQTLDGNGVFVLNTNVAAGQNDQLRVTGRADGQ |
| | HRVLVRNAGGEADSRGARLGLVHTQGQGNATFRLANVGKA |
| | VDLGTWRYSLAEDPKTHVWSLQRAGQALSGAANAAVNAAD |
| | LSSIALAESNALDKRLGELRLRADAGGPWARTFSERQQIS |
| | NRHARAYDQTVSGLEIGLDRGWSASGGRWYAGGLLGYTYA |
| | DRTYPGDGGGKVKGLHVGGYAAYVGDGGYYLDTVLRLGRY |
| | DQQYNIAGTDGGRVTADYRTSGAAWSLEGGRRFELPNDWF |
| | AEPQAEVMLWRTSGKRYRASNGLRVKVDANTATLGRLGLR |
| | FGRRIALAGGNIVQPYARLGWTQEFKSTGDVRTNGIGHAG |
| | AGRHGRVELGAGVDAALGKGHNLYASYEYAAGDRINIPWS |
| | FHAGYRYSF |

TABLE 10 PT(O) ANT 8—Overlapping peptides covering the entire sequence of NP_879664.1 NADH-quinone oxidoreductase subunit N [B. pertussis Tohama I].

TABLE 10
PT(O) ANT 8-Overlapping peptides covering
the entire sequence of NP_879664.1 NADH-
quinone subunit N [B. pertussis Tohama I]

| SEQ ID | | | |
|---|---|---|---|
| NO: | Peptide | Start | End |
| 1372 | MMQSHLDFALATPEI | 1 | 15 |
| 1373 | LDFALATPEILLLVL | 6 | 20 |
| 1374 | ATPEILLLVLGLAIL | 11 | 25 |
| 1375 | LLLVLGLAILLIDAV | 16 | 30 |
| 1376 | GLAILLIDAVSSHPE | 21 | 35 |
| 1377 | LIDAVSSHPERKTTF | 26 | 40 |
| 1378 | SSHPERKTTFVLTLA | 31 | 45 |
| 1379 | RKTTFVLTLATLAAL | 36 | 50 |
| 1380 | VLTLATLAALTVVSL | 41 | 55 |
| 1381 | TLAALTVVSLLQWRD | 46 | 60 |
| 1382 | TVVSLLQWRDGVEGQ | 51 | 65 |
| 1383 | LOWRDGVEGQTENGL | 56 | 70 |
| 1384 | GVEGQTFNGLYVTDS | 61 | 75 |
| 1385 | TFNGLYVTDSLAHLL | 66 | 80 |
| 1386 | YVTDSLAHLLKVASY | 71 | 85 |
| 1387 | LAHLLKVASYIAVAA | 76 | 90 |
| 1388 | KVASYIAVAATLVYG | 81 | 95 |
| 1389 | IAVAATLVYGRIYAQ | 86 | 100 |
| 1390 | TLVYGRIYAQQRDMM | 91 | 105 |
| 1391 | RIYAQQRDMMQRGGE | 96 | 110 |
| 1392 | QRDMMQRGGELYVLT | 101 | 115 |
| 1393 | QRGGELYVLTLFALL | 106 | 120 |
| 1394 | LYVLTLFALLGQMVM | 111 | 125 |
| 1395 | LFALLGQMVMISAGN | 116 | 130 |
| 1396 | GQMVMISAGNLISIY | 121 | 135 |
| 1397 | ISAGNLISIYLGLEL | 126 | 140 |
| 1398 | LISIYLGLELMSLAL | 131 | 145 |
| 1399 | LGLELMSLALYALIA | 136 | 150 |
| 1400 | MSLALYALIALRRED | 141 | 155 |
| 1401 | YALIALRREDKVATE | 146 | 160 |
| 1402 | LRREDKVATEAAMKY | 151 | 165 |
| 1403 | KVATEAAMKYFVLGA | 156 | 170 |
| 1404 | AAMKYFVLGALASGF | 161 | 175 |
| 1405 | FVLGALASGFLLYGM | 166 | 180 |
| 1406 | LASGFLLYGMSMVYG | 171 | 185 |
| 1407 | LLYGMSMVYGATGHL | 176 | 190 |
| 1408 | SMVYGATGHLDLAKI | 181 | 195 |
| 1409 | ATGHLDLAKIAEVIA | 186 | 200 |
| 1410 | DLAKIAEVIASGQAK | 191 | 205 |
| 1411 | AEVIASGQAKQLPLV | 196 | 210 |
| 1412 | SGQAKQLPLVFGVVF | 201 | 215 |
| 1413 | QLPLVFGVVFLVSGL | 206 | 220 |
| 1414 | FGVVFLVSGLAFKLG | 211 | 225 |
| 1415 | LVSGLAFKLGAVPFH | 216 | 230 |
| 1416 | AFKLGAVPFHMWLPD | 221 | 235 |
| 1417 | AVPFHMWLPDVYQGS | 226 | 240 |
| 1418 | MWLPDVYQGSPTAVT | 231 | 245 |
| 1419 | VYQGSPTAVTLILGA | 236 | 250 |
| 1420 | PTAVTLILGAAPKLA | 241 | 255 |
| 1421 | LILGAAPKLAAFAIT | 246 | 260 |
| 1422 | APKLAAFAITLRLLV | 251 | 265 |
| 1423 | AFAITLRLLVDGLHG | 256 | 270 |
| 1424 | LRLLVDGLHGLAADW | 261 | 275 |
| 1425 | DGLHGLAADWQPMLM | 266 | 280 |
| 1426 | LAADWQPMLMILAVL | 271 | 285 |
| 1427 | QPMLMILAVLSLAIG | 276 | 290 |
| 1428 | ILAVLSLAIGNLTAI | 281 | 295 |
| 1429 | SLAIGNLTAIVQTNF | 286 | 300 |
| 1430 | NLTAIVQTNFKRMLA | 291 | 305 |
| 1431 | VQTNFKRMLAYSTIS | 296 | 310 |
| 1432 | KRMLAYSTISHTGFV | 301 | 315 |
| 1433 | YSTISHTGFVLLGLM | 306 | 320 |
| 1434 | HTGFVLLGLMAGVVD | 311 | 325 |
| 1435 | LLGLMAGVVDGKPDA | 316 | 330 |
| 1436 | AGVVDGKPDAAASAY | 321 | 335 |
| 1437 | GKPDAAASAYGAALF | 326 | 340 |
| 1438 | AASAYGAALFYMLTY | 331 | 345 |
| 1439 | GAALFYMLTYVLTTL | 336 | 350 |
| 1440 | YMLTYVLTTLGTFGI | 341 | 355 |
| 1441 | VLTTLGTFGIILLLA | 346 | 360 |
| 1442 | GTFGIILLLARQGFE | 351 | 365 |
| 1443 | ILLLARQGFECEQID | 356 | 370 |
| 1444 | RQGFECEQIDDLKGL | 361 | 375 |
| 1445 | CEQIDDLKGLNRRNP | 366 | 380 |
| 1446 | DLKGLNRRNPWHAAI | 371 | 385 |
| 1447 | NRRNPWHAAIVLLLM | 376 | 390 |
| 1448 | WHAAIVLLLMFSLAG | 381 | 395 |
| 1449 | VLLLMFSLAGIPPLV | 386 | 400 |
| 1450 | FSLAGIPPLVGFYAK | 391 | 405 |
| 1451 | IPPLVGFYAKLAVLQ | 396 | 410 |
| 1452 | GFYAKLAVLQALVEA | 401 | 415 |
| 1453 | LAVLQALVEAGHVAL | 406 | 420 |
| 1454 | ALVEAGHVALAVVAV | 411 | 425 |
| 1455 | GHVALAVVAVMFSLI | 416 | 430 |
| 1456 | AVVAVMFSLIGAFYY | 421 | 435 |
| 1457 | MFSLIGAFYYLRVVK | 426 | 440 |
| 1458 | GAFYYLRVVKVVYFD | 431 | 445 |
| 1459 | LRVVKVVYFDDPVDQ | 436 | 450 |
| 1460 | VVYFDDPVDQPAALA | 441 | 455 |
| 1461 | DPVDQPAALAVTAGQ | 446 | 460 |
| 1462 | PAALAVTAGQRSILS | 451 | 465 |
| 1463 | VTAGQRSILSLNGAL | 456 | 470 |
| 1464 | RSILSLNGALILVLG | 461 | 475 |
| 1465 | LNGALILVLGILPGG | 466 | 480 |
| 1466 | ILVLGILPGGLMALC | 471 | 485 |
| 1467 | ILPGGLMALCVQVIQ | 476 | 490 |
| 1468 | GLMALCVQVIQASLG | 480 | 494 |

| | |
|---|---|
| | Full Sequence |
| 2606 | MMQSHLDFALATPEILLLVLGLAILLIDAVSSHPERKTTF |
| | VLTLATLAALTVVSLLQWRDGVEGQTFNGLYVTDSLAHLL |
| | KVASYIAVAATLVYGRIYAQQRDMMQRGGELYVLTLFALL |
| | GQMVMISAGNLISIYLGLELMSLALYALIALRREDKVATE |
| | AAMKYFVLGALASGFLLYGMSMVYGATGHLDLAKIAEVIA |
| | SGQAKQLPLVFGVVFLVSGLAFKLGAVPFHMWLPDVYQGS |
| | PTAVTLILGAAPKLAAFAITLRLLVDGLHGLAADWQPMLM |
| | ILAVLSLAIGNLTAIVQTNFKRMLAYSTISHTGFVLLGLM |
| | AGVVDGKPDAAASAYGAALFYMLTYVLTTLGTFGIILLLA |
| | RQGFECEQIDDLKGLNRRNPWHAAIVLLLMFSLAGIPPLV |
| | GFYAKLAVLQALVEAGHVALAVVAVMFSLIGAFYYLRVVK |
| | VVYFDDPVDQPAALAVTAGQRSILSLNGALILVLGILPGG |
| | LMALCVQVIQASLG |

TABLE 11 PT(O) ANT 9—Overlapping peptides covering the entire sequence of NP_882154.1 thiol:disulfide interchange protein [B. pertussis Tohama I].

TABLE 11
PT(O) ANT 9-Overlapping peptides covering the entire sequence of NP_882154.1
thiol:disulfide interchange protein [B. pertussis Tohama I]

| SEQ | | | |
|---|---|---|---|
| ID | | | |
| NO | Peptide | Start | End |
| 1469 | MMQYGHASATGHGAS | 1 | 15 |
| 1470 | HASATGHGASATGRA | 6 | 20 |
| 1471 | GHGASATGRAAAAGR | 11 | 25 |
| 1472 | ATGRAAAAGRWLAWM | 16 | 30 |
| 1473 | AAAGRWLAWMLALAL | 21 | 35 |
| 1474 | WLAWMLALALVLFAR | 26 | 40 |
| 1475 | LALALVLFARPAAAL | 31 | 45 |
| 1476 | VLFARPAAALTEDDF | 36 | 50 |
| 1477 | PAAALTEDDFLPPEQ | 41 | 55 |
| 1478 | TEDDFLPPEQAFVFS | 46 | 60 |
| 1479 | LPPEQAFVFSAAMAD | 51 | 65 |
| 1480 | AFVFSAAMADPATLV | 56 | 70 |
| 1481 | AAMADPATLVLNYRI | 61 | 75 |
| 1482 | PATLVLNYRIAPEYY | 66 | 80 |
| 1483 | LNYRIAPEYYMYRER | 71 | 85 |
| 1484 | APEYYMYRERFGLSA | 76 | 90 |
| 1485 | MYRERFGLSASPAQA | 81 | 95 |
| 1486 | FGLSASPAQAVTLGE | 86 | 100 |
| 1487 | SPAQAVTLGEAAYPQ | 91 | 105 |
| 1488 | VTLGEAAYPQGKVKY | 96 | 110 |
| 1489 | AAYPQGKVKYDPTFD | 101 | 115 |
| 1490 | GKVKYDPTFDKDMEV | 106 | 120 |
| 1491 | DPTFDKDMEVFYGTV | 111 | 125 |
| 1492 | KDMEVFYGTVAVRVP | 116 | 130 |
| 1493 | FYGTVAVRVPLSQGN | 121 | 135 |
| 1494 | AVRVPLSQGNGQPFT | 126 | 140 |
| 1495 | LSQGNGQPFTLTVTS | 131 | 145 |
| 1496 | GQPFTLTVTSQGCAD | 136 | 150 |
| 1497 | LTVTSQGCADAGLCY | 141 | 155 |
| 1498 | QGCADAGLCYPPMDN | 146 | 160 |
| 1499 | AGLCYPPMDNTVQLT | 151 | 165 |
| 1500 | PPMDNTVQLTPVTGG | 156 | 170 |
| 1501 | TVQLTPVTGGYALAA | 161 | 175 |
| 1502 | PVTGGYALAAGTASA | 166 | 180 |
| 1503 | YALAAGTASAPQGGG | 171 | 185 |
| 1504 | GTASAPQGGGTFDSL | 176 | 190 |
| 1505 | PQGGGTFDSLLEAGD | 181 | 195 |
| 1506 | TFDSLLEAGDTRLAD | 186 | 200 |
| 1507 | LEAGDTRLADFIGGG | 191 | 205 |
| 1508 | TRLADFIGGGGWLKT | 196 | 210 |
| 1509 | FIGGGGWLKTAGVFL | 201 | 215 |
| 1510 | GWLKTAGVFLLLGML | 206 | 220 |
| 1511 | AGVFLLLGMLLAFTP | 211 | 225 |
| 1512 | LLGMLLAFTPCVLPM | 216 | 230 |
| 1513 | LAFTPCVLPMVPILS | 221 | 235 |
| 1514 | CVLPMVPILSSIVLG | 226 | 240 |
| 1515 | VPILSSIVLGGAQAQ | 231 | 245 |
| 1516 | SIVLGGAQAQRPSRW | 236 | 250 |
| 1517 | GAQAQRPSRWRGLGL | 241 | 255 |
| 1518 | RPSRWRGLGLAAAYV | 246 | 260 |
| 1519 | RGLGLAAAYVFGMSV | 251 | 265 |
| 1520 | AAAYVFGMSVVYTAL | 256 | 270 |
| 1521 | FGMSVVYTALGVAAG | 261 | 275 |
| 1522 | VYTALGVAAGLSGAG | 266 | 280 |
| 1523 | GVAAGLSGAGLAAWL | 271 | 285 |
| 1524 | LSGAGLAAWLQTPWI | 276 | 290 |
| 1525 | LAAWLQTPWILSLFA | 281 | 295 |
| 1526 | QTPWILSLFAILLAV | 286 | 300 |
| 1527 | LSLFAILLAVLALAM | 291 | 305 |
| 1528 | ILLAVLALAMFGAFT | 296 | 310 |
| 1529 | LALAMFGAFTFQMPA | 301 | 315 |
| 1530 | FGAFTFQMPAGLQAR | 306 | 320 |
| 1531 | FQMPAGLQARLSERS | 311 | 325 |
| 1532 | GLQARLSERSNRIPG | 316 | 330 |
| 1533 | LSERSNRIPGGRVTG | 321 | 335 |
| 1534 | NRIPGGRVTGALVMG | 326 | 340 |
| 1535 | GRVTGALVMGALSAL | 331 | 345 |
| 1536 | ALVMGALSALIVGPC | 336 | 350 |
| 1537 | ALSALIVGPCVAAPL | 341 | 355 |
| 1538 | IVGPCVAAPLAGALL | 346 | 360 |
| 1539 | VAAPLAGALLYISQT | 351 | 365 |
| 1540 | AGALLYISQTGDVIL | 356 | 370 |
| 1541 | YISQTGDVILGGAAL | 361 | 375 |
| 1542 | GDVILGGAALFAMAW | 366 | 380 |
| 1543 | GGAALFAMAWGMGIP | 371 | 385 |
| 1544 | FAMAWGMGIPLLLVG | 376 | 390 |
| 1545 | GMGIPLLLVGASAGT | 381 | 395 |
| 1546 | LLLVGASAGTLLPRT | 386 | 400 |
| 1547 | ASAGTLLPRTGPWME | 391 | 405 |
| 1548 | LLPRTGPWMESVKRV | 396 | 410 |
| 1549 | GPWMESVKRVFGMLL | 401 | 415 |
| 1550 | SVKRVFGMLLLGTAW | 406 | 420 |
| 1551 | FGMLLLGTAWWMLIP | 411 | 425 |
| 1552 | LGTAWWMLIPVVPTW | 416 | 430 |
| 1553 | WMLIPVVPTWVQMLG | 421 | 435 |
| 1554 | VVPTWVQMLGWSFLA | 426 | 440 |
| 1555 | VQMLGWSFLAVVGAV | 431 | 445 |
| 1556 | WSFLAVVGAVMLRAF | 436 | 450 |
| 1557 | VVGAVMLRAFDALPA | 441 | 455 |
| 1558 | MLRAFDALPAGSGAP | 446 | 460 |
| 1559 | DALPAGSGAPRMFAK | 451 | 465 |
| 1560 | GSGAPRMFAKGLGLL | 456 | 470 |
| 1561 | RMFAKGLGLLLALAG | 461 | 475 |
| 1562 | GLGLLLALAGAAWLI | 466 | 480 |
| 1563 | LALAGAAWLIGALSG | 471 | 485 |
| 1564 | AAWLIGALSGGRDVL | 476 | 490 |
| 1565 | GALSGGRDVLAPLSH | 481 | 495 |
| 1566 | GRDVLAPLSHLAARA | 486 | 500 |
| 1567 | APLSHLAARAPAGGA | 491 | 505 |
| 1568 | LAARAPAGGAVAAAG | 496 | 510 |
| 1569 | PAGGAVAAAGPAAVD | 501 | 515 |
| 1570 | VAAAGPAAVDKTRFV | 506 | 520 |
| 1571 | PAAVDKTRFVRVRSN | 511 | 525 |
| 1572 | KTRFVRVRSNAELDA | 516 | 530 |
| 1573 | RVRSNAELDALLARS | 521 | 535 |
| 1574 | AELDALLARSTQPVM | 526 | 540 |
| 1575 | LLARSTQPVMLDFYA | 531 | 545 |
| 1576 | TQPVMLDFYADWCVS | 536 | 550 |
| 1577 | LDFYADWCVSCREME | 541 | 555 |
| 1578 | DWCVSCREMEHFTFS | 546 | 560 |
| 1579 | CREMEHFTFSDPTVA | 551 | 565 |
| 1580 | HFTFSDPTVAARMSQ | 556 | 570 |
| 1581 | DPTVAARMSQMLLVQ | 561 | 575 |
| 1582 | ARMSQMLLVQADVTK | 566 | 580 |
| 1583 | MLLVQADVTKNNADD | 571 | 585 |
| 1584 | ADVTKNNADDRALLK | 576 | 590 |
| 1585 | NNADDRALLKRFRLF | 581 | 595 |
| 1586 | RALLKRFRLFGPPGI | 586 | 600 |
| 1587 | RFRLFGPPGIMFFEP | 591 | 605 |
| 1588 | GPPGIMFFEPGGKLI | 596 | 610 |
| 1589 | MFFEPGGKLIEDIRV | 601 | 615 |
| 1590 | GGKLIEDIRVVGFQD | 606 | 620 |
| 1591 | EDIRVVGFQDARRFA | 611 | 625 |
| 1592 | VGFQDARRFAGVLEQ | 616 | 630 |
| 1593 | ARRFAGVLEQVADRS | 621 | 635 |
| 1594 | GVLEQVADRSGAPGP | 626 | 640 |
| 1595 | VADRSGAPGPAQAGS | 631 | 645 |
| | Full Sequence | | |

| | |
|---|---|
| 2607 | MMQYGHASATGHGASATGRAAAAGRWLAWMLALALVLFARPAAALTEDDFLPPE |
| | QAFVFSAAMADPATLVLNYRIAPEYYMYRERFGLSASPAQAVTLGEAAYPQGKVKY |
| | DPTFDKDMEVFYGTVAVRVPLSQGNGQPFTLTVTSQGCADAGLCYPPMDNTVQLTP |
| | VTGGYALAAGTASAPQGGGTFDSLLEAGDTRLADFIGGGGWLKTAGVFLLLGMLLA |
| | FTPCVLPMVPILSSIVLGGAQAQRPSRWRGLGLAAAYVFGMSVVYTALGVAAGLSG |
| | AGLAAWLQTPWILSLFAILLAVLALAMFGAFTFQMPAGLQARLSERSNRIPGGRVTG |
| | ALVMGALSALIVGPCVAAPLAGALLYISQTGDVILGGAALFAMAWGMGIPLLLVGA |
| | SAGTLLPRTGPWMESVKRVFGMLLLGTAWWMLIPVVPTWVQMLGWSFLAVVGAV |
| | MLRAFDALPAGSGAPRMFAKGLGLLLALAGAAWLIGALSGGRDVLAPLSHLAARAP |
| | AGGAVAAAGPAAVDKTRFVRVRSNAELDALLARSTQPVMLDFYADWCVSCREME |
| | HFTFSDPTVAARMSQMLLVQADVTKNNADDRALLKRFRLFGPPGIMFFEPGGKLIED |
| | IRVVGFQDARRFAGVLEQVADRSGAPGPAQAGS |

TABLE 12 PT(O) ANT 10—Overlapping peptides covering the entire sequence of NP_879578.1 bifunctional hemolysin-adenylate cyclase [B. pertussis Tohama I].

TABLE 12
PT(O) ANT 10-Overlapping peptides covering the entire sequence of NP_879578.1
bifunctional hemolysin-adenylate cyclase [B. pertussis Tohama I]

| SEQ ID | | | |
|---|---|---|---|
| NO: | Peptide | Start | End |
| 1596 | MQQSHQAGYANAADR | 1 | 15 |
| 1597 | QAGYANAADRESGIP | 6 | 20 |
| 1598 | NAADRESGIPAAVLD | 11 | 25 |
| 1599 | ESGIPAAVLDGIKAV | 16 | 30 |
| 1600 | AAVLDGIKAVAKEKN | 21 | 35 |
| 1601 | GIKAVAKEKNATLMF | 26 | 40 |
| 1602 | AKEKNATLMFRLVNP | 31 | 45 |
| 1603 | ATLMFRLVNPHSTSL | 36 | 50 |
| 1604 | RLVNPHSTSLIAEGV | 41 | 55 |
| 1605 | HSTSLIAEGVATKGL | 46 | 60 |
| 1606 | IAEGVATKGLGVHAK | 51 | 65 |
| 1607 | ATKGLGVHAKSSDWG | 56 | 70 |
| 1608 | GVHAKSSDWGLQAGY | 61 | 75 |
| 1609 | SSDWGLQAGYIPVNP | 66 | 80 |
| 1610 | LQAGYIPVNPNLSKL | 71 | 85 |
| 1611 | IPVNPNLSKLFGRAP | 76 | 90 |
| 1612 | NLSKLFGRAPEVIAR | 81 | 95 |
| 1613 | FGRAPEVIARADNDV | 86 | 100 |
| 1614 | EVIARADNDVNSSLA | 91 | 105 |
| 1615 | ADNDVNSSLAHGHTA | 96 | 110 |
| 1616 | NSSLAHGHTAVDLTL | 101 | 115 |
| 1617 | HGHTAVDLTLSKERL | 106 | 120 |
| 1618 | VDLTLSKERLDYLRQ | 111 | 125 |
| 1619 | SKERLDYLRQAGLVT | 116 | 130 |
| 1620 | DYLRQAGLVTGMADG | 121 | 135 |
| 1621 | AGLVTGMADGVVASN | 126 | 140 |
| 1622 | GMADGVVASNHAGYE | 131 | 145 |
| 1623 | VVASNHAGYEQFEFR | 136 | 150 |
| 1624 | HAGYEQFEFRVKETS | 141 | 155 |
| 1625 | QFEFRVKETSDGRYA | 146 | 160 |
| 1626 | VKETSDGRYAVQYRR | 151 | 165 |
| 1627 | DGRYAVQYRRKGGDD | 156 | 170 |
| 1628 | VQYRRKGGDDFEAVK | 161 | 175 |
| 1629 | KGGDDFEAVKVIGNA | 166 | 180 |
| 1630 | FEAVKVIGNAAGIPL | 171 | 185 |
| 1631 | VIGNAAGIPLTADID | 176 | 190 |
| 1632 | AGIPLTADIDMFAIM | 181 | 195 |
| 1633 | TADIDMFAIMPHLSN | 186 | 200 |
| 1634 | MFAIMPHLSNFRDSA | 191 | 205 |
| 1635 | PHLSNFRDSARSSVT | 196 | 210 |
| 1636 | FRDSARSSVTSGDSV | 201 | 215 |
| 1637 | RSSVTSGDSVTDYLA | 206 | 220 |
| 1638 | SGDSVTDYLARTRRA | 211 | 225 |
| 1639 | TDYLARTRRAASEAT | 216 | 230 |
| 1640 | RTRRAASEATGGLDR | 221 | 235 |
| 1641 | ASEATGGLDRERIDL | 226 | 240 |
| 1642 | GGLDRERIDLLWKIA | 231 | 245 |
| 1643 | ERIDLLWKIARAGAR | 236 | 250 |
| 1644 | LWKIARAGARSAVGT | 241 | 255 |
| 1645 | RAGARSAVGTEARRQ | 246 | 260 |
| 1646 | SAVGTEARRQFRYDG | 251 | 265 |
| 1647 | EARRQFRYDGDMNIG | 256 | 270 |
| 1648 | FRYDGDMNIGVITDF | 261 | 275 |
| 1649 | DMNIGVITDFELEVR | 266 | 280 |
| 1650 | VITDFELEVRNALNR | 271 | 285 |
| 1651 | ELEVRNALNRRAHAV | 276 | 290 |
| 1652 | NALNRRAHAVGAQDV | 281 | 295 |
| 1653 | RAHAVGAQDVVQHGT | 286 | 300 |
| 1654 | GAQDVVQHGTEQNNP | 291 | 305 |
| 1655 | VQHGTEQNNPFPEAD | 296 | 310 |
| 1656 | EQNNPFPEADEKIFV | 301 | 315 |
| 1657 | FPEADEKIFVVSATG | 306 | 320 |
| 1658 | EKIFVVSATGESQML | 311 | 325 |
| 1659 | VSATGESQMLTRGQL | 316 | 330 |
| 1660 | ESQMLTRGQLKEYIG | 321 | 335 |
| 1661 | TRGQLKEYIGQQRGE | 326 | 340 |
| 1662 | KEYIGQQRGEGYVFY | 331 | 345 |
| 1663 | QQRGEGYVFYENRAY | 336 | 350 |
| 1664 | GYVFYENRAYGVAGK | 341 | 355 |
| 1665 | ENRAYGVAGKSLFDD | 346 | 360 |
| 1666 | GVAGKSLFDDGLGAA | 351 | 365 |
| 1667 | SLFDDGLGAAPGVPS | 356 | 370 |
| 1668 | GLGAAPGVPSGRSKF | 361 | 375 |
| 1669 | PGVPSGRSKFSPDVL | 366 | 380 |
| 1670 | GRSKFSPDVLETVPA | 371 | 385 |
| 1671 | SPDVLETVPASPGLR | 376 | 390 |
| 1672 | ETVPASPGLRRPSLG | 381 | 395 |
| 1673 | SPGLRRPSLGAVERQ | 386 | 400 |
| 1674 | RPSLGAVERQDSGYD | 391 | 405 |
| 1675 | AVERQDSGYDSLDGV | 396 | 410 |
| 1676 | DSGYDSLDGVGSRSF | 401 | 415 |
| 1677 | SLDGVGSRSFSLGEV | 406 | 420 |
| 1678 | GSRSFSLGEVSDMAA | 411 | 425 |
| 1679 | SLGEVSDMAAVEAAE | 416 | 430 |
| 1680 | SDMAAVEAAELEMTR | 421 | 435 |
| 1681 | VEAAELEMTRQVLHA | 426 | 440 |
| 1682 | LEMTRQVLHAGARQD | 431 | 445 |
| 1683 | QVLHAGARQDDAEPG | 436 | 450 |
| 1684 | GARQDDAEPGVSGAS | 441 | 455 |
| 1685 | DAEPGVSGASAHWGQ | 446 | 460 |
| 1686 | VSGASAHWGQRALQG | 451 | 465 |
| 1687 | AHWGQRALQGAQAVA | 456 | 470 |
| 1688 | RALQGAQAVAAAQRL | 461 | 475 |
| 1689 | AQAVAAAQRLVHAIA | 466 | 480 |
| 1690 | AAQRLVHAIALMTQF | 471 | 485 |
| 1691 | VHAIALMTQFGRAGS | 476 | 490 |
| 1692 | LMTQFGRAGSTNTPQ | 481 | 495 |
| 1693 | GRAGSTNTPQEAASL | 486 | 500 |
| 1694 | TNTPQEAASLSAAVF | 491 | 505 |
| 1695 | EAASLSAAVFGLGEA | 496 | 510 |
| 1696 | SAAVFGLGEASSAVA | 501 | 515 |
| 1697 | GLGEASSAVAETVSG | 506 | 520 |
| 1698 | SSAVAETVSGFFRGS | 511 | 525 |
| 1699 | ETVSGFFRGSSRWAG | 516 | 530 |
| 1700 | FFRGSSRWAGGFGVA | 521 | 535 |
| 1701 | SRWAGGFGVAGGAMA | 526 | 540 |
| 1702 | GFGVAGGAMALGGGI | 531 | 545 |
| 1703 | GGAMALGGGIAAAVG | 536 | 550 |
| 1704 | LGGGIAAAVGAGMSL | 541 | 555 |
| 1705 | AAAVGAGMSLTDDAP | 546 | 560 |
| 1706 | AGMSLTDDAPAGQKA | 551 | 565 |
| 1707 | TDDAPAGQKAAAGAE | 556 | 570 |
| 1708 | AGQKAAAGAEIALQL | 561 | 575 |
| 1709 | AAGAEIALQLTGGTV | 566 | 580 |
| 1710 | IALQLTGGTVELASS | 571 | 585 |
| 1711 | TGGTVELASSIALAL | 576 | 590 |
| 1712 | ELASSIALALAAARG | 581 | 595 |
| 1713 | IALALAAARGVTSGL | 586 | 600 |
| 1714 | AAARGVTSGLQVAGA | 591 | 605 |
| 1715 | VTSGLQVAGASAGAA | 596 | 610 |
| 1716 | QVAGASAGAAAGALA | 601 | 615 |
| 1717 | SAGAAAGALAAALSP | 606 | 620 |
| 1718 | AGALAAALSPMEIYG | 611 | 625 |
| 1719 | AALSPMEIYGLVQQS | 616 | 630 |
| 1720 | MEIYGLVQQSHYADQ | 621 | 635 |
| 1721 | LVQQSHYADQLDKLA | 626 | 640 |
| 1722 | HYADQLDKLAQESSA | 631 | 645 |
| 1723 | LDKLAQESSAYGYEG | 636 | 650 |
| 1724 | QESSAYGYEGDALLA | 641 | 655 |
| 1725 | YGYEGDALLAQLYRD | 646 | 660 |
| 1726 | DALLAQLYRDKTAAE | 651 | 665 |
| 1727 | QLYRDKTAAEGAVAG | 656 | 670 |
| 1728 | KTAAEGAVAGVSAVL | 661 | 675 |
| 1729 | GAVAGVSAVLSTVGA | 666 | 680 |
| 1730 | VSAVLSTVGAAVSIA | 671 | 685 |
| 1731 | STVGAAVSIAAAASV | 676 | 690 |
| 1732 | AVSIAAAASVVGAPV | 681 | 695 |
| 1733 | AAASVVGAPVAVVTS | 686 | 700 |
| 1734 | VGAPVAVVTSLLTGA | 691 | 705 |
| 1735 | AVVTSLLTGALNGIL | 696 | 710 |
| 1736 | LLTGALNGILRGVQQ | 701 | 715 |
| 1737 | LNGILRGVQQPIIEK | 706 | 720 |
| 1738 | RGVQQPIIEKLANDY | 711 | 725 |
| 1739 | PIIEKLANDYARKID | 716 | 730 |
| 1740 | LANDYARKIDELGGP | 721 | 735 |
| 1741 | ARKIDELGGPQAYFE | 726 | 740 |
| 1742 | ELGGPQAYFEKNLQA | 731 | 745 |
| 1743 | QAYFEKNLQARHEQL | 736 | 750 |
| 1744 | KNLQARHEQLANSDG | 741 | 755 |
| 1745 | RHEQLANSDGLRKML | 746 | 760 |
| 1746 | ANSDGLRKMLADLQA | 751 | 765 |
| 1747 | LRKMLADLQAGWNAS | 756 | 770 |
| 1748 | ADLQAGWNASSVIGV | 761 | 775 |
| 1749 | GWNASSVIGVQTTEI | 766 | 780 |
| 1750 | SVIGVQTTEISKSAL | 771 | 785 |
| 1751 | QTTEISKSALELAAI | 776 | 790 |
| 1752 | SKSALELAAITGNAD | 781 | 795 |
| 1753 | ELAAITGNADNLKSV | 786 | 800 |
| 1754 | TGNADNLKSVDVFVD | 791 | 805 |
| 1755 | NLKSVDVFVDRFVQG | 796 | 810 |
| 1756 | DVFVDRFVQGERVAG | 801 | 815 |
| 1757 | RFVQGERVAGQPVVL | 806 | 820 |
| 1758 | ERVAGQPVVLDVAAG | 811 | 825 |
| 1759 | QPVVLDVAAGGIDIA | 816 | 830 |
| 1760 | DVAAGGIDIASRKGE | 821 | 835 |
| 1761 | GIDIASRKGERPALT | 826 | 840 |
| 1762 | SRKGERPALTFITPL | 831 | 845 |
| 1763 | RPALTFITPLAAPGE | 836 | 850 |
| 1764 | FITPLAAPGEEQRRR | 841 | 855 |
| 1765 | AAPGEEQRRRTKTGK | 846 | 860 |
| 1766 | EQRRRTKTGKSEFTT | 851 | 865 |
| 1767 | TKTGKSEFTTFVEIV | 856 | 870 |
| 1768 | SEFTTFVEIVGKQDR | 861 | 875 |
| 1769 | FVEIVGKQDRWRIRD | 866 | 880 |
| 1770 | GKQDRWRIRDGAADT | 871 | 885 |
| 1771 | WRIRDGAADTTIDLA | 876 | 890 |
| 1772 | GAADTTIDLAKVVSQ | 881 | 895 |
| 1773 | TIDLAKVVSQLVDAN | 886 | 900 |
| 1774 | KVVSQLVDANGVLKH | 891 | 905 |
| 1775 | LVDANGVLKHSIKLD | 896 | 910 |
| 1776 | GVLKHSIKLDVIGGD | 901 | 915 |
| 1777 | SIKLDVIGGDGDDVV | 906 | 920 |
| 1778 | VIGGDGDDVVLANAS | 911 | 925 |
| 1779 | GDDVVLANASRIHYD | 916 | 930 |
| 1780 | LANASRIHYDGGAGT | 921 | 935 |
| 1781 | RIHYDGGAGTNTVSY | 926 | 940 |
| 1782 | GGAGTNTVSYAALGR | 931 | 945 |
| 1783 | NTVSYAALGRQDSIT | 936 | 950 |
| 1784 | AALGRQDSITVSADG | 941 | 955 |
| 1785 | QDSITVSADGERFNV | 946 | 960 |
| 1786 | VSADGERFNVRKQLN | 951 | 965 |
| 1787 | ERFNVRKQLNNANVY | 956 | 970 |
| 1788 | RKQLNNANVYREGVA | 961 | 975 |
| 1789 | NANVYREGVATQTTA | 966 | 980 |
| 1790 | REGVATQTTAYGKRT | 971 | 985 |
| 1791 | TQTTAYGKRTENVQY | 976 | 990 |
| 1792 | YGKRTENVQYRHVEL | 981 | 995 |
| 1793 | ENVQYRHVELARVGQ | 986 | 1000 |
| 1794 | RHVELARVGQLVEVD | 991 | 1005 |
| 1795 | ARVGQLVEVDTLEHV | 996 | 1010 |
| 1796 | LVEVDTLEHVQHIIG | 1001 | 1015 |
| 1797 | TLEHVQHIIGGAGND | 1006 | 1020 |
| 1798 | QHIIGGAGNDSITGN | 1011 | 1025 |
| 1799 | GAGNDSITGNAHDNF | 1016 | 1030 |
| 1800 | SITGNAHDNFLAGGS | 1021 | 1035 |
| 1801 | AHDNFLAGGSGDDRL | 1026 | 1040 |
| 1802 | LAGGSGDDRLDGGAG | 1031 | 1045 |
| 1803 | GDDRLDGGAGNDTLV | 1036 | 1050 |
| 1804 | DGGAGNDTLVGGEGQ | 1041 | 1055 |
| 1805 | NDTLVGGEGQNTVIG | 1046 | 1060 |
| 1806 | GGEGQNTVIGGAGDD | 1051 | 1065 |
| 1807 | NTVIGGAGDDVFLQD | 1056 | 1070 |
| 1808 | GAGDDVFLQDLGVWS | 1061 | 1075 |
| 1809 | VFLQDLGVWSNQLDG | 1066 | 1080 |
| 1810 | LGVWSNQLDGGAGVD | 1071 | 1085 |
| 1811 | NQLDGGAGVDTVKYN | 1076 | 1090 |
| 1812 | GAGVDTVKYNVHQPS | 1081 | 1095 |
| 1813 | TVKYNVHQPSEERLE | 1086 | 1100 |
| 1814 | VHQPSEERLERMGDT | 1091 | 1105 |
| 1815 | EERLERMGDTGIHAD | 1096 | 1110 |
| 1816 | RMGDTGIHADLQKGT | 1101 | 1115 |
| 1817 | GIHADLQKGTVEKWP | 1106 | 1120 |
| 1818 | LQKGTVEKWPALNLF | 1111 | 1125 |
| 1819 | VEKWPALNLFSVDHV | 1116 | 1130 |
| 1820 | ALNLFSVDHVKNIEN | 1121 | 1135 |
| 1821 | SVDHVKNIENLHGSR | 1126 | 1140 |
| 1822 | KNIENLHGSRLNDRI | 1131 | 1145 |
| 1823 | LHGSRLNDRIAGDDQ | 1136 | 1150 |
| 1824 | LNDRIAGDDQDNELW | 1141 | 1155 |
| 1825 | AGDDQDNELWGHDGN | 1146 | 1160 |
| 1826 | DNELWGHDGNDTIRG | 1151 | 1165 |
| 1827 | GHDGNDTIRGRGGDD | 1156 | 1170 |
| 1828 | DTIRGRGGDDILRGG | 1161 | 1175 |
| 1829 | RGGDDILRGGLGLDT | 1166 | 1180 |
| 1830 | ILRGGLGLDTLYGED | 1171 | 1185 |
| 1831 | LGLDTLYGEDGNDIF | 1176 | 1190 |
| 1832 | LYGEDGNDIFLQDDE | 1181 | 1195 |
| 1833 | GNDIFLQDDETVSDD | 1186 | 1200 |
| 1834 | LQDDETVSDDIDGGA | 1191 | 1205 |
| 1835 | TVSDDIDGGAGLDTV | 1196 | 1210 |
| 1836 | IDGGAGLDTVDYSAM | 1201 | 1215 |
| 1837 | GLDTVDYSAMIHPGR | 1206 | 1220 |
| 1838 | DYSAMIHPGRIVAPH | 1211 | 1225 |
| 1839 | IHPGRIVAPHEYGFG | 1216 | 1230 |
| 1840 | IVAPHEYGFGIEADL | 1221 | 1235 |
| 1841 | EYGFGIEADLSREWV | 1226 | 1240 |
| 1842 | IEADLSREWVRKASA | 1231 | 1245 |
| 1843 | SREWVRKASALGVDY | 1236 | 1250 |
| 1844 | RKASALGVDYYDNVR | 1241 | 1255 |
| 1845 | LGVDYYDNVRNVENV | 1246 | 1260 |
| 1846 | YDNVRNVENVIGTSM | 1251 | 1265 |
| 1847 | NVENVIGTSMKDVLI | 1256 | 1270 |
| 1848 | IGTSMKDVLIGDAQA | 1261 | 1275 |
| 1849 | KDVLIGDAQANTLMG | 1266 | 1280 |
| 1850 | GDAQANTLMGQGGDD | 1271 | 1285 |
| 1851 | NTLMGQGGDDTVRGG | 1276 | 1290 |
| 1852 | QGGDDTVRGGDGDDL | 1281 | 1295 |
| 1853 | TVRGGDGDDLLFGGD | 1286 | 1300 |
| 1854 | DGDDLLFGGDGNDML | 1291 | 1305 |
| 1855 | LFGGDGNDMLYGDAG | 1296 | 1310 |
| 1856 | GNDMLYGDAGNDTLY | 1301 | 1315 |
| 1857 | YGDAGNDTLYGGLGD | 1306 | 1320 |
| 1858 | NDTLYGGLGDDTLEG | 1311 | 1325 |
| 1859 | GGLGDDTLEGGAGND | 1316 | 1330 |
| 1860 | DTLEGGAGNDWFGQT | 1321 | 1335 |
| 1861 | GAGNDWFGQTQAREH | 1326 | 1340 |
| 1862 | WFGQTQAREHDVLRG | 1331 | 1345 |
| 1863 | QAREHDVLRGGDGVD | 1336 | 1350 |
| 1864 | DVLRGGDGVDTVDYS | 1341 | 1355 |
| 1865 | GDGVDTVDYSQTGAH | 1346 | 1360 |
| 1866 | TVDYSQTGAHAGIAA | 1351 | 1365 |
| 1867 | QTGAHAGIAAGRIGL | 1356 | 1370 |
| 1868 | AGIAAGRIGLGILAD | 1361 | 1375 |
| 1869 | GRIGLGILADLGAGR | 1366 | 1380 |
| 1870 | GILADLGAGRVDKLG | 1371 | 1385 |
| 1871 | LGAGRVDKLGEAGSS | 1376 | 1390 |
| 1872 | VDKLGEAGSSAYDTV | 1381 | 1395 |
| 1873 | EAGSSAYDTVSGIEN | 1386 | 1400 |
| 1874 | AYDTVSGIENVVGTE | 1391 | 1405 |
| 1875 | SGIENVVGTELADRI | 1396 | 1410 |
| 1876 | VVGTELADRITGDAQ | 1401 | 1415 |
| 1877 | LADRITGDAQANVLR | 1406 | 1420 |
| 1878 | TGDAQANVLRGAGGA | 1411 | 1425 |
| 1879 | ANVLRGAGGADVLAG | 1416 | 1430 |
| 1880 | GAGGADVLAGGEGDD | 1421 | 1435 |
| 1881 | DVLAGGEGDDVLLGG | 1426 | 1440 |
| 1882 | GEGDDVLLGGDGDDQ | 1431 | 1445 |
| 1883 | VLLGGDGDDQLSGDA | 1436 | 1450 |
| 1884 | DGDDQLSGDAGRDRL | 1441 | 1455 |
| 1885 | LSGDAGRDRLYGEAG | 1446 | 1460 |
| 1886 | GRDRLYGEAGDDWFF | 1451 | 1465 |
| 1887 | YGEAGDDWFFQDAAN | 1456 | 1470 |
| 1888 | DDWFFQDAANAGNLL | 1461 | 1475 |
| 1889 | QDAANAGNLLDGGDG | 1466 | 1480 |
| 1890 | AGNLLDGGDGRDTVD | 1471 | 1485 |
| 1891 | DGGDGRDTVDFSGPG | 1476 | 1490 |
| 1892 | RDTVDFSGPGRGLDA | 1481 | 1495 |
| 1893 | FSGPGRGLDAGAKGV | 1486 | 1500 |
| 1894 | RGLDAGAKGVFLSLG | 1491 | 1505 |
| 1895 | GAKGVFLSLGKGFAS | 1496 | 1510 |
| 1896 | FLSLGKGFASLMDEP | 1501 | 1515 |
| 1897 | KGFASLMDEPETSNV | 1506 | 1520 |
| 1898 | LMDEPETSNVLRNIE | 1511 | 1525 |
| 1899 | ETSNVLRNIENAVGS | 1516 | 1530 |
| 1900 | LRNIENAVGSARDDV | 1521 | 1535 |
| 1901 | NAVGSARDDVLIGDA | 1526 | 1540 |
| 1902 | ARDDVLIGDAGANVL | 1531 | 1545 |
| 1903 | LIGDAGANVLNGLAG | 1536 | 1550 |
| 1904 | GANVLNGLAGNDVLS | 1541 | 1555 |
| 1905 | NGLAGNDVLSGGAGD | 1546 | 1560 |
| 1906 | NDVLSGGAGDDVLLG | 1551 | 1565 |
| 1907 | GGAGDDVLLGDEGSD | 1556 | 1570 |
| 1908 | DVLLGDEGSDLLSGD | 1561 | 1575 |
| 1909 | DEGSDLLSGDAGNDD | 1566 | 1580 |
| 1910 | LLSGDAGNDDLFGGQ | 1571 | 1585 |
| 1911 | AGNDDLFGGQGDDTY | 1576 | 1590 |
| 1912 | LFGGQGDDTYLFGVG | 1581 | 1595 |
| 1913 | GDDTYLFGVGYGHDT | 1586 | 1600 |
| 1914 | LFGVGYGHDTIYESG | 1591 | 1605 |
| 1915 | YGHDTIYESGGGHDT | 1596 | 1610 |
| 1916 | IYESGGGHDTIRINA | 1601 | 1615 |
| 1917 | GGHDTIRINAGADQL | 1606 | 1620 |
| 1918 | IRINAGADQLWFARQ | 1611 | 1625 |
| 1919 | GADQLWFARQGNDLE | 1616 | 1630 |
| 1920 | WFARQGNDLEIRILG | 1621 | 1635 |
| 1921 | GNDLEIRILGTDDAL | 1626 | 1640 |
| 1922 | IRILGTDDALTVHDW | 1631 | 1645 |
| 1923 | TDDALTVHDWYRDAD | 1636 | 1650 |
| 1924 | TVHDWYRDADHRVEI | 1641 | 1655 |
| 1925 | YRDADHRVEIIHAAN | 1646 | 1660 |
| 1926 | HRVEIIHAANQAVDQ | 1651 | 1665 |
| 1927 | IHAANQAVDQAGIEK | 1656 | 1670 |
| 1928 | QAVDQAGIEKLVEAM | 1661 | 1675 |
| 1929 | AGIEKLVEAMAQYPD | 1666 | 1680 |
| 1930 | LVEAMAQYPDPGAAA | 1671 | 1685 |
| 1931 | AQYPDPGAAAAAPPA | 1676 | 1690 |
| 1932 | PGAAAAAPPAARVPD | 1681 | 1695 |
| 1933 | AAPPAARVPDTLMQS | 1686 | 1700 |
| 1934 | ARVPDTLMQSLAVNW | 1691 | 1705 |
| 1935 | RVPDTLMQSLAVNWR | 1692 | 1706 |
| | Full Sequence | | |

| | |
|---|---|
| 2608 | MQQSHQAGYANAADRESGIPAAVLDGIKAVAKEKNATLMFRLVNPHSTSLIAEGV |
| | ATKGLGVHAKSSDWGLQAGYIPVNPNLSKLFGRAPEVIARADNDVNSSLAHGHTA |
| | VDLTLSKERLDYLRQAGLVTGMADGVVASNHAGYEQFEFRVKETSDGRYAVQYR |
| | RKGGDDFEAVKVIGNAAGIPLTADIDMFAIMPHLSNFRDSARSSVTSGDSVTDYLA |
| | RTRRAASEATGGLDRERIDLLWKIARAGARSAVGTEARRQFRYDGDMNIGVITDFE |
| | LEVRNALNRRAHAVGAQDVVQHGTEQNNPFPEADEKIFVVSATGESQMLTRGQLK |
| | EYIGQQRGEGYVFYENRAYGVAGKSLFDDGLGAAPGVPSGRSKFSPDVLETVPASP |
| | GLRRPSLGAVERQDSGYDSLDGVGSRSFSLGEVSDMAAVEAAELEMTRQVLHAGA |
| | RQDDAEPGVSGASAHWGQRALQGAQAVAAAQRLVHAIALMTQFGRAGSTNTPQE |
| | AASLSAAVFGLGEASSAVAETVSGFFRGSSRWAGGFGVAGGAMALGGGIAAAVG |
| | AGMSLTDDAPAGQKAAAGAEIALQLTGGTVELASSIALALAAARGVTSGLQVAGA |
| | SAGAAAGALAAALSPMEIYGLVQQSHYADQLDKLAQESSAYGYEGDALLAQLYR |
| | DKTAAEGAVAGVSAVLSTVGAAVSIAAAASVVGAPVAVVTSLLTGALNGILRGVQ |
| | QPIIEKLANDYARKIDELGGPQAYFEKNLQARHEQLANSDGLRKMLADLQAGWNA |
| | SSVIGVQTTEISKSALELAAITGNADNLKSVDVFVDRFVQGERVAGQPVVLDVAAG |
| | GIDIASRKGERPALTFITPLAAPGEEQRRRTKTGKSEFTTFVEIVGKQDRWRIRDGAA |
| | DTTIDLAKVVSQLVDANGVLKHSIKLDVIGGDGDDVVLANASRIHYDGGAGTNTV |
| | SYAALGRQDSITVSADGERFNVRKQLNNANVYREGVATQTTAYGKRTENVQYRH |
| | VELARVGQLVEVDTLEHVQHIIGGAGNDSITGNAHDNFLAGGSGDDRLDGGAGND |
| | TLVGGEGQNTVIGGAGDDVFLQDLGVWSNQLDGGAGVDTVKYNVHQPSEERLER |
| | MGDTGIHADLQKGTVEKWPALNLFSVDHVKNIENLHGSRLNDRIAGDDQDNELW |
| | GHDGNDTIRGRGGDDILRGGLGLDTLYGEDGNDIFLQDDETVSDDIDGGAGLDTVD |
| | YSAMIHPGRIVAPHEYGFGIEADLSREWVRKASALGVDYYDNVRNVENVIGTSMK |
| | DVLIGDAQANTLMGQGGDDTVRGGDGDDLLFGGDGNDMLYGDAGNDTLYGGLG |
| | DDTLEGGAGNDWFGQTQAREHDVLRGGDGVDTVDYSQTGAHAGIAAGRIGLGIL |
| | ADLGAGRVDKLGEAGSSAYDTVSGIENVVGTELADRITGDAQANVLRGAGGADVL |
| | AGGEGDDVLLGGDGDDQLSGDAGRDRLYGEAGDDWFFQDAANAGNLLDGGDGR |
| | DTVDFSGPGRGLDAGAKGVFLSLGKGFASLMDEPETSNVLRNIENAVGSARDDVLI |
| | GDAGANVLNGLAGNDVLSGGAGDDVLLGDEGSDLLSGDAGNDDLFGGQGDDTY |
| | LFGVGYGHDTIYESGGGHDTIRINAGADQLWFARQGNDLEIRILGTDDALTVHDWY |
| | RDADHRVEIIHAANQAVDQAGIEKLVEAMAQYPDPGAAAAAPPAARVPDTLMQSL |
| | AVNWR |

TABLE 13 PT(O) ANT 11—Overlapping peptides covering the entire sequence of NP_882012.1 serum resistance protein [B. pertussis Tohama I].

TABLE 13
PT(O) ANT 11-Overlapping peptides covering the entire sequence of NP_882012.1
serum resistance protein [B. pertussis Tohama I]

| SEQ ID | | | |
|---|---|---|---|
| NO: | Peptide | Start | End |
| 1936 | MRLPRQIRLPHLRDI | 1 | 15 |
| 1937 | QIRLPHLRDIGALLT | 6 | 20 |
| 1938 | HLRDIGALLTDSARE | 11 | 25 |
| 1939 | GALLTDSAREWSRHR | 16 | 30 |
| 1940 | DSAREWSRHRASSKG | 21 | 35 |
| 1941 | WSRHRASSKGAALSL | 26 | 40 |
| 1942 | ASSKGAALSLYMVFS | 31 | 45 |
| 1943 | AALSLYMVFSLAPML | 36 | 50 |
| 1944 | YMVFSLAPMLILVIA | 41 | 55 |
| 1945 | LAPMLILVIAVAGAF | 46 | 60 |
| 1946 | ILVIAVAGAFFGEEA | 51 | 65 |
| 1947 | VAGAFFGEEAVRSEL | 56 | 70 |
| 1948 | FGEEAVRSELFSQVR | 61 | 75 |
| 1949 | VRSELFSQVRDLTGE | 66 | 80 |
| 1950 | FSQVRDLTGERGAEV | 71 | 85 |
| 1951 | DLTGERGAEVIQTVL | 76 | 90 |
| 1952 | RGAEVIQTVLASAHE | 81 | 95 |
| 1953 | IQTVLASAHESGSGW | 86 | 100 |
| 1954 | ASAHESGSGWLAALL | 91 | 105 |
| 1955 | SGSGWLAALLSICVL | 96 | 110 |
| 1956 | LAALLSICVLVFSAT | 101 | 115 |
| 1957 | SICVLVFSATTAFAE | 106 | 120 |
| 1958 | VFSATTAFAELKASL | 111 | 125 |
| 1959 | TAFAELKASLDELWD | 116 | 130 |
| 1960 | LKASLDELWDVKEDK | 121 | 135 |
| 1961 | DELWDVKEDKSGLQG | 126 | 140 |
| 1962 | VKEDKSGLQGLVRSR | 131 | 145 |
| 1963 | SGLQGLVRSRMLSFG | 136 | 150 |
| 1964 | LVRSRMLSFGLVLVL | 141 | 155 |
| 1965 | MLSFGLVLVLALFLL | 146 | 160 |
| 1966 | LVLVLALFLLLSLTL | 151 | 165 |
| 1967 | ALFLLLSLTLNAALG | 156 | 170 |
| 1968 | LSLTLNAALGAAKGY | 161 | 175 |
| 1969 | NAALGAAKGYYGDLW | 166 | 180 |
| 1970 | AAKGYYGDLWSTSAF | 171 | 185 |
| 1971 | YGDLWSTSAFAMAAD | 176 | 190 |
| 1972 | STSAFAMAADWLSNL | 181 | 195 |
| 1973 | AMAADWLSNLFSFAV | 186 | 200 |
| 1974 | WLSNLFSFAVVTALF | 191 | 205 |
| 1975 | FSFAVVTALFAVVYK | 196 | 210 |
| 1976 | VTALFAVVYKLLPSK | 201 | 215 |
| 1977 | AVVYKLLPSKRIPWL | 206 | 220 |
| 1978 | LLPSKRIPWLDVIPG | 211 | 225 |
| 1979 | RIPWLDVIPGAIVTA | 216 | 230 |
| 1980 | DVIPGAIVTAALFLA | 221 | 235 |
| 1981 | AIVTAALFLAGKWGI | 226 | 240 |
| 1982 | ALFLAGKWGIGLYLG | 231 | 245 |
| 1983 | GKWGIGLYLGRGAAV | 236 | 250 |
| 1984 | GLYLGRGAAVSAYGA | 241 | 255 |
| 1985 | RGAAVSAYGAAGSLI | 246 | 260 |
| 1986 | SAYGAAGSLIALLLW | 251 | 265 |
| 1987 | AGSLIALLLWIYYSA | 256 | 270 |
| 1988 | ALLLWIYYSAQIFFF | 261 | 275 |
| 1989 | IYYSAQIFFFGAVFT | 266 | 280 |
| 1990 | QIFFFGAVFTRQFAE | 271 | 285 |
| 1991 | GAVFTRQFAERFGSL | 276 | 290 |
| 1992 | RQFAERFGSLRRAAP | 281 | 295 |
| 1993 | QFAERFGSLRRAAPA | 282 | 296 |
| | Full Sequence | | |

| | |
|---|---|
| 2609 | MRLPRQIRLPHLRDIGALLTDSAREWSRHRASSKGAALSLYMVFSLAPMLILVIAVA |
| | GAFFGEEAVRSELFSQVRDLTGERGAEVIQTVLASAHESGSGWLAALLSICVLVFSA |
| | TTAFAELKASLDELWDVKEDKSGLQGLVRSRMLSFGLVLVLALFLLLSLTLNAALG |
| | AAKGYYGDLWSTSAFAMAADWLSNLFSFAVVTALFAVVYKLLPSKRIPWLDVIPG |
| | AIVTAALFLAGKWGIGLYLGRGAAVSAYGAAGSLIALLLWIYYSAQIFFFGAVFTR |
| | QFAERFGSLRRAAPA |

TABLE 14 PT(O) ANT 12—Overlapping peptides covering the entire sequence of NP_880865.1 inner membrane protein [B. pertussis Tohama I].

TABLE 14
PT(O) ANT 12-Overlapping peptides covering the entire sequence of NP_880865.1
inner membrane protein [B. pertussis Tohama I]

| SEQ ID | | | |
|---|---|---|---|
| NO: | Peptide | Start | End |
| 1994 | MKNESSTTTADLEQL | 1 | 15 |
| 1995 | STTTADLEQLVAEAD | 6 | 20 |
| 1996 | DLEQLVAEADRGGRH | 11 | 25 |
| 1997 | VAEADRGGRHAGGVA | 16 | 30 |
| 1998 | RGGRHAGGVAGATLA | 21 | 35 |
| 1999 | AGGVAGATLAAGALV | 26 | 40 |
| 2000 | GATLAAGALVWSLFQ | 31 | 45 |
| 2001 | AGALVWSLFQLWYAS | 36 | 50 |
| 2002 | WSLFQLWYASPLPFS | 41 | 55 |
| 2003 | LWYASPLPFSLHWGV | 46 | 60 |
| 2004 | PLPFSLHWGVENDTE | 51 | 65 |
| 2005 | LHWGVENDTEARALH | 56 | 70 |
| 2006 | FNDTEARALHLGTAM | 61 | 75 |
| 2007 | ARALHLGTAMFLGYL | 66 | 80 |
| 2008 | LGTAMFLGYLAYPAT | 71 | 85 |
| 2009 | FLGYLAYPATKRSAR | 76 | 90 |
| 2010 | AYPATKRSARDRMPW | 81 | 95 |
| 2011 | KRSARDRMPWYDWVL | 86 | 100 |
| 2012 | DRMPWYDWVLALAAG | 91 | 105 |
| 2013 | YDWVLALAAGFCGAY | 96 | 110 |
| 2014 | ALAAGFCGAYLYLFY | 101 | 115 |
| 2015 | FCGAYLYLFYNELAI | 106 | 120 |
| 2016 | LYLFYNELAIRPGQP | 111 | 125 |
| 2017 | NELAIRPGQPTSMDV | 116 | 130 |
| 2018 | RPGQPTSMDVATAVA | 121 | 135 |
| 2019 | TSMDVATAVAGLLLL | 126 | 140 |
| 2020 | ATAVAGLLLLLEVTR | 131 | 145 |
| 2021 | GLLLLLEVTRRALGL | 136 | 150 |
| 2022 | LEVTRRALGLPMTVL | 141 | 155 |
| 2023 | RALGLPMTVLGAVFV | 146 | 160 |
| 2024 | PMTVLGAVFVLYALA | 151 | 165 |
| 2025 | GAVFVLYALAGPWLP | 156 | 170 |
| 2026 | LYALAGPWLPDVLAH | 161 | 175 |
| 2027 | GPWLPDVLAHRGASI | 166 | 180 |
| 2028 | DVLAHRGASIERLMS | 171 | 185 |
| 2029 | RGASIERLMSHMWLT | 176 | 190 |
| 2030 | ERLMSHMWLTTEGVY | 181 | 195 |
| 2031 | HMWLTTEGVYGVALG | 186 | 200 |
| 2032 | TEGVYGVALGVSVSY | 191 | 205 |
| 2033 | GVALGVSVSYIFIFV | 196 | 210 |
| 2034 | VSVSYIFIFVLLGSL | 201 | 215 |
| 2035 | IFIFVLLGSLLDKCG | 206 | 220 |
| 2036 | LLGSLLDKCGAGNYM | 211 | 225 |
| 2037 | LDKCGAGNYMMQVSF | 216 | 230 |
| 2038 | AGNYMMQVSFALLGH | 221 | 235 |
| 2039 | MQVSFALLGHLRGGP | 226 | 240 |
| 2040 | ALLGHLRGGPAKVAV | 231 | 245 |
| 2041 | LRGGPAKVAVVSSAV | 236 | 250 |
| 2042 | AKVAVVSSAVNGLVS | 241 | 255 |
| 2043 | VSSAVNGLVSASSVA | 246 | 260 |
| 2044 | NGLVSASSVANVVTG | 251 | 265 |
| 2045 | ASSVANVVTGGIFTI | 256 | 270 |
| 2046 | NVVTGGIFTIPLMKK | 261 | 275 |
| 2047 | GIFTIPLMKKAGYGG | 266 | 280 |
| 2048 | PLMKKAGYGGVRAGA | 271 | 285 |
| 2049 | AGYGGVRAGAIETAS | 276 | 290 |
| 2050 | VRAGAIETASSVNGQ | 281 | 295 |
| 2051 | IETASSVNGQIMPPV | 286 | 300 |
| 2052 | SVNGQIMPPVMGAAA | 291 | 305 |
| 2053 | IMPPVMGAAAFLMIE | 296 | 310 |
| 2054 | MGAAAFLMIEYVGIP | 301 | 315 |
| 2055 | FLMIEYVGIPYTDII | 306 | 320 |
| 2056 | YVGIPYTDIIRHAIL | 311 | 325 |
| 2057 | YTDIIRHAILPASIS | 316 | 330 |
| 2058 | RHAILPASISYIALF | 321 | 335 |
| 2059 | PASISYIALFYSVHL | 326 | 340 |
| 2060 | YIALFYSVHLEALKL | 331 | 345 |
| 2061 | YSVHLEALKLGIEPM | 336 | 350 |
| 2062 | EALKLGIEPMMAAGK | 341 | 355 |
| 2063 | GIEPMMAAGKPRTPL | 346 | 360 |
| 2064 | MAAGKPRTPLQKLAG | 351 | 365 |
| 2065 | PRTPLQKLAGWGMGI | 356 | 370 |
| 2066 | QKLAGWGMGISGTLI | 361 | 375 |
| 2067 | WGMGISGTLIAMGLV | 366 | 380 |
| 2068 | SGTLIAMGLVYWIGV | 371 | 385 |
| 2069 | AMGLVYWIGVGVQAV | 376 | 390 |
| 2070 | YWIGVGVQAVAGAAA | 381 | 395 |
| 2071 | GVQAVAGAAAIWILL | 386 | 400 |
| 2072 | AGAAAIWILLAMLVA | 391 | 405 |
| 2073 | IWILLAMLVALNIWL | 396 | 410 |
| 2074 | AMLVALNIWLLRVAA | 401 | 415 |
| 2075 | LNIWLLRVAARHPDL | 406 | 420 |
| 2076 | LRVAARHPDLPTEID | 411 | 425 |
| 2077 | RHPDLPTEIDVNHPV | 416 | 430 |
| 2078 | PTEIDVNHPVRPEPW | 421 | 435 |
| 2079 | VNHPVRPEPWPTVRA | 426 | 440 |
| 2080 | RPEPWPTVRAGLHFL | 431 | 445 |
| 2081 | PTVRAGLHFLIPIGI | 436 | 450 |
| 2082 | GLHFLIPIGILVWCL | 441 | 455 |
| 2083 | IPIGILVWCLSVEEL | 446 | 460 |
| 2084 | LVWCLSVEELSAGLS | 451 | 465 |
| 2085 | SVEELSAGLSAFWAA | 456 | 470 |
| 2086 | SAGLSAFWAAAATLL | 461 | 475 |
| 2087 | AFWAAAATLLQMVTQ | 466 | 480 |
| 2088 | AATLLQMVTQRPLTA | 471 | 485 |
| 2089 | QMVTQRPLTAWFRGQ | 476 | 490 |
| 2090 | RPLTAWFRGQAIAPA | 481 | 495 |
| 2091 | WFRGQAIAPAALLGW | 486 | 500 |
| 2092 | AIAPAALLGWRDAIG | 491 | 505 |
| 2093 | ALLGWRDAIGGLQDD | 496 | 510 |
| 2094 | RDAIGGLQDDARNMI | 501 | 515 |
| 2095 | GLQDDARNMIGIAIA | 506 | 520 |
| 2096 | ARNMIGIAIACGTAG | 511 | 525 |
| 2097 | GIAIACGTAGLIVGA | 516 | 530 |
| 2098 | CGTAGLIVGAITLTG | 521 | 535 |
| 2099 | LIVGAITLTGLGLRM | 526 | 540 |
| 2100 | ITLTGLGLRMTAFVE | 531 | 545 |
| 2101 | LGLRMTAFVELVSMG | 536 | 550 |
| 2102 | TAFVELVSMGNVLLM | 541 | 555 |
| 2103 | LVSMGNVLLMLIFTA | 546 | 560 |
| 2104 | NVLLMLIFTAIVCLI | 551 | 565 |
| 2105 | LIFTAIVCLILGLGM | 556 | 570 |
| 2106 | IVCLILGLGMPTTAN | 561 | 575 |
| 2107 | LGLGMPTTANYILMA | 566 | 580 |
| 2108 | PTTANYILMATLMAP | 571 | 585 |
| 2109 | YILMATLMAPVVVEL | 576 | 590 |
| 2110 | TLMAPVVVELGAQNG | 581 | 595 |
| 2111 | VVVELGAQNGLIIPL | 586 | 600 |
| 2112 | GAQNGLIIPLIAVHM | 591 | 605 |
| 2113 | LIIPLIAVHMFVFYY | 596 | 610 |
| 2114 | IAVHMFVFYYGIMAD | 601 | 615 |
| 2115 | FVFYYGIMADITPPV | 606 | 620 |
| 2116 | GIMADITPPVGLATF | 611 | 625 |
| 2117 | ITPPVGLATFAAAAI | 616 | 630 |
| 2118 | GLATFAAAAISGADP | 621 | 635 |
| 2119 | AAAAISGADPIKTGV | 626 | 640 |
| 2120 | SGADPIKTGVQGVTY | 631 | 645 |
| 2121 | IKTGVQGVTYALRTA | 636 | 650 |
| 2122 | QGVTYALRTAVLPFM | 641 | 655 |
| 2123 | ALRTAVLPFMFVFNP | 646 | 660 |
| 2124 | VLPFMFVFNPLLLLI | 651 | 665 |
| 2125 | FVFNPLLLLIDVNSW | 656 | 670 |
| 2126 | LLLLIDVNSWTELIL | 661 | 675 |
| 2127 | DVNSWTELILVAGSA | 666 | 680 |
| 2128 | TELILVAGSATLASL | 671 | 685 |
| 2129 | VAGSATLASLTFASA | 676 | 690 |
| 2130 | TLASLTFASATLGWF | 681 | 695 |
| 2131 | TFASATLGWFRVRCT | 686 | 700 |
| 2132 | TLGWFRVRCTMLEIV | 691 | 705 |
| 2133 | RVRCTMLEIVVLLAV | 696 | 710 |
| 2134 | MLEIVVLLAVTFMLF | 701 | 715 |
| 2135 | VLLAVTFMLFRPDWL | 706 | 720 |
| 2136 | TFMLFRPDWLLDQVS | 711 | 725 |
| 2137 | RPDWLLDQVSERYQA | 716 | 730 |
| 2138 | LDQVSERYQARPAAE | 721 | 735 |
| 2139 | ERYQARPAAEVVQTA | 726 | 740 |
| 2140 | RPAAEVVQTAAALPH | 731 | 745 |
| 2141 | VVQTAAALPHNGRLV | 736 | 750 |
| 2142 | AALPHNGRLVAVLRG | 741 | 755 |
| 2143 | NGRLVAVLRGINLEG | 746 | 760 |
| 2144 | AVLRGINLEGDELTK | 751 | 765 |
| 2145 | INLEGDELTKTVAVA | 756 | 770 |
| 2146 | DELTKTVAVALPALD | 761 | 775 |
| 2147 | TVAVALPALDEGETL | 766 | 780 |
| 2148 | LPALDEGETLQGEAA | 771 | 785 |
| 2149 | EGETLQGEAAGRKRL | 776 | 790 |
| 2150 | QGEAAGRKRLTDAGL | 781 | 795 |
| 2151 | GRKRLTDAGLTIVAL | 786 | 800 |
| 2152 | TDAGLTIVALGDQVQ | 791 | 805 |
| 2153 | TIVALGDQVQIGGVR | 796 | 810 |
| 2154 | GDQVQIGGVRFGSTA | 801 | 815 |
| 2155 | IGGVRFGSTARRAGW | 806 | 820 |
| 2156 | FGSTARRAGWEQGWD | 811 | 825 |
| 2157 | RRAGWEQGWDVLELR | 816 | 830 |
| 2158 | EQGWDVLELRVPNPA | 821 | 835 |
| 2159 | VLELRVPNPARPAEF | 826 | 840 |
| 2160 | VPNPARPAEFWAYLP | 831 | 845 |
| 2161 | RPAEFWAYLPGLVLL | 836 | 850 |
| 2162 | WAYLPGLVLLALVWF | 841 | 855 |
| 2163 | GLVLLALVWFAQGRR | 846 | 860 |
| 2164 | ALVWFAQGRRQRAAA | 851 | 865 |
| 2165 | LVWFAQGRRQRAAAR | 852 | 866 |
| | Full Sequence | | |

| | |
|---|---|
| 2610 | MKNESSTTTADLEQLVAEADRGGRHAGGVAGATLAAGALVWSLFQLWYASP |
| | LPFSLHWGVENDTEARALHLGTAMFLGYLAYPATKRSARDRMPWYDWVLAL |
| | AAGFCGAYLYLFYNELAIRPGQPTSMDVATAVAGLLLLLEVTRRALGLPMTVL |
| | GAVFVLYALAGPWLPDVLAHRGASIERLMSHMWLTTEGVYGVALGVSVSYIFI |
| | FVLLGSLLDKCGAGNYMMQVSFALLGHLRGGPAKVAVVSSAVNGLVSASSVA |
| | NVVTGGIFTIPLMKKAGYGGVRAGAIETASSVNGQIMPPVMGAAAFLMIEYVGI |
| | PYTDIIRHAILPASISYIALFYSVHLEALKLGIEPMMAAGKPRTPLQKLAGWGMG |
| | ISGTLIAMGLVYWIGVGVQAVAGAAAIWILLAMLVALNIWLLRVAARHPDLPT |
| | EIDVNHPVRPEPWPTVRAGLHFLIPIGILVWCLSVEELSAGLSAFWAAAATLLQ |
| | MVTQRPLTAWFRGQAIAPAALLGWRDAIGGLQDDARNMIGIAIACGTAGLIVG |
| | AITLTGLGLRMTAFVELVSMGNVLLMLIFTAIVCLILGLGMPTTANYILMATLM |
| | APVVVELGAQNGLIIPLIAVHMFVFYYGIMADITPPVGLATFAAAAISGADPIKT |
| | GVQGVTYALRTAVLPFMFVFNPLLLLIDVNSWTELILVAGSATLASLTFASATL |
| | GWFRVRCTMLEIVVLLAVTFMLFRPDWLLDQVSERYQARPAAEVVQTAAALP |
| | HNGRLVAVLRGINLEGDELTKTVAVALPALDEGETLQGEAAGRKRLTDAGLTI |
| | VALGDQVQIGGVRFGSTARRAGWEQGWDVLELRVPNPARPAEFWAYLPGLVL |
| | LALVWFAQGRRQRAAAR |

TABLE 15 PT(O) ANT 13—Overlapping peptides covering the entire sequence of NP_880575.1 filamentous hemagglutinin transporter protein FhaC [B. pertussis Tohama I].

TABLE 15
PT(O) ANT 13-Overlapping peptides covering the entire sequence of NP_880575.1
hemagglutinin transporter protein FhaC [B. pertussis Tohama I]

| SEQ ID | | | |
|---|---|---|---|
| NO: | Peptide | Start | End |
| 2166 | MTDATNRFRPGLVGR | 1 | 15 |
| 2167 | NRFRPGLVGRALVRA | 6 | 20 |
| 2168 | GLVGRALVRAGLLFA | 11 | 25 |
| 2169 | ALVRAGLLFAVAACA | 16 | 30 |
| 2170 | GLLFAVAACAQAQLL | 21 | 35 |
| 2171 | VAACAQAQLLPGARD | 26 | 40 |
| 2172 | QAQLLPGARDLNRID | 31 | 45 |
| 2173 | PGARDLNRIDDRQRK | 36 | 50 |
| 2174 | LNRIDDRQRKEQLQR | 41 | 55 |
| 2175 | DRQRKEQLQRDIERA | 46 | 60 |
| 2176 | EQLQRDIERALTRPP | 51 | 65 |
| 2177 | DIERALTRPPVELNP | 56 | 70 |
| 2178 | LTRPPVELNPQSEAA | 61 | 75 |
| 2179 | VELNPQSEAAAPARK | 66 | 80 |
| 2180 | QSEAAAPARKPDATS | 71 | 85 |
| 2181 | APARKPDATSGHTVT | 76 | 90 |
| 2182 | PDATSGHTVTVHAVD | 81 | 95 |
| 2183 | GHTVTVHAVDLDFGV | 86 | 100 |
| 2184 | VHAVDLDFGVEGRLF | 91 | 105 |
| 2185 | LDFGVEGRLFDPAPL | 96 | 110 |
| 2186 | EGRLFDPAPLVQDYL | 101 | 115 |
| 2187 | DPAPLVQDYLNRPLD | 106 | 120 |
| 2188 | VQDYLNRPLDNEQLF | 111 | 125 |
| 2189 | NRPLDNEQLFLLVKA | 116 | 130 |
| 2190 | NEQLFLLVKALSAAL | 121 | 135 |
| 2191 | LLVKALSAALYDRGY | 126 | 140 |
| 2192 | LSAALYDRGYATSIV | 131 | 145 |
| 2193 | YDRGYATSIVTFVPP | 136 | 150 |
| 2194 | ATSIVTFVPPGVVDG | 141 | 155 |
| 2195 | TFVPPGVVDGVLKLK | 146 | 160 |
| 2196 | GVVDGVLKLKVEWGR | 151 | 165 |
| 2197 | VLKLKVEWGRIKGWL | 156 | 170 |
| 2198 | VEWGRIKGWLIDGKP | 161 | 175 |
| 2199 | IKGWLIDGKPLEGTR | 166 | 180 |
| 2200 | IDGKPLEGTRDRMMV | 171 | 185 |
| 2201 | LEGTRDRMMVFSAMP | 176 | 190 |
| 2202 | DRMMVFSAMPGWQDK | 181 | 195 |
| 2203 | FSAMPGWQDKVLNVF | 186 | 200 |
| 2204 | GWQDKVLNVFDIDQA | 191 | 205 |
| 2205 | VLNVFDIDQAIYNIN | 196 | 210 |
| 2206 | DIDQAIYNINNGGKT | 201 | 215 |
| 2207 | IYNINNGGKTGNITI | 206 | 220 |
| 2208 | NGGKTGNITIVPADE | 211 | 225 |
| 2209 | GNITIVPADEYGYSY | 216 | 230 |
| 2210 | VPADEYGYSYLDLQL | 221 | 235 |
| 2211 | YGYSYLDLQLQRRAL | 226 | 240 |
| 2212 | LDLQLQRRALPRVSL | 231 | 245 |
| 2213 | QRRALPRVSLGMDNS | 236 | 250 |
| 2214 | PRVSLGMDNSGPGTP | 241 | 255 |
| 2215 | GMDNSGPGTPENGRY | 246 | 260 |
| 2216 | GPGTPENGRYKYNAS | 251 | 265 |
| 2217 | ENGRYKYNASVTAND | 256 | 270 |
| 2218 | KYNASVTANDLLGLN | 261 | 275 |
| 2219 | VTANDLLGLNDTLGL | 266 | 280 |
| 2220 | LLGLNDTLGLYIGNR | 271 | 285 |
| 2221 | DTLGLYIGNRYYRDA | 276 | 290 |
| 2222 | YIGNRYYRDAGHDAE | 281 | 295 |
| 2223 | YYRDAGHDAERNYDL | 286 | 300 |
| 2224 | GHDAERNYDLMYSVP | 291 | 305 |
| 2225 | RNYDLMYSVPLGRTR | 296 | 310 |
| 2226 | MYSVPLGRTRLDLQT | 301 | 315 |
| 2227 | LGRTRLDLQTGYSTY | 306 | 320 |
| 2228 | LDLQTGYSTYRNLLK | 311 | 325 |
| 2229 | GYSTYRNLLKTRYGQ | 316 | 330 |
| 2230 | RNLLKTRYGQYQSAG | 321 | 335 |
| 2231 | TRYGQYQSAGNSRSF | 326 | 340 |
| 2232 | YQSAGNSRSFGLKAT | 331 | 345 |
| 2233 | NSRSFGLKATRLLYR | 336 | 350 |
| 2234 | GLKATRLLYRDTRSQ | 341 | 355 |
| 2235 | RLLYRDTRSQFSVYG | 346 | 360 |
| 2236 | DTRSQFSVYGGLKLR | 351 | 365 |
| 2237 | FSVYGGLKLRQNKNY | 356 | 370 |
| 2238 | GLKLRQNKNYLAGTR | 361 | 375 |
| 2239 | QNKNYLAGTRLDVSS | 366 | 380 |
| 2240 | LAGTRLDVSSKHYSD | 371 | 385 |
| 2241 | LDVSSKHYSDVTVGM | 376 | 390 |
| 2242 | KHYSDVTVGMQYSTQ | 381 | 395 |
| 2243 | VTVGMQYSTQRGANA | 386 | 400 |
| 2244 | QYSTQRGANAYFGDL | 391 | 405 |
| 2245 | RGANAYFGDLSFTRG | 396 | 410 |
| 2246 | YFGDLSFTRGVGVNN | 401 | 415 |
| 2247 | SFTRGVGVNNGKYAA | 406 | 420 |
| 2248 | VGVNNGKYAAYDERG | 411 | 425 |
| 2249 | GKYAAYDERGPQGNV | 416 | 430 |
| 2250 | YDERGPQGNVSRFNG | 421 | 435 |
| 2251 | PQGNVSRFNGSLAWT | 426 | 440 |
| 2252 | SRFNGSLAWTRYMAL | 431 | 445 |
| 2253 | SLAWTRYMALAGQPI | 436 | 450 |
| 2254 | RYMALAGQPIQWASQ | 441 | 455 |
| 2255 | AGQPIQWASQLGFQY | 446 | 460 |
| 2256 | QWASQLGFQYSRQQL | 451 | 465 |
| 2257 | LGFQYSRQQLLNSYQ | 456 | 470 |
| 2258 | SRQQLLNSYQITVGD | 461 | 475 |
| 2259 | LNSYQITVGDEYTVR | 466 | 480 |
| 2260 | ITVGDEYTVRGYNLR | 471 | 485 |
| 2261 | EYTVRGYNLRTSQSG | 476 | 490 |
| 2262 | GYNLRTSQSGDSGVY | 481 | 495 |
| 2263 | TSQSGDSGVYLSNTL | 486 | 500 |
| 2264 | DSGVYLSNTLTVPVQ | 491 | 505 |
| 2265 | LSNTLTVPVQFSLLG | 496 | 510 |
| 2266 | TVPVQFSLLGKQASV | 501 | 515 |
| 2267 | FSLLGKQASVAPFVG | 506 | 520 |
| 2268 | KQASVAPFVGADVGA | 511 | 525 |
| 2269 | APFVGADVGALKSNH | 516 | 530 |
| 2270 | ADVGALKSNHPDART | 521 | 535 |
| 2271 | LKSNHPDARTIRMAG | 526 | 540 |
| 2272 | PDARTIRMAGLAAGV | 531 | 545 |
| 2273 | IRMAGLAAGVRFDLP | 536 | 550 |
| 2274 | LAAGVRFDLPYARMS | 541 | 555 |
| 2275 | RFDLPYARMSFTYSK | 546 | 560 |
| 2276 | YARMSFTYSKPVGAQ | 551 | 565 |
| 2277 | FTYSKPVGAQPGGAP | 556 | 570 |
| 2278 | PVGAQPGGAPRAPVW | 561 | 575 |
| 2279 | PGGAPRAPVWLYINA | 566 | 580 |
| 2280 | PRAPVWLYINAGLSF | 570 | 584 |
| | Full Sequence | | |

| 2611 | MTDATNRFRPGLVGRALVRAGLLFAVAACAQAQLLPGARDLNRIDDRQRKEQ |
|---|---|
| | LQRDIERALTRPPVELNPQSEAAAPARKPDATSGHTVTVHAVDLDFGVEGRLF |
| | DPAPLVQDYLNRPLDNEQLFLLVKALSAALYDRGYATSIVTFVPPGVVDGVLK |
| | LKVEWGRIKGWLIDGKPLEGTRDRMMVFSAMPGWQDKVLNVFDIDQAIYNIN |
| | NGGKTGNITIVPADEYGYSYLDLQLQRRALPRVSLGMDNSGPGTPENGRYKYN |
| | ASVTANDLLGLNDTLGLYIGNRYYRDAGHDAERNYDLMYSVPLGRTRLDLQT |
| | GYSTYRNLLKTRYGQYQSAGNSRSFGLKATRLLYRDTRSQFSVYGGLKLRQNK |
| | NYLAGTRLDVSSKHYSDVTVGMQYSTQRGANAYFGDLSFTRGVGVNNGKYA |
| | AYDERGPQGNVSRFNGSLAWTRYMALAGQPIQWASQLGFQYSRQQLLNSYQI |
| | TVGDEYTVRGYNLRTSQSGDSGVYLSNTLTVPVQFSLLGKQASVAPFVGADVG |
| | ALKSNHPDARTIRMAGLAAGVRFDLPYARMSFTYSKPVGAQPGGAPRAPVWL |
| | YINAGLSF |

TABLE 16 PT(O) ANT 14—Overlapping peptides covering the entire sequence of NP_882038.1 membrane protein [B. pertussis Tohama I].

TABLE 16
PT(O) ANT 14-Overlapping peptides covering the entire sequence of NP_882038.1
membrane protein [B. pertussis Tohama I]

| SEQ ID | | | |
|---|---|---|---|
| NO: | Peptide | Start | End |
| 2281 | MQAVLTAALPVFALI | 1 | 15 |
| 2282 | TAALPVFALILTGWL | 6 | 20 |
| 2283 | VFALILTGWLAARWR | 11 | 25 |
| 2284 | LTGWLAARWRVLGPS | 16 | 30 |
| 2285 | AARWRVLGPSATDAL | 21 | 35 |
| 2286 | VLGPSATDALNRYVV | 26 | 40 |
| 2287 | ATDALNRYVVYLSLP | 31 | 45 |
| 2288 | NRYVVYLSLPALLFR | 36 | 50 |
| 2289 | YLSLPALLFRAMAQA | 41 | 55 |
| 2290 | ALLFRAMAQADLRQL | 46 | 60 |
| 2291 | AMAQADLRQLADYWD | 51 | 65 |
| 2292 | DLRQLADYWDFTAAV | 56 | 70 |
| 2293 | ADYWDFTAAVAGGIA | 61 | 75 |
| 2294 | FTAAVAGGIALTFGA | 66 | 80 |
| 2295 | AGGIALTFGAAILAC | 71 | 85 |
| 2296 | LTFGAAILACRRDGA | 76 | 90 |
| 2297 | AILACRRDGARLTDL | 81 | 95 |
| 2298 | RRDGARLTDLSLEGL | 86 | 100 |
| 2299 | RLTDLSLEGLATSYG | 91 | 105 |
| 2300 | SLEGLATSYGNAGYM | 96 | 110 |
| 2301 | ATSYGNAGYMGIPLC | 101 | 115 |
| 2302 | NAGYMGIPLCLALLG | 106 | 120 |
| 2303 | GIPLCLALLGPASLA | 111 | 125 |
| 2304 | LALLGPASLAPAIIT | 116 | 130 |
| 2305 | PASLAPAIITTLLTA | 121 | 135 |
| 2306 | PAIITTLLTACVLFG | 126 | 140 |
| 2307 | TLLTACVLFGVAIAL | 131 | 145 |
| 2308 | CVLFGVAIALIEFDQ | 136 | 150 |
| 2309 | VAIALIEFDQHRDRH | 141 | 155 |
| 2310 | IEFDQHRDRHWSATL | 146 | 160 |
| 2311 | HRDRHWSATLLKVAR | 151 | 165 |
| 2312 | WSATLLKVARALLRN | 156 | 170 |
| 2313 | LKVARALLRNPLLAA | 161 | 175 |
| 2314 | ALLRNPLLAAPLLGL | 166 | 180 |
| 2315 | PLLAAPLLGLACAAA | 171 | 185 |
| 2316 | PLLGLACAAAGITLP | 176 | 190 |
| 2317 | ACAAAGITLPAGLDN | 181 | 195 |
| 2318 | GITLPAGLDNYAALL | 186 | 200 |
| 2319 | AGLDNYAALLGASAS | 191 | 205 |
| 2320 | YAALLGASASPCALV | 196 | 210 |
| 2321 | GASASPCALVTIGLF | 201 | 215 |
| 2322 | PCALVTIGLFLAQSQ | 206 | 220 |
| 2323 | TIGLFLAQSQPGGDR | 211 | 225 |
| 2324 | LAQSQPGGDRGTVGL | 216 | 230 |
| 2325 | PGGDRGTVGLMVGGK | 221 | 235 |
| 2326 | GTVGLMVGGKLLLHP | 226 | 240 |
| 2327 | MVGGKLLLHPAVTAV | 231 | 245 |
| 2328 | LLLHPAVTAVLAFAV | 236 | 250 |
| 2329 | AVTAVLAFAVFDMPP | 241 | 255 |
| 2330 | LAFAVFDMPPLWAWC | 246 | 260 |
| 2331 | FDMPPLWAWCAVLMA | 251 | 265 |
| 2332 | LWAWCAVLMAALPIG | 256 | 270 |
| 2333 | AVLMAALPIGTGPFM | 261 | 275 |
| 2334 | ALPIGTGPFMLAQLY | 266 | 280 |
| 2335 | TGPFMLAQLYGRDAR | 271 | 285 |
| 2336 | LAQLYGRDARPSSRA | 276 | 290 |
| 2337 | GRDARPSSRAILLST | 281 | 295 |
| 2338 | PSSRAILLSTVLSVP | 286 | 300 |
| 2339 | ILLSTVLSVPTITAL | 291 | 305 |
| 2340 | VLSVPTITALVAWIG | 296 | 310 |
| 2341 | TITALVAWIGRQPLG | 301 | 315 |
| | Full Sequence | | |

| | |
|---|---|
| 2612 | MQAVLTAALPVFALILTGWLAARWRVLGPSATDALNRYVVYLSLPALLFRAM |
| | AQADLRQLADYWDFTAAVAGGIALTFGAAILACRRDGARLTDLSLEGLATSYG |
| | NAGYMGIPLCLALLGPASLAPAIITTLLTACVLFGVAIALIEFDQHRDRHWSATL |
| | LKVARALLRNPLLAAPLLGLACAAAGITLPAGLDNYAALLGASASPCALVTIGL |
| | FLAQSQPGGDRGTVGLMVGGKLLLHPAVTAVLAFAVFDMPPLWAWCAVLMA |
| | ALPIGTGPFMLAQLYGRDARPSSRAILLSTVLSVPTITALVAWIGRQPLG |

TABLE 17 PT(O) ANT 15—Overlapping peptides covering the entire sequence of NP_879835.1 S-adenosylmethionine—tRNA ribosyltransferase-isomerase [B. pertussis Tohama 1].

TABLE 17
PT(O) ANT 15-Overlapping peptides covering the entire sequence of NP_879835.1
S-adenosylmethionine--tRNA ribosyltransferase-isomerase [B. pertussis Tohama I]

| SEQ ID | | | |
|---|---|---|---|
| NO: | Peptide | Start | End |
| 2342 | MPTPLTLADFDYHLP | 1 | 15 |
| 2343 | TLADFDYHLPPELIA | 6 | 20 |
| 2344 | DYHLPPELIAQSPAA | 11 | 25 |
| 2345 | PELIAQSPAAERGGS | 16 | 30 |
| 2346 | QSPAAERGGSRLLHL | 21 | 35 |
| 2347 | ERGGSRLLHLDAASR | 26 | 40 |
| 2348 | RLLHLDAASRLHDRR | 31 | 45 |
| 2349 | DAASRLHDRRFPDLA | 36 | 50 |
| 2350 | LHDRRFPDLAGLLRP | 41 | 55 |
| 2351 | FPDLAGLLRPHDLLV | 46 | 60 |
| 2352 | GLLRPHDLLVFNDTR | 51 | 65 |
| 2353 | HDLLVFNDTRVIKAR | 56 | 70 |
| 2354 | FNDTRVIKARLTGQK | 61 | 75 |
| 2355 | VIKARLTGQKATGGK | 66 | 80 |
| 2356 | LTGQKATGGKVEVLV | 71 | 85 |
| 2357 | ATGGKVEVLVERITA | 76 | 90 |
| 2358 | VEVLVERITAPDRAL | 81 | 95 |
| 2359 | ERITAPDRALAHVRA | 86 | 100 |
| 2360 | PDRALAHVRASKSPG | 91 | 105 |
| 2361 | AHVRASKSPGPGMRL | 96 | 110 |
| 2362 | SKSPGPGMRLRLAEA | 101 | 115 |
| 2363 | PGMRLRLAEAFEAEV | 106 | 120 |
| 2364 | RLAEAFEAEVLGREG | 111 | 125 |
| 2365 | FEAEVLGREGELFDL | 116 | 130 |
| 2366 | LGREGELFDLRFPAP | 121 | 135 |
| 2367 | ELFDLRFPAPVLDLL | 126 | 140 |
| 2368 | RFPAPVLDLLDAHGA | 131 | 145 |
| 2369 | VLDLLDAHGATPLPP | 136 | 150 |
| 2370 | DAHGATPLPPYITHA | 141 | 155 |
| 2371 | TPLPPYITHAADATD | 146 | 160 |
| 2372 | YITHAADATDERRYQ | 151 | 165 |
| 2373 | ADATDERRYQTVYAR | 156 | 170 |
| 2374 | ERRYQTVYAREPGAV | 161 | 175 |
| 2375 | TVYAREPGAVAAPTA | 166 | 180 |
| 2376 | EPGAVAAPTAGLHFD | 171 | 185 |
| 2377 | AAPTAGLHFDQPMLE | 176 | 190 |
| 2378 | GLHFDQPMLEQLAAQ | 181 | 195 |
| 2379 | QPMLEQLAAQGVQRA | 186 | 200 |
| 2380 | QLAAQGVQRAFVTLH | 191 | 205 |
| 2381 | GVQRAFVTLHVGAGT | 196 | 210 |
| 2382 | FVTLHVGAGTFQPVR | 201 | 215 |
| 2383 | VGAGTFQPVRVQNLA | 206 | 220 |
| 2384 | FQPVRVQNLAEHIMH | 211 | 225 |
| 2385 | VQNLAEHIMHAEWYT | 216 | 230 |
| 2386 | EHIMHAEWYTVPEAT | 221 | 235 |
| 2387 | AEWYTVPEATVAAIA | 226 | 240 |
| 2388 | VPEATVAAIARARAH | 231 | 245 |
| 2389 | VAAIARARAHGGRIV | 236 | 250 |
| 2390 | RARAHGGRIVAVGTT | 241 | 255 |
| 2391 | GGRIVAVGTTSVRAL | 246 | 260 |
| 2392 | AVGTTSVRALESAAA | 251 | 265 |
| 2393 | SVRALESAAAQAQDG | 256 | 270 |
| 2394 | ESAAAQAQDGPLAAA | 261 | 275 |
| 2395 | QAQDGPLAAAQGDTR | 266 | 280 |
| 2396 | PLAAAQGDTRLFITP | 271 | 285 |
| 2397 | QGDTRLFITPGYRYR | 276 | 290 |
| 2398 | LFITPGYRYRAVDAL | 281 | 295 |
| 2399 | GYRYRAVDALLTNFH | 286 | 300 |
| 2400 | AVDALLTNFHLPQST | 291 | 305 |
| 2401 | LTNFHLPQSTLLMLV | 296 | 310 |
| 2402 | LPQSTLLMLVSALAG | 301 | 315 |
| 2403 | LLMLVSALAGVEPIR | 306 | 320 |
| 2404 | SALAGVEPIRRAYAH | 311 | 325 |
| 2405 | VEPIRRAYAHAVAER | 316 | 330 |
| 2406 | RAYAHAVAERYRFFS | 321 | 335 |
| 2407 | AVAERYRFFSYGDAM | 326 | 340 |
| 2408 | YRFFSYGDAMFIETP | 331 | 345 |
| 2409 | FFSYGDAMFIETPAP | 333 | 347 |
| | Full Sequence | | |

| | |
|---|---|
| 2613 | MPTPLTLADFDYHLPPELIAQSPAAERGGSRLLHLDAASRLHDRRFPDLAGLLR |
| | PHDLLVENDTRVIKARLTGQKATGGKVEVLVERITAPDRALAHVRASKSPGPG |
| | MRLRLAEAFEAEVLGREGELFDLRFPAPVLDLLDAHGATPLPPYITHAADATD |
| | ERRYQTVYAREPGAVAAPTAGLHFDQPMLEQLAAQGVQRAFVTLHVGAGTF |
| | QPVRVQNLAEHIMHAEWYTVPEATVAAIARARAHGGRIVAVGTTSVRALESA |
| | AAQAQDGPLAAAQGDTRLFITPGYRYRAVDALLTNFHLPQSTLLMLVSALAG |
| | VEPIRRAYAHAVAERYRFFSYGDAMFIETPAP |

TABLE 18 PT(O) ANT 16—Overlapping peptides covering the entire sequence of NP_879350.1 LysR family transcriptional regulator [B. pertussis Tohama I].

TABLE 18
PT(O) ANT 16-Overlapping peptides covering the entire sequence of NP_879350.1
LysR family transcriptional regulator [B. pertussis Tohama I]

| SEQ ID | | | |
|---|---|---|---|
| NO: | Peptide | Start | End |
| 2410 | MQDLNDLYYFAQVVE | 1 | 15 |
| 2411 | DLYYFAQVVEQGGFS | 6 | 20 |
| 2412 | AQVVEQGGFSAASRV | 11 | 25 |
| 2413 | QGGFSAASRVLDVPK | 16 | 30 |
| 2414 | AASRVLDVPKSRLSR | 21 | 35 |
| 2415 | LDVPKSRLSRRISQL | 26 | 40 |
| 2416 | SRLSRRISQLEDRLG | 31 | 45 |
| 2417 | RISQLEDRLGVRLLQ | 36 | 50 |
| 2418 | EDRLGVRLLQRTTRR | 41 | 55 |
| 2419 | VRLLQRTTRRLRLTT | 46 | 60 |
| 2420 | RTTRRLRLTTAGERY | 51 | 65 |
| 2421 | LRLTTAGERYLHYCQ | 56 | 70 |
| 2422 | AGERYLHYCQEMTAS | 61 | 75 |
| 2423 | LHYCQEMTASARAAE | 66 | 80 |
| 2424 | EMTASARAAEDAMRQ | 71 | 85 |
| 2425 | ARAAEDAMRQLQSAP | 76 | 90 |
| 2426 | DAMRQLQSAPAGPVV | 81 | 95 |
| 2427 | LQSAPAGPVVVSCPV | 86 | 100 |
| 2428 | AGPVVVSCPVSIAQQ | 91 | 105 |
| 2429 | VSCPVSIAQQMLAPL | 96 | 110 |
| 2430 | SIAQQMLAPLLPEFL | 101 | 115 |
| 2431 | MLAPLLPEFLDAWPS | 106 | 120 |
| 2432 | LPEFLDAWPSVSVQL | 111 | 125 |
| 2433 | DAWPSVSVQLLVTNR | 116 | 130 |
| 2434 | VSVQLLVTNRRVDVI | 121 | 135 |
| 2435 | LVTNRRVDVIREGVD | 126 | 140 |
| 2436 | RVDVIREGVDLALRV | 131 | 145 |
| 2437 | REGVDLALRVRTKLD | 136 | 150 |
| 2438 | LALRVRTKLDTDAEL | 141 | 155 |
| 2439 | RTKLDTDAELVVKHL | 146 | 160 |
| 2440 | TDAELVVKHLGIASG | 151 | 165 |
| 2441 | VVKHLGIASGTLVAS | 156 | 170 |
| 2442 | GIASGTLVASPAYLQ | 161 | 175 |
| 2443 | TLVASPAYLQRHGTP | 166 | 180 |
| 2444 | PAYLQRHGTPETPQE | 171 | 185 |
| 2445 | RHGTPETPQELASHR | 176 | 190 |
| 2446 | ETPQELASHRTLSFN | 181 | 195 |
| 2447 | LASHRTLSFNDPQNE | 186 | 200 |
| 2448 | TLSFNDPQNEVRWPL | 191 | 205 |
| 2449 | DPQNEVRWPLTNQRG | 196 | 210 |
| 2450 | VRWPLTNQRGESVEV | 201 | 215 |
| 2451 | TNQRGESVEVAVQPV | 206 | 220 |
| 2452 | ESVEVAVQPVLASND | 211 | 225 |
| 2453 | AVQPVLASNDFIVLT | 216 | 230 |
| 2454 | LASNDFIVLTQAAVR | 221 | 235 |
| 2455 | FIVLTQAAVRGRGIA | 226 | 240 |
| 2456 | QAAVRGRGIALLPSM | 231 | 245 |
| 2457 | GRGIALLPSMASEAE | 236 | 250 |
| 2458 | LLPSMASEAELRRGE | 241 | 255 |
| 2459 | ASEAELRRGELVRVL | 246 | 260 |
| 2460 | LRRGELVRVLPDWRS | 251 | 265 |
| 2461 | LVRVLPDWRSPEGIV | 256 | 270 |
| 2462 | PDWRSPEGIVHCIYP | 261 | 275 |
| 2463 | PEGIVHCIYPSRRGM | 266 | 280 |
| 2464 | HCIYPSRRGMMPAVR | 271 | 285 |
| 2465 | SRRGMMPAVRAFLDF | 276 | 290 |
| 2466 | MPAVRAFLDFLAKRV | 281 | 295 |
| 2467 | AFLDFLAKRVPPLVR | 286 | 300 |
| 2468 | LAKRVPPLVRQSDTA | 291 | 305 |
| 2469 | KRVPPLVRQSDTARP | 293 | 307 |
| 2614 | Full Sequence | | |

MQDLNDLYYFAQVVEQGGFSAASRVLDVPKSRLSRRISQLEDRLGVRLLQRTT
RRLRLTTAGERYLHYCQEMTASARAAEDAMRQLQSAPAGPVVVSCPVSIAQQ
MLAPLLPEFLDAWPSVSVQLLVTNRRVDVIREGVDLALRVRTKLDTDAELVV
KHLGIASGTLVASPAYLQRHGTPETPQELASHRTLSFNDPQNEVRWPLTNQRG
ESVEVAVQPVLASNDFIVLTQAAVRGRGIALLPSMASEAELRRGELVRVLPDW
RSPEGIVHCIYPSRRGMMPAVRAFLDFLAKRVPPLVRQSDTARP

TABLE 19 PT(O) ANT 17—Overlapping peptides covering the entire sequence of NP_882010.1 NADH dehydrogenase [B. pertussis Tohama I].

TABLE 19
PT(O) ANT 17-Overlapping peptides covering the entire sequence of NP_882010.1
NADH dehydrogenase [B. pertussis Tohama I]

| SEQ ID NO: | Peptide | Start | End |
|---|---|---|---|
| 2470 | MTQTPNTGSPHRVVI | 1 | 15 |
| 2471 | NTGSPHRVVIVGGGA | 6 | 20 |
| 2472 | HRVVIVGGGAGGLEL | 11 | 25 |
| 2473 | VGGGAGGLELAAKLG | 16 | 30 |
| 2474 | GGLELAAKLGRAHGR | 21 | 35 |
| 2475 | AAKLGRAHGRERVTL | 26 | 40 |
| 2476 | RAHGRERVTLVDSRP | 31 | 45 |
| 2477 | ERVTLVDSRPFHIWK | 36 | 50 |
| 2478 | VDSRPFHIWKPSLHE | 41 | 55 |
| 2479 | FHIWKPSLHEAAAGT | 46 | 60 |
| 2480 | PSLHEAAAGTLDIHQ | 51 | 65 |
| 2481 | AAAGTLDIHQEGLSY | 56 | 70 |
| 2482 | LDIHQEGLSYLMLAN | 61 | 75 |
| 2483 | EGLSYLMLANMCNFT | 66 | 80 |
| 2484 | LMLANMCNFTFAQGE | 71 | 85 |
| 2485 | MCNFTFAQGELQGIE | 76 | 90 |
| 2486 | FAQGELQGIERERRQ | 81 | 95 |
| 2487 | LQGIERERRQIQVGP | 86 | 100 |
| 2488 | RERRQIQVGPVADPS | 91 | 105 |
| 2489 | IQVGPVADPSGQQVL | 96 | 110 |
| 2490 | VADPSGQQVLPPREL | 101 | 115 |
| 2491 | GQQVLPPRELSYDTL | 106 | 120 |
| 2492 | PPRELSYDTLVLAMG | 111 | 125 |
| 2493 | SYDTLVLAMGSTSNF | 116 | 130 |
| 2494 | VLAMGSTSNFFNTPG | 121 | 135 |
| 2495 | STSNFFNTPGAAEHA | 126 | 140 |
| 2496 | FNTPGAAEHAVTLDT | 131 | 145 |
| 2497 | AAEHAVTLDTTENAE | 136 | 150 |
| 2498 | VILDTTENAEQFRLT | 141 | 155 |
| 2499 | TENAEQFRLTMLKAM | 146 | 160 |
| 2500 | QFRLTMLKAMVQVDL | 151 | 165 |
| 2501 | MLKAMVQVDLRKVHD | 156 | 170 |
| 2502 | VQVDLRKVHDPSARL | 161 | 175 |
| 2503 | RKVHDPSARLDLVIV | 166 | 180 |
| 2504 | PSARLDLVIVGGGAT | 171 | 185 |
| 2505 | DLVIVGGGATGVELA | 176 | 190 |
| 2506 | GGGATGVELAVELIE | 181 | 195 |
| 2507 | GVELAVELIEASHVV | 186 | 200 |
| 2508 | VELIEASHVVSAYGL | 191 | 205 |
| 2509 | ASHVVSAYGLPNFRA | 196 | 210 |
| 2510 | SAYGLPNFRADRDLV | 201 | 215 |
| 2511 | PNFRADRDLVITLVE | 206 | 220 |
| 2512 | DRDLVITLVEGAPRI | 211 | 225 |
| 2513 | ITLVEGAPRILSALP | 216 | 230 |
| 2514 | GAPRILSALPEKISR | 221 | 235 |
| 2515 | LSALPEKISRATHAR | 226 | 240 |
| 2516 | EKISRATHARLTELG | 231 | 245 |
| 2517 | ATHARLTELGVRVET | 236 | 250 |
| 2518 | LTELGVRVETDCRVA | 241 | 255 |
| 2519 | VRVETDCRVAEVGAD | 246 | 260 |
| 2520 | DCRVAEVGADHVVTA | 251 | 265 |
| 2521 | EVGADHVVTADGRRF | 256 | 270 |
| 2522 | HVVTADGRRFEATMC | 261 | 275 |
| 2523 | DGRRFEATMCLWAAG | 266 | 280 |
| 2524 | EATMCLWAAGIEGPP | 271 | 285 |
| 2525 | LWAAGIEGPPLFRQL | 276 | 290 |
| 2526 | IEGPPLFRQLGLPLN | 281 | 295 |
| 2527 | LFRQLGLPLNRLGQL | 286 | 300 |
| 2528 | GLPLNRLGQLEVNER | 291 | 305 |
| 2529 | RLGQLEVNERQESPD | 296 | 310 |
| 2530 | EVNERQESPDPHILA | 301 | 315 |
| 2531 | QESPDPHILALGDCC | 306 | 320 |
| 2532 | PHILALGDCCAAPWK | 311 | 325 |
| 2533 | LGDCCAAPWKDGRTV | 316 | 330 |
| 2534 | AAPWKDGRTVPARAQ | 321 | 335 |
| 2535 | DGRTVPARAQAAHQQ | 326 | 340 |
| 2536 | PARAQAAHQQADYLA | 331 | 345 |
| 2537 | AAHQQADYLARKLTA | 336 | 350 |
| 2538 | ADYLARKLTARLRNA | 341 | 355 |
| 2539 | RKLTARLRNAAEPTE | 346 | 360 |
| 2540 | RLRNAAEPTEAYAYH | 351 | 365 |
| 2541 | AEPTEAYAYHDHGSL | 356 | 370 |
| 2542 | AYAYHDHGSLVSLGQ | 361 | 375 |
| 2543 | DHGSLVSLGQGSGVG | 366 | 380 |
| 2544 | VSLGQGSGVGSLMGK | 371 | 385 |
| 2545 | GSGVGSLMGKLAGRG | 376 | 390 |
| 2546 | SLMGKLAGRGLFVSG | 381 | 395 |
| 2547 | LAGRGLFVSGTLARL | 386 | 400 |
| 2548 | LFVSGTLARLMYMSL | 391 | 405 |
| 2549 | TLARLMYMSLHLMHH | 396 | 410 |
| 2550 | MYMSLHLMHHRAVLG | 401 | 415 |
| 2551 | HLMHHRAVLGISRTA | 406 | 420 |
| 2552 | RAVLGISRTATLALA | 411 | 425 |
| 2553 | ISRTATLALARLLMR | 416 | 430 |
| 2554 | TLALARLLMRRTRPR | 421 | 435 |
| 2555 | ARLLMRRTRPRVKLH | 425 | 439 |
| | Full Sequence | | |

| | |
|---|---|
| 2615 | MTQTPNTGSPHRVVIVGGGAGGLELAAKLGRAHGRERVTLVDSRPFHIWKPS |
| | LHEAAAGTLDIHQEGLSYLMLANMCNFTFAQGELQGIERERRQIQVGPVADPS |
| | GQQVLPPRELSYDTLVLAMGSTSNFFNTPGAAEHAVTLDTTENAEQFRLTMLK |
| | AMVQVDLRKVHDPSARLDLVIVGGGATGVELAVELIEASHVVSAYGLPNFRA |
| | DRDLVITLVEGAPRILSALPEKISRATHARLTELGVRVETDCRVAEVGADHVVT |
| | ADGRRFEATMCLWAAGIEGPPLFRQLGLPLNRLGQLEVNERQESPDPHILALG |
| | DCCAAPWKDGRTVPARAQAAHQQADYLARKLTARLRNAAEPTEAYAYHDH |
| | GSLVSLGQGSGVGSLMGKLAGRGLFVSGTLARLMYMSLHLMHHRAVLGISRT |
| | ATLALARLLMRRTRPRVKLH |

TABLE 20. PT(O) ANT 18—Overlapping peptides covering the entire sequence of NP_880884.1 type III secretion system protein [B. pertussis Tohama I]

TABLE 20
PT(O) ANT 18-Overlapping peptides covering the entire sequence of NP_880884.1
type III secretion system protein [B. pertussis Tohama I]

| SEQ ID NO: | Peptide | Start | End |
|---|---|---|---|
| 2556 | MSDTDPFSLALFLAL | 1 | 15 |
| 2557 | PFSLALFLALLALVP | 6 | 20 |
| 2558 | LFLALLALVPLIVVM | 11 | 25 |
| 2559 | LALVPLIVVMTTSFL | 16 | 30 |
| 2560 | LIVVMTTSFLKIAVV | 21 | 35 |
| 2561 | TTSFLKIAVVLALVR | 26 | 40 |
| 2562 | KIAVVLALVRNALGV | 31 | 45 |
| 2563 | LALVRNALGVQQVPP | 36 | 50 |
| 2564 | NALGVQQVPPNMALY | 41 | 55 |
| 2565 | QQVPPNMALYGLALI | 46 | 60 |
| 2566 | NMALYGLALILSAYV | 51 | 65 |
| 2567 | GLALILSAYVMAPVV | 56 | 70 |
| 2568 | LSAYVMAPVVHRIGT | 61 | 75 |
| 2569 | MAPVVHRIGTEVQAL | 66 | 80 |
| 2570 | HRIGTEVQALTAQAG | 71 | 85 |
| 2571 | EVQALTAQAGESGTA | 76 | 90 |
| 2572 | TAQAGESGTAAPMAL | 81 | 95 |
| 2573 | ESGTAAPMALDAVLG | 86 | 100 |
| 2574 | APMALDAVLGVAERG | 91 | 105 |
| 2575 | DAVLGVAERGVGPLR | 96 | 110 |
| 2576 | VAERGVGPLRAFMLR | 101 | 115 |
| 2577 | VGPLRAFMLRNSQPA | 106 | 120 |
| 2578 | AFMLRNSQPAQRDFF | 111 | 125 |
| 2579 | NSQPAQRDFFLRTAR | 116 | 130 |
| 2580 | QRDFFLRTARHLWGE | 121 | 135 |
| 2581 | LRTARHLWGEEASRD | 126 | 140 |
| 2582 | HLWGEEASRDLSEDN | 131 | 145 |
| 2583 | EASRDLSEDNLLVLT | 136 | 150 |
| 2584 | LSEDNLLVLTPAFLV | 141 | 155 |
| 2585 | LLVLTPAFLVSELTA | 146 | 160 |
| 2586 | PAFLVSELTAAFQLG | 151 | 165 |
| 2587 | SELTAAFQLGFLLYL | 156 | 170 |
| 2588 | AFQLGFLLYLPFIII | 161 | 175 |
| 2589 | FLLYLPFIIIDLIVS | 166 | 180 |
| 2590 | PFIIIDLIVSNILLA | 171 | 185 |
| 2591 | DLIVSNILLAMGMMM | 176 | 190 |
| 2592 | NILLAMGMMMVSPVT | 181 | 195 |
| 2593 | MGMMMVSPVTISMPL | 186 | 200 |
| 2594 | VSPVTISMPLKLFLF | 191 | 205 |
| 2595 | ISMPLKLFLFVMVDG | 196 | 210 |
| 2596 | KLFLFVMVDGWTRLI | 201 | 215 |
| 2597 | VMVDGWTRLIQGLVL | 206 | 220 |
| 2598 | DGWTRLIQGLVLSYR | 209 | 223 |
| | Full Sequence | | |

| | |
|---|---|
| 2616 | MSDTDPFSLALFLALLALVPLIVVMTTSFLKIAVVLALVRNALGVQQVPPN |
| | MALYGLALILSAYVMAPVVHRIGTEVQALTAQAGESGTAAPMALDAVLG |
| | VAERGVGPLRAFMLRNSQPAQRDFFLRTARHLWGEEASRDLSEDNLLVLT |
| | PAFLVSELTAAFQLGFLLYLPFIIIDLIVSNILLAMGMMMVSPVTISMPLKLF |
| | LFVMVDGWTRLIQGLVLSYR |

TABLE 21
List of the immunodominant antigens with reactivity and frequency
of recognition equal or better than aP vaccine antigens.

| | | | % of | % of |
|---|---|---|---|---|
| | NCBI Reference | Length | Total | Donor |
| ORF Description in order | Sequence | (a.a.) | magnitude | response |
| D-alanyl-D-alanine | WP_003809423.1 | 478 | 1.74 | 25 |
| carboxypeptidase | | | | |
| Membrane protein | WP_010927254 | 563 | 1.35 | 17.5 |
| insertase YidC | | | | |
| Maltose alpha-D- | WP_010930308.1 | 1113 | 0.82 | 15 |
| glucosyltransferase | | | | |
| Virulence sensor | WP_010930608 | 1238 | 0.76 | 15 |
| protein BvgS | | | | |
| 1,4-alpha-glucan branching | WP_010930307 | 731 | 0.53 | 15 |
| protein GIgB | | | | |
| EI24 domain-containing protein | WP_010931453 | 266 | 0.52 | 15 |
| NADH-quinone oxidoreductase | WP_003813916.1 | 494 | 0.45 | 12.5 |
| subunit N | | | | |
| BrkA autotransporter | WP_010931506.1 | 1010 | 0.44 | 20 |
| Bifunctional hemolysin- | WP_010929995.1 | 1706 | 0.40 | 22.5 |
| adenylate cyclase toxin | | | | |
| YihY/virulence factor BrkB | WP_003808614.1 | 296 | 0.34 | 12.5 |
| family protein | | | | |
| Filamentous hemagglutinin | WP_010930614.1 | 584 | 0.29 | 15 |
| transporter protein FhaC | | | | |
| Antioxidant protein | WP_003813333.1 | 213 | 0.27 | 15 |
| (Peroxiredoxin) | | | | |
| S-adenosylmethionine--tRNA | WP_010930156 | 347 | 0.27 | 17.5 |
| ribosyltransferase-isomerase QueA | | | | |
| TRAP transporter fused | WP_010930804.1 | 866 | 0.26 | 12.5 |
| permease subunit | | | | |
| LysR family transcriptional | WP_010929840.1 | 307 | 0.25 | 12.5 |
| regulator | | | | |

### Example 2

Experimentally defined BP epitope pools can be used to detect BP-specific responses in vaccinated individuals.

Here, the inventors report that BP-specific CD4+ T cell responses are detected in vaccinated individuals irrespective of the type of vaccine administered in childhood by activation induced marker (AIM) or intracellular cytokine staining (ICS) assays.

As shown in FIGS. 12A-12C, a set of 132 peptides from aP vaccine antigens (PT(E)VAC) (Table 1) previously experimentally defined (Bancroft et al., 2016) and thoroughly characterized (da Silva Antunes et al., 2018; da Silva Antunes et al., 2021; da Silva Antunes et al., 2020) was combined into a pool of epitopes (megapool) and used in short-culture stimulation of PBMCs in AIM or ICS assays. The PT(E)VAC megapool includes peptides from 5 antigens [Filamentous hemagglutinin (FHA), pertactin (PRN), pertussis toxin (PT), and fimbrial proteins 2 and 3 (Fim2/3)], which are the only 5 antigens contained in the current acellular (aP) vaccine administered in the United States. In parallel, an epitope pool of 170 experimentally defined peptides (PT(E)R) (Table 2) covering the most immunogenic peptides across the entire BP genome, and not including peptides derived from aP vaccine antigens, was tested in the same cohort. This megapool was developed from the findings of an ongoing NIH Pertussis T cell contract (75N93019C00066) that spearheaded the identification of novel T cell epitope targets and novel immunogenic antigens (da Silva Antunes et al. in preparation). Interestingly, BP-specific reactivity was similar between the 2 megapools using an AIM assay after 24 h of stimulation (FIG. 12A). Using a threshold for positivity of 100 AIM+ cells (indicated by the dotted line), 20/20 (100%) of donors showed positive responses after PT(E)VAC stimulation, and 16/20 (80%) of donors showed positive responses after (PT(E)R) stimulation. Surprisingly, BP-specific responses were observed regardless of vaccine used in childhood immunization (FIG. 12B). Detection of responses for both PT(E)VAC and PT(E)R epitopes pools were also observed using an ICS assay (FIG. 12C). As expected, IFNλ, TNFα and IL-2 responses were the most prevalent.

### Example 3

Focusing on individual selected antigens.

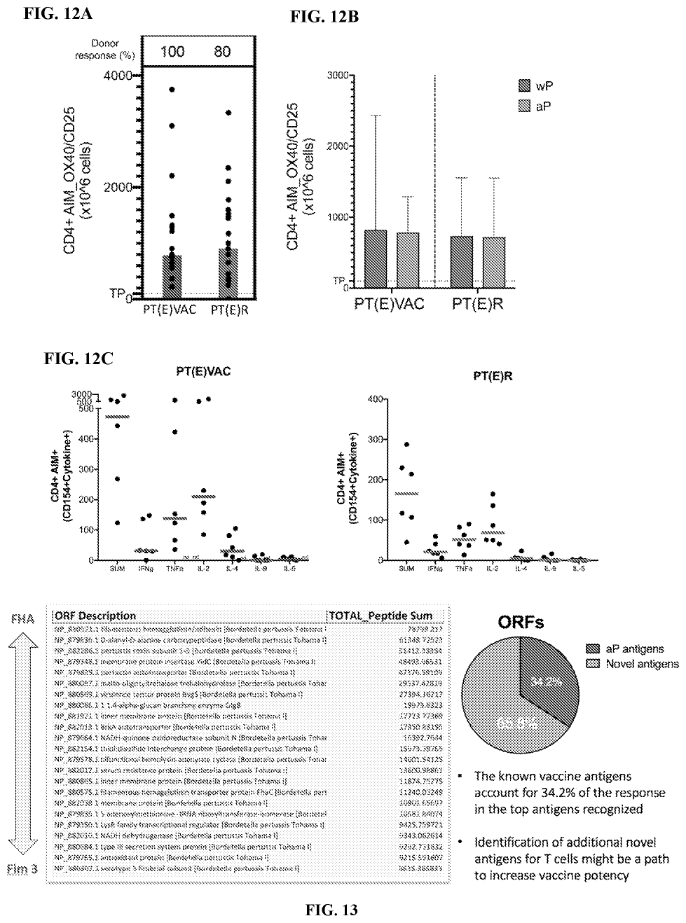

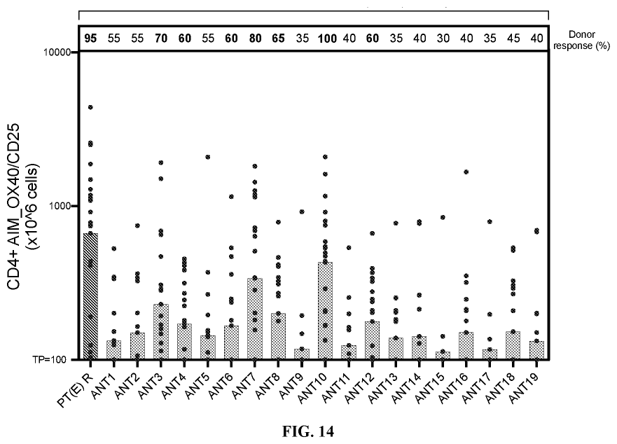

Individual antigen responses for the most immunodominant antigens (FIG. 13), identified in a BP genome-wide screening study (da Silva Antunes et al. in preparation) can also be detected in vaccinated individuals using the AIM assay (FIG. 14).

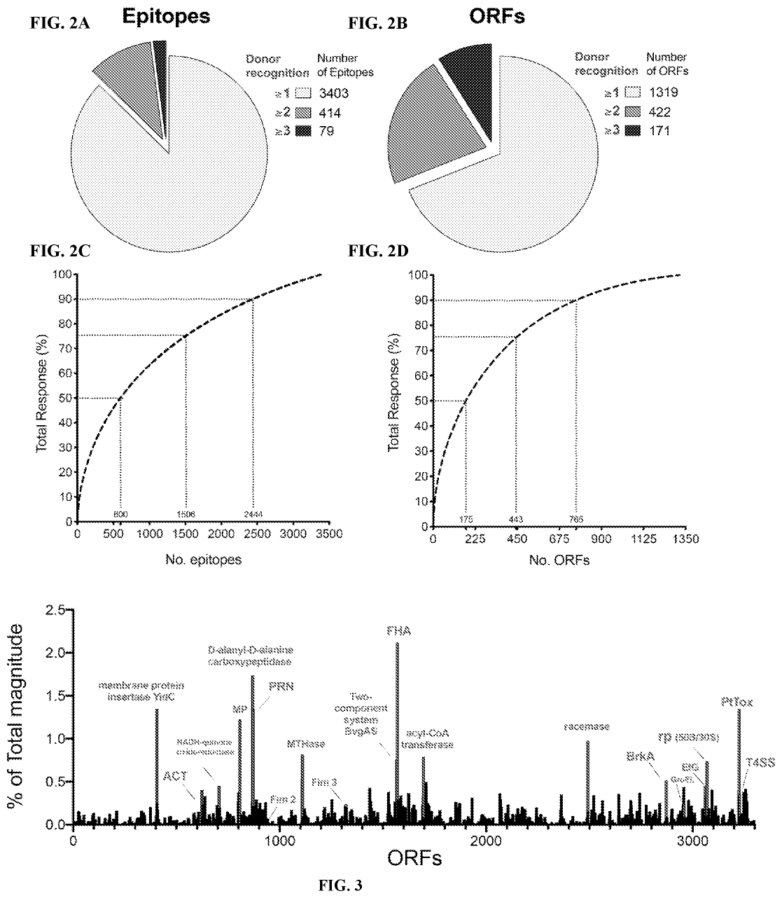

Briefly, following a genome-wide map of BP-specific CD4 T cell reactivity and identification of novel epitopes and their antigen of origin (or ORFs), the inventorsdefined a high immunogenicity threshold based on the interval of the known aP vaccine antigen responses both in terms of magnitude or donor response (frequency of recognition>12.5%). This strategy allowed us to identify 19 novel antigens with equal or better reactivity than aP vaccine antigens (FIG. 13—Table insert). Among the top novel antigens, adenylate cyclase toxin (ACT), BvgS and BrkA, known to be strongly associated with virulence and BP infection in mouse models, were identified, as well as a type III secretion system protein (T3SS) or proteins involved in regulation, serum resistance, or DNA-binding. Moreover, several enzymes involved in roles such as cell wall and cell membrane assembly (D-alanyl-D-alanine carboxypeptidase and membrane protein insertase YidC, respectively), metabolic processes related with glycogen biosynthesis, oxidative phosphorylation or other catalytic processes were also highly reactive and elicited immunodominant responses. Interestingly, the reactivity to these antigens is about 2-fold higher than for vaccine antigens (FIGS. 2A-2D).

As shown in FIGS. 10A-10B, antigen-specific responses for the 19 BP most immunodominant antigens not included in the aP vaccine can also be detected, although with variable degree of reactivity and responsiveness. The epitope pools for ACT (ANT10) and BrKA (ANT7) elected strong responses, and were the highest immunoreactive targets with 100% (20/20), and 80% (16/20) of positive donor response, respectively. Interestingly, and similar to what observed for (PT(E)VAC) and (PT(E)R) stimulation, responses to all antigens were detected irrespectively of the vaccine administered in childhood immunization (not shown).

The inventors demonstrate that through the use of peptide pools described in this disclosure, detection and quantification of BP-specific T cells can be easily and rapidly accomplished with high sensitivity in vaccinated cohorts, irrespectively of the nature of their childhood BP vaccine immunization. The newly developed BP human T cell epitope pools can be further used to measure T cell responses against BP colonization in naturally infected, and clinically diagnosed acute or convalescent cohorts. The disclosure identifies novel immunogenic targets that are crucial in the design of vaccines for superior control of BP infection and induction of long-lasting protection.

### Example 4

Experimental design for a genome-wide screen of Bordetella pertussis human T cell epitopes.

Previous studies characterized human CD4+ T cell reactivity to the four main antigens contained in the acellular pertussis vaccine, but little to no information is available regarding responses to other BP antigens. Here, the inventors defined human CD4+ T cell reactivity spanning the entire BP proteome, by an approach previously used to draw a genome-wide map of human CD4+ T cell responses to Mycobacterium tuberculosis (MTB) (Lindestam Arlehamn et al., 2013).

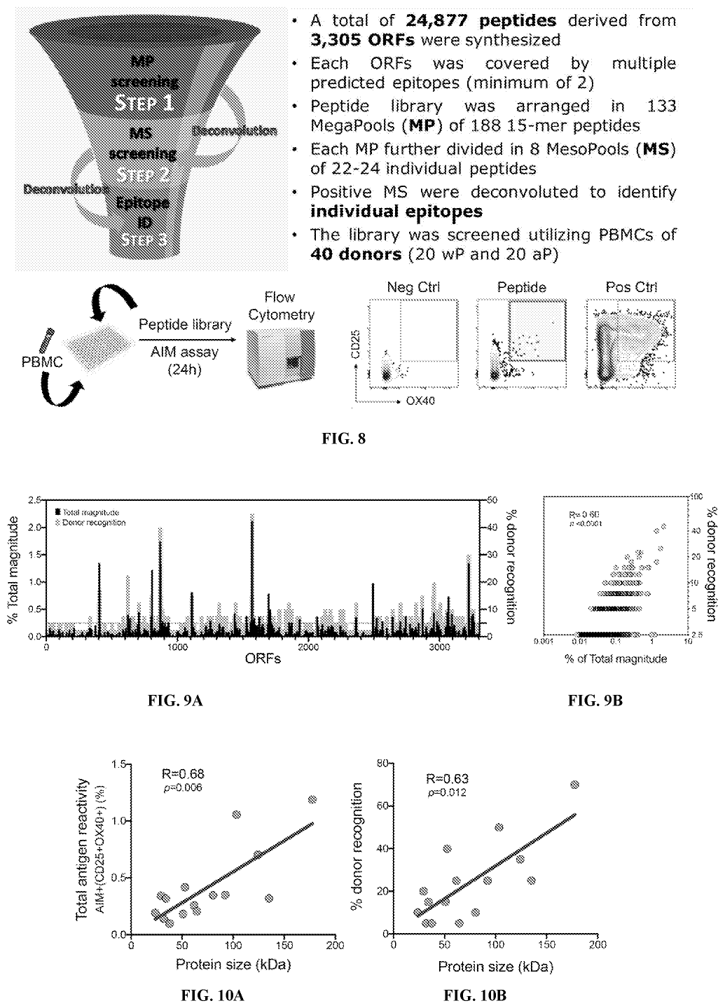

The approach is based on predicting potential dominant CD4+ T cell epitopes from each ORFs encoded in the bacterial genome, based on their predicted promiscuous binding to HLA class II molecules (Paul et al., 2015). Previous studies demonstrated that this approach identifies the most dominant and prevalent epitopes, corresponding to approximately 50% of the total overall response (Grifoni et al., 2020; Oseroff et al., 2010). Accordingly, the inventors synthetized a library encompassing a total of 24,877 peptides derived from 3,305 ORFs. The library was arranged in 133 pools of 188 15-mer peptides (hereafter called MegaPools; MP). Each MP was further divided in 8 pools of 22-24 individual peptides (hereafter called MesoPools; MS). A summary of the screening strategy is shown in FIG. 8.

The library was screened for CD4+ T cell reactivity utilizing PBMCs collected in the 2013 to 2021 period from 40 participants, 21 males and 19 females, of 18 to 40 years of age. Based on clinical records and year of birth, 20 of the participants were immunized in childhood with a whole-cell Pertussis (wP) vaccine, and 20 were originally immunized in childhood with an acellular Pertussis (aP) vaccine.

### Example 5

Large breadth of BP-specific CD4+ T cell responses in humans.

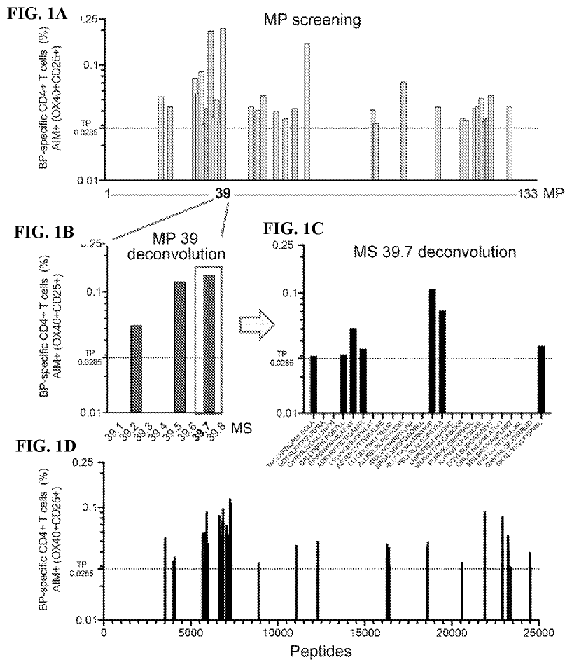

CD4+ T cell reactivity was assayed directly ex vivo using an Activation Induced Marker (AIM) assay (FIGS. 12A-12C), utilizing the combination of markers OX40+CD25+(Dan et al., 2016), previously validated for epitope identification in the context of BP (da Silva Antunes et al., 2020). As done previously the threshold of positivity (TP) was based on the median twofold standard deviation of T cell reactivity in negative controls, corresponding to 285 cells per million of CD4+ T cells (0.0285%), and values above TP and with a stimulation index (S.I.)>2 were considered positive as previously described (da Silva Antunes et al., 2020; Tarke et al., 2022). An example of screening of the whole genome-wide library in a representative donor is shown in FIGS. 1A-1D. In this particular donor, 32 positive MPs were identified (FIG. 1A). FIG. 1B illustrates the deconvolution of one representative MP (MP #39), which yielded 3 positive MS. FIG. 1C illustrates the deconvolution of the MS with the highest reactivity (MS #39.7), which identified 7 individual epitopes above the significance threshold. Overall, this particular donor recognized 148 different epitopes. FIG. 1D shows the position of each individual epitope identified across the aligned BP genome, using the Tohama I and D420 BP strains as reference.

To define the global pattern of immunodominance in the study cohort, the inventors tested PBMCs from each donor with sets of the same peptide library and recorded the number of donors in which a positive response was detected. The screening of the entire cohort revealed a total of 414 and 79 epitopes recognized by in at least 2 or 3 donors respectively (FIG. 2A). Each individual epitope was also mapped back to the individual BP ORFs of origin. A total of 422 and 171 ORFs recognized by more than 2 and 3 donors respectively (FIG. 2B). Parallel analyses quantified the total response across the entire cohort for each individual epitope or ORF. A total of 600 epitopes and 175 ORFs were required to account for 50% of the total response, 1506 epitopes or 443 ORFs accounted for 75% of the response, and 2444 epitopes or 765 ORFs were required to account for 90% of the total response (FIGS. 2C, 2D). Overall, the first quantitation of human CD4+ T cell responses to the whole BP genome revealed an unprecedented large breadth of antigens and epitopes recognized.

### Example 6

Immunodominance in BP responses.

The overall magnitude of response and localization of each recognized ORF/antigen across the entire cohort was next visualized summing all the reactivity of individual epitopes across all donors on a linear map of the BP genome (FIG. 3). As expected, the known aP vaccine antigens (i.e., pertactin, PRN; two serotypes of fimbriae, Fim2/3; filamentous hemagglutinin, FHA; and pertussis toxin, PtTox) were amongst the most dominant antigens (FIG. 3). In particular, FHA was the antigen with the highest magnitude (2.12% of total response) and the most frequently recognized (45.0% of donors) of all the BP antigens. PtTox (ORF 1-5) and PRN were also associated with a high reactivity (1.34% and 1.33% of total response, respectively) and high frequency of donor recognition (30.0% and 40%, respectively). Fim 3 and Fim 2 had the lowest reactivity (0.24% and 0.09% of total response, respectively) and were the least recognized antigens among the aP vaccine antigens (12.5% and 5% of donors, respectively). Strikingly, the cumulative response of the 5 aP vaccine antigens only accounted for a minor fraction of the total CD4+ T cell response (5.13% of the total response).

Conversely a high and broad reactivity was associated with non-aP vaccine antigens (FIG. 3), and a total of 15 antigens were associated with reactivity and frequency of recognition equal or better than aP vaccine antigens (Table 1). Together, these 15 antigens and the 5 vaccine antigens accounted for about 14% of the total response, underlining the extreme breadth and heterogeneity of responses. ORFs associated with the highest magnitude were also associated with the highest frequency of responses. The 15 antigens eliciting same level of responses than the vaccine antigens included ORFs from enzymes involved in roles such as cell wall and cell membrane assembly (D-alanyl-D-alanine carboxypeptidase and membrane protein insertase YidC, respectively) and from a transporter protein (Fha C) that mediates the secretion of aP vaccine antigen FHA. Among the dominant antigens were also adenylate cyclase toxin (ACT), BvgS and BrkA, which are associated with virulence and BP infection (DiVenere et al., 2022; Elder and Harvill, 2004; Moon et al., 2017). Overall, these results successfully re-identified aP vaccine antigens, and in addition greatly expanded the repertoire of antigens recognized by human BP-specific CD4 T cell responses.

### Example 7

Similar recognition of aP and non-aP vaccine antigens as a function of priming vaccination in infancy.

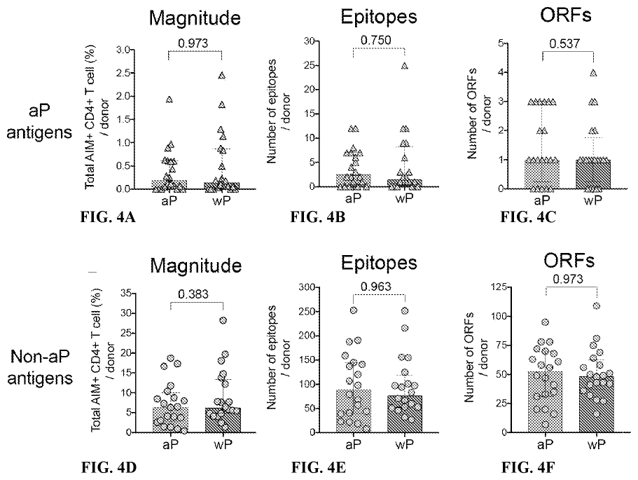

Half of the donor cohort was vaccinated in childhood with the wP vaccine, which is expected to generate responses targeting a wide range of BP antigens, while the other half was vaccinated in childhood with the aP vaccine, containing only four different antigens. To address whether the original priming would result in different repertoires of antigens recognized in adulthood, the inventors compared responses from participants primed in infancy with aP versus wP vaccines for recognition of aP and non-aP vaccine antigens. No difference was detected for aP vaccine antigens (FIGS. 4A-4C), considering either magnitude (sum of all reactivity of individual epitopes for a single donor) (FIG. 4A), number of epitopes (FIG. 4B) or ORFs recognized (FIG. 4C). Similar magnitude, number of epitopes and ORFs between aP- and wP-primed donors were also observed when only responses to non-aP vaccine antigens were considered (FIGS. 4D-4F). Overall, BP specific-CD4+ T cells responses did not differ as function of the original priming vaccination in infancy. The lack of significant differences between aP and wP originally primed donors, and the large breadth of responses, particularly in aP-primed donors is consistent with BP infection frequently occurring in vaccinated donors.

### Example 8

Sequence conservation and immunogenicity of BP peptides.

The degree of conservation of microbial sequences in different isolates and/or related species 5 influence immunodominance, as conserved sequences have implicitly more opportunities to be recognized (Bui et al., 2007; Westernberg et al., 2016). Here, the inventors performed a conservation analysis amongst different strains of BP or amongst different species of the genus Bordetella. Peptides were divided in subsets according to their immunogenicity or degree of conservation in different isolates or related species. In terms of immunogenicity, peptides were arbitrarily divided in not recognized, subdominant (recognized in 1 donor), or dominant (recognized in >=2 donors). In terms of conservation, peptides were classified as variable (<75% of homology), intermediate (75-95% of homology), or conserved (>95% of homology). Finally, peptides were further segregated as derived from aP vaccine antigens, or from non-aP vaccine antigens.

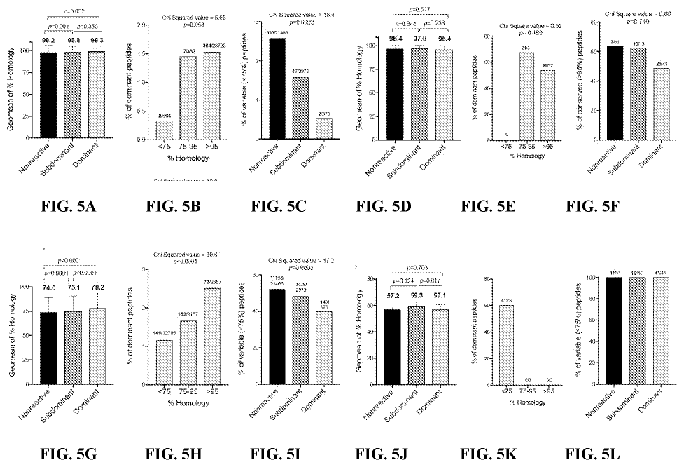

Twenty different BP strains with complete genomes and representative of the various clades of BP (Felice et al., 2022)) were analyzed. Peptides from non-vaccine antigens are highly conserved (98.2-99.3% range) regardless of whether they are recognized by T cells or not (FIG. 5A). However, dominant peptides are enriched in intermediate or conserved peptides (FIG. 5B), and non-reactive peptides are significantly enriched in variable peptides (FIG. 5C). Similar results were noted for peptides derived from aP vaccine antigens (FIGS. 5D-5F), but the analysis significance is limited by the small number of peptides, and the high number of “dominant” peptides.

The inventors next investigated 22 different genomes of different species of the genus Bordetella. Peptides from non-vaccine antigens have low conserved sequences (74.0-78.2% range) regardless of whether they are recognized by T cells or not (FIG. 5G). As in the case of the strain analysis, dominant peptides are enriched in conserved peptides, while non-reactive or subdominant peptides are enriched in variable peptides (FIGS. 5H-5I). Moreover, peptides from aP vaccine antigens are not conserved between species (<66.6%) with all peptides irrespectively of the T cell reactivity falling in the variable category (FIGS. 5J-5L). Overall, while peptides associated with sequence variations are less likely to be recognized, sequence conservation is high among different BP strains and low in different Bordetella species, and therefore sequence conservation does not appear to be a major driver of BP CD4+ T cell immunogenicity.

### Example 9

Phenotypes associated with recognition of non-aP vaccine antigens.

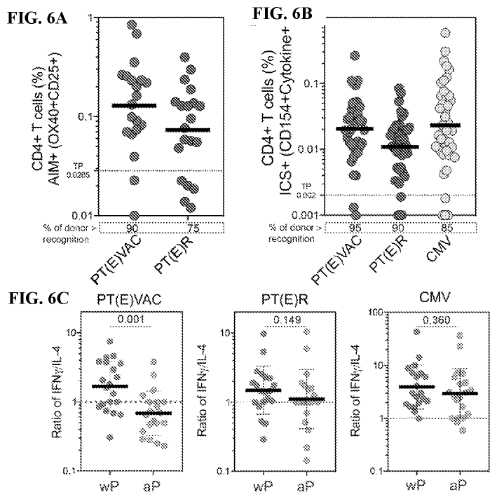

To characterize responses to the highest reactive non-aP vaccine antigens identified in this study, the inventors generated a pool encompassing 170 different epitopes (Dominant peptides tested positive in at least 2 donors with >0.06% total CD4+ T cell response), and hereafter denominated PT(E)R (Table 2). As control, the inventors used a previously described MP (Bancroft et al., 2016; da Silva Antunes et al., 2018), containing epitopes exclusive from aP vaccine antigens [PT(E)VAC]. These MPs were tested in replicates of 3 independent experiments with PBMC from 20 subjects (10 aP and 10 wP). As expected, the PT(E)R pool yielded vigorous responses, which were not statistically different when compared to the PT(E)VAC pool in the AIM assay (FIG. 6A), with 90% and 75% of donor recognition, respectively.

Previous studies highlight differences in polarization patterns of responses to the aP vaccine antigens as a function of the original priming, with aP original vaccination being associated with a Th2 pattern and wP priming being associated with a Th1 profile (da Silva Antunes et al., 2018; da Silva Antunes et al., 2020). Here the inventors addressed whether this difference in polarization was also noted with the non-aP vaccine antigens. Accordingly, CD4+ T cell responses to PT(E)VAC and PT(E)R were measured by intracellular cytokine staining (ICS) with phenotypic assessment of IFNγ, TNFα, IL-2 and IL-4 expression among intracellular CD154+ (CD40L) cells in PBMC from an additional cohort of 40 subjects (20 aP and 20 wP). A MP against the ubiquitous antigen CMV was used as an additional control of specificity.

As shown in FIG. 6B, antigen-specific CD4+ T cell responses to PT(E)VAC and PT(E)R pools were also readily detected in terms of cytokine secretion with 95% and 90% of donor recognition, respectively. Interestingly, while responses to the aP vaccine antigens were Th1 polarized in wP-primed donors and Th2 polarized in aP-primed donors (FIG. 6C), responses to the non-aP vaccine antigen pool PT(E)R were not polarized as function of priming vaccination in childhood, similar to CMV responses, which are known to be highly Th1 polarized (FIG. 6C). Overall these results show that the Th2 polarization is specific to the aP vaccine antigens, in individuals originally primed with aP vaccine.

### Example 10

CD4+ T cell responses against individual non-aP vaccine antigens.

To further characterize responses to the most immunodominant non-aP vaccine antigens identified (Table 1), the inventors synthetized 15 MPs using sets of peptides (15mers) overlapping by 10a.a. and encompassing the entire sequence of each individual antigen (ANT1-ANT15). This unbiased approach allowed us to determine the overall reactivity of each antigen irrespectively of the number of predicted peptides selected and tested from the initial screening library. As control, the inventors used the above-mentioned PT(E)VAC peptide pool. These MPs were tested in replicates of 3 independent experiments with PBMC from 20 subjects (10 aP and 10 wP) using the AIM assay.

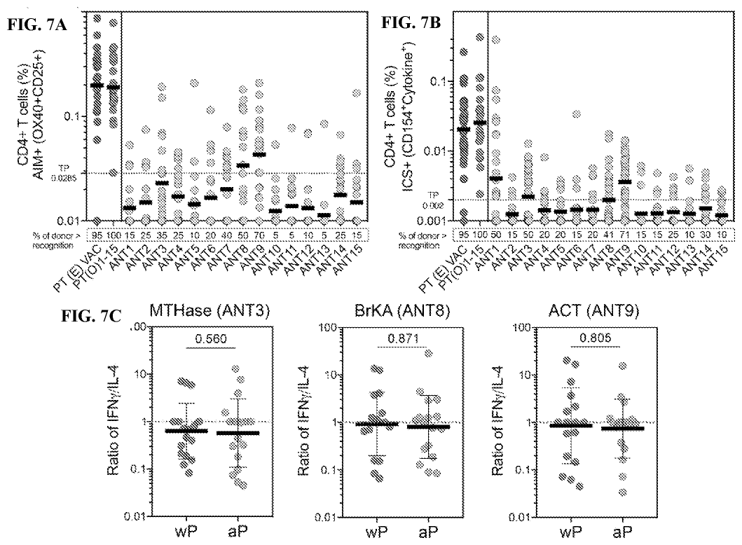

To capture the overall response of each donor to all the 15 antigens, the magnitude of the individual MPs was combined (PT(0)1-15). The results in FIG. 7A show that responses to PT(0)1-15 were robust and detected in all of donors, and yielded similar magnitude and donor recognition as PT(E)VAC. Among non-aP vaccine antigens, Maltose alpha-D-glucosyltransferase; MTHase (ANT3), BrKA (ANT8) and ACT (ANT9) were the most reactive antigens, with 35%, 50% and 70% of donors responding, respectively. These results could be in part attributed to the fact that in general, the most reactive antigens have the longest sequences (FIG. 14). Interestingly, responses to the different antigens were detected irrespectively of the vaccine administered in childhood immunization (not shown).

Detection of antigen-specific CD4+ T cell responses to the 15 non-aP vaccine antigens was also performed by ICS, in a subset of the previous cohort of 40. Similar results to the AIM assay (FIG. 7A) were observed for cytokine responses to all antigens combined (PT(0)1-15), and to the 15 individual antigens (FIG. 7B) with 50%, 41% and 71% of positive donor response for MTHase (ANT3), BrKA (ANT8) and ACT (ANT9), respectively. Consistent with the results shown above for the non-aP vaccine antigen pool PT(E)R, responses to non-aP vaccine individual antigens were not polarized as function of priming childhood vaccination (FIG. 7C). These results confirm that responses to non-aP vaccine antigens are not Th2 polarized.

### Example 11

Knowledge regarding human T cell responses to BP was previously largely limited to the four antigens contained in the aP vaccine, despite growing awareness of the importance of T cells in the control and prevention of symptomatic whopping cough disease (Chasaide and Mills, 2020; Fedele et al., 2015; Lambert et al., 2021; Solans and Locht, 2018; Warfel et al., 2014). Herein, the inventors provide the first in-depth characterization of human CD4+ T cell responses to the whole BP proteome, spanning over 3,000 different ORFs.

BP specific-CD4+ T cells in healthy young adults immunized with different pertussis vaccines are associated with a previously unrecognized and remarkable large breadth of responses. Tens of ORFs and hundreds of different peptides were recognized in the 40 donors studied. This remarkable large breadth of T cell responses parallels the large breadth of responses observed for another bacterial species (Mycobacterium tuberculosis: (Lindestam Arlehamn et al., 2013)), and it is a testament to the fact that T cell responses have the capacity to recognize most if not all foreign proteins.

Surprisingly, no differences were observed between wP- and aP-primed vaccinees in magnitude, breadth, or antigen repertoire of responses in both non-aP and aP vaccine antigens, indicating that the childhood priming does not drive the repertoire features detected in young adults. The broad responses to non-aP vaccine antigens in adults is consistent with the notion that repeated exposure and/or colonization might be a prevalent occurrence throughout lifetime as suggested by the high prevalence of asymptomatic BP infections in human populations (de Melker et al., 2006; Heininger et al., 2004b; Naeini et al., 2015; Palazzo et al., 2016; van Schuppen et al., 2022; Zhang et al., 2014) and of adults being a reservoir for ongoing circulation of BP (Blanchard-Rohner, 2022). Lastly, this hypothesis is further supported by the evidences in animal studies performed in baboon and mouse models that established that aP vaccination does not control infection, only symptomatic disease (Warfel et al., 2014; Wilk et al., 2019; Zeddeman et al., 2020). Since none of the donors participating in this study reported having experienced clinical whooping cough disease, the observed heterogeneity might reflect asymptomatic or sub-clinical infections.

While as expected, all the known aP vaccine antigens were re-identified, they accounted for a relatively minor fraction of the total CD4+ T cell response, and these studies identified fifteen different antigens that were recognized as vigorously as the aP vaccine antigens. These antigens included BrkA and ACT, known to play important roles in the pathogenesis of pertussis, as dominant targets of human T cell responses. Other antigens were associated with regulation of gene expression (LysR family transcriptional regulator) or virulence (virulence sensor protein BvgS, YihY/virulence factor BrkB family protein or Filamentous hemagglutinin transporter protein FhaC, that mediates the secretion of aP vaccine antigen FHA). Among other highly reactive targets the inventors found several antigens associated with Type I-IV secretion systems, a machinery system that translocate proteins and virulence factors (e.g. PtTox) from the cytoplasm to extracellular environment (Park et al., 2015). Indeed, virulence factors and bacterial secretory systems appear to be frequently recognized by human T cell responses, possibly reflective of their role in infection (Lindestam Arlehamn et al., 2013; Rolan and Tsolis, 2008; Saikh et al., 2006; Shepherd and McLaren, 2020).

What is the relation between the targets of CD4+ T cell and antibody recognition? The ACT antigen is also dominantly recognized by antibody responses un humans (Arciniega et al., 1991; Cherry et al., 2004), and additional CD4+ T cell antigens identified in this study are immunodominant for antibodies in mouse models (GroEL, EfG, and ribosomal proteins; (Raeven et al., 2015). In addition, a recent antigen discovery study using serum from convalescent baboons identified a total of 314 antigens targets of antibody responses, including several dominant targets of T cell responses in this study, such as BvgS, FhaC, Membrane protein insertase YidC, BrkA, and ACT. Because of the deterministic linkage between 10 antibody and CD4+ T cell responses for large pathogens (Sette et al., 2008), the inventors' findings identify antigens that are targets of both antibody and T cell reactivity and should be considered in the design of upcoming pertussis vaccines.

The identification of new CD4+ T cell epitopes and immunodominant antigens not included in the aP vaccine, enabled us to develop tools to detect and characterize BP non-aP vaccine responses. While responses to the aP antigens were Th1 polarized in wP-primed donors and Th2 polarized in aP-primed donors, responses to non-aP antigens were not polarized as function of childhood priming vaccination. These results indicate that a Th2 phenotype is specific to aP vaccine antigens in individuals originally primed with aP vaccine. These results also suggest that the dominant non-aP vaccine antigens identified in this study could be used to induce a more balanced Th1/Th2 imprinting of memory BP responses even in subjects in which a dominant, long lasting Th2 response to the aP antigens is imprinted as a result of childhood aP vaccination.

Remarkably each individual tended to recognize a partially overlapping yet unique set of antigenic and epitope targets. The reasons for this donor-donor heterogeneity are not apparent, but might include past infection history, differences in HLA types, and influences of the microbiome on the repertoire of recognition. In this context, conservation amongst different Bordetella species is not a major driver of the dominantly recognized epitopes as in general, peptide homology or sequence conservation between Bordetella isolates was low, with only a small fraction (<3%) of the highly conserved peptides associated with dominant responses. In addition, the majority of the Bordetella isolates studied are species non-infectious to humans, and therefore a high sequence homology devoid of relevance.

These newly established peptide pools will aid the research and characterization of CD4+ and CD8+ T cell responses in BP infection and controlled human BP infection/colonization initiatives in humans, and can be incorporated into novel compositions, therapeutics, and diagnostics as disclosed herein, including but not limited to whole-cell based vaccines candidates (Buddy Creech et al., 2022; Chasaide and Mills, 2020; Diks et al., 2023). Herein, the inventors characterized CD4+ T cells responses directly ex vivo using high-throughput screening methodology, thus introducing minimal physiological perturbations and bias as a result of in vitro clonal expansion, and in combination with bioinformatic predictions of potential dominant epitopes binding HLA class II. This approach should be generally applicable to the study of T cell immune responses to other complex bacterial pathogens. Altogether, the fact that similar and broad repertoires are detected in aP-versus wP-originally primed subjects, is compatible with the notion that asymptomatic infections occur similarly in the two groups. This appears to be independent of the reported difference between aP versus wP-originally primed subjects in terms of protection from symptomatic disease.

Study subjects. 62 healthy adults from San Diego, USA were recruited. All participants provided written informed consent for participation and relevant clinical medical history was collected and evaluated by the clinical coordinators through recording dates, type of pertussis vaccine, vaccination schedule, and questionnaires including if they ever experienced whooping cough disease. Individuals who had been diagnosed with BP infection at any given time in their life were excluded. All donors were from the San Diego area, and originally vaccinated with either DTwP or DTaP in childhood and followed the recommended vaccination regimen (which is also necessary for enrollment in the California school system), which entails a Tdap booster immunization at 11-12 years and then every 10 years. In both groups, male and female subjects were included equally.

PBMC isolation. PBMCs were isolated from whole blood by density gradient centrifugation according to manufacturer instructions (Ficoll-Hypaque, Amersham Biosciences, Uppsala, Sweden) and cryopreserved for further analysis.

Peptide prediction, synthesis, library assembly and pool preparation. Peptide selection was derived from either BP whole-genome predictions from the Tohama I strain or from a set of 256 unique open reading frames (ORF) from the recent clinical isolate D420 strain and not contained in Tohama I strain. These 256 unique ORFs have been identified in the lab of Dr. Tod Merkel (unpublished findings or Ref?). BP genome-wide identification from Tohama I strain was performed by scanning for the presence of predicted HLA class II promiscuous binding peptides. MHC-peptide binding predictions were performed using publicly available tools hosted by the Immune Epitope Database (IEDB) Analysis Resource (Dhanda et al., 2019). Specifically, the prediction of peptides was established by the 7-allele HLA class II restricted method and by using peptides 15 residues in length and overlapping by 10 residues. Additional filtering using an epitope cluster analysis tool was performed to include unique peptides across all proteins with median percentile rank (cut-off of 10) predicted peptides for each antigen or at least 2 peptides per ORF. To remove redundant peptides, all peptides overlapping by 9 residues or more were placed into “variant clusters”. The most commonly occurring peptide was marked as the “representative” and the less-common peptides were marked as “variants”. Variants in each cluster were sorted by their alignment-start position and only synthesized once. Using these combined approaches, the inventors selected and synthesized a total of 24,876 peptides, spanning 3,305 unique ORFs. The peptides were pooled and organized into a library of 1,064 MesoPools (MS) composed of 24 individual peptides, and also a library of 133 MegaPools (MP) composed of 8 MS. All individual peptides were synthesized by Mimotopes (Victoria, Australia) and resuspended to a final concentration of 1 mg/mL in DMSO.

Whole genome screening study design. CD4+ T cell reactivity was assayed directly ex vivo using an Activation induced marker (AIM) assay previously validated for BP epitope discovery (da Silva Antunes et al., 2020). A summary of the screening strategy is shown in FIG. 8. PBMCs from each donor were tested with sets of the same peptide library After screening and identification of library pools that resulted in AIM+ reactive responses for each donor and based on cell availability the inventors deconvoluted the top 30 MP and top 34 MS for each individual donor, which in preliminary analysis was shown to capture ≥75% and ≥90% of the total MP and MS library response, respectively. Overall, for each donor, an average of 764 peptides of the total library were tested and the position of each individual epitope identified mapped to the aligned BP genome using the Tohama I and D420 BP strains as reference. The total magnitude of response and localization of each recognized ORF/antigen across the entire cohort was performed by summing all the reactivity of individual epitopes across all donors.

FIGS. 9A-9B show the immunodominance is associated with both magnitude and donor recognition. (FIG. 9A) Overall map of CD4+ T cell responses at antigen (ORF) level by percent of total magnitude (black bars, left axis) or percent of donor recognition (grey bars, right axis), across the entire cohort (n=40). Each bar represents an individual ORF identified across the aligned BP genome, using the Tohama I and D420 BP strains as reference. Dotted line represents a frequency of recognition of 5%. (FIG. 9B) Graph shows correlation between percentages of total magnitude and donor recognition. Each circle represents an individual ORF. R and p value expresses Spearman's rank correlation coefficient test.

Generation of peptide pools for non-aP vaccine antigens. To validate and characterize responses to the most dominant non-aP vaccine epitopes identified in this study, the inventors generated a pool encompassing 170 peptides [PT(E)R](Table 2) by selecting the top immunodominant epitopes recognized in at least 2 donors. To test the reactivity of the 15-defined immunodominant non-aP vaccine antigens, the inventors generated MPs of 15-mer peptides overlapping by 10 a.a. spanning the entire sequences of each individual antigen [PT(O)ANT1-15]. As a control, the inventors also studied antigen-specific responses against a previously described MP (Bancroft et al., 2016; da Silva Antunes et al., 2018), containing epitopes exclusive from aP vaccine antigens [PT(E)VAC] and from the ubiquitous pathogen CMV (Yu et al., 2022). Individual peptides were synthesized by TC peptide lab (San Diego, CA).

Activation Induced Marker (AIM) and Intracellular staining (ICS) assays. CD4+ T cell reactivity was assayed directly ex vivo using an Activation induced marker (AIM) assay utilizing the combination of markers OX40+CD25+ as previously described (Dan et al., 2016). This assay detects cells that are activated as a result of antigen-specific stimulation by staining antigen-experienced CD4+ T cells for TCR-dependent upregulation of OX40 and CD25 (AIM25) after an optimal time of 18-24 h of culture. Briefly, cryopreserved PBMCs were thawed, and 1×10⁶ cells/condition were immediately cultured together with peptide pools (2 μg/mL), individual peptides (10 μg/mL), or PHA (10 μg/mL; Roche, San Diego, CA) and DMSO as positive and negative controls, respectively, in 5% human serum (Gemini Bio-Products) for 24 h. All samples were acquired on a ZE5 cell analyzer (Biorad laboratories, Hercules, CA) and analyzed with FlowJo software (Tree Star, Ashland, OR). AIM+ CD4+ T cells data were calculated as percentage of cells per million of CD4+ T cells. Background subtracted data were derived by subtracting the % of AIM+ cells percentage after each MP stimulation from the average of triplicate wells stimulated with DMSO. The Stimulation Index (SI) was calculated by dividing the % of AIM+ cells after peptide pool stimulation with the average % of AIM⁺ cells in the negative DMSO control. A positive response was defined as SI greater than 2 and AIM+ response above the threshold of positivity after background subtraction. The threshold of positivity (0.0285%) was calculated based on the median twofold standard deviation of T cell reactivity in negative DMSO controls according to previous published studies (da Silva Antunes et al., 2020; Tarke et al., 2022).

The intracellular cytokine staining (ICS) assay was performed as previously described (Tarke et al., 2022). PBMCs were cultured in the presence of antigen-specific MPs [1 mg/ml] in 96-well U-bottom plates at a concentration of 2×10⁶ PBMC per well. As a negative control, an equimolar amount of DMSO was used to stimulate the cells in triplicate wells and PHA (1 mg/ml) stimulated cells were used as positive controls. After incubation for 24 hours at 37° C. in 5% CO2, cells were incubated for additional 4 hours after adding Golgi-Plug containing brefeldin A, Golgi-Stop containing monensin (BD Biosciences, San Diego, CA) together with CD137 APC antibody (2:100; Biolegend, San Diego, CA). Cells were then stained on their surface for 30 min at 4° C. in the dark, after that fixed with 1% of paraformaldehyde (Sigma-Aldrich, St. Louis, MO), permeabilized, and blocked for 15 minutes followed by intracellular staining for 30 min at room temperature. All samples were acquired on a ZE5 5-laser cell analyzer (Biorad laboratories, Hercules, CA) and analyzed with FlowJo software (Tree Star, Ashland, OR). Specifically, lymphocytes were gated, followed by single cells determination. T cells were gated for being positive to CD3 and negative for a Dump channel including in the same colors CD14, CD19 and Live/Dead staining. CD3+CD4+ were further gated based on a combination of each cytokine (IFNγ, TNFα, IL-2, and IL-4) with CD40L (CD154). The total cytokine response and T cell functionality was calculated from Boolean gating of single cytokines that was applied to CD3+CD4+ cells. The background was removed from the data by subtracting the average of the % of Cytokine+ cells plated in triplicate wells stimulated with DMSO. CD4+ T cell cytokine responses were background subtracted individually and found positive only if fulfilling the criteria of an SI greater than 2 and above a threshold of positivity (TP) of 0.002% for overall CD4+Cytokine+ cells. The TP for ICS was considered to be a positive response based on the median twofold standard deviation of T cell reactivity in negative DMSO controls.

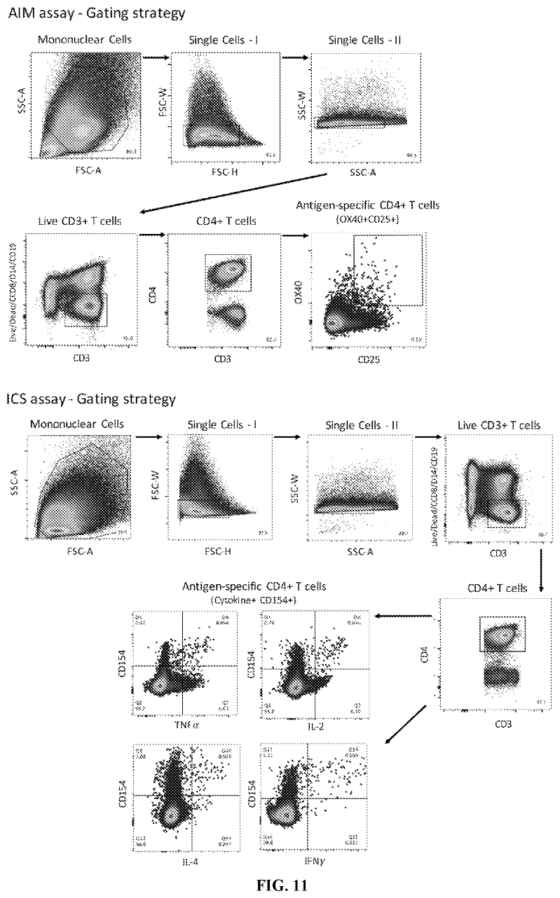

The detailed information of the gating strategy is listed in FIG. 11. Gates were drawn relative to the negative and positive controls for each donor.

Conservation analysis. The degree of conservation was performed amongst 20 strains representing different clades of BP or amongst 22 different species of the genus Bordetella. A bioinformatic analysis was conducted to ascertain the percent of homology for each individual peptide across all the different strains and species. Peptides were then divided in subsets according to their immunogenicity or degree of conservation in different isolates or related species. In terms of immunogenicity, peptides were arbitrarily divided in not recognized, subdominant (recognized in 1 donor), or dominant (recognized in >=2 donors). In terms of conservation, peptides were classified as variable (<75% of homology), intermediate (75-95% of homology), or conserved (>95% of homology). Finally, peptides were further segregated as derived from aP vaccine antigens, or from non-aP vaccine antigens. Results were plotted as geomean of percent homology or relative percent of the number of peptides in each subset.

Statistical analysis. Comparisons between groups were performed using the nonparametric two-tailed, and unpaired Mann-Whitney test or Kruskal-Wallis test adjusted with Dunn's test for multiple comparisons. Spearman's rank correlation coefficient test was used for association analysis. Prism 8.0.1 (GraphPad, San Diego, CA, USA) was used for these calculations. Values pertaining to significance and correlation coefficient (R) are noted in the respective figure, and P<0.05 defined as statistically significant.

Study approval. This study was performed with approvals from the Institutional Review Board at La Jolla Institute for Immunology (protocols; VD-101-0513 and VD-059-0813). All participants provided written informed consent for participation and clinical medical history was collected and evaluated.

### REFERENCES

Arciniega, J. L., Hewlett, E. L., Johnson, F. D., Deforest, A., Wassilak, S. G., Onorato, I. M., Manclark, C. R., and Burns, D. L. (1991). Human serologic response to envelope-associated proteins and adenylate cyclase toxin of Bordetella pertussis. J Infect Dis 163, 135-142.
Ausiello, C. M., Mascart, F., Corbiere, V., and Fedele, G. (2019). Human Immune Responses to Pertussis Vaccines. Adv Exp Med Biol 1183, 99-113.
Bancroft, T., Dillon, M. B., da Silva Antunes, R., Paul, S., Peters, B., Crotty, S., Lindestam Arlehamn, C. S., and Sette, A. (2016). Th1 versus Th2 T cell polarization by whole-cell and acellular childhood pertussis vaccines persists upon re-immunization in adolescence and adulthood. Cell Immunol 304-305, 35-43.
Blanchard-Rohner, G. (2022). Novel approaches to reactivate pertussis immunity. Expert Rev Vaccines 21, 1787-1797.
Buddy Creech, C., Jimenez-Truque, N., Kown, N., Sokolow, K., Brady, E. J., Yoder, S., Solovay, K., Rubin, K., Noviello, S., Hensel, E., et al. (2022). Safety and immunogenicity of live, attenuated intranasal Bordetella pertussis vaccine (BPZE1) in healthy adults. Vaccine 40, 6740-6746.
Bui, H. H., Sidney, J., Li, W., Fusseder, N., and Sette, A. (2007). Development of an epitope conservancy analysis tool to facilitate the design of epitope-based diagnostics and vaccines. BMC Bioinformatics 8, 361.
CDC (2023). Pertusis (Whooping Cough).
Chasaide, C. N., and Mills, K. H. G. (2020). Next-Generation Pertussis Vaccines Based on the Induction of Protective T Cells in the Respiratory Tract. Vaccines (Basel) 8.
Chen, Z., and He, Q. (2017). Immune persistence after pertussis vaccination. Hum Vaccin Immunother 13, 744-756.
Cherry, J. D., Xing, D. X., Newland, P., Patel, K., Heininger, U., and Corbel, M. J. (2004). Determination of serum antibody to Bordetella pertussis adenylate cyclase toxin in vaccinated and unvaccinated children and in children and adults with pertussis. Clin Infect Dis 38, 502-507.
Chit, A., Zivaripiran, H., Shin, T., Lee, J. K. H., Tomovici, A., Macina, D., Johnson, D. R., Decker, M. D., and Wu, J. (2018). Acellular pertussis vaccines effectiveness over time: A systematic review, meta-analysis and modeling study. PLoS One 13, e0197970.
Consortium, P. (2019). PERISCOPE: road towards effective control of pertussis. Lancet Infect Dis 19, e179-e186.
da Silva Antunes, R., Babor, M., Carpenter, C., Khalil, N., Cortese, M., Mentzer, A. J., Seumois, G., Petro, C. D., Purcell, L. A., Vijayanand, P., et al. (2018). Th1/Th17 polarization persists following whole-cell pertussis vaccination despite repeated acellular boosters. J Clin Invest 128, 3853-3865.
da Silva Antunes, R., Quiambao, L. G., Soldevila, F., Sutherland, A., Peters, B., and Sette, A. (2021). Lack of evidence supporting a role of IFN-beta and TGF-beta in differential polarization of Bordetella pertussis specific-T cell responses. Cytokine 137, 155313.
da Silva Antunes, R., Quiambao, L. G., Sutherland, A., Soldevila, F., Dhanda, S. K., Armstrong, S. K., Brickman, T. J., Merkel, T., Peters, B., and Sette, A. (2020). Development and Validation of a Bordetella pertussis Whole-Genome Screening Strategy. J Immunol Res 2020, 8202067.
Dalby, T., Petersen, J. W., Harboe, Z. B., and Krogfelt, K. A. (2010). Antibody responses to pertussis toxin display different kinetics after clinical Bordetella pertussis infection than after vaccination with an acellular pertussis vaccine. J Med Microbiol 59, 1029-1036.
Damron, F. H., Barbier, M., Dubey, P., Edwards, K. M., Gu, X. X., Klein, N. P., Lu, K., Mills, K. H. G., Pasetti, M. F., Read, R. C., et al. (2020). Overcoming Waning Immunity in Pertussis Vaccines: Workshop of the National Institute of Allergy and Infectious Diseases. J Immunol 205, 877-882.
Dan, J. M., Lindestam Arlehamn, C. S., Weiskopf, D., da Silva Antunes, R., Havenar-Daughton, C., Reiss, S. M., Brigger, M., Bothwell, M., Sette, A., and Crotty, S. (2016). A Cytokine-Independent Approach To Identify Antigen-Specific Human Germinal Center T Follicular Helper Cells and Rare Antigen-Specific CD4+ T Cells in Blood. J Immunol 197, 983-993.
de Melker, H. E., Versteegh, F. G., Schellekens, J. F., Teunis, P. F., and Kretzschmar, M. (2006). The incidence of Bordetella pertussis infections estimated in the population from a combination of serological surveys. J Infect 53, 106-113.
Dhanda, S. K., Mahajan, S., Paul, S., Yan, Z., Kim, H., Jespersen, M. C., Jurtz, V., Andreatta, M., Greenbaum, J. A., Marcatili, P., et al. (2019). IEDB-AR: immune epitope database-analysis resource in 2019. Nucleic Acids Res 47, W502-W506.
Diavatopoulos, D. A., and Edwards, K. M. (2017). What Is Wrong with Pertussis Vaccine Immunity? Why Immunological Memory to Pertussis Is Failing. Cold Spring Harb Perspect Biol 9.
Diks, A. M., de Graaf, H., Teodosio, C., Groenland, R. J., de Mooij, B., Ibrahim, M., Hill, A. R., Read, R. C., van Dongen, J. J., and Berkowska, M. A. (2023). Distinct early cellular kinetics in participants protected from colonization upon Bordetella pertussis challenge. J Clin Invest.
DiVenere, A. M., Amengor, D., Silva, R. P., Goldsmith, J. A., McLellan, J. S., and Maynard, J. A. (2022). Blockade of the Adenylate Cyclase Toxin Synergizes with Opsonizing Antibodies to Protect Mice against Bordetella pertussis. mBio 13, e0152722.
Domenech de Celles, M., Rohani, P., and King, A. A. (2019). Duration of Immunity and Effectiveness of Diphtheria-Tetanus-Acellular Pertussis Vaccines in Children. JAMA Pediatr 173, 588-594.
Elder, K. D., and Harvill, E. T. (2004). Strain-dependent role of BrkA during Bordetella pertussis infection of the murine respiratory tract. Infect Immun 72, 5919-5924.
Esposito, S., Stefanelli, P., Fry, N. K., Fedele, G., He, Q., Paterson, P., Tan, T., Knuf, M., Rodrigo, C., Weil Olivier, C., et al. (2019). Pertussis Prevention: Reasons for Resurgence, and Differences in the Current Acellular Pertussis Vaccines. Front Immunol 10, 1344.
Fedele, G., Cassone, A., and Ausiello, C. M. (2015). T-cell immune responses to Bordetella pertussis infection and vaccination. Pathog Dis 73.
Felice, A. G., Santos, L. N. Q., Kolossowski, I., Zen, F. L., Alves, L. G., Rodrigues, T. C. V., Prado, L. C. S., Jaiswal, A. K., Tiwari, S., Miranda, F. M., et al. (2022). Comparative genomics of Bordetella pertussis and prediction of new vaccines and drug targets. J Biomol Struct Dyn 40, 10136-10152.
Gregg, K. A., Wang, Y., Warfel, J., Schoenfeld, E., Jankowska, E., Cipollo, J. F., Mayho, M., Boinett, C., Prasad, D., Brickman, T. J., et al. (2022). “Antigen Discovery for Next-Generation Pertussis Vaccines using Immunoproteomics and Transposon Directed Insertion Sequencing”. J Infect Dis.
Grifoni, A., Weiskopf, D., Ramirez, S. I., Mateus, J., Dan, J. M., Moderbacher, C. R., Rawlings, S. A., Sutherland, A., Premkumar, L., Jadi, R. S., et al. (2020). Targets of T Cell Responses to SARS-CoV-2 Coronavirus in Humans with COVID-19 Disease and Unexposed Individuals. Cell 181, 1489-1501 e1415.
Heininger, U., Cherry, J. D., and Stehr, K. (2004a). Serologic response and antibody-titer decay in adults with pertussis. Clin Infect Dis 38, 591-594.
Heininger, U., Kleemann, W. J., Cherry, J. D., and Sudden Infant Death Syndrome Study, G. (2004b). A controlled study of the relationship between Bordetella pertussis infections and sudden unexpected deaths among German infants. Pediatrics 114, e9-15.
Higgs, R., Higgins, S. C., Ross, P. J., and Mills, K. H. (2012). Immunity to the respiratory pathogen Bordetella pertussis. Mucosal Immunol 5, 485-500.
Jenkinson, D. (1988). Duration of effectiveness of pertussis vaccine: evidence from a 10 year community study. Br Med J (Clin Res Ed) 296, 612-614.
Kandeil, W., Atanasov, P., Avramioti, D., Fu, J., Demarteau, N., and Li, X. (2019). The burden of pertussis in older adults: what is the role of vaccination? A systematic literature review. Expert Rev Vaccines 18, 439-455.
Kapil, P., and Merkel, T. J. (2019). Pertussis vaccines and protective immunity. Curr Opin Immunol 59, 72-78.
Klein, N. P., Bartlett, J., Fireman, B., Rowhani-Rahbar, A., and Baxter, R. (2013). Comparative effectiveness of acellular versus whole-cell pertussis vaccines in teenagers. Pediatrics 131, e1716-1722.
Kuehn, B. M. (2021). Resurgence of Pertussis Linked With Switch to Acellular Vaccine. JAMA 326, 300.
Lambert, E. E., van Twillert, I., Beckers, L., Poelen, M. C. M., Han, W. G. H., Pieren, D. K. J., and van Els, C. (2021). Reduced Bordetella pertussis-specific CD4(+) T-Cell Responses at Older Age. Front Aging 2, 737870.
Le, T., Cherry, J. D., Chang, S. J., Knoll, M. D., Lee, M. L., Barenkamp, S., Bernstein, D., Edelman, R., Edwards, K. M., Greenberg, D., et al. (2004). Immune responses and antibody decay after immunization of adolescents and adults with an acellular pertussis vaccine: the APERT Study. J Infect Dis 190, 535-544.
Lindestam Arlehamn, C. S., Gerasimova, A., Mele, F., Henderson, R., Swann, J., Greenbaum, J. A., Kim, Y., Sidney, J., James, E. A., Taplitz, R., et al. (2013). Memory T cells in latent Mycobacterium tuberculosis infection are directed against three antigenic islands and largely contained in a CXCR3+CCR6+ Th1 subset. PLoS Pathog 9, e1003130.
Macina, D., and Evans, K. E. (2021). Pertussis in Individuals with Co-morbidities: A Systematic Review. Infect Dis Ther 10, 1141-1170.
McAlister, S. M., van den Biggelaar, A. H. J., Woodman, T. L., Hutton, H., Thornton, R. B., and Richmond, P. C. (2021). An observational study of antibody responses to a primary or subsequent pertussis booster vaccination in Australian healthcare workers. Vaccine 39, 1642-1651.
Moon, K., Bonocora, R. P., Kim, D. D., Chen, Q., Wade, J. T., Stibitz, S., and Hinton, D. M. (2017). The BvgAS Regulon of Bordetella pertussis. mBio 8.
Naeini, A. E., Zaman, N., Khorvash, F., Naeini, S E., Khodadadi, H. A., Mokhtari, M., and Koushki, A. M. (2015). Does working in hospital increases seroprevalence and carrier state against Bordetella pertussis? Adv Biomed Res 4, 194.
Oseroff, C., Sidney, J., Kotturi, M. F., Kolla, R., Alam, R., Broide, D. H., Wasserman, S. I., Weiskopf, D., McKinney, D. M., Chung, J. L., et al. (2010). Molecular determinants of T cell epitope recognition to the common Timothy grass allergen. J Immunol 185, 943-955.
Palazzo, R., Carollo, M., Fedele, G., Rizzo, C., Rota, M. C., Giammanco, A., Iannazzo, S., Ausiello, C. M., and Sero-Epidemiology Working, G. (2016). Evidence of increased circulation of Bordetella pertussis in the Italian adult population from seroprevalence data (2012-2013). J Med Microbiol 65, 649-657.
Park, J., Zhang, Y., Chen, C., Dudley, E. G., and Harvill, E. T. (2015). Diversity of secretion systems associated with virulence characteristics of the classical bordetellae. Microbiology (Reading) 161, 2328-2340.
Paul, S., Lindestam Arlehamn, C. S., Scriba, T. J., Dillon, M. B., Oseroff, C., Hinz, D., McKinney, D. M., Carrasco Pro, S., Sidney, J., Peters, B., et al. (2015). Development and validation of a broad scheme for prediction of HLA class II restricted T cell epitopes. J Immunol Methods 422, 28-34.
Plotkin, S. A. (2018). Composition of pertussis vaccine given to infants determines long-term T cell polarization. J Clin Invest 128, 3742-3744.
Raeven, R. H., van der Maas, L., Tilstra, W., Uittenbogaard, J. P., Bindels, T. H., Kuipers, B., van der Ark, A., Pennings, J. L., van Riet, E., Jiskoot, W., et al. (2015). Immunoproteomic Profiling of Bordetella pertussis Outer Membrane Vesicle Vaccine Reveals Broad and Balanced Humoral Immunogenicity. J Proteome Res 14, 2929-2942.
Rolan, H. G., and Tsolis, R. M. (2008). Inactivation of the type IV secretion system reduces the Th1 polarization of the immune response to Brucella abortus infection. Infect Immun 76, 3207-3213.
Saikh, K. U., Kissner, T. L., Dyas, B., Tropea, J. E., Waugh, D. S., and Ulrich, R. G. (2006). Human cytolytic T cell recognition of Yersinia pestis virulence proteins that target innate immune responses. J Infect Dis 194, 1753-1760.
Saso, A., Kampmann, B., and Roetynck, S. (2021). Vaccine-Induced Cellular Immunity against Bordetella pertussis: Harnessing Lessons from Animal and Human Studies to Improve Design and Testing of Novel Pertussis Vaccines. Vaccines (Basel) 9.
Sette, A., Moutaftsi, M., Moyron-Quiroz, J., McCausland, M. M., Davies, D. H., Johnston, R. J., Peters, B., Rafii-El-Idrissi Benhnia, M., Hoffmann, J., Su, H. P., et al. (2008). Selective CD4+ T cell help for antibody responses to a large viral pathogen: deterministic linkage of specificities. Immunity 28, 847-858.
Shepherd, F. R., and McLaren, J. E. (2020). T Cell Immunity to Bacterial Pathogens: Mechanisms of Immune Control and Bacterial Evasion. Int J Mol Sci 21.
Solans, L., and Locht, C. (2018). The Role of Mucosal Immunity in Pertussis. Front Immunol 9, 3068.
Tarke, A., Coelho, C. H., Zhang, Z., Dan, J. M., Yu, E. D., Methot, N., Bloom, N. I., Goodwin, B., Phillips, E., Mallal, S., et al. (2022). SARS-CoV-2 vaccination induces immunological T cell memory able to cross-recognize variants from Alpha to Omicron. Cell 185, 847-859 e811.
Tian, Y., da Silva Antunes, R., Sidney, J., Lindestam Arlehamn, C. S., Grifoni, A., Dhanda, S. K., Paul, S., Peters, B., Weiskopf, D., and Sette, A. (2018). A Review on T Cell Epitopes Identified Using Prediction and Cell-Mediated Immune Models for Mycobacterium tuberculosis and Bordetella pertussis. Front Immunol 9, 2778.
van der Lee, S., Hendrikx, L. H., Sanders, E. A. M., Berbers, G. A. M., and Buisman, A. M. (2018). Whole-Cell or Acellular Pertussis Primary Immunizations in Infancy Determines Adolescent Cellular Immune Profiles. Front Immunol 9, 51.
van Schuppen, E., Froberg, J., Venkatasubramanian, P. B., Versteegen, P., de Graaf, H., Holubova, J., Gillard, J., van Gageldonk, P. G. M., Joosten, I., de Groot, R., et al. (2022). Prior exposure to B. pertussis shapes the mucosal antibody response to acellular pertussis booster vaccination. Nat Commun 13, 7429.
Warfel, J. M., Zimmerman, L. I., and Merkel, T. J. (2014). Acellular pertussis vaccines protect against disease but fail to prevent infection and transmission in a nonhuman primate model. Proc Natl Acad Sci USA 111, 787-792.
Wearing, H. J., and Rohani, P. (2009). Estimating the duration of pertussis immunity using epidemiological signatures. PLoS Pathog 5, e1000647.
Westernberg, L., Schulten, V., Greenbaum, J. A., Natali, S., Tripple, V., McKinney, D. M., Frazier, A., Hofer, H., Wallner, M., Sallusto, F., et al. (2016). T-cell epitope conservation across allergen species is a major determinant of immunogenicity. J Allergy Clin Immunol 138, 571-578 e577.
Wilk, M. M., Borkner, L., Misiak, A., Curham, L., Allen, A. C., and Mills, K. H. G. (2019). Immunization with whole cell but not acellular pertussis vaccines primes CD4 TRM cells that sustain protective immunity against nasal colonization with Bordetella pertussis. Emerg Microbes Infect 8, 169-185.
Wilkinson, T. M. A., Van den Steen, P., Cheuvart, B., Baudson, N., Dodet, M., Turriani, E., Harrington, L., Meyer, N., Rondini, S., Taddei, L., et al. (2021). Seroprevalence of Bordetella pertussis Infection in Patients With Chronic Obstructive Pulmonary Disease in England: Analysis of the AERIS Cohort. COPD 18, 341-348.
Wirsing von Konig, C. H., Postels-Multani, S., Bock, H. L., and Schmitt, H. J. (1995). Pertussis in adults: frequency of transmission after household exposure. Lancet 346, 1326-1329.
Yu, E. D., Narowski, T. M., Wang, E., Garrigan, E., Mateus, J., Frazier, A., Weiskopf, D., Grifoni, A., Premkumar, L., da Silva Antunes, R., et al. (2022). Immunological memory to common cold coronaviruses assessed longitudinally over a three-year period pre-COVID19 pandemic. Cell Host Microbe 30, 1269-1278 e1264.
Zeddeman, A., van Schuppen, E., Kok, K. E., van Gent, M., Heuvelman, K. J., Bart, M. J, van der Heide, H. G. J., Gillard, J., Simonetti, E., Eleveld, MI., et al. (2020). Effect of FHA and Pm on Bordetella pertussis colonization of mice is dependent on vaccine type and anatomical site. PLoS One 15, e0237394.
Zhang, Q., Yin, Z., Li, Y., Luo, H., Shao, Z., Gao, Y., Xu, L., Kan, B., Lu, S., Zhang, Y., et al. (2014). Prevalence of asymptomatic Bordetella pertussis and Bordetella parapertussis infections among school children in China as determined by pooled real-time PCR: a cross-sectional study. Scand J Infect Dis 46, 280-287.
Bancroft, T., Dillon, M. B., da Silva Antunes, R., Paul, S., Peters, B., Crotty, S., Lindestam Arlehamn, C. S., and Sette, A. (2016). Th1 versus Th2 T cell polarization by whole-cell and acellular childhood pertussis vaccines persists upon re-immunization in adolescence and adulthood. Cell Immunol 304-305, 35-43.
da Silva Antunes, R., Babor, M., Carpenter, C., Khalil, N., Cortese, M., Mentzer, Al., Seumois, G., Petro, C. D., Purcell, L. A., Vijayanand, P., et al. (2018). Th1/Th17 polarization persists following whole-cell pertussis vaccination despite repeated acellular boosters. J Clin Invest 128, 3853-3865.
da Silva Antunes, R., Quiambao, L. G., Soldevila, F., Sutherland, A., Peters, B., and Sette, A. (2021). Lack of evidence supporting a role of IFN-beta and TGF-beta in differential polarization of Bordetella pertussis specific-T cell responses. Cytokine 137, 155313.
da Silva Antunes, R., Quiambao, L. G., Sutherland, A., Soldevila, F., Dhanda, S. K., Armstrong, S. K., Brickman, T. J., Merkel, T., Peters, B., and Sette, A. (2020). Development and Validation of a Bordetella pertussis Whole-Genome Screening Strategy. J Immunol Res 2020, 8202067.

### Definitions

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules (e.g., sgRNA) may be detected by standard PCR or Northern blot methods well known in the art. See, Sambrook et al., 1989 Molecular Cloning: A Laboratory Manual, 18.1-18.88.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may, in embodiments, be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

Proteins and peptides include isolated and purified forms. Proteins and peptides also include those immobilized on a substrate, as well as amino acid sequences, subsequences, portions, homologues, variants, and derivatives immobilized on a substrate.

Proteins and peptides can be included in compositions, for example, a pharmaceutical composition. In particular embodiments, a pharmaceutical composition is suitable for specific or non-specific immunotherapy, or is a vaccine composition.

Isolated nucleic acid (including isolated nucleic acid) encoding the proteins and peptides are also provided. Cells expressing a protein or peptide are further provided. Such cells include eukaryotic and prokaryotic cells, such as mammalian, insect, fungal and bacterial cells.

Methods and uses and medicaments of proteins and peptides of the invention are included. Such methods, uses and medicaments include modulating immune activity of a cell against a pathogen, for example, a bacteria or bacteria.

The term “peptide mimetic” or “peptidomimetic” refers to protein-like chain designed to mimic a peptide or protein. Peptide mimetics may be generated by modifying an existing peptide or by designing a compound that mimic peptides, including peptoids and β-peptides.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure. The following eight groups each contain amino acids that are conservative substitutions for one another: (1) Alanine (A), Glycine (G); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); (6) Phenylalanine (F), Tyrosine (Y), Tryptophan (W); (7) Serine (S), Threonine (T); and (8) Cysteine (C), Methionine (M) (see, e.g., Creighton, Proteins (1984)).

A “percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site ncbi.nlm.nih.gov/BLAST/or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

An amino acid or nucleotide base “position” is denoted by a number that sequentially identifies each amino acid (or nucleotide base) in the reference sequence based on its position relative to the N-terminus (or 5-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

The term “multimer” refers to a complex comprising multiple monomers (e.g., a protein complex) associated by noncovalent bonds. The monomers be substantially identical monomers, or the monomers may be different. In embodiments, the multimer is a dimer, a trimer, a tetramer, or a pentamer.

As used herein, the term “Major Histocompatibility Complex” (MHC) is a generic designation meant to encompass the histocompatibility antigen systems described in different species including the human leucocyte antigens (HLA). Typically, MHC Class I or Class II multimers are well known in the art and include but are not limited to dimers, tetramers, pentamers, hexamers, heptamers and octamers.

As used herein, the term “MHC/peptide multimer” refers to a stable multimeric complex composed of MHC protein(s) subunits loaded with a peptide of the present invention. For example, an MHC/peptide multimer (also called herein MHC/peptide complex) include, but are not limited to, an MHC/peptide dimer, trimer, tetramer, pentamer or higher valency multimer. In humans there are three major different genetic loci that encode MHC class I molecules (the MHC molecules of the human are also designated human leukocyte antigens (HLA)): HLA-A, HLA-B, HLA-C, e.g., HLA-A*01, HLA-A*02, and HLA-A*11 are examples of different MHC class I alleles that can be expressed from these loci. Non-classical human MHC class I molecules such as HLA-E (homolog of mice Qa-1b) and MICA/B molecules are also encompassed by the present invention. In some embodiments, the MHC/peptide multimer is an HLA/peptide multimer selected from the group consisting of HLA-A/peptide multimer, HLA-B/peptide multimer, HLA-C/peptide multimer, HLA-E/peptide multimer, MICA/peptide multimer and MICB/peptide multimer.

In humans there are three major different genetic loci that encode MHC class II molecules: HLA-DR, HLA-DP, and HLA-DQ, each formed of two polypeptides, alpha and beta chains (A and B genes). For example, HLA-DQA1*01, HLA-DRB1*01, and HLA-DRB1*03 are different MHC class II alleles that can be expressed from these loci. It should be further noted that non-classical human MHC class II molecules such as HLA-DM and HL-DOA (homolog in mice is H2-DM and H2-O) are also encompassed by the present invention. In some embodiments, the MHC/peptide multimer is an HLA/peptide multimer selected from the group consisting of HLA-DP/peptide multimer, HLA-DQ/peptide multimer, HLA-DR/peptide multimer, HLA-DM/peptide multimer and HLA-DO/peptide multimer.

An MHC/peptide multimer may be a multimer where the heavy chain of the MHC is biotinylated, which allows combination as a tetramer with streptavidin. MHC-peptide tetramers have increased avidity for the appropriate T cell receptor (TCR) on T lymphocytes. The multimers can also be attached to paramagnetic particles or magnetic beads to facilitate removal of non-specifically bound reporter and cell sorting. Multimer staining does not kill the labelled cells, thus, cell integrity is maintained for further analysis. In some embodiments, the MHC/peptide multimer of the present invention is particularly suitable for isolating and/or identifying a population of CD8+ T cells having specificity for the peptide of the present invention (in a flow cytometry assay).

The peptides or MHC class I or class II multimer as described herein is particularly suitable for detecting T cells specific for one or more peptides of the present invention. The peptide(s) and/or the MHC/multimer complex of the present invention is particularly suitable for diagnosing Bordetella infection in a subject. For example, the method comprises obtaining a blood or PBMC sample obtained from the subject with an amount of a least peptide of the present invention and detecting at least one T cell displaying a specificity for the peptide. Another diagnostic method of the present invention involves the use of a peptide of the present invention that is loaded on multimers as described above, so that the isolated CD8+ or CD4+ T cells from the subject are brought into contact with the multimers, at which the binding, activation and/or expansion of the T cells is measured. For example, following the binding to antigen presenting cells, e.g., those having the MHC class I or class II multimer, the number of CD8+ and/or CD4+ cells binding specifically to the HLA-peptide multimer may be quantified by measuring the secretion of lymphokines/cytokines, division of the T cells, or standard flow cytometry methods, such as, for example, using fluorescence activated cell sorting (FACS). The multimers can also be attached to paramagnetic ferrous or magnetic beads to facilitate removal of non-specifically bound reporter and cell sorting. The MHC class I or class II peptide multimers as described herein can also be used as therapeutic agents. The peptide and/or the MHC class I or class II peptide multimers of the present invention are suitable for treating or preventing a Bordetella infection in a subject. The MHC Class I or Class II multimers can be administered in soluble form or loaded on nanoparticles.

The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The phrase “specifically (or selectively) binds” to an antibody or “specifically (or selectively) immunoreactive with,” when referring to a protein or peptide, refers to a binding reaction that is determinative of the presence of the protein or peptide, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

Antibodies are large, complex molecules (molecular weight of ˜150,000 or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region involved in binding the target antigen, and a constant (“C”) region that interacts with other components of the immune system. The light and heavy chain variable regions come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework (“FR”), which forms the environment for the CDRs.

The term “antibody” is used according to its commonly known meaning in the art. Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H1} by a disulfide bond. The F(ab)′₂ may be reduced under mild conditions to break the disulfide linkage in the hinge region, thereby converting the F(ab)′₂ dimer into a Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively. The Fc (i.e., fragment crystallizable region) is the “base” or “tail” of an immunoglobulin and is typically composed of two heavy chains that contribute two or three constant domains depending on the class of the antibody. By binding to specific proteins, the Fc region ensures that each antibody generates an appropriate immune response for a given antigen. The Fc region also binds to various cell receptors, such as Fc receptors, and other immune molecules, such as complement proteins.

As used herein, the term “antigen” and the term “epitope” refers to a molecule or substance capable of stimulating an immune response. In one example, epitopes include but are not limited to a polypeptide and a nucleic acid encoding a polypeptide, wherein expression of the nucleic acid into a polypeptide is capable of stimulating an immune response when the polypeptide is processed and presented on a Major Histocompatibility Complex (MHC) molecule. Generally, epitopes include peptides presented on the surface of cells non-covalently bound to the binding groove of Class I or Class II MHC, such that they can interact with T cell receptors and the respective T cell accessory molecules. However, antigens and epitopes also apply when discussing the antigen binding portion of an antibody, wherein the antibody binds to a specific structure of the antigen.

Proteolytic Processing of Antigens. Epitopes that are displayed by MHC on antigen presenting cells are cleavage peptides or products of larger peptide or protein antigen precursors. For MHC I epitopes, protein antigens are often digested by proteasomes resident in the cell. Intracellular proteasomal digestion produces peptide fragments of about 3 to 23 amino acids in length that are then loaded onto the MHC protein. Additional proteolytic activities within the cell, or in the extracellular milieu, can trim and process these fragments further. Processing of MHC Class II epitopes generally occurs via intracellular proteases from the lysosomal/endosomal compartment. The present invention includes, in one embodiment, pre-processed peptides that are attached to the anti-CD40 antibody (or fragment thereof) that directs the peptides against which an enhanced immune response is sought directly to antigen presenting cells.

The present invention includes methods for specifically identifying the epitopes within antigens most likely to lead to the immune response sought for the specific sources of antigen presenting cells and responder T cells.

As used herein, the term “T cell epitope” refers to a specific amino acid that when present in the context of a Major or Minor Histocompatibility Complex provides a reactive site for a T cell receptor. The T-cell epitopes or peptides that stimulate the cellular arm of a subject's immune system are short peptides of about 8-25 amino acids. T-cell epitopes are recognized by T cells from animals that are immune to the antigen of interest. These T-cell epitopes or peptides can be used in assays such as the stimulation of cytokine release or secretion or evaluated by constructing major histocompatibility (MHC) proteins containing or “presenting” the peptide. Such immunogenically active fragments are often identified based on their ability to stimulate lymphocyte proliferation in response to stimulation by various fragments from the antigen of interest.

As used herein, the term “immunological response” refers to an antigen or composition is the development in a subject of a humoral and/or a cellular immune response to an antigen present in the composition of interest. For purposes of the present disclosure, a “humoral immune response” refers to an immune response mediated by antibody molecules, while a “cellular immune response” is one mediated by T-lymphocytes and/or other white blood cells. One important aspect of cellular immunity involves an antigen-specific response by cytolytic T-cells (“CTL”s). CTLs have specificity for peptide antigens that are presented in association with proteins encoded by the major histocompatibility complex (MHC) and expressed on the surfaces of cells. CTLs help induce and promote the destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cellular immune response” also refers to the production of cytokines, chemokines and other such molecules produced by activated T-cells and/or other white blood cells, including those derived from CD4+ and CD8+ T-cells. Hence, an immunological response may include one or more of the following effects: the production of antibodies by B-cells; and/or the activation of effector and/or suppressor T-cells and/or gamma-delta T-cells directed specifically to an antigen or antigens present in the composition or vaccine of interest. These responses may serve to neutralize infectivity, and/or mediate antibody-complement, or antibody dependent cell cytotoxicity (ADCC) to provide protection to an immunized host. Such responses can be determined using standard immunoassays and neutralization assays, well known in the art.

As used herein, the term an “immunogenic composition” and “vaccine” refer to a composition that comprises an antigenic molecule where administration of the composition to a subject or patient results in the development in the subject of a humoral and/or a cellular immune response to the antigenic molecule of interest. “Vaccine” refers to a composition that can provide active acquired immunity to and/or therapeutic effect (e.g., treatment) of a particular disease or a pathogen. A vaccine typically contains one or more agents that can induce an immune response in a subject against a pathogen or disease, i.e., a target pathogen or disease. The immunogenic agent stimulates the body's immune system to recognize the agent as a threat or indication of the presence of the target pathogen or disease, thereby inducing immunological memory so that the immune system can more easily recognize and destroy any of the pathogen on subsequent exposure. Vaccines can be prophylactic (e.g., preventing or ameliorating the effects of a future infection by any natural or pathogen) or therapeutic (e.g., reducing symptoms or aberrant conditions associated with infection). The administration of vaccines is referred to vaccination.

In some examples, a vaccine composition can provide nucleic acid, e.g., mRNA that encodes antigenic molecules (e.g., peptides) to a subject. The nucleic acid that is delivered via the vaccine composition in the subject can be expressed into antigenic molecules and allow the subject to acquire immunity against the antigenic molecules. In the context of the vaccination against infectious disease, the vaccine composition can provide mRNA encoding antigenic molecules that are associated with a certain pathogen, e.g., one or more peptides that are known to be expressed in the pathogen (e.g., pathogenic bacterium or bacteria).

The present invention provides nucleic acid molecules, specifically polynucleotides, primary constructs and/or mRNA that encode one or more polynucleotides that express one or more peptides or proteins, comprising, consisting of, or consisting essentially of an amino acid sequence selected from any one of those sequences set forth in Tables 1-20, or a subsequence, portion, homologue, variant or derivative thereof for use in immune modulation. The term “nucleic acid” refers to any compound and/or substance that comprise a polymer of nucleotides, referred to herein as polynucleotides. Exemplary nucleic acids or polynucleotides of the invention include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs), including diastereomers of LNAs, functionalized LNAs, or hybrids thereof.

One method of immune modulation of the present invention includes direct or indirect gene transfer, i.e., local application of a preparation containing the one or more polynucleotides (DNA, RNA, mRNA, etc.) that expresses the one or more peptides or proteins, comprising, consisting of, or consisting essentially of an amino acid sequence selected from any one of those sequences set forth in Tables 1-20, or a subsequence, portion, homologue, variant or derivative thereof. A variety of well-known vectors can be used to deliver to cells the one or more polynucleotides or the peptides or proteins expressed by the polynucleotides, including but not limited to adenobacterial vectors and adeno-associated vectors. In addition, naked DNA, liposome delivery methods, or other novel vectors developed to deliver the polynucleotides to cells can also be beneficial. Any of a variety of promoters can be used to drive peptide or protein expression, including but not limited to endogenous promoters, constitutive promoters (e.g., cytomegalobacteria, adenobacteria, or SV40), inducible promoters (e.g., a cytokine promoter such as the interleukin-1, tumor necrosis factor-alpha, or interleukin-6 promoter), and tissue specific promoters to express the immunogenic peptides or proteins of the present invention.

The immunization may include adenobacteria, adeno-associated bacteria, herpes bacteria, vaccinia bacteria, retrobacteriaes, or other bacterial vectors with the appropriate tropism for cells likely to present the antigenic peptide(s) or protein(s) may be used as a gene transfer delivery system for a therapeutic peptide(s) or protein(s), comprising, consisting of, or consisting essentially of an amino acid sequence selected from any one of those sequences set forth in Tables 1-20, or a subsequence, portion, homologue, variant or derivative thereof, gene expression construct. Bacterial vectors which do not require that the target cell be actively dividing, such as adenobacterial and adeno-associated vectors, are particularly useful when the cells are accumulating, but not proliferative. Numerous vectors useful for this purpose are generally known (Miller, Human Gene Therapy 15-14, 1990; Friedman, Science 244:1275-1281, 1989; Eglitis and Anderson, BioTechniques 6:608-614, 1988; Tolstoshev and Anderson, Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; and Miller and Rosman, Bio Techniques 7:980-990, 1989; Le Gal La Salle et al., Science 259:988-990, 1993; and Johnson, Chest 107:77S-83S, 1995). Retrobacterial vectors are particularly well developed and have been used in clinical settings (Rosenberg et al., N. Engl. J. Med 323:370, 1990; Anderson et al., U.S. Pat. No. 5,399,346).

The immunization may also include inserting the one or more polynucleotides (DNA, RNA, mRNA, etc.) that express the one or more peptides or proteins, comprising, consisting of, or consisting essentially of an amino acid sequence selected from any one of those sequences set forth in Tables 1-20, or a subsequence, portion, homologue, variant or derivative thereof into the bacterial vector, along with another gene which encodes the ligand for a receptor on a specific target cell, for example, such that the vector is now target specific. Bacterial vectors can be made target specific by attaching, for example, a sugar, a glycolipid, or a protein. Targeting can also be accomplished by using an antibody to target the bacterial vector. Those of skill in the art will know of, or can readily ascertain without undue experimentation, specific polynucleotide sequences which can be inserted into the bacterial genome or attached to a bacterial envelope to allow target specific delivery of the bacterial vector containing the gene.

Since recombinant bacteriaes are defective, they require assistance in order to produce infectious vector particles. This assistance can be provided, for example, by using helper cell lines that contain plasmids encoding all of the structural genes of the bacteria under the control of regulatory sequences within the bacterial genome. These plasmids are missing a nucleotide sequence which enables the packaging mechanism to recognize a polynucleotide transcript for encapsidation. These cell lines produce empty virions, since no genome is packaged. If a bacterial vector is introduced into such cells in which the packaging signal is intact, but the structural genes are replaced by other genes of interest, the vector can be packaged and vector virion produced.

Bacterial or non-bacterial approaches may also be employed for the introduction of one or more therapeutic polynucleotides that express the one or more peptides or proteins, comprising, consisting of, or consisting essentially of an amino acid sequence selected from any one of those sequences set forth in Tables 1-20, or a subsequence, portion, homologue, variant or derivative thereof, into polynucleotide-encoding polynucleotide into antigen presenting cells. The polynucleotides may be DNA, RNA, mRNA that directly encode the one or more peptides or proteins of the present invention, or may be introduced as part of an expression vector.

Another example of an immunization includes colloidal dispersion systems that include macromolecule complexes, nanocapsules, microspheres, beads, and lipid-based systems including oil-in-water emulsions, micelles, mixed micelles, and liposomes and the one or more polynucleotides that express the one or more peptides or proteins, comprising, consisting of, or consisting essentially of an amino acid sequence selected from any one of those sequences set forth in Tables 1-20, or a subsequence, portion, homologue, variant or derivative thereof. One non-limiting example of a colloidal system for use with the present invention is a liposome. Liposomes are artificial membrane vesicles which are useful as delivery vehicles in vitro and in vivo. It has been shown that large unilamellar vesicles (LUV), which range in size from 0.2-4.0 micrometers that can encapsulate a substantial percentage of an aqueous buffer containing large macromolecules. RNA, DNA and intact virions can be encapsulated within the aqueous interior and be delivered to cells in a biologically active form (Fraley, et al., Trends Biochem. Sci., 6:77, 1981). In addition to mammalian cells, liposomes have been used for delivery of polynucleotides in plant, yeast and bacterial cells. In order for a liposome to be an efficient gene transfer vehicle, the following characteristics should be present: (Zakut and Givol, supra) encapsulation of the genes of interest at high efficiency while not compromising their biological activity; (Fearnhead, et al., supra) preferential and substantial binding to a target cell in comparison to non-target cells; (Korsmeyer, S. J., supra) delivery of the aqueous contents of the vesicle to the target cell cytoplasm at high efficiency; and (Kinoshita, et al., supra) accurate and effective expression of genetic information (Mannino, et al., Bio Techniques, 6:682, 1988).

The composition for immunizing the subject or patient may, in certain embodiments comprise a combination of phospholipid, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations. The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticuloendothelial system (RES) in organs which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization, specifically, cells that can become infected with a Bordetella or interact with the proteins, peptides, and/or gene products of a Bordetella, e.g., immune cells.

For any of the above approaches, the immune modulating polynucleotide construct, composition, or formulation is preferably applied to a site that will enhance the immune response. For example, the immunization may be intramuscular, intraperitoneal, enteral, parenteral, intranasal, intrapulmonary, or subcutaneous. In the gene delivery constructs of the instant invention, polynucleotide expression is directed from any suitable promoter (e.g., the human cytomegalobacteria, simian bacteria 40, actin or adenobacteria constitutive promoters; or the cytokine or metalloprotease promoters for activated synoviocyte specific expression).

In one example of the immune modifying peptide(s) or protein(s) include polynucleotides, constructs and/or mRNAs that express the one or more polynucleotides that express the one or more peptides or proteins, comprising, consisting of, or consisting essentially of an amino acid sequence selected from any one of those sequences set forth in Tables 1-20, or a subsequence, portion, homologue, variant or derivative thereof, that are designed to improve one or more of the stability and/or clearance in tissues, uptake and/or kinetics, cellular access by the peptide(s) or protein(s), translational, mRNA half-life, translation efficiency, immune evasion, protein production capacity, accessibility to circulation, peptide(s) or protein(s) half-life and/or presentation in the context of MHC on antigen presenting cells.

The present invention contemplates immunization for use in both active and passive immunization embodiments. Immunogenic compositions, proposed to be suitable for use as a vaccine, may be prepared most readily directly from immunogenic peptides, proteins, monomers, multimers and/or peptide-MHC complexes prepared in a manner disclosed herein. The antigenic material is generally processed to remove undesired contaminants, such as, small molecular weight molecules, incomplete proteins, or when manufactured in plant cells, plant components such as cell walls, plant proteins, and the like. Often, these immunizations are lyophilized for ease of transport and/or to increase shelf-life and can then be more readily dissolved in a desired vehicle, such as saline.

The preparation of immunizations (also referred to as vaccines) that contain the immunogenic proteins of the present invention as active ingredients is generally well understood in the art, as exemplified by U.S. Pat. Nos. 4,608,251; 4,601,903; 4,599,231; 4,599,230; 4,596,792; and 4,578,770, all incorporated herein by reference. Typically, such immunizations are prepared as injectables. The immunizations can be a liquid solution or suspension but may also be provided in a solid form suitable for solution in, or suspension in, liquid prior to injection may also be prepared. The preparation may also be emulsified. The active immunogenic ingredient is often mixed with excipients that are pharmaceutically acceptable and compatible with the active ingredient. Suitable excipients are, for example, water, saline, dextrose, glycerol, ethanol, buffers, or the like and combinations thereof. In addition, if desired, the immunization may contain minor amounts of auxiliary substances such as wetting or emulsifying agents, pH buffering agents, or adjuvants which enhance the effectiveness of the vaccines.

The immunization is/are administered in a manner compatible with the dosage formulation, and in such amount as will be therapeutically effective and immunogenic. The quantity to be administered depends on the subject to be treated, including, e.g., the capacity of the individual's immune system to synthesize antibodies, and the degree of protection desired. Precise amounts of active ingredient required to be administered depend on the judgment of the practitioner. However, suitable dosage ranges are of the order of several hundred micrograms active ingredient per vaccination. Suitable regimes for initial administration and booster shots are also variable but are typified by an initial administration followed by subsequent inoculations or other administrations.

The manner of application of the immunization may be varied widely. Any of the conventional methods for administration of a vaccine are applicable. These are believed to also include oral application on a solid physiologically acceptable base or in a physiologically acceptable dispersion, parenterally, by injection or the like. The dosage of the vaccine will depend on the route of administration and will vary according to the size of the host.

Various methods of achieving adjuvant effect for the vaccine includes use of agents such as aluminum hydroxide or phosphate (alum), commonly used as 0.05 to 0.1 percent solution in phosphate buffered saline, admixture with synthetic polymers of sugars (Carbopol) used as 0.25 percent solution, aggregation of the protein in the vaccine by heat treatment with temperatures ranging between 70° to 101° C. for 30 second to 2-minute periods respectively. Aggregation by reactivating with pepsin treated (Fab) antibodies to albumin, mixture with bacterial cells such as C. parvum or endotoxins or lipopolysaccharide components of gram-negative bacteria, emulsion in physiologically acceptable oil vehicles such as mannide mono-oleate (Aracel A) or emulsion with 20 percent solution of a perfluorocarbon (Fluosol-DA) used as a block substitute may also be employed.

In many instances, it will be desirable to have multiple administrations of the vaccine, usually not exceeding six to ten immunizations, more usually not exceeding four immunizations and preferably one or more, usually at least about three immunizations. The immunizations will normally be at from two to twelve-week intervals, more usually from three to five-week intervals. Periodic boosters at intervals of 1-5 years, usually three years, will be desirable to maintain protective levels of the antibodies. The course of the immunization may be followed by assays for antibodies for the supernatant antigens. The assays may be performed by labeling with conventional labels, such as radionuclides, enzymes, fluorescent agents, and the like. These techniques are well known and may be found in a wide variety of patents, such as Hudson and Cranage, Vaccine Protocols, 2003 Humana Press, relevant portions incorporated herein by reference.

Techniques and compositions for making useful dosage forms using the present invention are described in one or more of the following references: Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 2007; Goodman and Gilman, eds., The Pharmacological Basis of Therapeutics, Tenth Edition, McGraw Hill, 2001; Remington's Pharmaceutical Sciences, 20th Ed., Lippincott Williams & Wilkins., 2000, and updates thereto; Martindale, The Extra Pharmacopoeia, Thirty-Second Edition (The Pharmaceutical Press, London, 1999); all of which are incorporated by reference, and the like, relevant portions incorporated herein by reference.

Many suitable expression systems are commercially available, including, for example, the following: baculobacteria expression (Reilly, P. R., et al., BACULOBACTERIA EXPRESSION VECTORS: A LABORATORY MANUAL (1992); Beames, et al., Biotechniques 11:378 (1991); Pharmingen; Clontech, Palo Alto, Calif)), vaccinia expression systems (Earl, P. L., et al., “Expression of proteins in mammalian cells using vaccinia” In Current Protocols in Molecular Biology (F. M. Ausubel, et al. Eds.), Greene Publishing Associates & Wiley Interscience, New York (1991); Moss, B., et al., U.S. Pat. No. 5,135,855, issued Aug. 4, 1992), expression in bacteria (Ausubel, F. M., et al., CURRENT PROTOCOLS IN MOLECULAR BIOLOGY, John Wiley and Sons, Inc., Media Pa.; Clontech), expression in yeast (Rosenberg, S. and Tekamp-Olson, P., U.S. Pat. No. RE35,749, issued, Mar. 17, 1998, herein incorporated by reference; Shuster, J. R., U.S. Pat. No. 5,629,203, issued May 13, 1997, herein incorporated by reference; Gellissen, G., et al., Antonie Van Leeuwenhoek, 62(1-2):79-93 (1992); Romanos, M. A., et al., Yeast 8(6):423-488 (1992); Goeddel, D. V., Methods in Enzymology 185 (1990); Guthrie, C., and G. R. Fink, Methods in Enzymology 194 (1991)), expression in mammalian cells (Clontech; Gibco-BRL, Ground Island, N.Y.; e.g., Chinese hamster ovary (CHO) cell lines (Haynes, J., et al., Nuc. Acid. Res. 11:687-706 (1983); 1983, Lau, Y. F., et al., Mol. Cell. Biol. 4:1469-1475 (1984); Kaufman, R. J., “Selection and coamplification of heterologous genes in mammalian cells,” in Methods in Enzymology, vol. 185, pp 537-566. Academic Press, Inc., San Diego Calif. (1991)), and expression in plant cells (plant cloning vectors, Clontech Laboratories, Inc., Palo-Alto, Calif, and Pharmacia LKB Biotechnology, Inc., Pistcataway, N.J.; Hood, E., et al., J. Bacteriol. 168:1291-1301 (1986); Nagel, R., et al., FEMS Microbiol. Lett. 67:325 (1990); An, et al., “Binary Vectors”, and others in Plant Molecular Biology Manual A3:1-20 (1988); Miki, B. L. A., et al., pp. 249-265, and others in Plant DNA Infectious Agents (Hohn, T., et al., eds.) Springer-Verlag, Wien, Austria, (1987); Plant Molecular Biology: Essential Techniques, P. G. Jones and J. M. Sutton, New York, J. Wiley, 1997; Miglani, Gurbachan Dictionary of Plant Genetics and Molecular Biology, New York, Food Products Press, 1998; Henry, R. J., Practical Applications of Plant Molecular Biology, New York, Chapman & Hall, 1997), relevant portion incorporated herein by reference.

As used herein, the term “effective amount” or “effective dose” refers to that amount of the peptide or protein T cell epitopes of the invention sufficient to induce immunity, to prevent and/or ameliorate an infection or to reduce at least one symptom of an infection and/or to enhance the efficacy of another dose of peptide or protein T cell epitopes. An effective dose may refer to the amount of peptide or protein T cell epitopes sufficient to delay or minimize the onset of an infection. An effective dose may also refer to the amount of peptide or protein T cell epitopes that provides a therapeutic benefit in the treatment or management of an infection. Further, an effective dose is the amount with respect to peptide or protein T cell epitopes of the invention alone, or in combination with other therapies, that provides a therapeutic benefit in the treatment or management of an infection. An effective dose may also be the amount sufficient to enhance a subject's (e.g., a human's) own immune response against a subsequent exposure to an infectious agent. Levels of immunity can be monitored, e.g., by measuring amounts of neutralizing secretory and/or serum antibodies, e.g., by plaque neutralization, complement fixation, enzyme-linked immunosorbent, or microneutralization assay. In the case of a vaccine, an “effective dose” is one that prevents disease and/or reduces the severity of symptoms. A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms, in this case, an infectious disease, and more particularly, a Bordetella infection. The full prophylactic effect does not necessarily occur by administration of one dose, and may occur only after administration of a series of doses. Thus, a prophylactically effective amount may be administered in one or more administrations. Guidance can be found in the literature for appropriate dosages for given classes of pharmaceutical products. For example, for the given parameter, an effective amount will show an increase or decrease of at least 5%, 10%, 15%, 20%, 25%, 40%, 50%, 60%, 75%, 80%, 90%, or at least 100%. Efficacy can also be expressed as “-fold” increase or decrease. For example, a therapeutically effective amount can have at least a 1.2-fold, 1.5-fold, 2-fold, 5-fold, or more effect over a control. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins), relevant portions incorporated herein by reference.

As used herein, the term “immune stimulator” refers to a compound that enhances an immune response via the body's own chemical messengers (cytokines). These molecules comprise various cytokines, lymphokines and chemokines with immunostimulatory, immunopotentiating, and pro-inflammatory activities, such as interferons, interleukins (e.g., IL-1, IL-2, IL-3, IL-4, IL-12, IL-13); growth factors (e.g., granulocyte-macrophage (GM)-colony stimulating factor (CSF)); and other immunostimulatory molecules, such as macrophage inflammatory factor, Flt3 ligand, B7.1; B7.2, etc. The immune stimulator molecules can be administered in the same formulation as peptide or protein T cell epitopes s of the invention, or can be administered separately. Either the protein or an expression vector encoding the protein can be administered to produce an immunostimulatory effect.

As used herein, in certain embodiments, the term “protective immune response” or “protective response” refers to an immune response mediated by antibodies against an infectious agent, which is exhibited by a vertebrate (e.g., a human), which prevents or ameliorates an infection or reduces at least one symptom thereof. Peptide and protein T cell epitopes of the invention can stimulate the production of antibodies that, for example, neutralize infectious agents, blocks infectious agents from entering cells, blocks replication of said infectious agents, and/or protect host cells from infection and destruction. In other embodiments, the term can also refer to an immune response that is mediated by T-lymphocytes and/or other white blood cells against an infectious agent, exhibited by a vertebrate (e.g., a human), that prevents or ameliorates flavibacteria infection or reduces at least one symptom thereof. Peptide and protein T cell epitopes of the invention can stimulate the T cell responses that, for example, neutralize infectious agents, kill bacteria infected cells, blocks infectious agents from entering cells, blocks replication of said infectious agents, and/or protect host cells from infection and destruction.

The terms “biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

As used herein, a “cell” refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., Spodoptera) and human cells. Cells may be useful when they are naturally nonadherent or have been treated not to adhere to surfaces, for example by trypsinization.

As used herein, the term “contacting” is used in accordance with its plain ordinary meaning and refers to the process of allowing at least two distinct species to become sufficiently proximal to react, interact or physically touch. It should be appreciated, however, the resulting reaction product can be produced directly from a reaction between the added reagents or from an intermediate from one or more of the added reagents which can be produced in the reaction mixture. The term “contacting” may include allowing two species to react, interact, or physically touch, wherein the two species may be, for example, an amino acid sequence, protein, or peptide as provided herein and an immune cell, such as a T cell.

As used herein, a “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

The term “modulator” refers to a composition that increases or decreases the level of a target molecule or the function of a target molecule or the physical state of the target of the molecule relative to the absence of the modulator.

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

The terms “associated” or “associated with” in the context of a substance or substance activity or function associated with a disease (e.g. a protein associated disease, a cancer (e.g., cancer, inflammatory disease, autoimmune disease, or infectious disease)) means that the disease (e.g. cancer, inflammatory disease, autoimmune disease, or infectious disease) is caused by (in whole or in part), or a symptom of the disease is caused by (in whole or in part) the substance or substance activity or function. As used herein, what is described as being associated with a disease, if a causative agent, could be a target for treatment of the disease.

The term “aberrant” as used herein refers to different from normal. When used to describe enzymatic activity or protein function, aberrant refers to activity or function that is greater or less than a normal control or the average of normal non-diseased control samples. Aberrant activity may refer to an amount of activity that results in a disease, wherein returning the aberrant activity to a normal or non-disease-associated amount (e.g., by administering a compound or using a method as described herein), results in reduction of the disease or one or more disease symptoms.

The terms “subject” or “subject in need thereof” refers to a living organism who is at risk of or prone to having a disease or condition, or who is suffering from a disease or condition that can be treated by administration of a composition or pharmaceutical composition as provided herein. Non-limiting examples include humans and other primates, but also includes non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered. The system described above is intended for use in any of the above vertebrate species, since the immune systems of all of these vertebrates operate similarly.

The terms “disease” or “condition” refer to a state of being or health status of a patient or subject capable of being treated with a compound, pharmaceutical composition, or method provided herein. In embodiments, a patient or subject is human. In embodiments, the disease is Bordetella infection. In certain alternative embodiments, the disease is B. pertussis infection. In still other embodiments, the disease is whooping cough.

As used herein, “treatment” or “treating,” or “palliating” or “ameliorating” are used interchangeably herein. These terms refer to an approach for obtaining beneficial or desired results including but not limited to therapeutic benefit and/or a prophylactic benefit. By therapeutic benefit is meant eradication or amelioration of the underlying disorder being treated or the disorder resulting from bacterial infection. Also, a therapeutic benefit is achieved with the eradication or amelioration of one or more of the physiological symptoms associated with bacterial infection or the underlying disorder such that an improvement is observed in the patient, notwithstanding that the patient may still be afflicted with the underlying disorder or may still be infected. For prophylactic benefit, the compositions may be administered to a patient at risk of bacterial infection, of developing a particular disease, or to a patient reporting one or more of the physiological symptoms of a disease, even though a diagnosis of this disease may not have been made. Treatment includes preventing the infection or disease, that is, causing the clinical symptoms of the disease not to develop by administration of a protective composition prior to infection or the induction of the disease; suppressing the disease, that is, causing the clinical symptoms of the disease or infection not to develop by administration of a protective composition after the inductive event or infection but prior to the clinical appearance or reappearance of the disease; inhibiting the disease, that is, arresting the development of clinical symptoms by administration of a protective composition after their initial appearance; preventing re-occurring of the disease and/or relieving the disease, that is, causing the regression of clinical symptoms by administration of a protective composition after their initial appearance. “Treatment” can also refer to any of (i) the prevention of infection or reinfection, as in a traditional vaccine, (ii) the reduction or elimination of symptoms, and (iii) the substantial or complete elimination of the pathogen in question. Treatment may be affected prophylactically (prior to infection) or therapeutically (following infection).

In addition, in certain embodiments, “treatment,” “treat,” or “treating” refers to a method of reducing the effects of one or more symptoms of infection with a Bordetella. Thus, in the disclosed method, treatment can refer to a 10%, 20%, 30%, 40%, 50%, 60%, 70%, 80%, 90%, or 100% reduction in the severity of an established infection, disease, condition, or symptom of the infection, disease or condition. For example, a method for treating a disease is considered to be a treatment if there is a 10% reduction in one or more symptoms of the disease in a subject as compared to a control. Thus, the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition and/or complete prevention of infection. Further, as used herein, references to decreasing, reducing, or inhibiting include a change of 10%, 20%, 30%, 40%, 50%, 60%, 70%, 80%, 90% or greater as compared to a control level and such terms can include but do not necessarily include complete elimination.

As used herein the terms “diagnose” or “diagnosing” refers to recognition of an infection, disease or condition by signs and symptoms. Diagnosing can refer to determination of whether a subject has an infection or disease. Diagnosis may refer to determination of the type of disease or condition a subject has or the type of bacteria the subject is infected with.

Diagnostic agents provided herein include any such agent, which are well-known in the relevant art. Among imaging agents are fluorescent and luminescent substances, including, but not limited to, a variety of organic or inorganic small molecules commonly referred to as “dyes,” “labels,” or “indicators.” Examples include fluorescein, rhodamine, acridine dyes, Alexa dyes, and cyanine dyes. Enzymes that may be used as imaging agents in accordance with the embodiments of the disclosure include, but are not limited to, horseradish peroxidase, alkaline phosphatase, acid phosphatase, glucose oxidase, β-galactosidase, β-glucoronidase or β-lactamase. Such enzymes may be used in combination with a chromogen, a fluorogenic compound or a luminogenic compound to generate a detectable signal.

The peptide(s) or protein(s) of the present invention can also be used in binding assays including, but are not limited to, immunoassays such as competitive and non-competitive assay systems using techniques such as western blots, radioimmunoassays, ELISA (enzyme linked immunosorbent assay), “sandwich” immunoassays, Meso Scale Discovery (MSD, Gaithersburg, Md.), immunoprecipitation assays, ELISPOT, precipitin reactions, gel diffusion precipitin reactions, immunodiffusion assays, agglutination assays, complement-fixation assays, immunoradiometric assays, fluorescent immunoassays, and protein A immunoassays. Such assays are routine and well known in the art (see, e.g., Ausubel et al., eds, 1994, Current Protocols in Molecular Biology, Vol. 1, John Wiley & Sons, Inc., New York, relevant portions incorporated herein by reference).

Radioactive substances that may be used as imaging agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y ⁹⁰Y, ⁸⁹Sr, ⁸⁹Zr, ⁹⁴Tc, ⁹⁴Tc, ^{99m}Tc ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³m, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴I, ¹⁹⁸Au, ¹⁹⁹Au, ²¹¹At, ²¹¹Pb, ²¹²Bi, ²¹²Pb, ²¹³Bi, ²²³Ra and ²²⁵Ac. Paramagnetic ions that may be used as additional imaging agents in accordance with the embodiments of the disclosure include, but are not limited to, ions of transition and lanthanide metals (e.g., metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

When the imaging agent is a radioactive metal or paramagnetic ion, the agent may be reacted with another long-tailed reagent having a long tail with one or more chelating groups attached to the long tail for binding to these ions. The long tail may be a polymer such as a polylysine, polysaccharide, or other derivatized or derivatizable chain having pendant groups to which the metals or ions may be added for binding. Examples of chelating groups that may be used according to the disclosure include, but are not limited to, ethylenediaminetetraacetic acid (EDTA), diethylenetriaminepentaacetic acid (DTPA), DOTA, NOTA, NETA, TETA, porphyrins, polyamines, crown ethers, bis-thiosemicarbazones, polyoximes, and like groups.

The terms “dose” and “dosage” are used interchangeably herein. A dose refers to the amount of active ingredient given to an individual at each administration. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical or pharmaceutical composition, and depends on the route of administration. For example, a dosage form can be in a liquid form for nebulization, e.g., for inhalants, in a tablet or liquid, e.g., for oral delivery, or a saline solution, e.g., for injection.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

Formulations suitable for oral administration can consist of (a) liquid solutions, such as an effective amount of the antibodies provided herein suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

Pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes). Additionally, these carriers can function as immunostimulating agents (i.e., adjuvants).

The term “adjuvant” refers to a compound that when administered in conjunction with the compositions provided herein including embodiments thereof, augments the composition's immune response. Generally, adjuvants are non-toxic, have high-purity, are degradable, and are stable.

Adjuvants can augment an immune response by several mechanisms including lymphocyte recruitment, stimulation of B and/or T cells, and stimulation of macrophages. The adjuvant increases the titer of induced antibodies and/or the binding affinity of induced antibodies relative to the situation if the immunogen were used alone. A variety of adjuvants can be used in combination with the agents provided herein including embodiments thereof, to elicit an immune response. Preferred adjuvants augment the intrinsic response to an immunogen without causing conformational changes in the immunogen that affect the qualitative form of the response. Preferred adjuvants include aluminum hydroxide and aluminum phosphate, 3 De-O-acylated monophosphoryl lipid A (MPL™) (see GB 2220211 (RIBI ImmunoChem Research Inc., Hamilton, Montana, now part of Corixa). Stimulon™ QS-21 is a triterpene glycoside or saponin isolated from the bark of the Quillaja Saponaria Molina tree found in South America (see Kensil et al., in Vaccine Design: The Subunit and Adjuvant Approach (eds. Powell & Newman, Plenum Press, NY, 1995); U.S. Pat. No. 5,057,540), (Aquila BioPharmaceuticals, Framingham, MA). Other adjuvants are oil in water emulsions (such as squalene or peanut oil), optionally in combination with immune stimulants, such as monophosphoryl lipid A (see Stoute et al., N. Engl. J. Med. 336, 86-91 (1997)), pluronic polymers, and killed mycobacteria. Another adjuvant is CpG (WO 98/40100). Adjuvants can be administered as a component of a therapeutic composition with an active agent or can be administered separately, before, concurrently with, or after administration of the therapeutic agent.

Other adjuvants contemplated for the invention are saponin adjuvants, such as Stimulon™ (QS-21, Aquila, Framingham, MA) or particles generated therefrom such as ISCOMs (immunostimulating complexes) and ISCOMATRIX. Other adjuvants include RC-529, GM-CSF and Complete Freund's Adjuvant (CFA) and Incomplete Freund's Adjuvant (IFA). Other adjuvants include cytokines, such as interleukins (e.g., IL-1 α and β peptides, IL-2, IL-4, IL-6, IL-12, IL-13, and IL-15), macrophage colony stimulating factor (M-CSF), granulocyte-macrophage colony stimulating factor (GM-CSF), tumor necrosis factor (TNF), chemokines, such as MIP1α and β and RANTES. Another class of adjuvants is glycolipid analogues including N-glycosylamides, N-glycosylureas and N-glycosylcarbamates, each of which is substituted in the sugar residue by an amino acid, as immuno-modulators or adjuvants (see U.S. Pat. No. 4,855,283). Heat shock proteins, e.g., HSP70 and HSP90, may also be used as adjuvants.

Suitable formulations for rectal administration include, for example, suppositories, which consist of the packaged nucleic acid with a suppository base. Suitable suppository bases include natural or synthetic triglycerides or paraffin hydrocarbons. In addition, it is also possible to use gelatin rectal capsules which consist of a combination of the compound of choice with a base, including, for example, liquid triglycerides, polyethylene glycols, and paraffin hydrocarbons.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration, oral administration, and intravenous administration are the preferred methods of administration. The formulations of compounds can be presented in unit-dose or multi-dose sealed containers, such as ampules and vials.

Injection solutions and suspensions can be prepared from sterile powders, granules, and tablets of the kind previously described. Cells transduced by nucleic acids for ex vivo therapy can also be administered intravenously or parenterally as described above.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain other compatible therapeutic agents.

The combined administration contemplates co-administration, using separate formulations or a single pharmaceutical formulation, and consecutive administration in either order, wherein preferably there is a time period while both (or all) active agents simultaneously exert their biological activities.

Effective doses of the compositions provided herein vary depending upon many different factors, including means of administration, target site, physiological state of the patient, whether the patient is human or an animal, other medications administered, and whether treatment is prophylactic or therapeutic. However, a person of ordinary skill in the art would immediately recognize appropriate and/or equivalent doses looking at dosages of approved compositions for treating and preventing cancer for guidance.

As used herein, the term “pharmaceutically acceptable” is used synonymously with “physiologically acceptable” and “pharmacologically acceptable”. A pharmaceutical composition will generally comprise agents for buffering and preservation in storage, and can include buffers and carriers for appropriate delivery, depending on the route of administration. As used herein, the terms “pharmaceutically acceptable” or “pharmacologically acceptable” refer to a material which is not biologically or otherwise undesirable, i.e., the material may be administered to an individual in a formulation or composition without causing any unacceptable biological effects or interacting in a deleterious manner with any of the components of the composition in which it is contained.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances, and the like, that do not deleteriously react with the compounds of the invention. One of skill in the art will recognize that other pharmaceutical excipients are useful in the present invention.

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The pharmaceutical preparation is optionally in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The unit dosage form can be of a frozen dispersion.

The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In embodiments, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present invention can also be delivered as nanoparticles.

The present invention describes methods utilizing and compositions comprising or expressing T cell epitopes, T cell epitope-containing peptides, and T cell epitope-containing proteins associated with binding to a subset of the naturally occurring MHC Class II and/or MHC Class I molecules within the human population. Compositions comprising or expressing one or more of the disclosed peptides (e.g., the amino acid sequences set forth in any one of Tables 1-20) or polynucleotides encoding the same, covering different HLA Class II and/or MHC Class I alleles, capable of generating a treatment acting broadly on a population level are disclosed herein. As the antigen repertoire of MHC Class I and MHC Class II alleles varies from one individual to another and from one ethnic population to another, it is challenging to provide vaccines or peptide or epitopes-based immunotherapies that can be offered to subjects of any geographic region in the world or provide sufficient protection against infection across a wide segment of the populations unless numerous epitopes or peptides are included (e.g., in a vaccine). Taking into consideration the need for a single vaccine formulation that can provide protection across populations, if it desirable to provide a treatment containing or expressing proteins, peptides or epitopes that will provide protection against infection amongst the majority of the worldwide population. Also, taking into consideration the enormous costs and risks in the clinical development of new treatments and the increasing demands from regulatory bodies to meet high standards for toxicity testing, dose justification, safety and efficacy trials, it is desirable to provide treatments containing or expressing as few peptides as possible, but at the same time to be able to treat the majority of subjects in a worldwide population with a single immunotherapy. Such a product should comprise as a first requirement an expression or inclusion of combination of epitopes or peptides that are able to bind the worldwide MHC Class I and/or MHC Class II allele repertoire, and the resulting peptide-MHC complexes should as a second requirement be recognized by the T cells of the subject so as to induce the desired immunological reactions.

It is an object of claims of the present invention to provide improved epitope or peptide combinations for modulating an immune response, for treating a subject for an infection or aberrant immune response, and for use in diagnostic methods and kits comprising such peptide combinations. It is another object of the invention to provide epitope or peptide combinations exhibiting very good HLA Class I and Class II coverage in a worldwide population and being immunologically potent in a worldwide population. It is another object of the invention to provide epitope or peptide combinations having good cross reactivity to other strains, including co-circulating strains (for example, mutants) of Bordetella, including B. pertussis, etc. It is another object of the invention to provide epitope or peptide combinations of a relatively small number of epitopes or peptides yet obtaining at least 70%, and more preferably around 90-100% donor coverage in a donor cohort representative of a worldwide population. In certain embodiments, this is achieved by selecting one or more immunodominant and/or immunoprevalent proteins (e.g., a B. pertussis protein) or subsequences, portions, homologues, variants or derivatives thereof for use in the methods and compositions of the present disclosure, wherein said immunodominant and/or immunoprevalent proteins or subsequences, portions, homologues, variants or derivatives thereof comprise two or more epitopes that are immunodominant and/or immunoprevalant. In some embodiments, the two or more epitopes comprise two to ten epitopes and/or polynucleotides encoding the same. Another object of the invention is to provide epitope combinations which are so immunologically potent that even at very low doses of epitopes, the percentage of responding donors can be retained at a very high level in a donor cohort representative of a worldwide population. Another object of the invention is to provide epitope combinations which have minor risk of inducing IgE-mediated adverse events. An additional object of the invention is to provide proteins, peptides, or nucleic acids containing or expressing epitopes or combinations of such proteins, peptides or nucleic acids which have a sufficient solubility profile for being formulated in a pharmaceutical product, preferably which have acceptable estimated in vivo stability. One further objective of the invention is to select epitopes for use in the compositions and methods described herein, based on one or both of their immunodominance or immunoprevalence. A still further object of the invention is to select such epitopes and epitopes combinations not only in accordance with those embodiments previously described, but also those epitopes and epitope combinations capable of eliciting a B cell response and T cell response (e.g., selecting one or more peptides for use in the methods and compositions described herein capable of generating a T cell and antibody response in a subject).

Provided herein are methods and compositions for diagnosing, treating, and immunizing against a Bordetella, including methods and compositions of detecting an immune response or immune cells relevant to a Bordetella infection. These methods and compositions include vaccines, diagnostics, therapies, reagents and kits, for modulating, eliciting, or detecting T cells responsive to one or more Bordetella peptides or proteins. The proteins and peptides described herein comprise, consist of, or consist essentially of: one or more amino acid sequences selected from any one of those sequences set forth in Tables 1-20, or a subsequence, portion, homologue, variant or derivative thereof, a fusion protein comprising one or more amino acid sequences selected from any one of those sequences set forth in Tables 1-20; a pool of 2 or more peptides selected from the amino acid sequences set forth in any one of Tables 1-20, or a polynucleotide that encodes one or more peptides or proteins, comprising, consisting of, or consisting essentially of an amino acid sequence selected from any one of those sequences set forth in Tables 1-20, or a subsequence, portion, homologue, variant or derivative thereof. In certain preferred embodiments, the Bordetella is one or more of B. pertussis or a variant thereof. Further description and embodiments of such methods and compositions are provided in the definitions provided herein, and a person skilled in the art will recognize that the methods and compositions can be embodied in numerous variations, changes, and substitutions or as may occur to or be understood by one skilled in the art without departing from the invention.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown byway of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically, and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

What is claimed is:

1. A composition comprising:
one or more peptides or proteins, comprising, consisting of, or consisting essentially of an amino acid sequence selected from any one of those sequences set forth in Tables 1-20 (SEQ ID NOS: 1 to 2598), or a subsequence, portion, homologue, variant or derivative thereof;
a fusion protein comprising one or more amino acid sequences selected from any one of those sequences set forth in Tables 1-20; or
a pool of 2 or more or more peptides comprising, consisting of, or consisting essentially of amino acid sequences selected from any one of those sequences set forth in Tables 1-20; or
a polynucleotide that encodes one or more peptides or proteins, comprising, consisting of, or consisting essentially of an amino acid sequence selected from any one of those sequences set forth in Tables 1-20, or a subsequence, portion, homologue, variant or derivative thereof.

2. The composition of claim 1, wherein the one or more peptides or proteins comprises, or wherein the fusion protein comprises 2 or more or more amino acid sequences selected from any one of those sequences set forth in Tables 1-20, or a subsequence, portion, homologue, variant or derivative thereof.

3. The composition of claim 1 or claim 2, wherein the amino acid sequence is selected from a Bordetella T cell epitope selected from any one of those sequences set forth in Tables 1-20.

4. The composition of claim 1 or claim 2, wherein the composition comprises one or more B. pertussis peptides amino acid sequences selected from any one of those sequences set forth in Tables 1-20, or a subsequence, portion, homologue, variant or derivative thereof;
a fusion protein comprising one or more amino acid sequences selected from any one of those sequences set forth in Tables 1-20; or
a pool of 2 or more peptides selected from any one of those sequences set forth in Tables 1-20; or
a polynucleotide that encodes one or more peptides or proteins, comprising, consisting of, or consisting essentially of an amino acid sequence selected from any one of those sequences set forth in Tables 1-20, or a subsequence, portion, homologue, variant or derivative thereof.

5. The composition of any one of claims 1 to 5, wherein the peptide or protein comprises a Bordetella T cell epitope.

6. The composition of any one of claims 1 to 5, wherein the one or more peptides or proteins comprises a Bordetella CD8+ or CD4+ T cell epitope.

7. The composition of any one of claims 1 to 6, wherein the Bordetella is B. pertussis and the B. pertussis T cell epitope is not conserved in another Bordetella.

8. The composition of any one of claims 1 to 6, wherein the Bordetella is B. pertussis and the B. pertussis T cell epitope is conserved in another Bordetella.

9. The composition of any one of claims 1 to 8, wherein one or more peptides or proteins has a length from about 9-15, 15-20, 20-25, 25-30, 30-40, 40-50, 50-75 or 75-100 amino acids.

10. The composition of any one of claims 1 to 9, wherein the one or more peptides or proteins elicits, stimulates, induces, promotes, increases or enhances a T cell response to a Bordetella.

11. The composition of claim 10, wherein the one or more peptides or proteins that elicits, stimulates, induces, promotes, increases or enhances the T cell response to the Bordetella is a Bordetella protein or peptide, or a variant, homologue, derivative or subsequence thereof.

12. The composition of any one of claims 1 to 11, further comprising formulating the one or more peptides or proteins into an immunogenic formulation with an adjuvant.

13. The composition of claim 12, wherein the adjuvant is selected from the group consisting of adjuvant is selected from the group consisting of alum, aluminum hydroxide, aluminum phosphate, calcium phosphate hydroxide, cytosine-guanosine oligonucleotide (CpG-ODN) sequence, granulocyte macrophage colony stimulating factor (GM-CSF), monophosphoryl lipid A (MPL), poly(I:C), MF59, Quil A, N-acetyl muramyl-L-alanyl-D-isoglutamine (MDP), FIA, montanide, poly (DL-lactide-coglycolide), squalene, virosome, AS03, ASO4, IL-1, IL-2, IL-3, IL-4, IL-5, IL-6, IL-7, IL-8, IL-10, IL-12, IL-15, IL-17, IL-18, STING, CD40L, pathogen-associated molecular patterns (PAMPs), damage-associated molecular pattern molecules (DAMPs), Freund's complete adjuvant, Freund's incomplete adjuvant, transforming growth factor (TGF)-beta antibody or antagonists, A2aR antagonists, lipopolysaccharides (LPS), Fas ligand, Trail, lymphotactin, Mannan (M-FP), APG-2, Hsp70 and Hsp90, pattern recognition receptor ligands, TLR3 ligands, TLR4 ligands, TLR5 ligands, TLR7/8 ligands, and TLR9 ligands.

14. The composition of any one of claims 1 to 13, wherein the composition further comprises a modulator of immune response.

15. The composition of claim 14, wherein the modulator of immune response is a modulator of the innate immune response.

16. The composition of claim 14 or claim 15, wherein the modulator is Interleukin-6 (IL-6), Interferon-gamma (IFN-γ), Transforming growth factor beta (TGF-β), or Interleukin-10 (IL-10), or an agonist or antagonist thereof.

17. A composition comprising monomers or multimers of:
peptides or proteins comprising, consisting of, or consisting essentially of:
one or more amino acid sequences selected from any one ofthose sequences set forth in Tables 1-20,
concatemers, subsequences, portions, homologues, variants or derivatives thereof;
a fusion protein comprising one or more amino acid sequences selected from any one of those sequences set forth in Tables 1-20; or
a polynucleotide that encodes one or more peptides or proteins, comprising, consisting of, or consisting essentially of an amino acid sequence selected from any one of those sequences set forth in Tables 1-20, or a subsequence, portion, homologue, variant or derivative thereof.

18. A composition comprising one or more peptide-major histocompatibility complex (MHC) monomers or multimers, wherein the peptide-MHC monomer or multimer comprises a peptide comprising, consisting of, or consisting essentially of an amino acid sequence selected from any one of those sequences set forth in Tables 1-20, in a groove of the MHC monomer or multimer.

19. A composition comprising:
one or more peptides or proteins comprising, consisting of, or consisting essentially of an amino acid sequence selected from any one of those sequences set forth in Tables 1-20, or a subsequence, portion, homologue, variant or derivative thereof;
a fusion protein comprising one or more amino acid sequences selected from any one of those sequences set forth in Tables 1-20;
a pool of 2 or more peptides selected from any one of those sequences set forth in Tables 1-20; or
a polynucleotide that encodes one or more peptides or proteins, comprising, consisting of, or consisting essentially of an amino acid sequence selected from any one of those sequences set forth in Tables 1-20, or a subsequence, portion, homologue, variant or derivative thereof.

20. The composition of claim 19, wherein the one or more peptides or proteins comprises, or wherein the fusion protein comprises, 2 or more amino acid sequences selected from any one of those sequences set forth in Tables 1-20, or a subsequence, portion, homologue, variant or derivative thereof.

21. The composition of claim 19 or claim 20, wherein the protein or peptide comprises a B. pertussis T cell epitope.

22. The composition of any one of claims 19 to 21, wherein the one or more peptides or proteins comprises a B. pertussis CD8+ or CD4+ T cell epitope.

23. The composition of any one of claims 19 to 22, wherein the B. pertussis T cell epitope is not conserved in another Bordetella.

24. The composition of any one of claims 19 to 22, wherein the B. pertussis T cell epitope is conserved in another Bordetella.

25. The composition of any one of claims 19 to 24, wherein one or more peptides or proteins has a length from about 9-15, 15-20, 20-25, 25-30, 30-40, 40-50, 50-75 or 75-100 amino acids.

26. The composition of any one of claims 19 to 25, wherein the one or more peptides or proteins elicits, stimulates, induces, promotes, increases or enhances a T cell response to B. pertussis.

27. The composition of any one of claims 19 to 26, wherein the one or more peptides or proteins that elicits, stimulates, induces, promotes, increases or enhances the T cell response to B. pertussis is a B. pertussis protein or peptide, or a variant, homologue, derivative or subsequence thereof.

28. The composition of any one of claims 19 to 27, further comprising formulating the one or more peptides or proteins into an immunogenic formulation with an adjuvant.

29. The composition of claim 28, wherein the adjuvant is selected from the group consisting of adjuvant is selected from the group consisting of alum, aluminum hydroxide, aluminum phosphate, calcium phosphate hydroxide, cytosine-guanosine oligonucleotide (CpG-ODN) sequence, granulocyte macrophage colony stimulating factor (GM-CSF), monophosphoryl lipid A (MPL), poly(I:C), MF59, Quil A, N-acetyl muramyl-L-alanyl-D-isoglutamine (MDP), FIA, montanide, poly (DL-lactide-coglycolide), squalene, virosome, AS03, ASO4, IL-1, IL-2, IL-3, IL-4, IL-5, IL-6, IL-7, IL-8, IL-10, IL-12, IL-15, IL-17, IL-18, STING, CD40L, pathogen-associated molecular patterns (PAMPs), damage-associated molecular pattern molecules (DAMPs), Freund's complete adjuvant, Freund's incomplete adjuvant, transforming growth factor (TGF)-beta antibody or antagonists, A2aR antagonists, lipopolysaccharides (LPS), Fas ligand, Trail, lymphotactin, Mannan (M-FP), APG-2, Hsp70 and Hsp90, pattern recognition receptor ligands, TLR3 ligands, TLR4 ligands, TLR5 ligands, TLR7/8 ligands, and TLR9 ligands.

30. The composition of any one of claims 19 to 29, wherein the composition further comprises a modulator of immune response.

31. The composition of claim 30, wherein the modulator of immune response is a modulator of the innate immune response.

32. The composition of claim 30 or claim 31, wherein the modulator is Interleukin-6 (IL-6), Interferon-gamma (IFN-g), Transforming growth factor beta (TGF-B), or Interleukin-10 (IL-10), or an agonist or antagonist thereof.

33. A composition comprising monomers or multimers of:
one or more peptides or proteins comprising, consisting of, or consisting essentially of:
one or more B. pertussis amino acid sequences selected from any one of those sequences set forth in Tables 1-20, concatemers, subsequences, portions, homologues, variants or derivatives thereof;
a fusion protein comprising one or more amino acid sequences selected from any one of those sequences set forth in Tables 1-20; or
a polynucleotide that encodes one or more peptides or proteins, comprising, consisting of, or consisting essentially of an amino acid sequence selected from any one of those sequences set forth in Tables 1-20, or a subsequence, portion, homologue, variant or derivative thereof.

34. A composition comprising one or more peptide-major histocompatibility complex (MHC) monomers or multimers, wherein the peptide-MHC monomer or multimer comprises a peptide comprising, consisting of, or consisting essentially of an amino acid sequence selected from any one of those sequences set forth in Tables 1-20, in a groove of the (MHC) monomer or multimer.

35. A method for detecting the presence of: (i) a Bordetella or (ii) an immune response relevant to Bordetella infections, vaccines or therapies, including T cells responsive to one or more Bordetella peptides, comprising:
providing one or more proteins or peptides for detection of an amount or a relative amount of, and/or the activity of, and/or the state of antigen-specific T-cells;
contacting a biological sample suspected of having Bordetella-specific T-cells to one or more proteins or peptides for detection; and
detecting an amount or a relative amount of, and/or the activity of, and/or the state of antigen-specific T-cells in the biological sample, wherein the one or more proteins or peptides for detection comprise one or more amino acid sequences set forth in any one of Tables 1-20, or comprise a pool of 2 or more or more amino acid sequences set forth in any one of Tables 1-20.

36. The method of claim 35, wherein detecting the amount or a relative amount of, and/or activity of antigen-specific T-cells comprises one or more steps of identification or detection of the antigen-specific T-cells and measuring the amount of the antigen-specific T-cells.

37. The method of claim 35 or claim 36, wherein the one or more peptides or proteins comprises 2 or more amino acid sequences selected from those set forth in any one of Tables 1-20.

38. The method of any one of claims 35 to 37, wherein the detecting the amount or a relative amount of, and/or activity of antigen-specific T-cells comprises indirect detection and/or direct detection.

39. The method of any one of claims 35 to 38, wherein the method of detecting an immune response relevant to the Bordetella comprises the following steps:
providing an MHC monomer or an MHC multimer;
contacting a population T-cells to the MHC monomer or MHC multimer; and
measuring the number, activity or state of T-cells specific for the MHC monomer or MHC multimer.

40. The method of claim 39, wherein the MHC monomer or MHC multimer comprises a protein or peptide of the Bordetella.

41. The method of claim 35, wherein the protein or peptide comprises a CD8+ or CD4+ T cell epitope.

42. The method of claim 41, wherein the T cell epitope is not conserved in another Bordetella.

43. The method of claim 41, wherein the T cell epitope is conserved in another Bordetella.

44. The method of any one of claims 35 to 45, wherein the protein or peptide has a length from about 9-15, 15-20, 20-25, 25-30, 30-40, 40-50, 50-75 or 75-100 amino acids.

45. The method of any one of claims 35 to 44, wherein the proteins or peptides comprise 2 or more amino acid sequences selected from any one of those sequences set forth in Tables 1-20, or a subsequence, portion, homologue, variant or derivative thereof.

46. The method of any one of claims 35 to 45, further comprising detecting the presence or amount of the one or more peptides in a biological sample, or a response thereto, which is diagnostic of a Bordetella infection.

47. The method of any one of claims 35 to 46, wherein detecting an amount or a relative amount of, and/or the activity of, and/or the state of antigen-specific T-cells in the biological sample comprises measuring one or more of a cytokine or lymphokine secretion assay, T cell proliferation, immunoprecipitation, immunoassay, ELISA, radioimmunoassay, immunofluorescence assay, Western Blot, FACS analysis, a competitive immunoassay, a noncompetitive immunoassay, a homogeneous immunoassay a heterogeneous immunoassay, a bioassay, a reporter assay, a luciferase assay, a microarray, a surface plasmon resonance detector, a florescence resonance energy transfer, immunocytochemistry, or a cell mediated assay, or a cytokine proliferation assay.

48. The method of any one of claims 35 to 47, further comprising administering a treatment comprising the composition of any one of claims 1-34 to the subject from which the biological sample was drawn that increases the amount or relative amount of, and/or activity of the antigen-specific T-cells.

49. A method for detecting the presence of: (i) B. pertussis or (ii) an immune response relevant to B. pertussis infections, vaccines or therapies, including T cells responsive to one or more B. pertussis peptides, comprising:
providing one or more proteins or peptides for detection of an amount or a relative amount of, and/or the activity of, and/or the state of antigen-specific T-cells;
contacting a biological sample suspected of having B. pertussis-specific T-cells to one or more proteins or peptides for detection; and
detecting an amount or a relative amount of, and/or the activity of, and/or the state of antigen-specific T-cells in the biological sample, wherein the one or more proteins or peptides for detection comprise one or more amino acid sequences set forth in those sequences set forth in any one of Tables 1-20, or comprise a pool of 2 or more amino acid sequences set forth in those sequences set forth in any one of Tables 1-20.

50. The method of claim 49, wherein detecting the amount or a relative amount of, and/or activity of antigen-specific T-cells comprises one or more steps of identification or detection of the antigen-specific T-cells and measuring the amount of the antigen-specific T-cells.

51. The method of claim 49 or claim 50, wherein the one or more peptides or proteins comprises 2 or more amino acid sequences selected from any one of those sequences set forth in Tables 1-20.

52. The method of any one of claims 49 to 51, wherein the detecting the amount or a relative amount of, and/or activity of antigen-specific T-cells comprises indirect detection and/or direct detection.

53. The method of any one of claims 49 to 53, wherein the method of detecting an immune response relevant to B. pertussis comprises the following steps:
providing an MHC monomer or an MHC multimer;
contacting a population T-cells to the MHC monomer or MHC multimer; and
measuring the number, activity or state of T-cells specific for the MHC monomer or MHC multimer.

54. The method of claim 53, wherein the MHC monomer or MHC multimer comprises a protein or peptide of B. pertussis.

55. The method of claim 54, wherein the protein or peptide comprises a B. pertussis CD8+ or CD4+ T cell epitope.

56. The method of claim 55, wherein the B. pertussis T cell epitope is not conserved in another Bordetella.

57. The method of claim 55, wherein the B. pertussis T cell epitope is conserved in another Bordetella.

58. The method of any one of claims 49 to 57, wherein the protein or peptide has a length from about 9-15, 15-20, 20-25, 25-30, 30-40, 40-50, 50-75 or 75-100 amino acids.

59. The method of any one of claims 49 to 58, wherein the proteins or peptides comprise 2 or more amino acid sequences selected from any one of those sequences set forth in Tables 1-20, or a subsequence, portion, homologue, variant, or derivative thereof.

60. The method of any one of claims 49 to 59, further comprising detecting the presence or amount of the one or more peptides in a biological sample, or a response thereto, which is diagnostic of a B. pertussis infection.

61. The method of any one of claims 49 to 60, wherein detecting an amount or a relative amount of, and/or the activity of, and/or the state of antigen-specific T-cells in the biological sample comprises measuring one or more of a cytokine or lymphokine secretion assay, T cell proliferation, immunoprecipitation, immunoassay, ELISA, radioimmunoassay, immunofluorescence assay, Western Blot, FACS analysis, a competitive immunoassay, a noncompetitive immunoassay, a homogeneous immunoassay a heterogeneous immunoassay, a bioassay, a reporter assay, a luciferase assay, a microarray, a surface plasmon resonance detector, a florescence resonance energy transfer, immunocytochemistry, or a cell mediated assay, or a cytokine proliferation assay.

62. The method of any one of claims 49 to 61, further comprising administering a treatment comprising the composition of any one of claims 1-34 to the subject from which the biological sample was drawn that increases the amount or relative amount of, and/or activity of the antigen-specific T-cells.

63. A method detecting a Bordetella infection or exposure in a subject, the method comprising, consisting of, or consisting essentially of:
contacting a biological sample from a subject with a composition of any one of claims 1 to 36; and
determining if the composition elicits an immune response from the contacted cells, wherein the presence of an immune response indicates that the subject has been exposed to or infected with Bordetella.

64. The method of claim 63, wherein the sample comprises T cells.

65. The method of claim 63 or claim 64, wherein the response comprises inducing, increasing, promoting or stimulating anti-Bordetella activity of T cells.

66. The method of claim 63 or claim 65, wherein the T cells are CD8+ or CD4+ T cells.

67. The method of any one of claims 63 to 66, wherein the method comprises determining whether the subject has been infected by or exposed to the Bordetella more than once by determining if the subject elicits a secondary T cell immune response profile that is different from a primary T cell immune response profile.

68. The method of any one of claims 63 to 67, further comprising diagnosing a Bordetella infection or exposure in a subject, the method comprising contacting a biological sample from a subject with a composition of any one of claims 1 to 34, and determining if the composition elicits a T cell immune response, wherein the T cell immune response identifies that the subject has been infected with or exposed to a Bordetella.

69. The method of any one of claims 63 to 68, wherein the method is conducted three or more days following the date of suspected infection by or exposure to a Bordetella.

70. A method detecting B. pertussis infection or exposure in a subject, the method comprising, consisting of, or consisting essentially of:
contacting a biological sample from a subject with a composition of any one of claims 19 to 36; and
determining if the composition elicits an immune response from the contacted cells, wherein the presence of an immune response indicates that the subject has been exposed to or infected with B. pertussis.

71. The method of claim 70, wherein the sample comprises T cells.

72. The method of claim 70 or claim 71, wherein the response comprises inducing, increasing, promoting or stimulating anti-B. pertussis activity of T cells.

73. The method of claim 71 or claim 72, wherein the T cells are CD8+ or CD4+ T cells.

74. The method of any one of claims 70 to 73, wherein the method comprises determining whether the subject has been infected by or exposed to B. pertussis more than once by determining if the subject elicits a secondary T cell immune response profile that is different from a primary T cell immune response profile.

75. The method of any one of claims 70 to 74, further comprising diagnosing a B. pertussis infection or exposure in a subject, the method comprising contacting a biological sample from a subject with a composition of any one of claims 19 to 34; and determining if the composition elicits a T cell immune response, wherein the T cell immune response identifies that the subject has been infected with or exposed to B. pertussis.

76. The method of any one of claims 70 to 75, wherein the method is conducted three or more days following the date of suspected infection by or exposure to a Bordetella.

77. A kit for the detection of Bordetella or an immune response to Bordetella in a subject comprising, consisting of or consisting essentially of:
one or more T cells that specifically detect the presence of:
one or more amino acid sequences selected from any one ofthose sequences set forth in Tables 1-20, or a subsequence, portion, homologue, variant or derivative thereof, or
a fusion protein comprising one or more amino acid sequences selected from any one of those sequences set forth in Tables 1-20; or
a pool of 2 or more or more peptides selected from the amino acid sequences set forth in any one of Tables 1-20.

78. The kit of claim 77, wherein the one or more amino acid sequences are selected from a Bordetella T cell epitope set forth in any one of Tables 1-20.

79. The kit of claim 77 or claim 78, wherein the composition comprises:
one or more amino acid sequences selected from any one ofthose sequences set forth in Tables 1-20, or a subsequence, portion, homologue, variant or derivative thereof;
a fusion protein comprising one or more amino acid sequences selected from any one of those sequences set forth in Tables 1-20; or
a pool of 2 or more peptides selected from the amino acid sequences set forth in those sequences set forth in any one of Tables 1-20.

80. The kit of any one of claims 77 to 79, wherein the amino acid sequence comprises a Bordetella CD8+ or CD4+ T cell epitope.

81. The kit of claim 78 or claim 80, wherein the T cell epitope is not conserved in another Bordetella.

82. The kit of claim 78 or claim 80, wherein the T cell epitope is conserved in another Bordetella.

83. The kit of any one of claims 77 to 82, wherein the fusion protein has a length from about 9-15, 15-20, 20-25, 25-30, 30-40, 40-50, 50-75 or 75-100 amino acids.

84. The kit of any one of claims 77 to 83, wherein the kit includes instruction for a diagnostic method, a process, a composition, a product, a service or component part thereof for the detection of: (i) Bordetella or (ii) an immune response relevant to Bordetella infections, vaccines or therapies, including T cells responsive to Bordetella.

85. The kit of any one of claims 77 to 84, wherein the kit includes reagents for detecting an amount or a relative amount of, and/or the activity of, and/or the state of antigen-specific T-cells in the biological sample comprises measuring one or more of a cytokine or lymphokine secretion assay, T cell proliferation, immunoprecipitation, immunoassay, ELISA, radioimmunoassay, immunofluorescence assay, Western Blot, FACS analysis, a competitive immunoassay, a noncompetitive immunoassay, a homogeneous immunoassay a heterogeneous immunoassay, a bioassay, a reporter assay, a luciferase assay, a microarray, a surface plasmon resonance detector, a florescence resonance energy transfer, immunocytochemistry, or a cell mediated assay, or a cytokine proliferation assay.

86. The kit of any one of claims 77 to 85, wherein the kit includes reagents for determining a Human Leukocyte Antigen (HLA) profile of a subject, and selecting peptides that are presented by the HLA profile of the subject for detecting an immune response to Bordetella.

87. A kit for the detection of B. pertussis or an immune response to B. pertussis in a subject comprising, consisting of or consisting essentially of:
one or more T cells that specifically detect the presence of:
one or more amino acid sequences selected from any one ofthose sequences set forth in Tables 1-20, or a subsequence, portion, homologue, variant or derivative thereof;
a fusion protein comprising one or more amino acid sequences selected from any one of those sequences set forth in Tables 1-20; or
a pool of 2 or more peptides selected from the amino acid sequences set forth in those sequences set forth in any one of Tables 1-20.

88. The kit of claim 87, wherein the one or more amino acid sequences is selected from a B. pertussis CD4 T cell epitope selected from any one of Tables 1-20; or both.

89. The kit of claims 87 to 88, wherein the amino acid sequence comprises a B. pertussis CD8+ or CD4+ T cell epitope.

90. The kit of claim 89, wherein the B. pertussis T cell epitope is not conserved in another Bordetella.

91. The kit of claim 89, wherein the B. pertussis T cell epitope is conserved in another Bordetella.

92. The kit of any one of claims 87 to 91, wherein the fusion protein has a length from about 9-15, 15-20, 20-25, 25-30, 30-40, 40-50, 50-75 or 75-100 amino acids.

93. The kit of any one of claims 87 to 92, wherein the kit includes instruction for a diagnostic method, a process, a composition, a product, a service or component part thereof for the detection of: (i) B. pertussis or (ii) an immune response relevant to B. pertussis infections, vaccines or therapies, including T cells responsive to B. pertussis.

94. The kit of any one of claims 87 to 93, wherein the kit includes reagents for detecting an amount or a relative amount of, and/or the activity of, and/or the state of antigen-specific T-cells in the biological sample comprises measuring one or more of a cytokine or lymphokine secretion assay, T cell proliferation, immunoprecipitation, immunoassay, ELISA, radioimmunoassay, immunofluorescence assay, Western Blot, FACS analysis, a competitive immunoassay, a noncompetitive immunoassay, a homogeneous immunoassay a heterogeneous immunoassay, a bioassay, a reporter assay, a luciferase assay, a microarray, a surface plasmon resonance detector, a florescence resonance energy transfer, immunocytochemistry, or a cell mediated assay, or a cytokine proliferation assay.

95. The kit of any one of claims 87 to 94, wherein the kit includes reagents for determining a Human Leukocyte Antigen (HLA) profile of a subject, and selecting peptides that are presented by the HLA profile of the subject for detecting an immune response to B. pertussis.

96. A method of stimulating, inducing, promoting, increasing, or enhancing an immune response against a Bordetella in a subject, comprising:
administering a composition of claims 1 to 34, in an amount sufficient to stimulate, induce, promote, increase, or enhance an immune response against the Bordetella in the subject.

97. The method of claim 96, wherein the immune response provides the subject with protection against a Bordetella infection or pathology, or one or more physiological conditions, disorders, illnesses, diseases or symptoms caused by or associated with Bordetella infection or pathology.

98. The method of claim 96 or claim 97, wherein the immune response is specific to:
one or more B. pertussis peptides selected from the amino acid sequences set forth in any one of Tables 1-20, or a subsequence, portion, homologue, variant or derivative thereof.

99. A method of stimulating, inducing, promoting, increasing, or enhancing an immune response against B. pertussis in a subject, comprising:
administering a composition of claims to 19 to 34, in an amount sufficient to stimulate, induce, promote, increase, or enhance an immune response against B. pertussis in the subject.

100. The method of claim 99, wherein the immune response provides the subject with protection against a B. pertussis infection or pathology, or one or more physiological conditions, disorders, illnesses, diseases or symptoms caused by or associated with B. pertussis infection or pathology.

101. The method of claim 99 or claim 100, wherein the immune response is specific to:
one or more B. pertussis peptides selected from the amino acid sequences set forth in those sequences set forth in any one of Tables 1-20, or a subsequence, portion, homologue, variant or derivative thereof.

102. A method of stimulating, inducing, promoting, increasing, or enhancing an immune response against B. pertussis in a subject, comprising:
administering to a subject an amount of a protein or peptide or a polynucleotide that expresses the protein or peptide comprising, consisting of or consisting essentially of an amino acid sequence of the B. pertussis protein or peptide, or a variant, homologue, derivative or subsequence thereof, wherein the protein or peptide comprises at least two peptides selected from the amino acid sequences set forth in any one of Tables 1-20 or a subsequence, portion, homologue, variant or derivative thereof, in an amount sufficient to prevent, stimulate, induce, promote, increase, immunize against, or enhance an immune response against B. pertussis in the subject.

103. The method of claim 102, wherein the immune response provides the subject with protection against B. pertussis infection or pathology, or one or more physiological conditions, disorders, illnesses, diseases or symptoms caused by or associated with B. pertussis infection or pathology.

104. A method of treating, preventing, or immunizing a subject against B. pertussis infection, comprising administering to a subject an amount of a protein, peptide or a polynucleotide that expresses the protein or peptide comprising, consisting of, or consisting essentially of an amino acid sequence of a Bordetella protein or peptide, or a variant, homologue, derivative or subsequence thereof, wherein the protein or peptide comprises at least two amino acid sequences selected from any one of Tables 1-20 or a subsequence, portion, homologue, variant or derivative thereof, in an amount sufficient to treat, prevent, or immunize the subject for B. pertussis infection, wherein the protein or peptide comprises or consists of a Bordetella T cell epitope that elicits, stimulates, induces, promotes, increases, or enhances an anti-B. pertussis T cell immune response.

105. The method of claim 104, wherein the one or more amino acid sequences are selected from any one of those sequences set forth in Tables 1-20, or a subsequence, portion, homologue, variant or derivative thereof;
a fusion protein comprising one or more amino acid sequences selected from any one of those sequences set forth in Tables 1-20; or
a pool of 2 or more peptides selected from the amino acid sequences set forth in those sequences set forth in Tables 1-20.

106. The method of claim 104, wherein the anti-B. pertussis T cell response is a CD8+, a CD4+ T cell response, or both.

107. The method of any of claims 104 to 106, wherein the T cell epitope is conserved across two or more clinical isolates of B. pertussis or two or more circulating forms of B. pertussis.

108. The method of claim 107, wherein the B. pertussis infection is an acute infection.

109. The method of any one of claims 104 to 108, wherein the subject is a mammal or a human.

110. The method of any one of claims 104 to 109, wherein the method reduces B. pertussis bacterial titer, increases or stimulates B. pertussis bacterial clearance, reduces or inhibits B. pertussis bacterial proliferation, reduces or inhibits increases in B. pertussis bacterial titer or B. pertussis bacterial proliferation, reduces the amount of a B. pertussis bacterial protein or the amount of a B. pertussis bacterial nucleic acid, or reduces or inhibits synthesis of a B. pertussis bacterial protein or a B. pertussis bacterial nucleic acid.

111. The method of any one of claims 104 to 110, wherein the method reduces one or more adverse physiological conditions, disorders, illness, diseases, symptoms or complications caused by or associated with B. pertussis infection or pathology.

112. The method of any one of claims 104 to 111, wherein the method improves one or more adverse physiological conditions, disorders, illness, diseases, symptoms or complications caused by or associated with B. pertussis infection or pathology.

113. The method of claim 111 or 112, wherein the symptom is fever or chills, cough, shortness of breath or difficulty breathing, fatigue, muscle or body aches, headache, new loss of taste or smell, sore throat, congestion or runny nose, nausea or vomiting, or diarrhea.

114. The method of any one of claims 104 to 113, wherein the method reduces or inhibits susceptibility to B. pertussis infection or pathology.

115. The method of any one of claims 104 to 113, wherein the protein or peptide, or a subsequence, portion, homologue, variant or derivative thereof, is administered prior to, substantially contemporaneously with or following exposure to or infection of the subject with B. pertussis.

116. The method of any one of claims 104 to 115, wherein a plurality of B. pertussis T cell epitopes are administered prior to, substantially contemporaneously with or following exposure to or infection of the subject with B. pertussis.

117. The method of any one of claims 104 to 116, wherein the protein or peptide, or a subsequence, portion, homologue, variant or derivative thereof is administered within 2-72 hours, 2-48 hours, 4-24 hours, 4-18 hours, or 6-12 hours after a symptom of B. pertussis infection or exposure develops.

118. The method of any one of claims 104 to 117, wherein the protein or peptide, or a subsequence, portion, homologue, variant or derivative thereof is administered prior to exposure to or infection of the subject with B. pertussis.

119. The method of any one of claims 104 to 118, wherein the method further comprises administering a modulator of immune response prior to, substantially contemporaneously with or following the administration to the subject of an amount of a protein or peptide.

120. The method of claim 119, wherein the modulator of immune response is a modulator of the innate immune response.

121. The method of claim 119 or claim 120, wherein the modulator is IL-6, IFN-γ, TGF-β, or IL-10, or an agonist or antagonist thereof.

122. A method of treating, preventing, or immunizing a subject against B. pertussis infection, comprising administering to a subject the composition of any one of claims 1-36 in an amount sufficient to treat, prevent, or immunize the subject for B. pertussis infection.

123. The method of claim 122, wherein the B. pertussis infection is an acute infection.

124. The method of claim 127, wherein the method reduces B. pertussis bacterial titer, increases or stimulates B. pertussis bacterial clearance, reduces or inhibits B. pertussis bacterial proliferation, reduces or inhibits increases in B. pertussis bacterial titer or B. pertussis bacterial proliferation, reduces the amount of a B. pertussis bacterial protein or the amount of a B. pertussis bacterial nucleic acid, or reduces or inhibits synthesis of a B. pertussis bacterial protein or a B. pertussis bacterial nucleic acid.

125. The method of any one of claims 122 to 124, wherein the method reduces one or more adverse physiological conditions, disorders, illness, diseases, symptoms or complications caused by or associated with B. pertussis infection or pathology.

126. The method of any one of claims 122 to 125, wherein the method improves one or more adverse physiological conditions, disorders, illness, diseases, symptoms or complications caused by or associated with B. pertussis infection or pathology.

127. The method of claim 125 or claim 126, wherein the symptom is fever or chills, cough, shortness of breath or difficulty breathing, fatigue, muscle or body aches, headache, new loss of taste or smell, sore throat, congestion or runny nose, nausea, vomiting, or diarrhea.

128. The method of any one of claims 122 to 127, wherein the method reduces or inhibits susceptibility to B. pertussis infection or pathology.

129. The method of any one of claims 122 to 133, wherein the composition is administered prior to, substantially contemporaneously with or following exposure to or infection of the subject with B. pertussis.

130. The method of any one of claims 122 to 128, wherein the composition is administered prior to, substantially contemporaneously with or following exposure to or infection of the subject with B. pertussis.

131. The method of any one of claims 122 to 130, wherein the composition is administered within 2-72 hours, 2-48 hours, 4-24 hours, 4-18 hours, or 6-12 hours after a symptom of B. pertussis infection or exposure develops.

132. The method of any one of claims 122 to 130, wherein the composition is administered prior to exposure to or infection of the subject with B. pertussis.

133. A peptide or peptides that are immunoprevalent or immunodominant in a bacteria obtained by a method consisting of, or consisting essentially of:
obtaining an amino acid sequence of the bacteria;
determining one or more sets of overlapping peptides spanning one or more bacteria antigen using unbiased selection;
synthesizing one or more pools of bacteria peptides comprising the one or more sets of overlapping peptides;
combining the one or more pools of bacteria peptides with Class I major histocompatibility proteins (MHC), Class II MHC, or both Class I and Class II MHC to form peptide-MHC complexes;
contacting the peptide-MHC complexes with T cells from subjects exposed to the bacteria;
determining which pools triggered cytokine release by the T cells; and
deconvoluting from the pool of peptides that elicited cytokine release by the T cells, which peptide or peptides are immunoprevalent or immunodominant in the pool.

134. The peptide or peptides of claim 133, wherein the bacteria is a Bordetella.

135. The peptide or peptides of claim 134, wherein the Bordetella is B. pertussis.

136. The peptide or peptides of any one of claims 133 to 135, wherein the immunodominant peptides are selected from 1, 2 or more peptides selected from the amino acid sequences set forth in any one of Tables 1-20.

137. The peptide or peptides of any one of claims 133 to 136, wherein the immunodominant peptides are selected from 1, 2 or more peptides selected from the amino acid sequences set forth in those sequences set forth in any one of Tables 1-20.

138. A method of selecting an immunoprevalent or immunodominant peptide or protein of a bacteria comprising, consisting of, or consisting essentially of:
obtaining an amino acid sequence of the bacteria;
determining one or more sets of overlapping peptides spanning one or more bacteria antigen using unbiased selection;
synthesizing one or more pools of bacteria peptides comprising the one or more sets of overlapping peptides;
combining the one or more pools of bacteria peptides with Class I major histocompatibility proteins (MHC), Class II MHC, or both Class I and Class II MHC to form peptide-MHC complexes;
contacting the peptide-MHC complexes with T cells from subjects exposed to the bacteria;
determining which pools triggered cytokine release by the T cells; and
deconvoluting from the pool of peptides that elicited cytokine release by the T cells, which peptide or peptides are immunoprevalent or immunodominant in the pool.

139. The method of claim 138, wherein the bacteria is a Bordetella.

140. The method of claim 139, wherein the Bordetella is B. pertussis.

141. The method of any one of claim 138 to 140, wherein the immunodominant peptides are selected from 1, 2 or more peptides selected from the amino acid sequences set forth in any one of Tables 1-20.

142. The method of any one of claims 138 to 141, wherein the immunodominant peptides are selected from 1, 2 or more peptides selected from the amino acid sequences set forth in those sequences set forth in any one of Tables 1-20.

143. A polynucleotide that expresses one or more peptides or proteins, comprising, consisting of, or consisting essentially of an amino acid sequence selected from any one of those sequences set forth in Tables 1-20, or a subsequence, portion, homologue, variant or derivative thereof;
a fusion protein comprising one or more amino acid sequences selected from any one of those sequences set forth in Tables 1-20; or
a pool of 2 or more or more peptides comprising, consisting of, or consisting essentially of amino acid sequences selected from any one of those sequences set forth in Tables 1-20.

144. A vector that comprises the polynucleotide of claim 143.

145. The vector of claim 144, wherein the vector is a bacterial vector.

146. A host cell that comprises the vector of claim 144 or claim 145.

147. A polynucleotide that expresses:
one or more peptides or proteins comprising, consisting of, or consisting essentially of an amino acid sequence selected from any one of those sequences set forth in Tables 1-20, or a subsequence, portion, homologue, variant or derivative thereof;
a fusion protein comprising one or more amino acid sequences selected from any one of those sequences set forth in Tables 1-20; or
a pool of 2 or more peptides selected from any one of those sequences set forth in Tables 1-20.

148. A vector that comprises the polynucleotide of claim 147.

149. The vector of claim 148, wherein the vector is a bacterial vector.

150. A host cell that comprises the vector of claim 148 or claim 149.
